(19) United States
(12) Patent Application Publication
(10) Pub. No.: US 2022/0380805 A1
(43) Pub. Date: Dec. 1, 2022

(54) ARENAVIRUSES AS VECTORS

(71) Applicant: UNIVERSITÄT BASEL, Basel (CH)

(72) Inventors: Daniel PINSCHEWER, Binningen (CH); Weldi Bonilla PINSCHEWER, Binningen (CH)

(21) Appl. No.: 17/772,996

(22) PCT Filed: Nov. 6, 2020
(86) PCT No.: PCT/EP2020/081393

§ 371 Date: Apr. 28, 2022

Related U.S. Application Data
(60) Provisional application No. 62/932,214, filed on Nov. 7, 2019.

(51) Int. Cl. C12N 15/86; A61K 39/12

(52) U.S. Cl. CPC C12N 15/86; A61K 39/12; C12N 2760/10034; A61K 2039/51

(57) ABSTRACT

The present application relates to arenavirus particles containing a genome engineered such that an arenaviral open reading frame (“ORF”) is sequestered into two or more functional fragments and these fragments are expressed from two or more viral mRNA transcripts. The arenavirus particles described herein are genetically stable and provide high-level transgene expression. In certain embodiments, the arenavirus particles are tri-segmented. In particular, described herein is a nucleotide sequence comprising one or more ORFs comprising a nucleotide sequence encoding a functional fragment of arenavirus GP, NP, L or Z. Also described herein is an arenavirus particle containing a genome engineered such that an arenaviral ORF is sequestered into two or more functional fragments and these fragments are expressed from two or more viral mRNA transcripts. Also described herein is an arenavirus genomic or antigenomic segment engineered such that the transcription thereof results in one or more mRNA transcripts comprising a nucleotide sequence encoding a functional fragment of arenavirus GP, NP, L or Z. The arenavirus particles described herein may be suitable for vaccines and/or treatment of diseases and/or for the use in immunotherapies.

This application claims benefit of U.S. Provisional Patent Application No. 62/932,214, filed Nov. 7, 2019, the disclosure of which is incorporated by reference herein in its entirety.

### REFERENCE TO SEQUENCE LISTING SUBMITTED ELECTRONICALLY

This application incorporates by reference a Sequence Listing submitted with this application as text file entitled “Sequence_Listing_13194-050-228.TXT” created on Nov. 5, 2020 and having a size of 701,226 bytes.

### 1. INTRODUCTION

The present application relates to arenavirus particles engineered such that an arenaviral open reading frame (“ORF”) is separated over two or more mRNA transcripts. The arenavirus particles described herein are genetically stable and provide high-level transgene expression. In certain embodiments, the arenavirus particles are tri-segmented. In particular, described herein is a nucleotide sequence comprising one or more ORFs comprising a nucleotide sequence encoding a functional fragment of arenavirus GP, NP, L or Z. Also described herein is an arenavirus particle engineered such that an arenaviral ORF is separated over two or more mRNA transcripts. Also described herein is an arenavirus genomic or antigenomic segment engineered such that the transcription thereof results in one or more mRNA transcripts comprising a nucleotide sequence encoding a functional fragment of arenavirus GP, NP, L or Z. The arenavirus particles described herein may be suitable for vaccines and/or treatment of diseases and/or for the use in immunotherapies.

### 2. BACKGROUND

### 2.1 Replicating Viral Vector Systems

Replicating viral vector systems are optimally suited for delivering a target antigen of choice with the aim of inducing a potent immune response against it. In particular, viral vector systems are useful when aiming to elicit strong CD8+ T cell responses such as in tumor immunotherapy or with the intent of curing a persistent viral infection. To serve as a viral delivery system, wild-type viruses can be re-engineered to incorporate transgenic sequences, allowing them to induce immune responses against the respective translation production, i.e., the target of choice. Additionally, when based on viruses with a (known or insufficiently defined) disease-causing potential, re-engineering should confer the resulting vector with a defined degree of attenuation. On the other hand, immunogenicity of virally vectored delivery systems commonly depends on the vector dose administered. Accordingly, attenuation should not reduce viral growth in tissue culture systems to the point of rendering industrial production impractical. The usefulness of a replication-competent vector system for medical use therefore depends substantially on the following criteria:

1) good growth in cell culture, enabling the vector's production to high titers in industrial fermentation processes (production yields),

2) stable attenuation, i.e., the inability of the vector to revert to wild-type-like (more virulent) replication behavior in the vaccinated host (genetic stability),

3) high-level transgene expression to elicit strong immune responses against the desired target antigen(s) (transgene expression levels).

Arenaviruses such as lymphocytic choriomeningitis virus (LCMV) and Pichinde virus (PICV) have been re-engineered to serve as replicating yet attenuated delivery systems inducing immune responses against incorporated heterologous antigens (Cheng B Y, et al. (2015) J Virol 89, 7373-7384; Dhanwani R, et al. (2015) J Virol 90, 2551-2560; Emonet S F, et al. (2009) Proc Natl Acad Sci USA 106, 3473-3478; Kallert S M, et al. (2017) Nat Commun 8, 15327; Popkin D L, et al. (2011) J Virol 85, 7928-7932). The natural arenavirus genome consists of two RNA segments and contains open reading frames (ORFs) encoding for the glycoprotein and nucleoprotein, both encoded on the short (S) segment, as well as the polymerase L and matrix protein Z, which are encoded on the large (L) segment (FIG. 1A). In order to incorporate foreign transgenic sequences in addition to the four arenaviral ORFs, the S segment can be duplicated and the GP and NP ORFs of arenaviruses can be sequestered onto the resulting two S segments (S_{NP}, S_{GP}; FIGS. 1B-E; (Cheng B Y, et al. (2015) J Virol 89, 7373-7384; Emonet S F, et al. (2009) Proc Natl Acad Sci USA 106, 3473-3478; Kallert S M, et al. (2017) Nat Commun 8, 15327). The concept behind segregating NP and GP onto separate S segments is to force the virus to carry three genomic RNA segments in order to maintain its complete proteome. By consequence, there are four possibilities how to position NP, GP and two heterologous ORFs (designated as “h.p.” in FIGS. 1B-1G) on the two segments. These four possibilities here are referred to as r3LCMV, artLCMV, r3LCMVrev and artLCMVrev (FIGS. 1B-E). It has been demonstrated that r3LCMVrev and artLCMVrev grow to only very poor titers, much lower than r3LCMV or artLCMV (Cheng B Y, et al. (2015) J Virol 89, 7373-7384), and thus fail to meet the above criterion 1. Poor growth of r3LCMVrev and artLCMVrev supposedly results from NP being positioned next to the 5′ untranslated region (UTR), and thus being expressed under control of the respective (weaker) viral promoter sequences. Accordingly, the r3LCMVrev and artLCMVrev designs are not well suited for industrial production. Kallert et al. (Kallert S M, et al. (2017) Nat Commun 8, 15327) have further demonstrated that r3LCMV is genetically unstable: During infection of immunodeficient mice, r3LCMV underwent inter-segmental RNA recombination yielding a bi-segmented virus, which had reunited the NP and GP ORFs on one RNA segment. The resulting virus had lost (most of) its transgenes and no longer was attenuated (FIG. 1F), thus failed to meet the above criterion 2 owing to safety concerns. In summary, neither r3LCMV nor r3LCMVrev met all three criteria (production yields, genetic stability, transgene expression levels; FIG. 2). In contrast artLCMV was genetically stable even after prolonged periods of replication in immunodeficient animals. The underlying mechanistic explanation was that the NP and GP ORFs in artLCMV could hypothetically be recombined on one S segment (FIG. 1G), but such recombination could only occur at the expense of losing the 5′ UTR while duplicating the 3′ UTR on both ends of the resulting S segment. The 5′ UTR and the 3′ UTR are known to form a non-covalent panhandle structure by RNA base-pairing, which allows template recognition by the viral RNA-dependent RNA polymerase (RdRp) in both a sequence- and structure-dependent manner. Accordingly, biological activity requires a precise 5′ UTR and 3′ UTR, tolerating no deviation from the natural sequence (Perez and de la Torre (2003) J Virol 77, 1184-1194). Accordingly, a hypothetical recombination product as depicted in FIG. 1G could not serve as a template for recognition by the viral RdRp and would be biologically inactive. artLCMV not only is genetically stable but also grows well in cell culture (Cheng B Y, et al. (2015) J Virol 89, 7373-7384; Kallert S M, et al. (2017) Nat Commun 8, 15327; Popkin D L, et al. (2011) J Virol 85, 7928-7932). artLCMV therefore was the only tri-segmented arenavirus design amongst the four strategies outlined in FIGS. 1B-IE, which met the specifications 1 and 2 above, i.e. good production yields and genetic stability ensuring safety.

### 2.2 Glycoprotein and Precursors Thereof

The GP open reading frame encodes three functional protein subunits, which are expressed as one ribosomal translation product. During insertion of the nascending polypeptide into the endoplasmic reticulum (ER) the signal peptide is cleaved off from the subsequent GP1 domain by the signal peptidase (FIG. 3A). The remainder GP1-GP2 sequence is membrane-inserted and it is post-translationally cleaved into GP1 and GP2 subunits by the subtilisin kexin isozyme-1 (SKI-1)/site 1 protease (S1P, FIG. 3A) but the GP1 and GP2 domains remain non-covalently associated in the mature glycoprotein complex. Especially noteworthy is the essential nature of the arenavirus GP signal peptide (SP): Unlike most signal peptides which are approximately 20 amino acids in length and are rapidly degraded after signal peptidase cleavage, the arenavirus glycoprotein SPs typically are >50 amino acids long (Eichler R, et al. (2003) FEBS Lett 538, 203-206) and serve not only to insert the GP protein into the ER but they remain associated with the GP1/GP2 complex and are incorporated into mature virions. Furthermore, the arenavirus glycoprotein SP plays an essential role in the post-translational proteolytic cleavage of GP1 from GP2 by SKI-1/SIP. When the arenavirus SP was substituted for a foreign signal peptide in transient transfection experiments, the GP1/GP2 sequence was inserted into the ER, but it failed to undergo processing by SKI-1/SIP (Agnihothram, S S, et al. (2006) J Virol 80, 5189-5198; Eichler R, et al. (2003) EMBO Rep 4, 1084-1088; Schrempf S, et al. (2007) J Virol 81, 12515-12524). GP1/GP2 cleavage by SKI-1/SIP is, however, essential for rendering the glycoprotein complex biologically active (Pinschewer, D D, et al. (2003) Proc Natl Acad Sci USA 100, 7895-7900).

### 2.3 Engineered Arenavirus Particles and Arenavirus Genomic Segments

Recently, it has been shown that an infectious arenavirus particle can be engineered to contain a genome with the ability to amplify and express its genetic material in infected cells but unable to produce further progeny in normal, not genetically engineered cells (i.e., an infectious, replication-deficient arenavirus particle) (International Publication Nos.: WO 2009/083210 A1, WO 2014/140301 A1 and WO 2015/082570 A1). It has also been shown that a modified arenavirus genomic segment can be engineered to carry a viral ORF in a position other than the wild-type position of the ORF (International Publication No.: WO 2016/075250 A1 and Publication No.: US 2017/0319673 A1). It has further been shown that a modified Pichinde virus genomic segment can be engineered to carry a viral ORF in a position other than the wild-type position of the ORF (International Publication No.: WO 2017/198726 A1 and Publication No.: US 2019/0135875 A1).

There is an urgent need for compositions, such as the engineered arenavirus particles and related compositions, to be used in vaccines and immunotherapies of various diseases.

### 3. SUMMARY OF THE INVENTION

Provided herein are nucleotide sequences, arenavirus particles, arenavirus genomic or antigenomic segments, and related compositions. Also provided herein are methods of generating an arenavirus genomic or antigenomic RNA segment, methods of generating an arenavirus particle, and methods of rescuing an arenavirus particle from cDNA or RNA.

### 3.1 Nucleotide Sequences and Related Compositions and Methods

In one aspect, provided herein are nucleotide sequences. In certain embodiments, provided herein is a nucleotide sequence comprising a first open reading frame (ORF) and a second ORF, wherein one of the two ORFs is in sense orientation and the other ORF is in antisense orientation;

wherein the first ORF comprises a nucleotide sequence encoding a functional fragment of a first polypeptide, and the first ORF does not encode the full-length first polypeptide;
wherein the second ORF comprises a nucleotide sequence encoding:
a) a second polypeptide; or
b) a functional fragment of the first polypeptide, and the second ORF does not encode the full-length first polypeptide; or
c) a functional fragment of a second polypeptide, and the second ORF does not encode the full-length second polypeptide; or
d) a heterologous non-arenaviral polypeptide; and
   wherein the first and second polypeptides are different from each other and selected from the group consisting of arenavirus GP, NP, Z and L.

In certain embodiments, the first ORF and the second ORF are separated by an arenavirus intergenic region (IGR) and each ORF is under control of an arenavirus 3′ untranslated region (UTR) or an arenavirus 5′ UTR.

In certain embodiments, the first ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the second ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the first ORF is part of a transcription unit that further comprises another ORF, wherein the other ORF and the first ORF are separated by an internal ribosome entry site (IRES). In certain embodiments, the second ORF is part of a transcription unit that further comprises another ORF, wherein the other ORF and the second ORF are separated by an IRES. In certain embodiments, the first ORF and/or the second ORF comprises a nucleotide sequence encoding an arenavirus GP signal peptide or a functional fragment thereof and the other ORF comprises a nucleotide sequence encoding a heterologous non-arenaviral polypeptide or an arenavirus NP, Z, or L.

In certain embodiments, the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, provided herein is a nucleotide sequence comprising an open reading frame (ORF), wherein the ORF comprises a nucleotide sequence encoding
a) a functional fragment of a first polypeptide, and
b) a heterologous non-arenaviral polypeptide or a second polypeptide; wherein the ORF does not encode the full-length first polypeptide; and wherein the first and second polypeptides are different from each other and selected from the group consisting of arenavirus GP, NP, Z and L that are not from Lassa virus. In certain embodiments, the first and second polypeptides are selected from the group consisting of arenavirus GP, NP, Z and L of LCMV, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the ORF is a first ORF and the nucleotide sequence further comprises a second ORF. In certain embodiments, the second ORF comprises a nucleotide sequence encoding a third polypeptide, a functional fragment of the first polypeptide, a functional fragment of a third polypeptide, or a second heterologous non-arenaviral polypeptide; wherein the third polypeptide is different from the first polypeptide and the second polypeptide and selected from the group consisting of arenavirus GP, NP, Z and L; wherein one of the two ORFs is in sense orientation and the other ORF is in antisense orientation; wherein the second ORF does not encode the full-length first polypeptide; wherein the second ORF does not encode the full-length third polypeptide; and wherein the heterologous non-arenaviral polypeptide and the second heterologous non-arenaviral polypeptide are the same or different from each other. In certain embodiments, the second ORF comprises a nucleotide sequence encoding a functional fragment of a first polypeptide, and wherein the functional fragment encoded by the first ORF is different from the functional fragment encoded by the second ORF.

In certain embodiments, the nucleotide sequence does not further comprise a second ORF.

In certain embodiments, the nucleotide sequence is an arenavirus genomic or antigenomic S segment. In certain embodiments, the nucleotide sequence is an arenavirus genomic or antigenomic L segment.

In certain embodiments, the first ORF is under control of an arenavirus 3′ UTR, and the second ORF is under control of an arenavirus 5′ UTR. In certain embodiments, the first ORF is under control of an arenavirus 5′ UTR, and the second ORF is under control of an arenavirus 3′ UTR.

In certain embodiments, the first ORF comprises a nucleotide sequence encoding arenavirus GP signal peptide, either alone or fused to a heterologous non-arenaviral polypeptide. In certain embodiments, the second ORF comprises a nucleotide sequence encoding a heterologous non-arenaviral signal peptide and the arenavirus GP1 and GP2. In certain embodiments, the second ORF comprises a nucleotide sequence encoding a heterologous non-arenaviral polypeptide; wherein the heterologous non-arenaviral polypeptide encoded by the first ORF and the heterologous non-arenaviral polypeptide encoded by the second ORF are the same or different from each other. In certain embodiments, the first ORF is under control of an arenavirus 3′ UTR and the second ORF is under control of an arenavirus 5′ UTR.

In certain embodiments, the first ORF comprises a nucleotide sequence encoding a heterologous non-arenaviral signal peptide and the arenavirus GP1 and GP2. In certain embodiments, the second ORF comprises a nucleotide sequence encoding NP. In certain embodiments, the first ORF is under control of an arenavirus 5′ UTR and the second ORF is under control of an arenavirus 3′ UTR.

In certain embodiments, the first polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, Or SEQ ID NO:140; and the second polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140; and the third polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the first polypeptide comprises an amino acid sequence identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137.

In certain embodiments, the functional fragment encoded by the first ORF or the second ORF is an arenavirus GP signal peptide or a functional fragment thereof.

In certain embodiments, the heterologous non-arenaviral polypeptide, the second heterologous non-arenaviral polypeptide, or both heterologous non-arenaviral polypeptides are each an antigen derived from an infectious organism, tumor, or allergen. In certain embodiments, the antigen is selected from the group consisting of

(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-DI, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70\K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

In certain embodiments, the expression of the heterologous non-arenaviral polypeptide or the expression of the second heterologous non-arenaviral polypeptide is under control of an arenavirus 3′ UTR; and the expression level of the heterologous non-arenaviral polypeptide or the expression level of the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or the expression level of the same second heterologous non-arenaviral polypeptide expressed under control of the respective arenavirus 5′ UTR.

In certain embodiments, the expression of the heterologous non-arenaviral polypeptide is under control of an arenavirus 3′ UTR and the expression of the second heterologous non-arenaviral polypeptide is under control of an arenavirus 5′ UTR, wherein the proportion of cells expressing both the heterologous non-arenaviral polypeptide and the second heterologous non-arenaviral polypeptide, after an arenavirus particle containing a genome comprising the nucleotide sequence infects a population of cells, is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells expressing both the same heterologous non-arenaviral polypeptide and the same second heterologous non-arenaviral polypeptide expressed under control of arenavirus 5′ UTRs, after an arenavirus particle containing a genome comprising a nucleotide sequence encoding the same heterologous non-arenaviral polypeptide and the same second heterologous non-arenaviral polypeptide expressed under control of arenavirus 5′ UTRs infects a comparable population of cells.

In certain embodiments, provided herein is a translation product of the nucleotide sequence provided herein.

In certain embodiments, provided herein is an arenavirus particle containing a genome comprising the nucleotide sequence provided herein. In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the genome of the arenavirus particle consists of
a) an S segment that encodes the arenavirus GP signal peptide and a heterologous non-arenaviral polypeptide or the arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and wherein the two heterologous non-arenaviral polypeptides are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from a Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the genome of the arenavirus particle encodes the heterologous non-arenaviral polypeptide, and the growth or infectivity of the arenavirus particle is not inferior to a second arenavirus particle, wherein the genome of the second arenavirus particle encodes the same heterologous non-arenaviral polypeptide; and wherein all arenaviral GP, NP, Z and L in the second arenavirus particle are each expressed as one ORF.

In certain embodiments, the arenavirus particle is infectious and replication competent. In certain embodiments, the arenavirus particle is attenuated as compared to its parental virus. In certain embodiments, the arenavirus particle is infectious but unable to produce further infectious progeny in non-complementing cells.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to the subject as compared to after another arenavirus particle expressing the same heterologous non-arenaviral polypeptide under control of an arenavirus 5′ UTR is administered to the subject or to a comparable subject.

In certain embodiments, the nucleotide sequence is a DNA sequence, which can be transcribed into an arenavirus genomic or antigenomic segment. In certain embodiments, provided herein is a method of producing an arenavirus genomic or antigenomic RNA segment, wherein the method comprises transcribing the DNA sequence provided herein.

In certain embodiments, provided herein is a method of generating an arenavirus particle, wherein the method comprises:
a) transfecting into a host cell one or more DNA sequences provided herein or one or more RNA sequences each transcribed in vitro from the DNA sequence provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more DNA sequences are transcribed using a bidirectional promoter. In certain embodiments, the one or more DNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising the nucleotide sequence provided herein.

In certain embodiments, provided herein is a method of rescuing an arenavirus particle using the nucleotide sequence provided herein.

In certain embodiments, provided herein is a host cell comprising the nucleotide sequence provided herein, the translation product provided herein, the arenavirus particle provided herein, or the DNA expression vector provided herein.

In certain embodiments, provided herein is a vaccine comprising the nucleotide sequence provided herein, the translation product provided herein, the arenavirus particle provided herein, or the DNA expression vectorprovided herein, and a pharmaceutically acceptable carrier.

In certain embodiments, provided herein is a pharmaceutical composition comprising the nucleotide sequence provided herein, the translation product provided herein, the arenavirus particle provided herein, or the DNA expression vectorprovided herein, and a pharmaceutically acceptable carrier.

### 3.2 Arenavirus Particles and Related Compositions and Methods

In another aspect, provided herein are arenavirus particles. In certain embodiments, provided herein is an arenavirus particle engineered such that an arenaviral ORF is separated over two or more mRNA transcripts.

In certain embodiments, at least one of the mRNA transcripts comprises an internal ribosome entry site (IRES).

In certain embodiments, the mRNA transcripts can be transcribed from the arenavirus genomic or antigenomic segment. In certain embodiments, the arenavirus genomic or antigenomic segment is an S segment. In certain embodiments, the arenavirus genomic or antigenomic segment is an L segment.

In certain embodiments, the two or more mRNA transcripts are under control of an arenavirus 3′ UTR or an arenavirus 5′ UTR.

In certain embodiments, the arenaviral ORF encodes a polypeptide comprising an amino acid sequence that is at least 80%, 85%, 90%, 95%, 96%, 97%, 98%, 99% and 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the arenaviral ORF encodes arenavirus GP signal peptide, arenavirus GP1 and GP2 and the arenavirus GP signal peptide or a functional fragment thereof is expressed from a first mRNA transcript (e.g., viral mRNA transcript) and arenavirus GP1 and GP2 are expressed from a second mRNA transcript (e.g., viral mRNA transcript).

In certain embodiments, the first mRNA transcript is under control of an arenavirus 3′ UTR.

In certain embodiments, the second mRNA transcript further encodes a heterologous non-arenaviral signal peptide.

In certain embodiments, the heterologous non-arenaviral signal peptide is the signal peptide of the vesicular stomatitis virus serotype Indiana glycoprotein.

In certain embodiments, the first mRNA transcript further comprises a nucleotide sequence encoding a heterologous non-arenaviral polypeptide or arenavirus GP, NP, Z and L.

In certain embodiments, the heterologous non-arenaviral polypeptide is an antigen derived from an infectious organism, tumor, or allergen. In certain embodiments, the antigen is selected from the group consisting of

(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and (c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

In certain embodiments, the expression level of the heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR.

In certain embodiments, the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment, wherein the proportion of cells expressing both the two heterologous non-arenaviral polypeptides after the arenavirus particle infects a population of cells is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells expressing both the same two heterologous non-arenaviral polypeptides after an arenavirus particle expressing the same two heterologous non-arenaviral polypeptides under control of arenavirus 5′ UTRs infects a comparable population of cells, and wherein the expression of a first of the same two heterologous non-arenaviral polypeptides is under control of an arenavirus 5′ UTR in a first S segment and the expression of a second of the same two heterologous non-arenaviral polypeptides is under control of an arenavirus 5′ UTR in a second S segment.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the arenavirus particle comprises a genome organization as outlined in FIG. 4C. In certain embodiments, the arenavirus particle comprises a genome organization as outlined in FIG. 4E.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from a Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the arenavirus particle is infectious and replication competent. In certain embodiments, the arenavirus particle is attenuated as compared to its parental wild-type virus. In certain embodiments, the arenavirus particle is infectious but unable to produce further infectious progeny in non-complementing cells.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to the subject as compared to after another arenavirus particle expressing the same heterologous non-arenaviral polypeptide under control of an arenavirus 5′ UTR is administered to the subject or to a comparable subject.

In certain embodiments, provided herein is a translation product of the mRNA transcripts of the genome of the arenavirus particle provided herein.

In certain embodiments, provided herein is a cDNA of the mRNA transcript of the genome of the arenavirus particle provided herein, wherein the cDNA can be transcribed into an arenavirus genomic or antigenomic segment.

In certain embodiments, provided herein is a method of producing an arenavirus genomic or antigenomic segment, wherein the method comprises transcribing the cDNA provided herein.

In certain embodiments, provided herein is a method of generating an arenavirus particle, wherein the method comprises:
a) transfecting into a host cell one or more cDNA of the mRNA transcript of the genome of the arenavirus particle provided herein or one or more RNA sequences each transcribed in vitro from the cDNA of the mRNA transcript of the genome of the arenavirus particle provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more cDNA sequences are transcribed using a bidirectional promoter. In certain embodiments, the one or more cDNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising the DNA sequence encoding the mRNA transcript of the genome of the arenavirus particle provided herein.

In certain embodiments, provided herein is a method of rescuing an arenavirus particle using the mRNA transcript of the genome of the arenavirus particle provided herein or the cDNA sequence thereof.

In certain embodiments, provided herein is a host cell comprising the arenavirus particle provided herein, the translation product provided herein, the cDNA provided herein, or the DNA expression vector provided herein.

In certain embodiments, provided herein is a vaccine comprising the arenavirus particle provided herein, the translation product provided herein, the cDNA provided herein, or the DNA expression vector provided herein, and a pharmaceutically acceptable carrier.

In certain embodiments, provided herein is a pharmaceutical composition comprising the arenavirus particle provided herein, the translation product provided herein, the cDNA provided herein, or the DNA expression vector provided herein, and a pharmaceutically acceptable carrier.

### 3.3 Arenavirus Genomic or Antigenomic Segments and Related Compositions and Methods

In another aspect, provided herein are arenavirus genomic or antigenomic segments. In certain embodiments, provided herein is an arenavirus genomic or antigenomic segment engineered such that the viral transcription thereof results in a first mRNA transcript and a second mRNA transcript,

wherein the first mRNA transcript comprises a nucleotide sequence encoding a functional fragment of a first polypeptide, and the first mRNA transcript does not encode the full-length first polypeptide;
wherein the second mRNA transcript comprises a nucleotide sequence encoding:
a) a second polypeptide; or
b) a functional fragment of the first polypeptide, and the second mRNA transcript does not encode the full-length first polypeptide; or
c) a functional fragment of a second polypeptide, and the second mRNA transcript does not encode the full-length second polypeptide; or
d) a heterologous non-arenaviral polypeptide; and
   wherein the first and second polypeptides are different from each other and selected from the group consisting of arenavirus GP, NP, Z and L.

In certain embodiments, the first mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the second mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, provided herein is an arenavirus genomic or antigenomic segment engineered such that the viral transcription thereof results in an mRNA transcript encoding:
a) a functional fragment of a first polypeptide, and
b) a heterologous non-arenaviral polypeptide or a second polypeptide;
   wherein the mRNA transcript does not encode the full-length first polypeptide; and wherein the first and second polypeptides are different from each other and selected from the group consisting of arenavirus GP, NP, Z and L.

In certain embodiments, the mRNA transcript is a first mRNA transcript and the viral transcription of the arenavirus genomic or antigenomic segment further results in a second mRNA transcript; wherein the second mRNA transcript comprises a nucleotide sequence encoding a third polypeptide; a functional fragment of a third polypeptide; or a second heterologous non-arenaviral polypeptide; wherein the third polypeptide is different from the first and the second polypeptide and selected from the group consisting of arenavirus GP, NP, Z and L, and wherein the heterologous non-arenaviral polypeptide and the second heterologous non-arenaviral polypeptide are the same or different from each other.

In certain embodiments, the second mRNA transcript comprises a nucleotide sequence encoding a functional fragment of a first polypeptide, and wherein the functional fragment encoded by the first mRNA transcript is different from the functional fragment encoded by the second mRNA transcript.

In certain embodiments, the viral transcription of the arenavirus genomic or antigenomic segment does not further result in a second mRNA transcript.

In certain embodiments, the mRNA transcript comprises an internal ribosome entry site (IRES).

In certain embodiments, the arenavirus genomic or antigenomic segment is an S segment. In certain embodiments, the arenavirus genomic or antigenomic segment is an L segment.

In certain embodiments, the functional fragment is under control of an arenavirus 3′ UTR. In certain embodiments, the functional fragment is under control of an arenavirus 5′ UTR.

In certain embodiments, the first, second and third polypeptide each comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, or SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

In certain embodiments, the first polypeptide comprises an amino acid sequence identical to SEQ ID NO:1, SEQ ID NO:2, SEQ ID NO:3, or SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134.

In certain embodiments, the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137.

In certain embodiments, the functional fragment is an arenavirus GP signal peptide or a functional fragment thereof.

In certain embodiments, the heterologous non-arenaviral polypeptide, the second heterologous non-arenaviral polypeptide, or both heterologous non-arenaviral polypeptides are each an antigen derived from an infectious organism, tumor, or allergen. In certain embodiments, the antigen is selected from the group consisting of

(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

In certain embodiments, the first mRNA transcript comprises a nucleotide sequence encoding the arenavirus GP signal peptide, either alone or fused to a heterologous non-arenaviral polypeptide. In certain embodiments, the second mRNA transcript comprises a nucleotide sequence encoding a heterologous non-arenaviral signal peptide and the arenavirus GP1 and GP2. In certain embodiments, the first mRNA transcript is under control of an arenavirus 3′ UTR and the second mRNA transcript is under control of an arenavirus 5′ UTR.

In certain embodiments, the first mRNA transcript comprises a nucleotide sequence encoding the arenavirus GP signal peptide, either alone or fused to a heterologous non-arenaviral polypeptide. In certain embodiments, the second mRNA transcript comprises a nucleotide sequence encoding a heterologous non-arenaviral polypeptide; wherein the heterologous non-arenaviral polypeptide encoded by the first mRNA transcript and the heterologous non-arenaviral polypeptide encoded by the second mRNA transcript are the same or different from each other. In certain embodiments, the first mRNA transcript is under control of an arenavirus 3′ UTR and the second mRNA transcript is under control of an arenavirus 5′ UTR.

In certain embodiments, the first mRNA transcript comprises a nucleotide sequence encoding a heterologous non-arenaviral signal peptide and the arenavirus GP1 and GP2. In certain embodiments, the second mRNA transcript comprises a nucleotide sequence encoding NP. In certain embodiments, the first mRNA transcript is under control of an arenavirus 5′ UTR and the second mRNA transcript is under control of an arenavirus 3′ UTR.

In certain embodiments, the expression level of the heterologous non-arenaviral polypeptide expressed under control of an arenavirus 3′ UTR or the expression level of the second heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or higher than the expression level of the same second heterologous non-arenaviral polypeptide expressed under control of the respective arenavirus 5′ UTR.

In certain embodiments, the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment, wherein the proportion of cells expressing both the two heterologous non-arenaviral polypeptides after the arenavirus particle infects a population of cells is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells expressing both the same two heterologous non-arenaviral polypeptides after an arenavirus particle expressing the same two heterologous non-arenaviral polypeptides under control of arenavirus 5′ UTRs infects a comparable population of cells, and wherein the expression of a first of the same two heterologous non-arenaviral polypeptides is under control of an arenavirus 5′ UTR in a first S segment and the expression of a second of the same two heterologous non-arenaviral polypeptides is under control of an arenavirus 5′ UTR in a second S segment.

In certain embodiments, provided herein is a translation product of the arenavirus genomic or antigenomic segment provided herein.

In certain embodiments, provided herein is an arenavirus particle comprising the arenavirus genomic or antigenomic segment provided herein.

In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

In certain embodiments, the arenavirus particle is infectious and replication competent. In certain embodiments, the arenavirus particle is attenuated as compared to its parental wild-type virus. In certain embodiments, the arenavirus particle is infectious but unable to produce further infectious progeny in non-complementing cells.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to the subject as compared to after another arenavirus particle expressing the same heterologous non-arenaviral polypeptide under control of an arenavirus 5′ UTR is administered to the subject or to a comparable subject.

In certain embodiments, the arenavirus particle expresses two heterologous non-arenaviral polypeptides, wherein the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment, wherein the proportion of cells expressing both the two heterologous non-arenaviral polypeptides after the arenavirus particle infects a population of cells is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells expressing both the same two heterologous non-arenaviral polypeptides after an arenavirus particle expressing the same two heterologous non-arenaviral polypeptides under control of arenavirus 5′ UTRs infects a comparable population of cells, and wherein the expression of a first of the same two heterologous non-arenaviral polypeptides is under control of an arenavirus 5′ UTR in a first S segment and the expression of a second of the same two heterologous non-arenaviral polypeptides is under control of an arenavirus 5′ UTR in a second S segment.

In certain embodiments, provided herein is a cDNA of the arenavirus genomic or antigenomic segment provided herein.

In certain embodiments, provided herein is a method of producing an arenavirus genomic or antigenomic segment, wherein the method comprises transcribing the cDNA provided herein.

In certain embodiments, provided herein is a method of generating an arenavirus particle, wherein the method comprises:
a) transfecting into a host cell one or more cDNA sequences of the arenavirus genomic or antigenomic segment provided herein or one or more RNA sequences each transcribed in vitro from the cDNA sequence of the arenavirus genomic or antigenomic segment provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more cDNA sequences are transcribed using a bidirectional promoter. In certain embodiments, the one or more cDNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising a DNA sequence encoding the arenavirus genomic or antigenomic segment provided herein.

In certain embodiments, provided herein is a method of rescuing an arenavirus particle using the arenavirus genomic or antigenomic segment provided herein or a DNA sequence encoding the arenavirus genomic or antigenomic segment.

In certain embodiments, provided herein is a host cell comprising the arenavirus genomic or antigenomic segment provided herein, the translation product provided herein, the arenavirus particle provided herein, or the DNA expression vector provided herein.

In certain embodiments, provided herein is a vaccine comprising the arenavirus genomic or antigenomic segment provided herein, the translation product provided herein, the arenavirus particle provided herein, or the DNA expression vector provided herein, and a pharmaceutically acceptable carrier.

In certain embodiments, provided herein is a pharmaceutical composition comprising the arenavirus genomic or antigenomic segment provided herein, the translation product provided herein, the arenavirus particle provided herein, or the DNA expression vector provided herein, and a pharmaceutically acceptable carrier.

### 3.4 Definitions

As used herein and unless otherwise indicated, the term “GP” means both the arenavirus glycoprotein and any arenavirus glycoprotein precursor. In an exemplary embodiment, the arenavirus glycoprotein precursor can be post-translationally cleaved into a signal peptide, GP1 and GP2. In certain embodiments, the arenavirus glycoprotein or any arenavirus glycoprotein precursor may be wild-type. In other embodiments, the arenavirus glycoprotein or any arenavirus glycoprotein precursor may be recombinant. GP is further described in Section 5.1.

As used herein and unless otherwise indicated, the term “NP” means both the arenavirus nucleoprotein and any arenavirus nucleoprotein precursor. In certain embodiments, the arenavirus nucleoprotein or any arenavirus nucleoprotein precursor may be wild-type. In other embodiments, the arenavirus nucleoprotein or any arenavirus nucleoprotein precursor may be recombinant. NP is further described in Section 5.1.

As used herein and unless otherwise indicated, the term “Z” means both the arenavirus Z protein and any arenavirus Z protein precursor. In certain embodiments, the arenavirus Z protein or any arenavirus Z protein precursor may be wild-type. In other embodiments, the arenavirus Z protein or any arenavirus Z protein precursor may be recombinant. Z is further described in Section 5.1.

As used herein and unless otherwise indicated, the term “L” means both the arenavirus L protein and any arenavirus L protein precursor. In certain embodiments, the arenavirus L protein or any arenavirus L protein precursor may be wild-type. In other embodiments, the arenavirus L protein or any arenavirus L protein precursor may be recombinant. L is further described in Section 5.1.

As used herein and unless otherwise indicated, the term “functional fragment” means a fragment of a polypeptide. The functional fragment of a polypeptide as described herein is not the full-length polypeptide. In certain embodiments, the functional fragment may possess one or more functions that is known to a person of ordinary skills in the art. In certain embodiments, the functional fragment may possess the biological functions of the polypeptide from which the functional fragment is derived. In an exemplary embodiment, the functional fragment may be a signal peptide that possesses a function of mediating the insertion of glycoprotein precursor into the membrane of the endoplasmic reticulum (ER). In further embodiments, besides the function of mediating the insertion of glycoprotein precursor into the membrane of the ER, the signal peptide may possess other functions, for example mediating cleavage of the polypeptide and/or acting as a trans-acting maturation factor. In other embodiments, the functional fragment may not possess any function that is known to a person of ordinary skills in the art. Functional fragment is further described in Section 5.2.

As used herein and unless otherwise indicated, the term “heterologous non-arenaviral polypeptide” means a polypeptide that is not of arenavirus origin. In certain embodiments, the heterologous non-arenaviral polypeptide is a reporter protein (see Section 5.3.1). In other embodiments, the heterologous non-arenaviral polypeptide is a signal peptide (see Section 5.3.2). In other embodiments, the heterologous non-arenaviral polypeptide is an antigen (see Section 5.3.3).

### 3.5 Conventions and Abbreviations

| Abbreviation | Convention |
|---|---|
| art | Artificial |
| CAT | Chloramphenicol acetyltransferase |
| CMI | cell-mediated immunity |
| CD8 | Cluster of differentiation 8 |
| CD4 | Cluster of differentiation 4 |
| ER | Endoplasmic reticulum |
| GFP | Green fluorescent protein |
| GP | Glycoprotein and precursors thereof |
| GP1 | Glycoprotein-1 subunit |
| GP2 | Glycoprotein-2 subunit |
| h.p. | heterologous non-arenaviral polypeptide |
| IGR | Intergenic region |
| JUNV | Junin virus |
| LCMV | Lymphocytic choriomeningitis virus |
| L | L protein and precursors thereof |
| L protein | RNA-dependent RNA polymerase |
| L segment | Long segment |
| MHC | Major Histocompatibility Complex |
| Z | Matrix protein Z and precursors thereof |
| Z protein | Matrix protein Z |
| nat | Natural |
| NP | Nucleoprotein and precursors thereof |
| ORF | Open reading frame |
| PICV | Pichinde virus |
| RFP | Red fluorescent protein |
| r2JUNV | Recombinant bi-segmented JUNV |
| r3JUNV | Recombinant tri-segmented JUNV |
| r2LCMV | Recombinant bi-segmented LCMV |
| r3LCMV | Recombinant tri-segmented LCMV |
| S segment | Short segment |
| S1P | Site 1 protease |
| SKI-1 | Subtilisin kexin isozyme-1 |
| SP | Signal peptide |
| TOM | Tomato (red fluorescent protein) |
| UTR | Untranslated region |
| VSV | Vesicular Stomatitis Virus |
| wt | Wild-type |
| ALLV | Allpahuayo virus |
| AMAV | Amapari virus |
| MOBV | Mobala virus |
| MOPV | Mopeia virus |
| FLEV | Flexal virus |
| GTOV | Guanarito virus |
| LATV | Latino virus |
| MACV | Machupo virus |
| BCNV | Bear Canyon virus |
| PARV | Parana virus |
| PIRV | Pirital virus |
| SABV | Sabia virus |
| TAMV | Tamiami virus |
| TCRV | Tacaribe virus |
| WWAV | Whitewater Arroyo virus |
| IPPV | Ippy virus |
| ALXV | Alxa virus |
| BCNV | Bear Canyon virus |
| SBAV | Sabiá virus |
| CHAPV | Chapare vims |
| LIJV | Lìjiāng virus |
| CUPXV | Cupixi virus |
| GAIV | Gairo virus |
| LORV | Loei River virus |
| LUJV | Lujo virus |
| LUAV | Luna virus |
| LULV | Luli virus |
| LNKV | Lunk virus |
| MRLV | Mariental virus |
| MRWV | Merino Walk virus |
| MORV | Morogoro virus |
| OKAV | Okahandja virus |
| PRAV | Paraná´virus |
| APOV | Aporé virus |
| RYKV | Ryukyu virus |
| SOLV | Solwezi virus |
| SOUV | souris virus |
| TMMV | Tamiami virus |
| WENV | Wēnzhōu virus |
| BBRTV | Big Brushy Tank virus |
| CTNV | Catarina virus |
| SKTV | Skinner Tank virus |
| TTCV | Tonto Creek virus |
| XAPV | Xapuri virus |

### 4. BRIEF DESCRIPTION OF THE FIGURES

FIGS. 1A-1G: Published tri-segmented arenavirus vector genome design strategies, and molecular mechanism underlying r3LCMV phenotypic reversion and genetic stability of artLCMV. FIGS. 1A-IE show the schematic of the RNA segments forming the genomes of wild type LCMV (FIG. 1A), r3LCMV (FIG. 1B), artLCMV (FIG. 1C), r3LCMVrev (FIG. 1D) and artLCMVrev (FIG. 1E). Segment denominations are boxed and indicated in bold. UTR: untranslated region; IGR: intergenic region; ORFs are indicated by arrows. GPC: full-length glycoprotein ORF (including the natural signal peptide); NP: nucleoprotein; L: RNA-dependent RNA polymerase L; Z: Matrix protein; h.p.: heterologous non-arenaviral polypeptide. FIG. 1F shows a non-homologous inter-segmental RNA recombination event re-uniting NP and GPC on one RNA segment (Kallert S M, et al. (2017) Nat Commun 8, 15327). This process underlies the spontaneous reversion of r3LCMV to a bi-segmented viral genome with wildtype-like virulence. FIG. 1G shows a hypothetical recombination event in artLCMV reuniting GPC and NP on one single RNA segment, which is devoid of a 5′ UTR and thus lacks a functional viral promoter (Kallert S M, et al. (2017) Nat Commun 8, 15327).

FIG. 2: Characteristics of published tri-segmented arenavirus vector genome design strategies. Comparison of r3LCMV and r3LCMVrev in terms of cell culture growth, genetic stability and transgene expression levels from their S_{GP} segments. Superscript numbers refer to the published evidence for the rating. Genetic stability has not been tested for all vector design strategies (“untested”).

FIGS. 3A-3B: Domain structure of arenavirus glycoproteins, and strategy to segregate the signal peptide from the GP1 and GP2 domains into separate viral transcriptional units. FIG. 3A shows the schematic of the arenavirus glycoprotein (GPC), which is composed of a signal peptide (SP), the GP1 domain (outer globular receptor binding domain on the virion's glycoprotein complex) and the GP2 transmembrane stalk domain. Signal peptidase cleaves off the signal peptide concomitantly with polypeptide synthesis, whereas SKI-1/SIP (“S1P”) cuts post-translationally between GP1 and GP2, rendering the glycoprotein complex biologically active. FIG. 3B shows the segregation of the arenavirus GP signal peptide (SP) into one transcriptional unit (with the possibility of adding downstream in-frame with SP a heterologous non-arenaviral polypeptide (h.p.) of interest, which will be cleaved off from SP by signal peptidase concomitantly with translation by the ribosome), and GP1/GP2 into another separate transcriptional unit, where GP1/GP2 are expressed with an upstream heterologous non-arenaviral signal peptide for ER insertion.

FIGS. 4A-4F: Genome organization of Split-A, Split-B, Split-C, Split-D, Split-E and Split-F vector genomes and their X and Y variants. FIGS. 4A-4F shows the schematic description of the genome organizations of Split-A (FIG. 4A), Split-B (FIG. 4B), Split-C (FIG. 4C), Split-D (FIG. 4D), Split-E (FIG. 4E) and Split-F (FIG. 4F) vector genomes, each expressing two heterologous non-arenaviral polypeptides (“h.p.”), which may be the same or different from each other. X and Y variants (Split-Ax, Split-Ay, Split-Bx, Split-By etc.) carrying a transgene at only one of two possible sites are described for each one of the genome organizations displayed in FIGS. 4A-4F.

FIG. 5: DNA plasmid expression cassettes were used to generate by transient transfection infectious LCMV-based and Junin Candid #1-based Split-A, Split-C, Split-E, Split-F vectors, respectively, as well as artLCMV and artCAND vectors. The arenavirus RNA genome segments were transcribed from polymerase-I-driven expression cassettes (Pol-I-L, Pol-I-S1, Pol-I-S2) consisting of an upstream polymerase-I promoter (pol-I-P) and a downstream polymerase-I terminator (pol-I-T) flanking a cDNA of the viral segment of interest including its 5′ non-templated G (“G”). The viral segments contain elements as outlined in the legend to FIGS. 1A-1G. The minimal viral trans-acting factors NP and L were expressed from the plasmids denominated pC-NP and pC-L, which comprise an actin promoter (Act-P.) with CMV-enhancer (CMV-Enh.) with an artificial downstream intron, followed by a cDNA of the viral NP or L protein, respectively, and a polyadenylation site (poly-A).

FIGS. 6A-6E: Comparison of the cell culture growth of artLCMV and LCMV-based Split-A, Split-C, Split-E and Split-F vectors. LCMV-GP expressing BHK-21 cells (Flatz L, et al. Nat Med 2010; 16, 339-345) were transfected with plasmids as described in FIG. 5 to generate vectors containing genomes as depicted in FIGS. 6A-6B. Replication-competent vector titers on 3T3 cells were determined 6 days after transfection by immunofocus assay. Normal BHK-21 cells (not expressing LCMV-GP) were infected at MOI=0.01 with viral vectors and supernatants were collected after 48 hours (FIG. 6C). Infectious titers were determined on 3T3 cells by immunofocus assay based on the result from 1 (FIG. 6B and FIG. 6C) or the mean of two (FIG. 6A) cell culture wells. 293F suspension cells (not expressing LCMV-GP) were infected at MOI=0.001 with viral vectors, supernatants were collected at the indicated time points (e.g., 24 hrs, 48 hrs, 72 hrs, 96 hrs, or 120 hrs), and infectious titers were determined in the supernatant by immunofocus assay on 3T3 cells (FIG. 6D). The supernatants collected at 48h (FIG. 6D) were used to determine LCMV NP-expressing infectious titers by immunofocus assay. GP-pseudotyping 293T-GP cells or 3T3 cells were used as cell substrates (the values from the 3T3 cells are depicted in FIG. 6D). The ratio of 293T-GP:3T3 titer was calculated for each vector (artLCMV, Split-C and Split-E) as a surrogate of the proportion of particles packaging all three genomic segments (FIG. 6E). Black circles represent independent values from triplicate cell culture wells, bars show means+/−standard deviation.

FIG. 7: Comparison of GFP expression from Split-C(TOM/GFP) and artLCMV(TOM/GFP) vectors by fluorescence microscopy. BHK-21 cells were infected with Split-C(TOM/GFP) or artLCMV (TOM/GFP). Alternatively, some BHK-21 cells were not infected (“no virus”). 24 hours after infection, green fluorescence was visualized by fluorescence microscopy (FIG. 7).

FIGS. 8A-8E: Comparison of GFP and TOM expression from Split-C(TOM/GFP) and artLCMV(TOM/GFP) vectors by flow cytometry. BHK-21 cells were infected with Split-C(TOM/GFP) or artLCMV(TOM/GFP) as schematically depicted in FIG. 8A or left uninfected (“no virus”; same experiment as in FIG. 7). 32 hours after infection, the cells were analyzed using flow cytometric analysis of GFP and TOM expression. A distinct population of cells infected with Split-C(TOM/GFP), artLCMV(TOM/GFP), and GFP-expressing cells were gated for further analysis (FIG. 8B). Non-fluorescent un-infected cells were gated for comparison (FIG. 8B). Graph showing GFP and TOM fluorescence of cells as gated in FIG. 8B is shown in FIG. 8C. The geometric mean fluorescence intensity of the three cell populations in the GFP and TOM channels, respectively, is indicated. FIGS. 8D-8E show a comparison of GFP and TOM co-expression from Split-C(TOM/GFP), artLCMV(TOM/GFP) and artLCMV(GFP/TOM) vectors by flow cytometry. BHK-21 cells were infected with Split-C(TOM/GFP), artLCMV(TOM/GFP) or artLCMV(GFP/TOM), the genomes of which are schematically depicted in FIG. 8D, at a multiplicity of infection of 0.1 (MOI=0.1). 24 hours after infection, cells were processed for flow cytometric analysis of GFP and TOM expression (FIG. 8E). The percentage of cells exhibiting GFP and/or TOM fluorescence is indicated in the respective quadrants (FIG. 8E).

FIGS. 9A-9C: Genetic stability of LCMV-based Split-C vector but not of r3LCMV in persistently infected AGRAG mice. AGRAG mice were infected intravenously with 10e5 PFU of either an LCMV-based Split-C vector (expressing GFP and TOM, same vector as in FIG. 7 and FIGS. 8A-8E) or with r3LCMV(GFP) as described in Kallert et al. (Kallert S M, et al. (2017) Nat Commun 8, 15327), or with a bi-segmented wildtype LCMV (genome organization depicted in FIG. 1A) based on the Clone 13 backbone and expressing the WE strain glycoprotein. On the indicated time points, blood was collected to determine viral infectivity by detecting either the viral NP (as a measure of total infectivity, FIG. 9A) or the GFP transgene (as a measure of transgene-expressing infectivity, FIG. 9B). The ratio of NP:GFP infectivity was then calculated (FIG. 9C). FIGS. 9D-9F: Genetic stability of LCMV-based Split-C and artLCMV vectors but not of r3LCMV in persistently infected AGRAG mice. AGRAG mice were infected intravenously with 10e5 PFU of either i) an LCMV-based Split-C vector expressing GFP (Split-C(GFP)), ii) artLCMV(GFP), iii) r3LCMV(GFP) (as described in Kallert et al. (Kallert S M, et al. (2017) Nat Commun 8, 15327), or iv) with a bi-segmented wildtype LCMV (genome organization depicted in FIG. 1A based on the Clone 13 backbone and expressing the WE strain glycoprotein. On the indicated time points, blood was collected to determine viral infectivity by detecting either the viral NP (as a measure of total infectivity, FIG. 9D) or the GFP transgene (as a measure of transgene-expressing infectivity, FIG. 9E). The ratio of NP:GFP infectivity was then calculated (FIG. 9F). Symbols represent the mean+/−SEM of initially three (LCMVwt), five (r3LCMV(GFP)), four (artLCMV(GFP)), and six (Split-C) animals.

FIGS. 10A-10D: Immunogenicity of a cancer-testis antigen expressed from an LCMV-based Split-C vector's S1 segment in comparison to the S_{GP} segment of artLCMV. BALB/c mice were immunized with 5×10e5 PFU of either an LCMV-based Split-C vector expressing GFP and the cancer-testis antigen P1A or with an artLCMV vector delivering the same transgenes (i.e., cancer-testis antigen P1A). The respective vector genomes are depicted in FIG. 10A. Nine days after immunization, CD8+ T cell responses were determined in peripheral blood using MHC class I tetramers (H-2Ld) loaded with the LPYLGWLVF (SEQ ID NO: 43) peptide epitope derived from P1A (FIG. 10B) or with the immunodominant NP-derived backbone epitope RPQASGVYM (SEQ ID NO: 44) (NP118, FIG. 10C). The ratio of P1A:NP118 specific CD8+ T cells was calculated for each mouse as a measure of transgene immunodominance (FIG. 10D). Statistical significance was determined using unpaired two-tailed student's t tests. **: p<0.01, n.s.: p>0.05.

FIGS. 11A-11N: Immunogenicity of artificial HPV16 E7E6 fusion antigen expressed from an LCMV-based Split-C vector's S1 segment in comparison to the S_{GP} segment of artLCMV. C57BL/6 mice were immunized with 10e5 PFU of either an LCMV-based Split-C vector expressing GFP and an artificial HPV16 E7E6 fusion antigen or with an artLCMV vector delivering the same transgenes (i.e., GFP and artificial HPV16 E7E6 fusion antigen). The respective vector genomes are depicted in FIG. 11A. Control mice were left unimmunized (“none”). Eight days after immunization, CD8+ T cell responses were determined in peripheral blood and 22 days after immunization, CD8+ T cell responses were determined in the spleen. This experiment used MHC class I dextramers (H-2db) loaded with the RAHYNIVTF (SEQ ID NO: 45) peptide epitope derived from the HPV16 E7 protein (E7-Dex, FIGS. 11B, 11E, 11H, and 11J-11N) or with the immunodominant NP-derived backbone epitope FQPQNGQFI (SEQ ID NO: 46) (NP396-Tet, FIGS. 11C, 11F, and 11I). The ratio of P1A:NP118 specific CD8+ T cells was calculated as a measure of transgene immunodominance (FIG. 11D and FIG. 11G). E7-Dex+ and NP396-Tet+ cell frequencies are expressed as a percentage of CD8+B220− cells (FIGS. 11B, 11C, 11E, and 11F). Absolute numbers of E7-Dex+ or NP396−Tet+CD8+B220− cells per spleen are shown in FIG. 11H and FIG. 11I). Subsets of E7-Dex+CD8+B220− cells expressing the indicated markers are enumerated in FIGS. 11J-11N). Statistical significance was determined using unpaired two-tailed student's t tests. **: p<0.01, *: p<0.05, n.s.: p>0.05.

FIGS. 12A-12C: Assessment of cell culture growth of Split-C-CAND vectors. LCMV-GP expressing BHK-21 cells (Flatz L, et al. Nat Med 2010; 16, 339-345) were transfected with plasmids as described in FIG. 5 to generate Split-C-CAND(TOM/GFP) and Split-C-CAND(TOM/E7E6) vectors containing genomes as depicted in FIGS. 12A-12B. Five days after transfection, supernatants were blindly passaged on normal 293T cells (not expressing LCMV-GP). Culture supernatants were collected 2, 7 and 9 days after inoculation, and replication-competent vector titers were determined on 3T3 cells (FIG. 12C).

FIGS. 13A-13D: Comparison of GFP and TOM expression from Split-C-CAND(TOM/GFP) and artCAND(TOM/GFP) vectors by flow cytometry. 293T cells were infected with Split-C-CAND(TOM/GFP) or artCAND(TOM/GFP) as schematically depicted in FIG. 13A or left uninfected (“no virus”). 72 hours after infection, the cells were analyzed for GFP and TOM expression using flow cytometry (FIGS. 13B-13D). A distinct population of cells expressing GFP (FIG. 13C) or TOM (FIG. 13D) was detected in Split-C-CAND(TOM/GFP)- and artLCAND(TOM/GFP)-infected but not in uninfected control cell cultures, when plotting GFP and TOM against sideward scatter (SSC-H; FIGS. 13C-13D). The GFP mean fluorescence intensity (MFI; FIG. 13C) and TOM MFI (FIG. 13D) of virus-infected cultures are indicated.

FIGS. 14A-14D: Immunogenicity of an artificial HPV16 E7E6 fusion antigen expressed from a CAND-based Split-C vector's S1 segment in comparison to the S_{GP} segment of artCAND. C57BL/6 mice were immunized with 2×10e5 FFU of either an CAND-based Split-C vector (Split-C-CAND(TOM/E7E6)) expressing TOM and an artificial fusion antigen consisting of the E7 and E6 proteins of HPV16 (Cassetti et al. Vaccine 2004; 3-4, 520-527) or with an artCAND vector (artCAND(TOM/E7E6)) delivering the same transgenes (i.e., TOM and E7E6). The respective vector genomes are depicted in FIG. 14A. Nine days after immunization, CD8+ T cell responses were determined in peripheral blood using MHC class I dextramers (H-2db) loaded with the RAHYNIVTF (SEQ ID NO: 45) peptide epitope derived from the HPV16 E7 protein (E7-Dex, FIG. 14B) and MHC class I tetramers (H-2Kb) loaded with the NP-derived backbone epitope YTVKYPNL (SEQ ID NO: 47) (NP205, FIG. 14C). The ratio of E7:NP205 specific CD8+ T cells was calculated for each mouse as a measure of transgene immunodominance (FIG. 14D). Statistical significance was determined using unpaired two-tailed student's t tests. **: p<0.01, n.s.: p>0.05.

### 5. DETAILED DESCRIPTION OF THE INVENTION

Provided herein are arenavirus particles that are genetically stable and provide a high-level transgene expression. In certain embodiments, the arenavirus particles are tri-segmented. Also provided herein are nucleotide sequences and arenavirus genomic or antigenomic segments related to such arenavirus particles. In certain embodiments, provided herein is a nucleotide sequence (see Section 5.4) comprising one or more ORFs comprising a nucleotide sequence encoding a functional fragment (see Section 5.2) of arenavirus GP, NP, L or Z (see Section 5.1) and/or a heterologous non-arenaviral polypeptide (see Section 5.3). In certain embodiments, provided herein is an arenavirus particle engineered such that an arenaviral ORF is separated over two or more mRNA transcripts (see Section 5.5). In certain embodiments, provided herein is an arenavirus genomic or antigenomic segment engineered such that the transcription thereof results in one or more mRNA transcripts comprising a nucleotide sequence encoding a functional fragment of arenavirus GP, NP, L or Z (see Section 5.6). In certain embodiments, the arenavirus particles provided herein are genetically stable, i.e. do not revert to wild-type-like (more virulent) replication behavior in the host (genetic stability) (see Section 5.13). In certain embodiments, the arenavirus particles provided herein may show high-level transgene expression to elicit strong immune responses against the desired target antigen(s) (transgene expression levels) (see Section 5.13). In certain embodiments, the arenavirus particles provided herein may show good growth in cell culture, enabling the arenavirus particle's production to high titers in industrial fermentation processes (production yields) (see Section 5.13). In certain embodiments, the arenavirus particles provided herein may be suitable for vaccines, treatment of diseases, and/or for the use in immunotherapies (see Sections 5.11 and 5.12).

Arenaviruses for use with the methods and compositions provided herein can be Old World viruses such as, for example, Lassa virus, Lymphocytic choriomeningitis virus (LCMV), Mobala virus, Mopeia virus, or Ippy virus, or New World viruses such as, for example, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, Allpahuayo virus (ALLV), or Whitewater Arroyo virus. Arenaviruses for use with the methods and compositions provided herein can be, for example, arenaviruses, mammarenaviruses, Old World mammarenaviruses, New World mammarenaviruses, New World mammarenaviruses of Clade A, New World mammarenaviruses of Clade B, New World mammarenaviruses of Clade C, or New World mammarenaviruses of Clade D. Arenaviruses for use with the methods and compositions provided herein can be a mammarenavirus including, but not limited to, Allpahuayo virus, Alxa virus, Junin virus, Bear Canyon virus, Sabia virus, Pichinde virus, Chapare virus, Lijiang virus, Cupixi virus, Flexal virus, Gairo virus, Guanarito virus, Ippy virus, Lassa virus, Latino virus, Loei River virus, Lujo virus, Luna virus, Luli virus, Lunk virus, lymphocytic choriomeningitis virus, Machupo virus, Mariental virus, Merino Walk virus, Mobala virus, Mopeia virus, Morogoro virus, Okahandja virus, Oliveros virus, Parana virus, Pirital virus, Aporé virus, Ryukyu virus, Amapari virus, Solwezi virus, souris virus, Tacaribe virus, Tamiami virus, Wenzhou virus, Whitewater Arroyo virus, Big Brushy Tank virus, Catarina virus, Skinner Tank virus, Tonto Creek virus, or Xapuri virus.

5.1 Polypeptide Selected from the Group Consisting of Arenavirus GP, NP, Z and L

In certain embodiments, provided herein is a nucleotide sequence comprising one or more ORFs comprising a nucleotide sequence encoding a functional fragment of a polypeptide. In certain embodiments, provided herein is an arenavirus genomic or antigenomic segment engineered such that transcription thereof results in one or more mRNA transcripts comprising a nucleotide sequence encoding a functional fragment of a polypeptide. In certain embodiments, the polypeptide as described herein is selected from the group consisting of arenavirus GP, NP, Z, and L, namely from the group consisting of arenavirus glycoprotein, nucleoprotein, Z protein, L protein, glycoprotein precursor, nucleoprotein precursor, Z protein precursor, and L protein precursor. In certain embodiments, the arenavirus GP, NP, Z, and L are wild-type. In other embodiments, the arenavirus GP, NP, Z, and L are recombinant. In certain embodiments, the arenavirus GP, NP, Z, and L are mutated. In certain embodiments, the arenavirus GP, NP, Z, and L are derived from an attenuated virus.

In certain embodiments, the polypeptide described herein is selected from the group consisting of wild-type arenavirus glycoprotein, nucleoprotein, Z protein, L protein, glycoprotein precursor, nucleoprotein precursor, Z protein precursor, and L protein precursor. In certain embodiments, the polypeptide described herein is a wild-type arenavirus glycoprotein precursor. In certain embodiments, the wild-type arenavirus glycoprotein precursor can be processed into a GP signal peptide, a GP1 and a GP2.

In certain embodiments, the polypeptide described herein is selected from the group consisting of recombinant arenavirus glycoprotein, nucleoprotein, Z protein, L protein, glycoprotein precursor, nucleoprotein precursor, Z protein precursor, and L protein precursor. In certain embodiments, the polypeptide described herein is a recombinant arenavirus glycoprotein precursor. In certain embodiments, the recombinant arenavirus glycoprotein precursor can be processed into a GP signal peptide, a GP1 and a GP2, one or more of which are recombinant.

In certain embodiments, the polypeptide described herein is selected from the group consisting of mutated arenavirus glycoprotein, nucleoprotein, Z protein, L protein, glycoprotein precursor, nucleoprotein precursor, Z protein precursor, and L protein precursor. In certain embodiments, the polypeptide described herein is a mutated arenavirus glycoprotein precursor. In certain embodiments, the mutated arenavirus glycoprotein precursor can be processed into a GP signal peptide, a GP1 and a GP2, one or more of which are mutated.

In certain embodiments, the polypeptide described herein is selected from the group consisting of arenavirus glycoprotein, nucleoprotein, Z protein, L protein, glycoprotein precursor, nucleoprotein precursor, Z protein precursor, and L protein precursor that are derived from an attenuated virus. In certain embodiments, the polypeptide described herein is an arenavirus glycoprotein precursor derived from an attenuated virus. In certain embodiments, the arenavirus glycoprotein precursor can be processed into a GP signal peptide, a GP1 and a GP2, one or more of which are derived from an attenuated virus.

In certain embodiments, the polypeptide described herein is selected from the group consisting of GP, NP, Z and L of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the polypeptide described herein comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

In certain embodiments, the polypeptide as described herein is arenavirus GP, namely arenavirus glycoprotein or any glycoprotein precursor. In certain embodiments, the polypeptide described herein is a wild-type arenavirus glycoprotein or any wild-type glycoprotein precursor. In other embodiments, the polypeptide described herein is a recombinant arenavirus glycoprotein or any recombinant glycoprotein precursor. In certain embodiments, the arenavirus glycoprotein precursor can be processed into a GP signal peptide, a GP1 and a GP2. In certain embodiments, the arenavirus GP is arenavirus glycoprotein or any glycoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus GP as described herein comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the GP as described herein comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the GP as described herein comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134.

In certain embodiments, the polypeptide as described herein is arenavirus NP, namely arenavirus nucleoprotein or any nucleoprotein precursor. In certain embodiments, the polypeptide described herein is a wild-type arenavirus nucleoprotein or any wild-type nucleoprotein precursor. In other embodiments, the polypeptide described herein is a recombinant arenavirus nucleoprotein or any recombinant nucleoprotein precursor. In certain embodiments, the arenavirus NP is arenavirus nucleoprotein or any nucleoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus NP as described herein comprises an amino acid sequence that is at least 80%, 85%, 90%, 95%, 96%, 97%, 98%, 99% or 100% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 80% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 85% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 90% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 95% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 99% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 100% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 80% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 85% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 90% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 95% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 99% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138.

In certain embodiments, the polypeptide as described herein is arenavirus Z, namely arenavirus Z protein or any Z protein precursor. In certain embodiments, the polypeptide described herein is a wild-type arenavirus Z protein or any wild-type Z protein precursor. In other embodiments, the polypeptide described herein is a recombinant arenavirus Z protein or any recombinant Z protein precursor. In certain embodiments, the arenavirus Z is arenavirus Z protein or any Z protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus Z as described herein comprises an amino acid sequence that is at least 80%, 85%, 90%, 95%, 96%, 97%, 98%, 99% or 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139.

In certain embodiments, the polypeptide as described herein is arenavirus L, namely arenavirus L protein or any L protein precursor. In certain embodiments, the polypeptide described herein is a wild-type arenavirus L protein or any wild-type L protein precursor. In other embodiments, the polypeptide described herein is a recombinant arenavirus L protein or any recombinant L protein precursor. In certain embodiments, the arenavirus L is arenavirus L protein or any L protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus L as described herein comprises an amino acid sequence that is at least 80%, 85%, 90%, 95%, 96%, 97%, 98%, 99% or 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

### 5.2 Functional Fragment Provided Herein

The functional fragment to be used in the compositions and methods described herein is a fragment of a polypeptide. The functional fragment of a polypeptide as described herein is not the full-length polypeptide.

In certain embodiments, the functional fragment may possess one or more functions that is known to a person of ordinary skills in the art. In certain embodiments, the functional fragment may possess the biological functions of the polypeptide from which the functional fragment is derived, for example the biological functions required for growth of the arenavirus particle. In certain embodiments, the functional fragment can support the biological functions of the polypeptide from which it is derived as a separated fragment independent of the context of the full-length polypeptide. In an exemplary embodiment, the functional fragment may be a signal peptide that possesses a function of mediating the insertion of glycoprotein precursor into the membrane of the endoplasmic reticulum (ER). In further embodiments, besides mediating the insertion of glycoprotein precursor into the membrane of the ER, the signal peptide may possess other functions, for example mediating cleavage of the polypeptide and/or acting as a trans-acting maturation factor. In other embodiments, the functional fragment may not possess any function that is known to a person of ordinary skills in the art.

In certain embodiments, the functional fragment may be 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, or 300 amino acids in length. In certain embodiments, the functional fragment is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In certain embodiments, the functional fragment does not exceed 2500 amino acids in length.

In certain embodiments, the functional fragment may consist of 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, or 300 amino-acid residues. In certain embodiments, the functional fragment may consist of 58 amino-acid residues. In certain embodiments, the functional fragment may consist of 193 amino-acid residues. In certain embodiments, the functional fragment may consist of 200 amino-acid residues. In certain embodiments, the functional fragment may consist of 201 amino-acid residues. In certain embodiments, the functional fragment may consist of 207 amino-acid residues. In certain embodiments, the functional fragment may consist of 215 amino-acid residues. In certain embodiments, the functional fragment may consist of 225 amino-acid residues. In certain embodiments, the functional fragment may consist of 232 amino-acid residues. In certain embodiments, the functional fragment may consist of 233 amino-acid residues. In certain embodiments, the functional fragment may consist of 234 amino-acid residues. In certain embodiments, the functional fragment may consist of 235 amino-acid residues. In certain embodiments, the functional fragment may consist of 15 amino-acid residues. In certain embodiments, the functional fragment may consist of 20 amino-acid residues. In certain embodiments, the functional fragment may consist of 1 to 15 amino-acid residues. In certain embodiments, the functional fragment may consist of 2 to 20 amino-acid residues.

In certain embodiments, the functional fragment is a fragment of arenavirus GP, NP, Z, or L, namely a fragment of arenavirus glycoprotein, nucleoprotein, Z protein, L protein, glycoprotein precursor, nucleoprotein precursor, Z protein precursor, or L protein precursor. In certain embodiments, the functional fragment of arenavirus GP, NP, Z, or L is wild-type. In other embodiments, the functional fragment of arenavirus GP, NP, Z, and L is recombinant. In certain embodiments, the functional fragment of arenavirus GP, NP, Z, or L is mutated. In certain embodiments, the functional fragment of arenavirus GP, NP, Z, or L is derived from an attenuated virus.

In certain embodiments, the functional fragment is a fragment of wild-type arenavirus glycoprotein, nucleoprotein, Z protein, or L protein. In certain embodiments, the functional fragment is a fragment of wild-type arenavirus glycoprotein. In certain embodiments, the functional fragment is a fragment of wild-type arenavirus nucleoprotein. In certain embodiments, the functional fragment is a fragment of wild-type arenavirus Z protein. In certain embodiments, the functional fragment is a fragment of wild-type arenavirus L protein.

In certain embodiments, the functional fragment is a fragment of wild-type arenavirus glycoprotein precursor, nucleoprotein precursor, Z protein precursor, or L protein precursor. In certain embodiments, the functional fragment is a fragment of wild-type arenavirus glycoprotein precursor. In certain embodiments, the wild-type arenavirus glycoprotein precursor can be processed into a GP signal peptide, a GP1 and a GP2. In certain embodiments, the functional fragment is a fragment of wild-type arenavirus nucleoprotein precursor. In certain embodiments, the functional fragment is a fragment of wild-type arenavirus Z protein precursor. In certain embodiments, the functional fragment is a fragment of wild-type arenavirus L protein precursor.

In certain embodiments, the functional fragment is a fragment of recombinant arenavirus glycoprotein, nucleoprotein, Z protein or L protein. In certain embodiments, the functional fragment is a fragment of recombinant arenavirus glycoprotein. In certain embodiments, the functional fragment is a fragment of recombinant arenavirus nucleoprotein. In certain embodiments, the functional fragment is a fragment of recombinant arenavirus Z protein. In certain embodiments, the functional fragment is a fragment of recombinant arenavirus L protein.

In certain embodiments, the functional fragment is recombinant arenavirus glycoprotein precursor, nucleoprotein precursor, Z protein precursor or L protein precursor. In certain embodiments, the functional fragment is a fragment of recombinant arenavirus glycoprotein precursor. In certain embodiments, the arenavirus glycoprotein precursor can be processed into a GP signal peptide, a GP1 and a GP2, one or more of which may be recombinant. In certain embodiments, the functional fragment is a fragment of recombinant arenavirus nucleoprotein precursor. In certain embodiments, the functional fragment is a fragment of recombinant arenavirus Z protein precursor. In certain embodiments, the functional fragment is a fragment of recombinant arenavirus L protein precursor.

In certain embodiments, the functional fragment is a fragment of mutated arenavirus glycoprotein, nucleoprotein, Z protein or L protein. In certain embodiments, the functional fragment is a fragment of mutated arenavirus glycoprotein. In certain embodiments, the functional fragment is a fragment of mutated arenavirus nucleoprotein. In certain embodiments, the functional fragment is a fragment of mutated arenavirus Z protein. In certain embodiments, the functional fragment is a fragment of mutated arenavirus L protein.

In certain embodiments, the functional fragment is mutated arenavirus glycoprotein precursor, nucleoprotein precursor, Z protein precursor or L protein precursor. In certain embodiments, the functional fragment is a fragment of mutated arenavirus glycoprotein precursor. In certain embodiments, the arenavirus glycoprotein precursor can be processed into a GP signal peptide, a GP1 and a GP2, one or more of which may be mutated. In certain embodiments, the functional fragment is a fragment of mutated arenavirus nucleoprotein precursor. In certain embodiments, the functional fragment is a fragment of mutated arenavirus Z protein precursor. In certain embodiments, the functional fragment is a fragment of mutated arenavirus L protein precursor.

In certain embodiments, the functional fragment is a fragment of arenavirus glycoprotein, nucleoprotein, Z protein or L protein that is derived from an attenuated virus. In certain embodiments, the functional fragment is a fragment of arenavirus glycoprotein derived from an attenuated virus. In certain embodiments, the functional fragment is a fragment of arenavirus nucleoprotein derived from an attenuated virus. In certain embodiments, the functional fragment is a fragment of arenavirus Z protein derived from an attenuated virus. In certain embodiments, the functional fragment is a fragment of arenavirus L protein derived from an attenuated virus.

In certain embodiments, the functional fragment is arenavirus glycoprotein precursor, nucleoprotein precursor, Z protein precursor or L protein precursor that is derived from an attenuated virus. In certain embodiments, the functional fragment is a fragment of arenavirus glycoprotein precursor derived from an attenuated virus. In certain embodiments, the arenavirus glycoprotein precursor can be processed into a GP signal peptide, a GP1 and a GP2, one or more of which may be derived from an attenuated virus. In certain embodiments, the functional fragment is a fragment of arenavirus nucleoprotein precursor derived from an attenuated virus. In certain embodiments, the functional fragment is a fragment of arenavirus Z protein precursor derived from an attenuated virus. In certain embodiments, the functional fragment is a fragment of arenavirus L protein precursor derived from an attenuated virus.

In certain embodiments, the functional fragment is a fragment of arenavirus glycoprotein precursor. In certain embodiments, the functional fragment is arenavirus GP signal peptide. In certain embodiments, the arenavirus GP signal peptide as described herein comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 80% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 85% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 90% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 95% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 96% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 97% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 98% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is at least 99% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 80% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 85% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 90% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 95% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 96% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 97% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 98% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135. In certain embodiments, the arenavirus GP signal peptide as described herein comprises an amino acid sequence that is 99% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, or SEQ ID NO:135.

In certain embodiments, the functional fragment is GP1. In certain embodiments, the GP1 as described herein comprises an amino acid sequence that is at least 80%, 85%, 90%, 95%, 96%, 97%, 98%, 99% or 100% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, or SEQ ID NO:136. In certain embodiments, the arenavirus GP1 as described herein comprises an amino acid sequence that is at least 80% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, or SEQ ID NO:136. In certain embodiments, the arenavirus GP1 as described herein comprises an amino acid sequence that is at least 85% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is at least 90% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is at least 95% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is at least 96% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is at least 97% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, or SEQ ID NO:136. In certain embodiments, the arenavirus GP1 as described herein comprises an amino acid sequence that is at least 98% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, or SEQ ID NO:136. In certain embodiments, the arenavirus GP1 as described herein comprises an amino acid sequence that is at least 99% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 100% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 80% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 85% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 90% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 95% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 96% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 97% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, or SEQ ID NO:136. In certain embodiments, the arenavirus GP1 as described herein comprises an amino acid sequence that is 98% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136. In certain embodiments, the arenavirus GP1 as described herein comprises an amino acid sequence that is 99% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, or SEQ ID NO:136.

In certain embodiments, the functional fragment is GP2. In certain embodiments, the GP2 as described herein comprises an amino acid sequence that is at least 80%, 85%, 90%, 95%, 96%, 97%, 98%, 99% or 100% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137. In certain embodiments, the arenavirus GP2 as described herein comprises an amino acid sequence that is at least 80% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 85% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 90% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 95% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 96% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 97% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 98% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is at least 99% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is 100% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is 80% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is 85% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137. In certain embodiments, the arenavirus GP2 as described herein comprises an amino acid sequence that is 90% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137. In certain embodiments, the arenavirus GP2 as described herein comprises an amino acid sequence that is 95% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137. In certain embodiments, the arenavirus GP2 as described herein comprises an amino acid sequence that is 96% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137. In certain embodiments, the arenavirus GP2 as described herein comprises an amino acid sequence that is 97% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137. In certain embodiments, the arenavirus GP2 as described herein comprises an amino acid sequence that is 98% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137. In certain embodiments, the arenavirus GP2 as described herein comprises an amino acid sequence that is 99% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137.

In certain embodiments, the functional fragment may consist of more than one of a GP signal peptide, GP1 and GP2. In certain embodiments, the functional fragment may consist of GP signal peptide and GP1. In certain embodiments, the functional fragment may consist of GP signal peptide and GP2. In certain embodiments, the functional fragment may consist of GP1 and GP2. In certain embodiments, the functional fragment is not the full-length glycoprotein precursor.

In certain embodiments, the functional fragment is a fragment of an arenavirus GP signal peptide. In certain embodiments, the arenavirus GP signal peptide is wild-type. In other embodiments, the arenavirus GP signal peptide is recombinant. In certain embodiments, the arenavirus GP signal peptide is selected from the group consisting of GP signal peptide of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the functional fragment is selected from the group consisting of the n region, hydrophobic region 1 (h-1 region) and hydrophobic region 2 (h-2 region) of the arenavirus GP signal peptide. In certain embodiments, the functional fragment is the n region of the arenavirus GP signal peptide. In certain embodiments, the functional fragment is the h-1 region of the arenavirus GP signal peptide. In certain embodiments, the functional fragment is the h-2 region of the arenavirus GP signal peptide.

In certain embodiments, the functional fragment as described herein is a fragment of a polypeptide that comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

In certain embodiments, the functional fragment as described herein comprises an amino acid that is at least 80% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 85% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 90% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 95% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 96% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 97% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 98% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is at least 99% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 100% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 80% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 85% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 90% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 95% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 96% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 97% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 98% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein comprises an amino acid that is 99% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

### 5.3 Heterologous Non-Arenaviral Polypeptide

As described herein, in certain embodiments, the heterologous non-arenaviral polypeptide may be not of arenavirus origin.

In certain embodiments, the nucleotide sequence provided herein (see Section 5.4) comprises a first open reading frame (ORF) that comprises a nucleotide sequence encoding a functional fragment of a first polypeptide selected from the group consisting of arenavirus GP, NP, Z and L, and a second ORF that comprises a nucleotide sequence encoding the heterologous non-arenaviral polypeptide provided herein. In certain embodiments, the nucleotide sequence provided herein comprises an open reading frame (ORF), and the ORF comprises a nucleotide sequence encoding a functional fragment of a first polypeptide selected from the group consisting of arenavirus GP, NP, Z and L that are not from Lassa virus, and a heterologous non-arenaviral polypeptide. In certain embodiments, the arenavirus particle described herein comprises a genome encoding a heterologous polypeptide of arenavirus origin, but derived from a type of arenavirus that is different from the type of arenavirus from which the arenavirus particle comprises GP, NP, Z as well as L. In an exemplary embodiment, the heterologous polypeptide is from Lassa virus, whereas the arenavirus particle comprises the GP, NP, Z as well as L of LCMV or Pichinde virus.

In certain embodiments, the arenavirus particle provided herein is engineered such that an arenaviral ORF is separated over two or more mRNA transcripts (see Section 5.5). In certain embodiments, one of the mRNA transcripts further comprises a nucleotide sequence encoding a heterologous non-arenaviral polypeptide. In certain embodiments, one of the mRNA transcripts further comprises a nucleotide sequence encoding heterologous polypeptide of arenavirus origin, but derived from a type of arenavirus that is different from the type of arenavirus from which the arenavirus particle comprises GP, NP, Z as well as L. In an exemplary embodiment, the heterologous polypeptide is from Lassa virus, whereas the arenavirus particle comprises the GP, NP, Z as well as L of LCMV or Pichinde virus.

In certain embodiments, the arenavirus genomic or antigenomic segment provided herein (see Section 5.6) is engineered such that the viral transcription thereof results in a first mRNA transcript comprising a nucleotide sequence encoding a functional fragment of a first polypeptide selected from the group consisting of arenavirus GP, NP, Z and L and a second mRNA transcript comprising a nucleotide sequence encoding a heterologous non-arenaviral polypeptide. In certain embodiments, the arenavirus genomic or antigenomic segment provided herein is engineered such that the viral transcription thereof results in an mRNA transcript encoding a functional fragment of a first polypeptide selected from the group consisting of arenavirus GP, NP, Z and L, and a heterologous non-arenaviral polypeptide or a second polypeptide. In certain embodiments, the arenavirus genomic or antigenomic segment provided herein is engineered such that the viral transcription thereof results in an mRNA transcript encoding a functional fragment of a first polypeptide selected from the group consisting of arenavirus GP, NP, Z and L, and a heterologous polypeptide of arenavirus origin, but derived from a type of arenavirus that is different from the type of arenavirus from which the arenavirus genome comprises GP, NP, Z as well as L. In an exemplary embodiment, the heterologous polypeptide is from Lassa virus, whereas the arenavirus genome comprises the GP, NP, Z as well as L of LCMV or Pichinde virus.

In certain embodiments, the heterologous non-arenaviral polypeptide is about, at most about, or at least about 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, 300, 350, 400, 450, 500, 550, 600, 650, 700, 750, 800, 850, 900, 950, 1000, or more than 1000 amino acids in length. In certain embodiments, the heterologous non-arenaviral polypeptide is about, at least about, or at most about 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 300 to 500 amino acids in length, 350 to 600 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In certain embodiments, the heterologous non-arenaviral polypeptide does not exceed 500 amino acids in length. In certain embodiments, the heterologous non-arenaviral polypeptide does not exceed 750 amino acids in length. In certain embodiments, the heterologous non-arenaviral polypeptide does not exceed 1000 amino acids in length. In certain embodiments, the heterologous non-arenaviral polypeptide does not exceed 2500 amino acids in length.

In certain embodiments, the heterologous non-arenaviral polypeptide consists of about, at least about, or at most about 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, 300, 350, 400, 450, 500, 550, 600, 650, 700, 750, 800, 850, 900, 950, or 1000 amino-acid residues. In certain embodiments, the heterologous non-arenaviral polypeptide may consist of about, at least about, or at most about 58 amino-acid residues. In certain embodiments, the heterologous non-arenaviral polypeptide may consist of about, at least about, or at most about 193 amino-acid residues. In certain embodiments, the heterologous non-arenaviral polypeptide may consist of about, at least about, or at most about 200 amino-acid residues. In certain embodiments, the heterologous non-arenaviral polypeptide may consist of about, at least about, or at most about 480 amino-acid residues. In certain embodiments, the heterologous non-arenaviral polypeptide may consist of about, at least about, or at most about 450 amino-acid residues. In certain embodiments, the heterologous non-arenaviral polypeptide may consist of about, at least about, or at most about 500 amino-acid residues. In certain embodiments, the heterologous non-arenaviral polypeptide may consist of about, at least about, or at most about 350 amino-acid residues. In certain embodiments, the heterologous non-arenaviral polypeptide may consist of about, at least about, or at most about 400 amino-acid residues. In certain embodiments, the heterologous non-arenaviral polypeptide may consist of about, at least about, or at most about 550 amino-acid residues. In certain embodiments, the heterologous non-arenaviral polypeptide may consist of about, at least about, or at most about 600 amino-acid residues. In certain embodiments, the heterologous non-arenaviral polypeptide may consist of about, at least about, or at most about 15 amino-acid residues. In certain embodiments, the heterologous non-arenaviral polypeptide may consist of about, at least about, or at most about 20 amino-acid residues. In certain embodiments, the heterologous non-arenaviral polypeptide may consist of about, at least about, or at most about 1 to 20 amino-acid residues. In certain embodiments, the heterologous non-arenaviral polypeptide may consist of about, at least about, or at most about 200 to 600 amino-acid residues.

In certain embodiments, the heterologous non-arenaviral polypeptide is a reporter protein (see Section 5.3.1). In certain embodiments, the heterologous non-arenaviral polypeptide is a heterologous non-arenaviral signal peptide (see Section 5.3.2). In certain embodiments, the heterologous non-arenaviral polypeptide is an antigen (see Section 5.3.3).

### 5.3.1 Reporter Protein

In certain embodiments, the heterologous non-arenaviral polypeptide described herein is a reporter protein or a fluorescent protein. In certain embodiments, the reporter protein is a fluorescent protein. In certain embodiments, the reporter protein is green fluorescent protein (GFP). GFP emits bright green light when exposed to UV or blue like. In other embodiments, the reporter protein is a red fluorescent protein (RFP). In an exemplary embodiments, the report protein is TOM.

Reporter protein and genes would be readily recognized by one of skill in the art. Non-limiting examples of reporter proteins include various enzymes, such as, but not to β-galactosidase, chloramphenicol acetyltransferase, neomycin phosphotransferase, luciferase or RFP.

In certain embodiments, the reporter protein is capable of expression at the same time as the antigen described herein. Ideally, expression is visible in normal light or other wavelengths of light. In certain embodiments, the intensity of the effect created by the reporter protein can be used to directly measure and monitor the arenavirus particle or tri-segmented arenavirus particle.

### 5.3.2 Heterologous Non-Arenaviral Signal Peptide

In certain embodiments, the heterologous non-arenaviral polypeptide described herein is a heterologous non-arenaviral signal peptide. In certain embodiments, the heterologous non-arenaviral signal peptide is a signal peptide of a glycoprotein. In certain embodiments, the heterologous non-arenaviral signal peptide is from a virus family selected from the group consisting of adenoviridae (e.g., mastadenovirus and aviadenovirus), herpesviridae (e.g., herpes simplex virus 1, herpes simplex virus 2, herpes simplex virus 5, herpes simplex virus 6, Epstein-Barr virus, HHV6-HHV8, cytomegalovirus, and varicella zoster virus), leviviridae (e.g., levivirus, enterobacteria phase MS2, allolevirus), orthomyxoviridae (e.g., influenzavirus A, influenzavirus B, influenzavirus C), parvoviridae (e.g., Parvovirus B19), filoviridae (e.g., Ebola virus, Marburg virus), hantaviridae, poxviridae (e.g., chordopoxviridae, parapoxvirus, avipoxvirus, capripoxvirus, leporiipoxvirus, suipoxvirus, molluscipoxvirus, and entomopoxyirinae), polyomaviridae, papillomaviridae (e.g., human papillomavirus), paramyxoviridae (e.g., paramyxovirus, parainfluenza virus 1, mobillivirus (e.g., measles virus), rubulavirus (e.g., mumps virus)), pneumoviridae (e.g., pneumovirus, human respiratory syncytial virus and metapneumovirus (e.g., avian pneumovirus and human metapneumovirus)), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus (HIV) 1 and HIV-2 (e.g., HIV gp160)), spumavirus), flaviviridae (e.g., hepatitis C virus, dengue virus, West Nile virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, mobala virus, mopeia virus, amapari virus, flexal virus, guanarito virus, junin virus, latino virus, machupo virus, oliveros virus, parana virus, pichinde virus, pirital virus, sabia virus, tacaribe virus, tamiami virus, bear canyon virus, whitewater arroyo virus, allpahuayo virus (ALLV), and lassa virus), and coronaviridae (e.g., coronavirus and torovirus). In certain embodiments, the heterologous non-arenaviral signal peptide is the signal peptide of the vesicular stomatitis virus serotype Indiana glycoprotein.

### 5.3.3 Antigen

In certain embodiments, the heterologous non-arenaviral polypeptide is an antigen. In certain embodiments, the antigen is derived from an infectious organism, tumor, or allergen. In one embodiment, the antigen is of an infectious pathogen or associated with any disease that is capable of eliciting an immune response. In certain embodiments, the heterologous non-arenaviral polypeptide is an antigen derived from a virus, a bacterium, a fungus, a parasite, or can be expressed in a tumor or tumor associated disease (i.e., cancer), an autoimmune disease, a degenerative disease, an inherited disease, substance dependency, obesity, or an allergic disease.

In certain embodiments, the heterologous non-arenaviral polypeptide is a viral antigen. Non-limiting examples of viral antigens include antigens from adenoviridae (e.g., mastadenovirus and aviadenovirus), herpesviridae (e.g., herpes simplex virus 1, herpes simplex virus 2, herpes simplex virus 5, herpes simplex virus 6, Epstein-Barr virus, HHV6-HHV8, cytomegalovirus, and varicella zoster virus), leviviridae (e.g., levivirus, enterobacteria phase MS2, allolevirus), orthomyxoviridae (e.g., influenzavirus A, influenzavirus B, influenzavirus C), parvoviridae (e.g., Parvovirus B19), filoviridae (e.g., Ebola virus, Marburg virus), hantaviridae, poxviridae (e.g., chordopoxviridae, parapoxvirus, avipoxvirus, capripoxvirus, leporiipoxvirus, suipoxvirus, molluscipoxvirus, and entomopoxyirinae), polyomaviridae, papillomaviridae (e.g., human papillomavirus), paramyxoviridae (e.g., paramyxovirus, parainfluenza virus 1, mobillivirus (e.g., measles virus), rubulavirus (e.g., mumps virus)), pneumoviridae (e.g., pneumovirus, human respiratory syncytial virus and metapneumovirus (e.g., avian pneumovirus and human metapneumovirus)), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus (HIV) 1 and HIV-2 (e.g., HIV gp160)), spumavirus), flaviviridae (e.g., hepatitis C virus, dengue virus, West Nile virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, mobala virus, mopeia virus, amapari virus, flexal virus, guanarito virus, junin virus, latino virus, machupo virus, oliveros virus, parana virus, pichinde virus, pirital virus, sabia virus, tacaribe virus, tamiami virus, bear canyon virus, whitewater arroyo virus, allpahuayo virus (ALLV), and lassa virus), and coronaviridae (e.g., coronavirus and torovirus). In certain embodiments, the arenavirus particle described herein comprises a genome encoding an antigen from a mammarenavirus (e.g., Allpahuayo virus, Alxa virus, Junin virus, Bear Canyon virus, Sabia virus, Pichinde virus, Chapare virus, Lijiang virus, Cupixi virus, Flexal virus, Gairo virus, Guanarito virus, Ippy virus, Lassa virus, Latino virus, Loei River virus, Lujo virus, Luna virus, Luli virus, Lunk virus, lymphocytic choriomeningitis virus, Machupo virus, Mariental virus, Merino Walk virus, Mobala virus, Mopeia virus, Morogoro virus, Okahandja virus, Oliveros virus, Parana virus, Pirital virus, Aporé virus, Ryukyu virus, Amapari virus, Solwezi virus, souris virus, Tacaribe virus, Tamiami virus, Wenzhou virus, Whitewater Arroyo virus, Big Brushy Tank virus, Catarina virus, Skinner Tank virus, Tonto Creek virus, or Xapuri virus), wherein the mammarenavirus is different from the species of arenavirus from which the arenavirus particle comprises GP, NP, Z as well as L. In a specific embodiment the viral antigen, is HIV gp120, gp41, HIV Nef, RSV F glycoprotein, RSV G glycoprotein, HTLV tax, herpes simplex virus glycoprotein (e.g., gB, gC, gD, and gE) or hepatitis B surface antigen, hepatitis C virus E protein or coronavirus spike protein. In one embodiment, the viral antigen is not an HIV antigen.

In other embodiments, the heterologous non-arenaviral polypeptide is a bacterial antigen (e.g., bacterial coat protein). Non-limiting examples of bacterial antigens include antigens from a bacteria family selected from the group consisting of the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Envinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcus (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, Yersinia family, Bacillus antracis and Vampirovibrio family.

In other embodiments, the heterologous non-arenaviral polypeptide is a parasitic antigen (e.g., a protozoan antigen). Non-limiting examples of parasite antigens include antigens from a parasite such as an amoeba, a malarial parasite, Plasmodium, Trypanosoma cruzi.

In yet other embodiments, the heterologous non-arenaviral polypeptide is a fungal antigen. Non-limiting examples of fungal antigens include antigens from fungus of Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kern, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

In some embodiments, the heterologous non-arenaviral polypeptide is a tumor neoantigen. A “neoantigen,” used herein, means an antigen that arises by mutation in a tumor cell and such an antigen is not generally expressed in normal cells or tissue. Without being bound by theory, because healthy tissues generally do not possess these antigens, neoantigens represent a preferred target. Additionally, without being bound by theory, in the context of the present invention, since the T cells that recognize the neoantigen may not have undergone negative thymic selection or functionally impacted by peripheral tolerance mechanisms, such cells can have high functional avidity to the antigen and mount a strong immune response against tumors, while lacking the risk to induce destruction of normal tissue and autoimmune damage. In certain embodiments, the neoantigen is an MHC class I-restricted neoantigen. In certain embodiments, the neoantigen is an MHC class II-restricted neoantigen. In certain embodiments, a mutation in a tumor cell of the patient results in a novel protein that produces the neoantigen. In certain embodiments, the heterologous non-arenaviral polypeptide is a tumor neo-epitope. In certain embodiments, the neo-epitope is an MHC class I-restricted neo-epitope. In certain embodiments, the neo-epitope is an MHC class II-restricted neo-epitope.

In some embodiments, the heterologous non-arenaviral polypeptide is a tumor antigen or tumor associated antigen. In some embodiments, the tumor antigen or tumor associated antigen includes antigens from tumor associated diseases including acute lymphoblastic leukemia, acute myeloid leukemia, adrenocortical carcinoma, childhood adrenocortical carcinoma, AIDS-Related Cancers, Kaposi Sarcoma, anal cancer, appendix cancer, astrocytomas, atypical teratoid/rhabdoid tumor, basal-cell carcinoma, bile duct cancer, extrahepatic (see cholangiocarcinoma), bladder cancer, bone osteosarcoma/malignant fibrous histiocytoma, brainstem glioma, brain cancer, brain tumor, cerebellar astrocytoma, cerebral astrocytoma/malignant glioma brain tumor, ependymoma, medulloblastoma, supratentorial primitive neuroectodermal tumors, visual pathway and hypothalamic glioma, breast cancer, bronchial adenomas/carcinoids, burkitt's lymphoma, carcinoid tumor, carcinoid gastrointestinal tumor, carcinoma of unknown primary, central nervous system lymphoma, primary, cerebellar astrocytoma, cerebral astrocytoma/malignant glioma, cervical cancer, childhood cancers, chronic bronchitis, chronic lymphocytic leukemia, chronic myelogenous leukemia, chronic myeloproliferative disorders, colon cancer, cutaneous T-cell lymphoma, desmoplastic small round cell tumor, emphysema, endometrial cancer, ependymoma, esophageal cancer, ewing's sarcoma in the Ewing family of tumors, extracranial germ cell tumor, extragonadal germ cell tumor, extrahepatic bile duct cancer, intraocular melanoma, retinoblastoma, gallbladder cancer, gastric (stomach) cancer, gastrointestinal carcinoid tumor, gastrointestinal stromal tumor, germ cell tumor: extracranial, extragonadal, or ovarian gestational trophoblastic tumor, glioma of the brain stem, glioma, childhood cerebral astrocytoma, childhood visual pathway and hypothalamic, gastric carcinoid, hairy cell leukemia, head and neck cancer, heart cancer, hepatocellular (liver) cancer, hodgkin lymphoma, hypopharyngeal cancer, hypothalamic and visual pathway glioma, intraocular melanoma, islet cell carcinoma (endocrine pancreas), kaposi sarcoma, kidney cancer (renal cell cancer), laryngeal cancer, acute lymphoblastic lymphoma, acute lymphocytic leukemia, acute myelogenous leukemia, chronic lymphocytic leukemia, chronic myeloid leukemia, lip and oral cavity cancer, liposarcoma, liver cancer (primary), lung cancer, non-small cell, small cell, AIDS-related lymphoma, Burkitt lymphoma, cutaneous T-cell lymphoma, hodgkin lymphoma, non-hodgkin lymphoma, lymphoma, primary central nervous system, macroglobulinemia, Waldenström, male breast cancer, malignant fibrous histiocytoma of bone/osteosarcoma, medulloblastoma, melanoma, intraocular (eye), merkel cell cancer, mesothelioma, adult malignant, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myelogenous leukemia, chronic, myeloid leukemia, adult acute, myeloid leukemia, childhood acute, myeloma, multiple (cancer of the bone-marrow), myeloproliferative disorders, chronic, nasal cavity and paranasal sinus cancer, nasopharyngeal carcinoma, neuroblastoma, non-small cell lung cancer, oligodendroglioma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, islet cell, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma, childhood, salivary gland cancer, sarcoma, Ewing family of tumors, Kaposi sarcoma, soft tissue sarcoma, uterine sarcoma, sezary syndrome, skin cancer (non-melanoma), skin cancer (melanoma), merkel cell skin carcinoma, small cell lung cancer, small intestine cancer, soft tissue sarcoma, squamous cell carcinoma—see skin cancer (non-melanoma), squamous neck cancer with occult primary, metastatic, stomach cancer, supratentorial primitive neuroectodermal tumor, T-Cell lymphoma, cutaneous—see Mycosis Fungoides and Sezary syndrome, testicular cancer, throat cancer, thymoma and thymic carcinoma, thyroid cancer, childhood transitional cell cancer of the renal pelvis and ureter, gestational trophoblastic tumor, unknown primary site, carcinoma of, adult unknown primary site, cancer of childhood, ureter and renal pelvis, transitional cell cancer, rethral cancer, uterine cancer, endometrial uterine sarcoma, bronchial tumor, central nervous system embryonal tumor; childhood chordoma, colorectal cancer, craniopharyngioma, ependymoblastoma, langerhans cell histiocytosis, acute lymphoblastic leukemia, acute myeloid leukemia (adult/childhood), small cell lung cancer, medulloepithelioma, oral cavity cancer, papillomatosis, pineal parenchymal tumors of intermediate differentiation, pituary tumor, respiratory tract carcinoma involving the NUT gene on chromosome 15, spinal cord tumor, thymoma, thyroid cancer, vaginal cancer; vulvar cancer, and Wilms tumor.

Non-limiting examples of tumor or tumor associated antigens include Adipophilin, AIM-2, ALDH1A1, BCLX (L), BING-4, CALCA, CD45, CPSF, cyclin D1, DKK1, ENAH (hMena), EpCAM, EphA3, EZH2, FGF5, glypican-3, G250/MN/CAIX, HER-2/neu, IDO1, IGF2B3, IL 13Ralpha2, Intestinal carboxyl esterase, alpha-fetoprotein, Kallikrein 4, KIF20A, Lengsin, M-CSF, MCSP, mdm-2, Meloe, MMP-2, MMP-7, MUC1, MUC5AC, p53, PAX5, PBF, PRAME, PSMA, RAGE-1, RGS5, RhoC, RNF43, RU2AS, secernin 1, SOX10, STEAP1, survivinn, Telomerase, VEGF, or WT1, EGF-R, CEA, CD52, gp 100 protein, MELANA/MART1, NY-ESO-1, p53, MAGE1, MAGE3 and CDK4, alpha-actinin-4, ARTC1, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, K-ras, N-ras, RBAF600, SIRT2, SNRPD1, SYT-SSX1 or -SSX2 fusion protein, TGF-betaRII, Triosephosphate isomerase, Lengsin, M-CSF, MCSP, or mdm-2.

In some embodiments, the heterologous non-arenaviral polypeptide is a respiratory pathogen antigen. In a specific embodiment, the respiratory pathogen is a virus such as RSV, coronavirus, human metapneumovirus, parainfluenza virus, hendra virus, nipah virus, adenovirus, rhinovirus, or PRRSV. Non-limiting examples of respiratory viral antigens include Respiratory Syncytial virus F, G and M2 proteins, Coronavirus (SARS, HuCoV) spike proteins (S), human metapneumovirus fusion proteins, Parainfluenza virus fusion and hemagglutinin proteins (F, HN), Hendra virus (HeV) and Nipah virus (NiV) attachment glycoproteins (G and F), Adenovirus capsid proteins, Rhinovirus proteins, and PRRSV wild type or modified GP5 and M proteins.

In a specific embodiment, the respiratory pathogen is a bacteria such as Bacillus anthracis, Mycobacterium tuberculosis, Bordetella pertussis, Streptococcus pneumoniae, Yersinia pestis, Staphylococcus aureus, Francisella tularensis, Legionella pneumophila, Chlamydia pneumoniae, Pseudomonas aeruginosa, Neisseria meningitides, and Haemophilus influenzae. Non-limiting examples of respiratory bacterial antigens include Bacillus anthracis Protective antigen PA, Mycobacterium tuberculosis mycobacterial antigen 85A and heat shock protein (Hsp65), Bordetella pertussis pertussis toxoid (PT) and filamentous hemagglutinin (FHA), Streptococcus pneumoniae sortase A and surface adhesin A (PsaA), Yersinia pestis F1 and V subunits, and proteins from Staphylococcus aureus, Francisella tularensis, Legionella pneumophila, Chlamydia pneumoniae, Pseudomonas aeruginosa, Neisseria meningitides, and Haemophilus influenzae.

In some embodiments, the heterologous non-arenaviral polypeptide is a T-cell epitope. In other embodiments, the heterologous ORF encodes a cytokine or growth factor.

In other embodiments, the heterologous non-arenaviral polypeptide is an antigen expressed in an autoimmune disease. In more specific embodiments, the autoimmune disease can be type I diabetes, multiple sclerosis, rheumatoid arthritis, lupus erythmatosus, and psoriasis. Non-limiting examples of autoimmune disease antigens include Ro60, dsDNA, or RNP.

In other embodiments, the heterologous non-arenaviral polypeptide is an antigen expressed in an allergic disease. In more specific embodiments, the allergic disease can include but is not limited to seasonal and perennial rhinoconjunctivitis, asthma, and eczema. Non-limiting examples of allergy antigens include Bet v 1 and Fel d 1.

### 5.4 Nucleotide Sequence Provided Herein

In one aspect, provided herein are nucleotide sequences. In certain embodiments, provided herein is a nucleotide sequence comprising a first open reading frame (ORF) and a second ORF, wherein one of the two ORFs is in sense orientation and the other ORF is in antisense orientation;

wherein the first ORF comprises a nucleotide sequence encoding a functional fragment of a first polypeptide, and the first ORF does not encode the full-length first polypeptide;
wherein the second ORF comprises a nucleotide sequence encoding:
a) a second polypeptide; or
b) a functional fragment of the first polypeptide, and the second ORF does not encode the full-length first polypeptide; or
c) a functional fragment of a second polypeptide, and the second ORF does not encode the full-length second polypeptide; or
d) a heterologous non-arenaviral polypeptide; and
   wherein the first and second polypeptides are different from each other and selected from the group consisting of arenavirus GP, NP, Z and L.

In certain embodiments, the first ORF and the second ORF are separated by an arenavirus intergenic region (IGR) and each ORF is under control of an arenavirus 3′ untranslated region (UTR) or an arenavirus 5′ UTR.

In certain embodiments, the first ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the second ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the first ORF is part of a transcription unit that further comprises another ORF, wherein the other ORF and the first ORF are separated by an internal ribosome entry site (IRES). In certain embodiments, the second ORF is part of a transcription unit that further comprises another ORF, wherein the other ORF and the second ORF are separated by an IRES. In certain embodiments, the first ORF and/or the second ORF comprises a nucleotide sequence encoding an arenavirus GP signal peptide or a functional fragment thereof and the other ORF comprises a nucleotide sequence encoding a heterologous non-arenaviral polypeptide or an arenavirus NP, Z, or L.

In certain embodiments, the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, provided herein is a nucleotide sequence comprising an open reading frame (ORF), wherein the ORF comprises a nucleotide sequence encoding
a) a functional fragment of a first polypeptide, and
b) a heterologous non-arenaviral polypeptide or a second polypeptide;
   wherein the ORF does not encode the full-length first polypeptide; and wherein the first and second polypeptides are different from each other and selected from the group consisting of arenavirus GP, NP, Z and L that are not from Lassa virus. In certain embodiments, the first and second polypeptides are selected from the group consisting of arenavirus GP, NP, Z and L of LCMV, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the ORF is a first ORF and the nucleotide sequence further comprises a second ORF. In certain embodiments, the second ORF comprises a nucleotide sequence encoding a third polypeptide, a functional fragment of the first polypeptide, a functional fragment of a third polypeptide, or a second heterologous non-arenaviral polypeptide; wherein the third polypeptide is different from the first polypeptide and the second polypeptide and selected from the group consisting of arenavirus GP, NP, Z and L; wherein one of the two ORFs is in sense orientation and the other ORF is in antisense orientation; wherein the second ORF does not encode the full-length first polypeptide; wherein the second ORF does not encode the full-length third polypeptide; and wherein the heterologous non-arenaviral polypeptide and the second heterologous non-arenaviral polypeptide are the same or different from each other. In certain embodiments, the second ORF comprises a nucleotide sequence encoding a functional fragment of a first polypeptide, and wherein the functional fragment encoded by the first ORF is different from the functional fragment encoded by the second ORF.

In certain embodiments, the nucleotide sequence does not further comprise a second ORF.

In certain embodiments, the nucleotide sequence is an arenavirus genomic or antigenomic S segment. In certain embodiments, the nucleotide sequence is an arenavirus genomic or antigenomic L segment.

In certain embodiments, the first ORF is under control of an arenavirus 3′ UTR, and the second ORF is under control of an arenavirus 5′ UTR. In certain embodiments, the first ORF is under control of an arenavirus 5′ UTR, and the second ORF is under control of an arenavirus 3′ UTR.

In certain embodiments, the first ORF comprises a nucleotide sequence encoding arenavirus GP signal peptide, either alone or fused to a heterologous non-arenaviral polypeptide. In certain embodiments, the second ORF comprises a nucleotide sequence encoding a heterologous non-arenaviral signal peptide and the arenavirus GP1 and GP2. In certain embodiments, the second ORF comprises a nucleotide sequence encoding a heterologous non-arenaviral polypeptide; wherein the heterologous non-arenaviral polypeptide encoded by the first ORF and the heterologous non-arenaviral polypeptide encoded by the second ORF are the same or different from each other. In certain embodiments, the first ORF is under control of an arenavirus 3′ UTR and the second ORF is under control of an arenavirus 5′ UTR.

In certain embodiments, the first ORF comprises a nucleotide sequence encoding a heterologous non-arenaviral signal peptide and the arenavirus GP1 and GP2. In certain embodiments, the second ORF comprises a nucleotide sequence encoding NP. In certain embodiments, the first ORF is under control of an arenavirus 5′ UTR and the second ORF is under control of an arenavirus 3′ UTR.

In certain embodiments, the first polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140; and the second polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140; and wherein the third polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the first polypeptide comprises an amino acid sequence identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, or SEQ ID NO:137.

In certain embodiments, the functional fragment encoded by the first ORF or the second ORF is an arenavirus GP signal peptide or a functional fragment thereof.

In certain embodiments, the heterologous non-arenaviral polypeptide, the second heterologous non-arenaviral polypeptide, or both heterologous non-arenaviral polypeptides are each an antigen derived from an infectious organism, tumor, or allergen. In certain embodiments, the antigen is selected from the group consisting of
(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

In certain embodiments, the heterologous non-arenaviral polypeptide or the second heterologous non-arenaviral polypeptide is expressed under control of an arenavirus 3′ UTR; and the expression level of the heterologous non-arenaviral polypeptide or the expression level of the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or higher than the expression level of the same second heterologous non-arenaviral polypeptide expressed under control of the respective arenavirus 5′ UTR.

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the combined expression level of both the heterologous non-arenaviral polypeptides is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of both the heterologous non-arenaviral polypeptides expressed under control of arenavirus 5′ UTRs.

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the heterologous non-arenaviral polypeptide is expressed under control of an arenavirus 3′ UTR and the second heterologous non-arenaviral polypeptide is expressed under control of an arenavirus 5′ UTR. In certain embodiments, the heterologous non-arenaviral polypeptide is expressed under control of an arenavirus 5′ UTR and the second heterologous non-arenaviral polypeptide is expressed under control of an arenavirus 3′ UTR. In certain embodiments, the combined expression level of both the heterologous non-arenaviral polypeptide and the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR and the same second heterologous non-arenaviral polypeptide is expressed under control of an arenavirus 5′ UTR).

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the heterologous non-arenaviral polypeptide is expressed under control of an arenavirus 3′ UTR and the second heterologous non-arenaviral polypeptide is expressed under control of an arenavirus 5′ UTR. In certain embodiments, the proportion of cells that express both the heterologous non-arenaviral polypeptide and the second heterologous non-arenaviral polypeptide (e.g., cells that co-express the heterologous non-arenaviral polypeptide and the second heterologous non-arenaviral polypeptide) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells that express both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR and the same second heterologous non-arenaviral polypeptide is expressed under control of an arenavirus 5′ UTR). In some embodiments, the proportion of cells is determined after a population of cells are infected with an arenavirus of the present disclosure (e.g., one heterologous non-arenaviral polypeptide is expressed under control of an arenavirus 3′ UTR and the other heterologous non-arenaviral polypeptide is expressed under control of an arenavirus 5′ UTR). In some embodiments, the proportion of cells is determined after a population of cells are infected with another arenavirus (e.g., both heterologous non-arenaviral polypeptides expressed under control of an arenavirus 5′ UTR).

In certain embodiments, one heterologous non-arenaviral polypeptide is expressed under control of an arenavirus 5′ UTR and the other heterologous non-arenaviral polypeptide is expressed under control of an arenavirus 3′ UTR. In certain embodiments, the heterologous non-arenaviral polypeptide is expressed under control of an arenavirus 5′ UTR and the second heterologous non-arenaviral polypeptide is expressed under control of an arenavirus 3′ UTR. In certain embodiments, the heterologous non-arenaviral polypeptide is expressed under control of an arenavirus 5′ UTR in one S segment and the second heterologous non-arenaviral polypeptide is expressed under control of an arenavirus 3′ UTR in another S segment. In certain embodiments, the proportion of cells that express both the heterologous non-arenaviral polypeptide and the second heterologous non-arenaviral polypeptide (e.g., cells that co-express the heterologous non-arenaviral polypeptide and the second heterologous non-arenaviral polypeptide) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells that express both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR in one S segment and the same second heterologous non-arenaviral polypeptide is expressed under control of an arenavirus 5′ UTR in another S segment). In some embodiments, the proportion of cells is determined after a population of cells are infected with an arenavirus of the present disclosure (e.g., one heterologous non-arenaviral polypeptide is expressed under control of an arenavirus 3′ UTR and the other heterologous non-arenaviral polypeptide is expressed under control of an arenavirus 5′ UTR). In some embodiments, the proportion of cells is determined after a population of cells are infected with another arenavirus (e.g., both heterologous non-arenaviral polypeptides expressed under control of an arenavirus 5′ UTR).

In certain embodiments, the nucleotide sequence provided herein comprises a first open reading frame (ORF) and a second ORF, and one of the two ORFs is in sense orientation and the other ORF is in antisense orientation. In certain embodiments, the first ORF is in sense orientation and the second ORF is in antisense orientation. In other embodiments, the first ORF is in antisense orientation and the second ORF is in sense orientation. In certain embodiments, the first ORF and/or the second ORF is part of a transcription unit that further comprises another ORF, wherein the other ORF and each of the first ORF and/or the second ORF are separated by an internal ribosome entry site (IRES).

In certain embodiment, the functional fragment is encoded alone by the first ORF or the second ORF. In certain embodiments, the functional fragment is fused to a heterologous non-arenaviral polypeptide or a polypeptide selected from an arenavirus GP, NP, Z and L. In certain embodiment, the functional fragment is under control of an arenavirus 3′ UTR. In other embodiments, the functional fragment is under control of an arenavirus 5′ UTR. In certain embodiment, the functional fragment encoded by the first ORF is different from the functional fragment encoded by the second ORF.

In certain embodiments, the functional fragment encoded by the first ORF or the second ORF is arenavirus GP signal peptide. In certain embodiments, the arenavirus GP signal peptide is not fused to an arenavirus GP, NP, Z or L.

In certain embodiments, the nucleotide provided herein is an mRNA. In certain embodiments, the nucleotide provided herein is an mRNA comprising an internal ribosome entry site (IRES). In other embodiments, the nucleotide provided herein is a DNA. In certain embodiments, the nucleotide provided herein can be translated into a product, for example a polypeptide. In certain embodiments, the nucleotide provided herein is a DNA that can be transcribed to produce an arenavirus genomic or antigenomic RNA segment. In certain embodiments, the nucleotide sequence as described herein can be part of a DNA expression vector.

Non-limiting examples of the polypeptides encoded by the first and second ORFs are illustration in Table 1.

TABLE 1
Non-limiting examples of the polypeptides encoded
by the first and second ORFs.

| | 1^{st} ORF | | |
|---|---|---|---|
| | Functional | | Functional |
| | fragment of a first | | fragment of a |
| | polypeptide and a | | first |
| | heterologous | | polypeptide |
| | non-arenaviral | | and a third |
| | polypeptide | Functional | polypeptide |
| | (fused together | fragment | (fused together |
| | or separated | of a first | or separated |
| 2^{nd} ORF | by an IRES) | polypeptide | by an IRES) |
| Functional fragment | Exemplary | Exemplary | Exemplary |
| of a first polypeptide | combination | combination | combination |
| and a heterologous | | | |
| non-arenaviral | | | |
| polypeptide | | | |
| (fused together or | | | |
| separated by an | | | |
| IRES) | | | |
| Functional fragment | Exemplary | Exemplary | Exemplary |
| of a first polypeptide | combination | combination | combination |
| Functional fragment | Exemplary | Exemplary | Exemplary |
| of a first polypeptide | combination | combination | combination |
| and a third | | | |
| polypeptide | | | |
| (fused together or | | | |
| separated by an | | | |
| IRES) | | | |
| A heterologous non- | Exemplary | Exemplary | Exemplary |
| arenaviral | combination | combination | combination |
| polypeptide | | | |
| A second | Exemplary | Exemplary | Exemplary |
| polypeptide | combination | combination | combination |
| Functional fragment | Exemplary | Exemplary | Exemplary |
| of a second | combination | combination | combination |
| polypeptide and a | | | |
| heterologous non- | | | |
| arenaviral | | | |
| polypeptide | | | |
| (fused together or | | | |
| separated by an | | | |
| IRES) | | | |
| Functional fragment | Exemplary | Exemplary | Exemplary |
| of a second | combination | combination | combination |
| polypeptide | | | |
| Functional fragment | Exemplary | Exemplary | Exemplary |
| of a second | combination | combination | combination |
| polypeptide and a | | | |
| third polypeptide | | | |
| (fused together or | | | |
| separated by an | | | |
| IRES) | | | |

In certain embodiments, the functional fragment is selected from the group consisting of arenavirus GP signal peptide, arenavirus GP1 and arenavirus GP2. Non-limiting examples of the polypeptides encoded by the first and second ORFs related to functional fragments of GP are illustrated in Table 2.

TABLE 2
Non-limiting examples of the polypeptides encoded by the first and second
ORFs related to functional fragments of GP.

| First ORF | Second ORF |
|---|---|
| GP signal peptide and a heterologous non- | A heterologous non-arenaviral |
| arenaviral polypeptide | signal peptide, arenavirus GP1, |
| | and arenavirus GP2 |
| GP signal peptide and a heterologous non- | A heterologous non-arenaviral |
| arenaviral polypeptide | polypeptide |
| GP signal peptide and a heterologous non- | NP |
| arenaviral polypeptide | |
| GP signal peptide and a heterologous non- | Z |
| arenaviral polypeptide | |
| GP signal peptide and a heterologous non- | L |
| arenaviral polypeptide | |
| GP signal peptide and a heterologous non- | No second ORF |
| arenaviral polypeptide | |
| GP signal peptide | A heterologous non-arenaviral |
| | signal peptide, arenavirus GP1, |
| | and arenavirus GP2 |
| GP signal peptide | A heterologous non-arenaviral |
| | polypeptide |
| GP signal peptide | NP |
| GP signal peptide | Z |
| GP signal peptide | L |
| GP signal peptide | No second ORF |
| GP signal peptide and NP | A heterologous non-arenaviral |
| | signal peptide, arenavirus GP1, |
| | and arenavirus GP2 |
| GP signal peptide and NP | A heterologous non-arenaviral |
| | polypeptide |
| GP signal peptide and NP | Z |
| GP signal peptide and NP | L |
| GP signal peptide and NP | No second ORF |
| GP signal peptide and Z | A heterologous non-arenaviral |
| | signal peptide, arenavirus GP1, |
| | and arenavirus GP2 |
| GP signal peptide and Z | A heterologous non-arenaviral |
| | polypeptide |
| GP signal peptide and Z | NP |
| GP signal peptide and Z | L |
| GP signal peptide and Z | No second ORF |
| GP signal peptide and L | A heterologous non-arenaviral |
| | signal peptide, arenavirus GP1, |
| | and arenavirus GP2 |
| GP signal peptide and L | A heterologous non- |
| | arenaviral polypeptide |
| GP signal peptide and L | NP |
| GP signal peptide and L | Z |
| GP signal peptide and L | No second ORF |
| GP1, GP2 and a heterologous non- | GP signal peptide and a |
| arenaviral signal peptide | heterologous |
| | non-arenaviral polypeptide |
| GP1, GP2 and a heterologous non- | GP signal peptide |
| arenaviral signal peptide | |
| GP1, GP2 and a heterologous non- | A heterologous non- |
| arenaviral signal peptide | arenaviral polypeptide |
| GP1, GP2 and a heterologous non- | NP |
| arenaviral signal peptide | |
| GP1, GP2 and a heterologous non- | Z |
| arenaviral signal peptide | |
| GP1, GP2 and a heterologous non- | L |
| arenaviral signal peptide | |
| GP1, GP2 and a heterologous non- | No second ORF |
| arenaviral signal peptide | |

### 5.4.1 Arenavirus Genomic or Antigenomic Segment Related to the Nucleotide Sequence Provided Herein

In certain embodiments, the nucleotide sequence provided herein is an arenavirus genomic or antigenomic segment. In certain embodiments, the nucleotide sequence is an arenavirus genomic or antigenomic S segment. In certain embodiments, the nucleotide sequence is an arenavirus genomic or antigenomic L segment. In certain embodiments, the nucleotide sequence provided herein can be derived from an arenavirus genomic or antigenomic segment.

In certain embodiments, the first ORF and the second ORF of the nucleotide sequence as described herein are in an S segment or derived from an S segment and separated by an arenavirus intergenic region (IGR). In certain embodiments, the first ORF and the second ORF of the nucleotide sequence as described herein are in an L segment or derived from an L segment and separated by an arenavirus intergenic region (IGR). In certain embodiments, the first ORF in an S segment is under control of an arenavirus 3′ untranslated region (UTR) whereas the second ORF in the S segment is under control of an arenavirus 5′ UTR. In certain embodiments, the first ORF in an L segment is under control of an arenavirus 3′ untranslated region (UTR) whereas the second ORF in the L segment is under control of an arenavirus 5′ UTR. In certain embodiments, the first ORF in an S segment is under control of an arenavirus 5′ UTR whereas the second ORF in the S segment is under control of an arenavirus 3′ UTR. In certain embodiments, the first ORF in an L segment is under control of an arenavirus 5′ UTR whereas the second ORF in the L segment is under control of an arenavirus 3′ UTR.

More details on the arenavirus genomic or antigenomic segment provided herein are described in Section 5.6.

### 5.4.2 Arenavirus Particles Comprising the Nucleotide Sequence Provided Herein

In certain embodiments, provided herein is an arenavirus particle containing a genome comprising the nucleotide sequence provided herein. In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

In certain embodiments, the arenavirus particle is infectious and replication competent. In certain embodiments, the arenavirus particle is attenuated as compared to its parental virus. In certain embodiments, the arenavirus particle is infectious but unable to produce further infectious progeny in non-complementing cells.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide than another arenavirus particle expressing the same heterologous non-arenaviral polypeptide under control of an arenavirus 5′ UTR. In some embodiments, an immune response (e.g., a higher immune response) is obtained after an arenavirus particle of the present disclosure (e.g., arenavirus particle expressing a heterologous non-arenaviral polypeptide under control of an arenavirus 3′ UTR) is administered to a subject. In some embodiments, an immune response is obtained after an arenavirus particle expressing the heterologous non-arenaviral polypeptide under control of an arenavirus 5′ UTR is administered to a subject or to a comparable subject. In some embodiments, an immune response obtained after an arenavirus particle of the present disclosure is administered to a subject is compared to an immune response obtained after another arenavirus particle (e.g., arenavirus particle expressing the heterologous non-arenaviral polypeptide under control of an arenavirus 5′ UTR) is administered to a subject or to a comparable subject. In some embodiments, the subject and/or the comparable subject is a subject in need of treatment. In some embodiments, the subject and/or the comparable subject is a subject with a disease and/or with symptoms of a disease. In some embodiments, the comparable subject is a healthy subject. In some embodiments, the comparable subject is a subject without the disease. In some embodiments, the comparable subject is a subject not in need of treatment.

In certain embodiments, provided herein is an arenavirus particle comprising the nucleotide sequence provided herein. In certain embodiments, the arenavirus particle described herein is genetically stable. In certain embodiments, the arenavirus particle described herein provides high-level transgene expression. In certain embodiments, the arenavirus particle is bi-segmented, namely consists of two arenavirus genomic or antigenomic segments. In certain embodiments, the two arenavirus genomic or antigenomic segments are one S segment and one L segment. In certain embodiments, the arenavirus particle is tri-segmented, namely consists of three arenavirus genomic or antigenomic segments. In certain embodiments, the three arenavirus genomic or antigenomic segments are two S segments and one L segment. In other embodiments, the three arenavirus genomic or antigenomic segments are one S segment and two L segments. More details on the arenavirus genomic or antigenomic segment provided herein are described in Section 5.6.

### 5.4.3 Compositions and Methods Related to the Nucleotide Sequence Provided Herein

In certain embodiments, provided herein is a translation product of the nucleotide sequence provided herein.

In certain embodiments, the nucleotide sequence is a DNA sequence, which can be transcribed into an arenavirus genomic or antigenomic segment. In certain embodiments, provided herein is a method of producing an arenavirus genomic or antigenomic RNA segment, wherein the method comprises transcribing the DNA sequence provided herein.

In certain embodiments, provided herein is a method of generating an arenavirus particle, wherein the method comprises:
a) transfecting into a host cell one or more DNA sequences provided herein or one or more RNA sequences each transcribed in vitro from the DNA sequence provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more DNA sequences are transcribed using a bidirectional promoter. In certain embodiments, the one or more DNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising the nucleotide sequence provided herein.

In certain embodiments, provided herein is a method of rescuing an arenavirus particle using the nucleotide sequence provided herein.

In certain embodiments, provided herein is a host cell comprising the nucleotide sequence provided herein, the translation product provided herein, the arenavirus particle provided herein, or the DNA expression vector provided herein.

In certain embodiments, provided herein is a vaccine comprising the nucleotide sequence provided herein, the translation product provided herein, the arenavirus particle provided herein, and a pharmaceutically acceptable carrier.

In certain embodiments, provided herein is a pharmaceutical composition comprising the nucleotide sequence provided herein, the translation product provided herein, the arenavirus particle provided herein, and a pharmaceutically acceptable carrier.

### 5.5 Arenavirus Particle Provided Herein

In another aspect, provided herein are arenavirus particles. In certain embodiments, provided herein is an arenavirus particle engineered such that an arenaviral ORF is separated over two or more mRNA transcripts.

In certain embodiments, at least one of the mRNA transcripts comprises an internal ribosome entry site (IRES).

In certain embodiments, the mRNA transcripts can be transcribed from the arenavirus genomic or antigenomic segment. In certain embodiments, the arenavirus genomic or antigenomic segment is an S segment. In certain embodiments, the arenavirus genomic or antigenomic segment is an L segment.

In certain embodiments, the two or more mRNA transcripts are under control of an arenavirus 3′ UTR or an arenavirus 5′ UTR.

In certain embodiments, the arenaviral ORF encodes a polypeptide comprising an amino acid sequence that is at least 80%, 85%, 90%, 95%, 96%, 97%, 98%, 99% and 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134.

In certain embodiments, the arenaviral ORF encodes arenavirus GP signal peptide, arenavirus GP1 and GP2 and the arenavirus GP signal peptide or a functional fragment thereof is expressed from a first mRNA transcript (e.g., viral mRNA transcript) and arenavirus GP1 and GP2 are expressed from a second mRNA transcript (e.g., viral mRNA transcript).

In certain embodiments, the first mRNA transcript is under control of an arenavirus 3′ UTR.

In certain embodiments, the second mRNA transcript further encodes a heterologous non-arenaviral signal peptide.

In certain embodiments, the heterologous non-arenaviral signal peptide is the signal peptide of the vesicular stomatitis virus serotype Indiana glycoprotein.

In certain embodiments, the first mRNA transcript further comprises a nucleotide sequence encoding a heterologous non-arenaviral polypeptide or arenavirus GP, NP, Z and L.

In certain embodiments, the heterologous non-arenaviral polypeptide is an antigen derived from an infectious organism, tumor, or allergen. In certain embodiments, the antigen is selected from the group consisting of

(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-DI, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

In certain embodiments, the expression level of the heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express a heterologous non-arenaviral polypeptide after an arenavirus particle of the present disclosure (e.g., an arenavirus expressing a heterologous non-arenaviral polypeptide under control of an arenavirus 3′ UTR) is introduced to (or infects) the cells (e.g., to a population of cells). In certain embodiments, the proportion of cells that express a heterologous non-arenaviral polypeptide after an arenavirus particle of the present disclosure (e.g., expressing a heterologous non-arenaviral polypeptide under control of an arenavirus 3′ UTR) is introduced to (or infects) a population of cells is higher compared to the proportion of cells that express the heterologous non-arenaviral polypeptide after an arenavirus particle expressing the same heterologous non-arenaviral polypeptide expressed under control of an arenavirus 5′ UTR is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express a heterologous non-arenaviral polypeptide after an arenavirus particle of the present disclosure (e.g., expressing a heterologous non-arenaviral polypeptide under control of an arenavirus 3′ UTR) is introduced to (or infects) a population of cells as compared to the proportion of cells that express the heterologous non-arenaviral polypeptides after an arenavirus particle expressing the same heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the expression level of a heterologous non-arenaviral polypeptide expressed under control of an arenavirus 3′ UTR and/or the expression level of a second heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR and/or higher than the expression level of the same second heterologous non-arenaviral polypeptide expressed under control of the respective arenavirus 5′ UTR. In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express the heterologous non-arenaviral polypeptide under control of an arenavirus 3′ UTR and/or express the second heterologous non-arenaviral polypeptide under control of an arenavirus 3′ UTR after an arenavirus particle of the present disclosure (e.g., an arenavirus expressing the heterologous non-arenaviral polypeptide under control of an arenavirus 3′ UTR and/or an arenavirus expressing the second heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptide and/or express the second heterologous non-arenaviral polypeptide after an arenavirus particle of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells is higher compared to the proportion of cells that express the heterologous non-arenaviral polypeptide and/or express the second heterologous non-arenaviral polypeptide after an arenavirus particle expressing the same heterologous non-arenaviral polypeptide under control of an arenavirus 5′ UTR and/or expressing the same second heterologous non-arenaviral polypeptide under control of an arenavirus 5′ UTR is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptide and/or express the second heterologous non-arenaviral polypeptide after an arenavirus particle of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells as compared to the proportion (or amount) of cells that express the heterologous non-arenaviral polypeptides and/or express the second heterologous non-arenaviral polypeptides after an arenavirus particle expressing the same heterologous non-arenaviral polypeptide under control of an arenavirus 5′ UTR and/or expressing the same second heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the arenavirus particle expresses two heterologous non-arenaviral polypeptides. In certain embodiments, the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment. In certain embodiments, the expression of a first of the two heterologous non-arenaviral polypeptides is under control of an arenavirus 5′ UTR in a first S segment and the expression of a second of the two heterologous non-arenaviral polypeptides is under control of an arenavirus 3′ UTR in a second S segment. In certain embodiments, the combined expression level of the two heterologous non-arenaviral polypeptides is at least about, or about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptide expressed under control of arenavirus 5′ UTRs. In certain embodiments, the expression of a first of the same two heterologous non-arenaviral polypeptides is under control of an arenavirus 5′ UTR in a first S segment and the expression of a second of the same two heterologous non-arenaviral polypeptides is under control of an arenavirus 5′ UTR in a second S segment.

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the heterologous non-arenaviral polypeptide is expressed under control of an arenavirus 3′ UTR and the second heterologous non-arenaviral polypeptide is expressed under control of an arenavirus 5′ UTR. In certain embodiments, the heterologous non-arenaviral polypeptide is expressed under control of an arenavirus 3′ UTR in one S segment and the second heterologous non-arenaviral polypeptide is expressed under control of an arenavirus 5′ UTR in another S segment. In certain embodiments, the heterologous non-arenaviral polypeptide is expressed under control of an arenavirus 5′ UTR in one S segment and the second heterologous non-arenaviral polypeptide is expressed under control of an arenavirus 3′ UTR in another S segment. In certain embodiments, the proportion of cells that express both the heterologous non-arenaviral polypeptide and the second heterologous non-arenaviral polypeptide (e.g., cells that co-express the heterologous non-arenaviral polypeptide and the second heterologous non-arenaviral polypeptide) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells that express both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR in one S segment and the same second heterologous non-arenaviral polypeptide is expressed under control of an arenavirus 5′ UTR in another S segment). In some embodiments, the proportion of cells is determined after a population of cells are infected with an arenavirus of the present disclosure (e.g., one heterologous non-arenaviral polypeptide is expressed under control of an arenavirus 3′ UTR and the other heterologous non-arenaviral polypeptide is expressed under control of an arenavirus 5′ UTR). In some embodiments, the proportion of cells is determined after a population of cells are infected with another arenavirus (e.g., both heterologous non-arenaviral polypeptides expressed under control of an arenavirus 5′ UTR).

In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) the cells (e.g., to a population of cells). In certain embodiments, the proportion of cells that express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells is higher as compared to the proportion of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells as compared to the proportion (or amount) of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a comparable population of cells.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

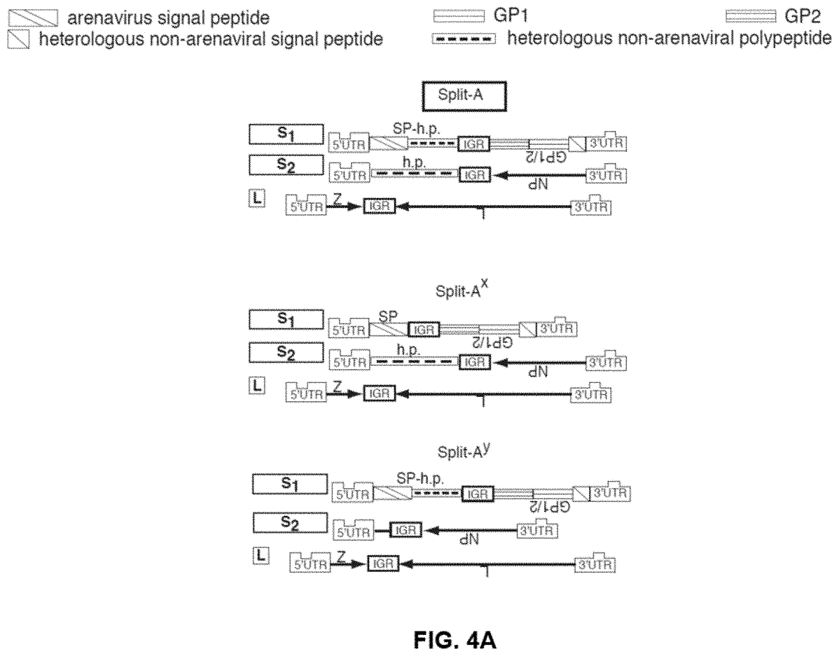

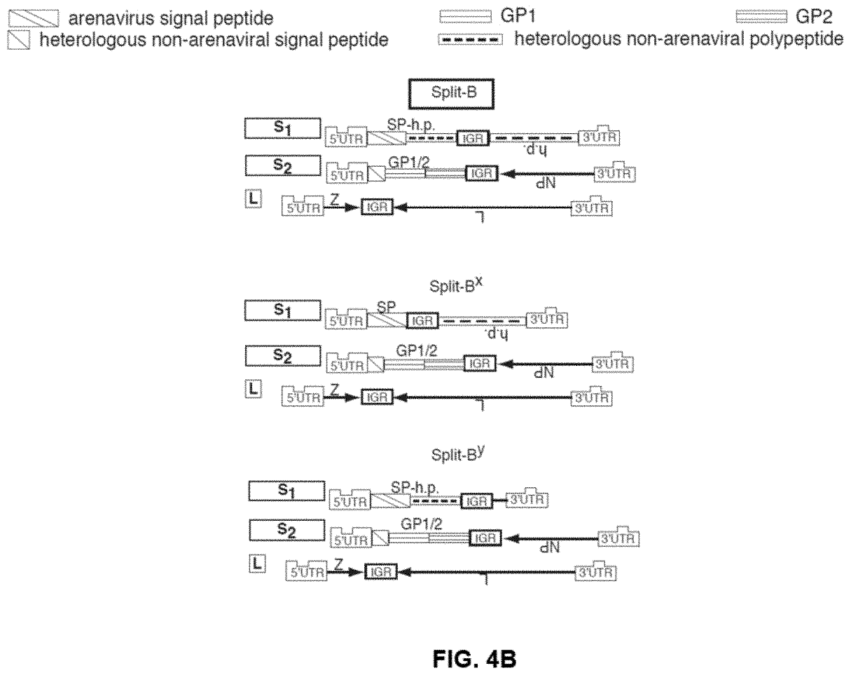

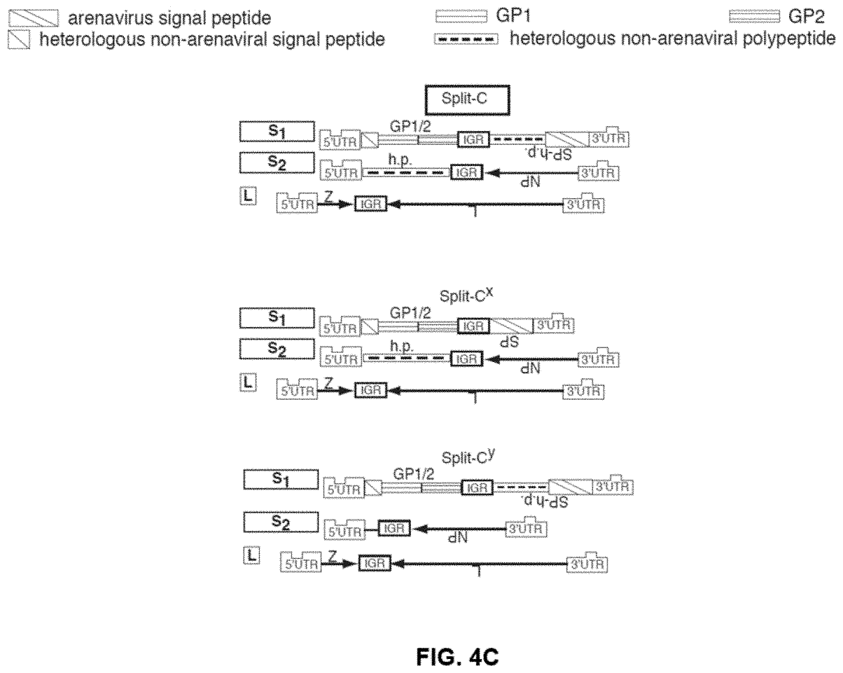

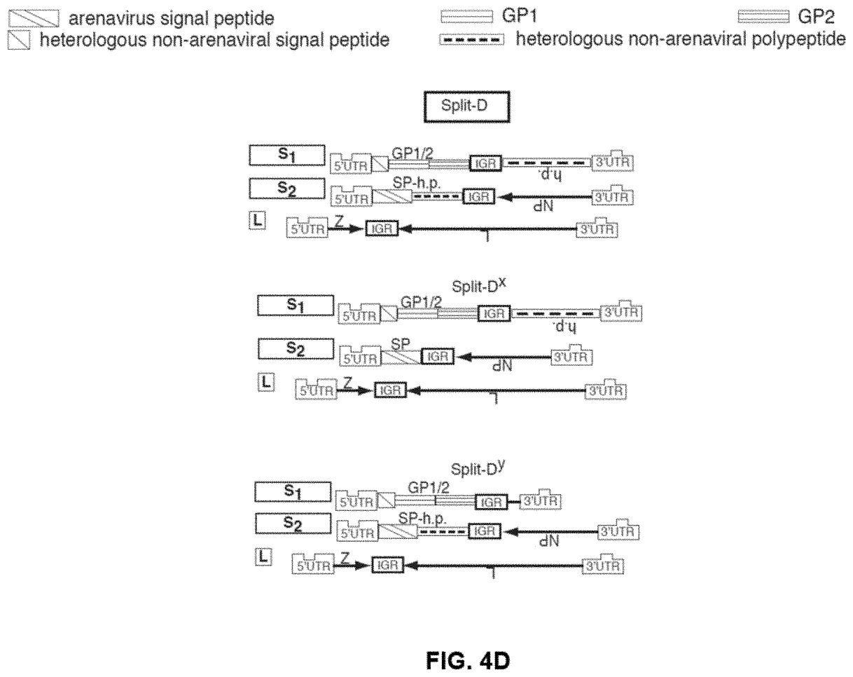

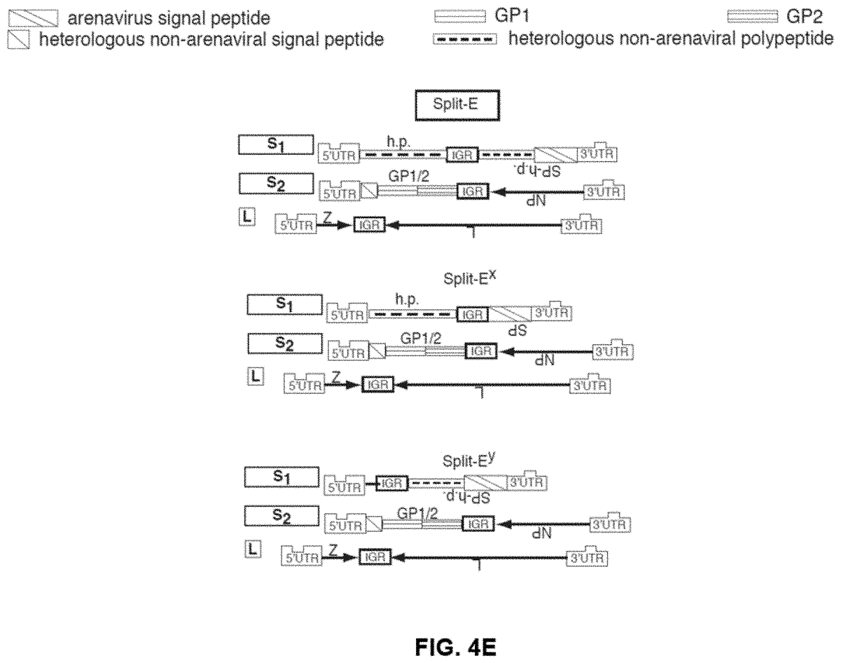

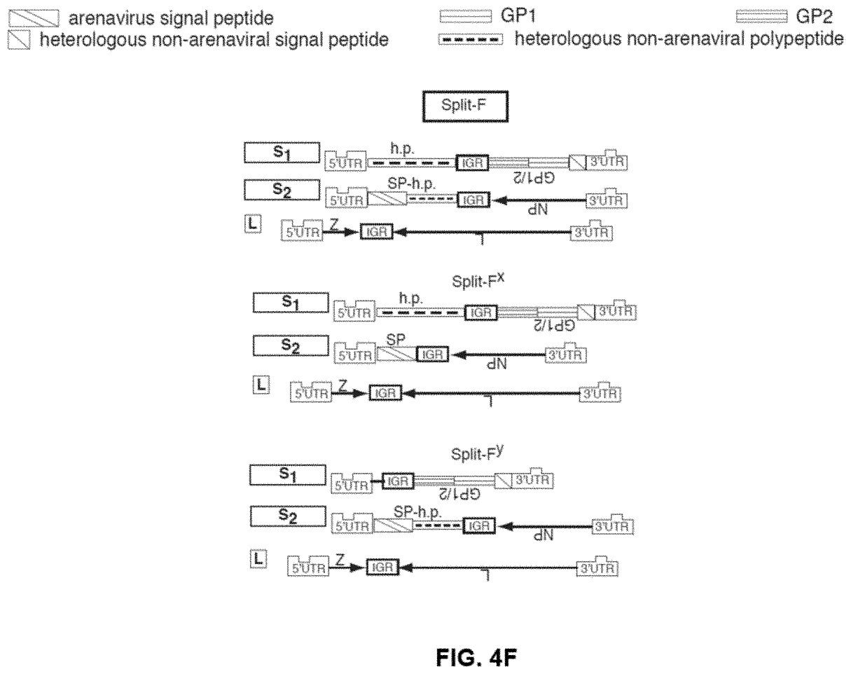

In certain embodiments, the arenavirus particle comprises a genome organization as outlined in FIG. 4C. In certain embodiments, the arenavirus particle comprises a genome organization as outlined in FIG. 4E.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the arenavirus particle is infectious and replication competent. In certain embodiments, the arenavirus particle is attenuated as compared to its parental wild-type virus. In certain embodiments, the arenavirus particle is infectious but unable to produce further infectious progeny in non-complementing cells.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide than another arenavirus particle expressing the same heterologous non-arenaviral polypeptide under control of an arenavirus 5′ UTR. In some embodiments, an immune response (e.g., a higher immune response) is obtained after an arenavirus particle of the present disclosure (e.g., arenavirus particle expressing a heterologous non-arenaviral polypeptide under control of an arenavirus 3′ UTR) is administered to a subject. In some embodiments, an immune response is obtained after an arenavirus particle expressing the heterologous non-arenaviral polypeptide under control of an arenavirus 5′ UTR is administered to a subject or to a comparable subject. In some embodiments, an immune response obtained after an arenavirus particle of the present disclosure is administered to a subject is compared to an immune response obtained after another arenavirus particle (e.g., arenavirus particle expressing the heterologous non-arenaviral polypeptide under control of an arenavirus 5′ UTR) is administered to a subject or to a comparable subject. In some embodiments, the subject and/or the comparable subject is a subject in need of treatment. In some embodiments, the subject and/or the comparable subject is a subject with a disease and/or with symptoms of a disease. In some embodiments, the comparable subject is a healthy subject. In some embodiments, the comparable subject is a subject without the disease. In some embodiments, the comparable subject is a subject not in need of treatment.

In certain embodiments, provided herein is an arenavirus particle engineered such that an arenaviral ORF is separated over two or more mRNA transcripts. In certain embodiments, provided herein is an arenavirus particle comprising the nucleotide sequence provided herein (see Section 5.4). In certain embodiments, provided herein is an arenavirus particle comprising the arenavirus genomic or antigenomic segment provided herein (see Section 5.6). The arenavirus particles described herein are genetically stable and provide high-level transgene expression.

In certain embodiments, the arenavirus particle is tri-segmented, namely consists of three arenavirus genomic or antigenomic segments. In certain embodiments, the three arenavirus genomic or antigenomic segments are two S segments and one L segment. In other embodiments, the three arenavirus genomic or antigenomic segments are one S segment and two L segments. In certain embodiments, the tri-segmented arenavirus particle comprises arenaviral ORFs encoding arenavirus GP, NP, Z and L. In certain embodiments, the tri-segmented arenavirus particle expresses one or more heterologous non-arenaviral polypeptide. In certain embodiments, the tri-segmented arenavirus particle described herein may consist of six positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus S segment 5′ UTR; position 4 is under the control of an arenavirus S segment 3′ UTR; position 5 is under the control of an arenavirus L segment 5′ UTR; position 6 is under the control of an arenavirus L segment 3′ UTR. In certain embodiments, positions 1-6 may encode the same polypeptide in two or more positions. In other embodiments, positions 1-6 may each encodes a different polypeptide. In certain embodiments, the tri-segmented arenavirus particle comprising positions 1-6 in the arenavirus genomic or antigenomic segments is replication competent. In other embodiments, the tri-segmented arenavirus particle comprising positions 1-6 in the arenavirus genomic or antigenomic segments is replication-defective.

In certain embodiments, the arenavirus particle is bi-segmented, namely consists of two arenavirus genomic or antigenomic segments. In certain embodiments, the two arenavirus genomic or antigenomic segments are one S segment and one L segment. In certain embodiments, the genome of a bi-segmented arenavirus particle may consist of four positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus L segment 5′ UTR; position 4 is under the control of an arenavirus L segment 3′ UTR. In certain embodiments, positions 1-4 may encode the same polypeptide in two or more positions. In other embodiments, positions 1-4 may each encode a different polypeptide. In certain embodiments, the bi-segmented arenavirus particle comprising positions 1-4 in the arenavirus genomic or antigenomic segments is replication competent. In other embodiments, the bi-segmented arenavirus particle comprising positions 1-4 in the arenavirus genomic or antigenomic segments is replication-defective.

In certain embodiments, the arenavirus particle is derived from a Lymphocytic choriomeningitis virus (LCMV). In certain embodiments, the LCMV is a MP strain, an Armstrong strain, or an Armstrong Clone 13 strain. In other embodiments, the arenavirus particle is derived from a Lassa virus. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the growth or infectivity of the arenavirus particle comprising the nucleotide sequence described herein is not inferior to a second arenavirus particle carrying one or more of the same heterologous non-arenaviral polypeptides, and all arenaviral GP, NP, Z and L in the second arenavirus particle are each expressed as one ORF.

### 5.5.1 Arenaviral Open Reading Frame

In certain embodiments, the arenaviral ORF encodes a polypeptide selected from the group consisting of arenavirus GP, NP, Z, and L (see Section 5.1).

In certain embodiments, the arenaviral ORF encodes a polypeptide selected from the group consisting of arenavirus GP, NP, Z, and L, namely from the group consisting of arenavirus glycoprotein, nucleoprotein, Z protein, L protein, glycoprotein precursor, nucleoprotein precursor, Z protein precursor, and L protein precursor. In certain embodiments, the arenavirus GP, NP, Z, and L are wild-type. In other embodiments, the arenavirus GP, NP, Z, and L are recombinant. In certain embodiments, the arenavirus GP, NP, Z, and L are mutated. In certain embodiments, the arenavirus GP, NP, Z, and L are derived from an attenuated virus.

In certain embodiments, the arenaviral ORF encodes a polypeptide selected from the group consisting of wild-type arenavirus glycoprotein, nucleoprotein, Z protein, L protein, glycoprotein precursor, nucleoprotein precursor, Z protein precursor, and L protein precursor. In certain embodiments, the arenaviral ORF encodes a wild-type arenavirus glycoprotein precursor. In certain embodiments, the wild-type arenavirus glycoprotein precursor can be processed into a GP signal peptide, a GP1 and a GP2.

In certain embodiments, the arenaviral ORF encodes a polypeptide selected from the group consisting of recombinant arenavirus glycoprotein, nucleoprotein, Z protein, L protein, glycoprotein precursor, nucleoprotein precursor, Z protein precursor, and L protein precursor. In certain embodiments, the arenaviral ORF encodes a recombinant arenavirus glycoprotein precursor. In certain embodiments, the recombinant arenavirus glycoprotein precursor can be processed into a GP signal peptide, a GP1 and a GP2, one or more of which are recombinant.

In certain embodiments, the arenaviral ORF encodes a polypeptide selected from the group consisting of mutated arenavirus glycoprotein, nucleoprotein, Z protein, L protein, glycoprotein precursor, nucleoprotein precursor, Z protein precursor, and L protein precursor. In certain embodiments, the arenaviral ORF encodes a mutated arenavirus glycoprotein precursor. In certain embodiments, the mutated arenavirus glycoprotein precursor can be processed into a GP signal peptide, a GP1 and a GP2, one or more of which are mutated.

In certain embodiments, the arenaviral ORF encodes a polypeptide selected from the group consisting of arenavirus glycoprotein, nucleoprotein, Z protein, L protein, glycoprotein precursor, nucleoprotein precursor, Z protein precursor, and L protein precursor that are derived from an attenuated virus. In certain embodiments, the arenaviral ORF encodes arenavirus glycoprotein precursor derived from an attenuated virus. In certain embodiments, the arenavirus glycoprotein precursor derived from an attenuated virus can be processed into a GP signal peptide, a GP1 and a GP2, one or more of which are derived from an attenuated virus.

In certain embodiments, the arenaviral ORF encodes a polypeptide selected from the group consisting of GP, NP, Z and L of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the arenaviral ORF encodes a polypeptide comprising an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

In certain embodiments, the arenaviral ORF encodes arenavirus GP, namely arenavirus glycoprotein or any glycoprotein precursor. In certain embodiments, the arenaviral ORF encodes a wild-type arenavirus glycoprotein or any wild-type glycoprotein precursor. In other embodiments, the arenaviral ORF encodes a recombinant arenavirus glycoprotein or any recombinant glycoprotein precursor. In certain embodiments, the arenavirus glycoprotein precursor can be processed into a GP signal peptide, a GP1 and a GP2. In certain embodiments, the arenavirus GP is arenavirus glycoprotein or any glycoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus GP as described herein comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the GP as described herein comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the GP as described herein comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the arenaviral ORF encodes arenavirus NP, namely arenavirus nucleoprotein or any nucleoprotein precursor. In certain embodiments, the arenaviral ORF encodes a wild-type arenavirus nucleoprotein or any wild-type nucleoprotein precursor. In other embodiments, the arenaviral ORF encodes a recombinant arenavirus nucleoprotein or any recombinant nucleoprotein precursor. In certain embodiments, the arenavirus NP is arenavirus nucleoprotein or any nucleoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus NP as described herein comprises an amino acid sequence that is at least 80%, 85%, 90%, 95%, 96%, 97%, 98%, 99% or 100% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 80% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 85% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 90% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 95% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 99% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 100% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 80% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 85% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 90% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 95% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 99% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138.

In certain embodiments, the arenaviral ORF encodes arenavirus Z, namely arenavirus Z protein or any Z protein precursor. In certain embodiments, the polypeptide described herein is a wild-type arenavirus Z protein or any wild-type Z protein precursor. In certain embodiments, the arenaviral ORF encodes a recombinant arenavirus Z protein or any recombinant Z protein precursor. In certain embodiments, the arenavirus Z is arenavirus Z protein or any Z protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus Z as described herein comprises an amino acid sequence that is at least 80%, 85%, 90%, 95%, 96%, 97%, 98%, 99% or 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, or SEQ ID NO:139.

In certain embodiments, the arenaviral ORF encodes arenavirus L, namely arenavirus L protein or any L protein precursor. In certain embodiments, the arenaviral ORF encodes a wild-type arenavirus L protein or any wild-type L protein precursor. In certain embodiments, the arenaviral ORF encodes a recombinant arenavirus L protein or any recombinant L protein precursor. In certain embodiments, the arenavirus L is arenavirus L protein or any L protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus L as described herein comprises an amino acid sequence that is at least 80%, 85%, 90%, 95%, 96%, 97%, 98%, 99% or 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

### 5.5.2 Arenavirus Genomic or Antigenomic Segments of the Arenavirus Particle Provided Herein

In certain embodiments, the arenavirus particle is bi-segmented, namely consists of two arenavirus genomic or antigenomic segments. In certain embodiments, the two arenavirus genomic or antigenomic segments are one S segment and one L segment. In certain embodiments, the arenavirus particle is tri-segmented, namely consists of three arenavirus genomic or antigenomic segments. In certain embodiments, the three arenavirus genomic or antigenomic segments are two S segments and one L segment. In other embodiments, the three arenavirus genomic or antigenomic segments are one S segment and two L segments.

In certain embodiments, the genome of the arenavirus particle may consist of a) an S segment that encodes a heterologous non-arenaviral signal peptide, arenavirus GP1 and arenavirus GP2 under the control of 3′ UTR, and arenavirus GP signal peptide and a heterologous non-arenaviral polypeptide or arenavirus GP signal peptide alone under the control of 5′ UTR; b) an S segment that encodes NP under the control of 3′ UTR, and another heterologous non-arenaviral polypeptide or no polypeptide under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 4A.

In certain embodiments, the genome of the arenavirus particle may consist of a) an S segment that encodes a heterologous non-arenaviral polypeptide or no polypeptide under the control of 3′ UTR, and arenavirus GP signal peptide and a heterologous non-arenaviral polypeptide or arenavirus GP signal peptide alone under the control of 5′ UTR; b) an S segment that encodes NP under the control of 3′ UTR, and a heterologous non-arenaviral signal peptide, arenavirus GP1 and arenavirus GP2 under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 4B.

In certain embodiments, the genome of the arenavirus particle may consist of a) an S segment that encodes arenavirus GP signal peptide and a heterologous non-arenaviral polypeptide or an arenavirus GP signal peptide alone under the control of 3′ UTR, and a heterologous non-arenaviral signal peptide, arenavirus GP1 and arenavirus GP2 under the control of 5′ UTR; b) an S segment that encodes NP under the control of 3′ UTR, and another heterologous non-arenaviral polypeptide or no polypeptide under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 4C.

In certain embodiments, the genome of the arenavirus particle may consist of a) an S segment that encodes a heterologous non-arenaviral polypeptide or no polypeptide under the control of 3′ UTR, and a heterologous non-arenaviral signal peptide, arenavirus GP1 and arenavirus GP2 under the control of 5′ UTR; b) an S segment that encodes NP under the control of 3′ UTR, and arenavirus GP signal peptide and a heterologous non-arenaviral polypeptide or arenavirus GP signal peptide alone under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 4D.

In certain embodiments, the genome of the arenavirus particle may consist of a) an S segment that encodes arenavirus GP signal peptide and a heterologous non-arenaviral polypeptide or arenavirus GP signal peptide alone under the control of 3′ UTR, and another heterologous non-arenaviral polypeptide or no polypeptide under the control of 5′ UTR; b) an S segment that encodes NP under the control of 3′ UTR, and a heterologous non-arenaviral signal peptide, arenavirus GP1 and arenavirus GP2 under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 4E.

In certain embodiments, the genome of the arenavirus particle may consist of a) an S segment that encodes a heterologous non-arenaviral signal peptide, arenavirus GP1 and arenavirus GP2 under the control of 3′ UTR, and a heterologous non-arenaviral polypeptide or no polypeptide under the control of 5′ UTR; b) an S segment that encodes NP under the control of 3′ UTR, and arenavirus GP signal peptide and a heterologous non-arenaviral polypeptide or arenavirus GP signal peptide alone under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 4F.

In certain embodiments, the arenavirus particle comprising the nucleotide sequence described herein is infectious and replication competent. In certain embodiments, the arenavirus particle comprising the nucleotide sequence described herein is attenuated as compared to its parental virus. In certain embodiments, the arenavirus particle comprising the nucleotide sequence described herein is infectious but unable to produce further infectious progeny in non-complementing cells.

In certain embodiments, the genome of a bi-segmented arenavirus particle may consist of four positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus L segment 5′ UTR; position 4 is under the control of an arenavirus L segment 3′ UTR. In certain embodiments, positions 1-4 may encode the same polypeptide in two or more positions. In other embodiments, positions 1-4 may each encode a different polypeptide. In certain embodiments, the bi-segmented arenavirus particle comprising positions 1-4 in the arenavirus genomic or antigenomic segments is replication competent. In other embodiments, the bi-segmented arenavirus particle comprising positions 1-4 in the arenavirus genomic or antigenomic segments is replication-defective.

In certain embodiments, the genome of a tri-segmented arenavirus particle may consist of six positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus S segment 5′ UTR; position 4 under the control of an arenavirus S segment 3′ UTR; position 5 is under the control of an arenavirus L segment 5′ UTR; position 6 is under the control of an arenavirus L segment 3′ UTR. In certain embodiments, positions 1-6 may encode the same polypeptide in two or more positions. In other embodiments, positions 1-6 may each encode a different polypeptide. In certain embodiments, the tri-segmented arenavirus particle comprising positions 1-6 in the arenavirus genomic or antigenomic segments is replication competent. In other embodiments, the tri-segmented arenavirus particle comprising positions 1-6 in the arenavirus genomic or antigenomic segments is replication-defective.

In certain embodiments, position 1 encodes a heterologous non-arenaviral signal peptide, arenavirus GP1 and GP2. In certain embodiments, position 1 encodes 1) arenavirus GP signal peptide and a heterologous non-arenaviral polypeptide; 2) arenavirus GP signal peptide alone; or 3) arenavirus GP signal peptide and NP, Z or L. In certain embodiments, position 1 encodes a heterologous non-arenaviral polypeptide. In certain embodiments, position 1 encodes NP. In certain embodiments, position 1 encodes Z. In certain embodiments, position 1 encodes L. In certain embodiments, position 1 does not encode a polypeptide.

In certain embodiments, position 2 encodes a heterologous non-arenaviral signal peptide, arenavirus GP1 and GP2. In certain embodiments, position 2 encodes 1) arenavirus GP signal peptide and a heterologous non-arenaviral polypeptide; 2) arenavirus GP signal peptide alone; or 3) arenavirus GP signal peptide and NP, Z or L. In certain embodiments, position 2 encodes a heterologous non-arenaviral polypeptide. In certain embodiments, position 2 encodes NP. In certain embodiments, position 2 encodes Z. In certain embodiments, position 2 encodes L. In certain embodiments, position 2 does not encode a polypeptide.

In certain embodiments, position 3 encodes a heterologous non-arenaviral signal peptide, arenavirus GP1 and GP2. In certain embodiments, position 3 encodes 1) arenavirus GP signal peptide and a heterologous non-arenaviral polypeptide; 2) arenavirus GP signal peptide alone; or 3) arenavirus GP signal peptide and NP, Z or L. In certain embodiments, position 3 encodes a heterologous non-arenaviral polypeptide. In certain embodiments, position 3 encodes NP. In certain embodiments, position 3 encodes Z. In certain embodiments, position 3 encodes L. In certain embodiments, position 3 does not encode a polypeptide.

In certain embodiments, position 4 encodes a heterologous non-arenaviral signal peptide, arenavirus GP1 and GP2. In certain embodiments, position 4 encodes 1) arenavirus GP signal peptide and a heterologous non-arenaviral polypeptide; 2) arenavirus GP signal peptide alone; or 3) arenavirus GP signal peptide and NP, Z or L. In certain embodiments, position 4 encodes a heterologous non-arenaviral polypeptide. In certain embodiments, position 4 encodes NP. In certain embodiments, position 4 encodes Z. In certain embodiments, position 4 encodes L. In certain embodiments, position 4 does not encode a polypeptide.

In certain embodiments, position 5 encodes a heterologous non-arenaviral signal peptide, arenavirus GP1 and GP2. In certain embodiments, position 5 encodes 1) arenavirus GP signal peptide and a heterologous non-arenaviral polypeptide; 2) arenavirus GP signal peptide alone; or 3) arenavirus GP signal peptide and NP, Z or L. In certain embodiments, position 5 encodes a heterologous non-arenaviral polypeptide. In certain embodiments, position 5 encodes NP. In certain embodiments, position 5 encodes Z. In certain embodiments, position 5 encodes L. In certain embodiments, position 5 does not encode a polypeptide.

In certain embodiments, position 6 encodes a heterologous non-arenaviral signal peptide, arenavirus GP1 and GP2. In certain embodiments, position 6 encodes 1) arenavirus GP signal peptide and a heterologous non-arenaviral polypeptide; 2) arenavirus GP signal peptide alone; or 3) arenavirus GP signal peptide and NP, Z or L. In certain embodiments, position 6 encodes a heterologous non-arenaviral polypeptide. In certain embodiments, position 6 encodes NP. In certain embodiments, position 6 encodes Z. In certain embodiments, position 6 encodes L. In certain embodiments, position 6 does not encode a polypeptide.

In certain embodiments, the arenavirus particle is derived from a Lymphocytic choriomeningitis virus (LCMV). In certain embodiments, the LCMV is a MP strain, an Armstrong strain, or an Armstrong Clone 13 strain. In other embodiments, the arenavirus particle is derived from a Lassa virus. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from a Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the growth or infectivity of the arenavirus particle described herein is not inferior to a second arenavirus particle carrying one or more of the same heterologous non-arenaviral polypeptides, and all arenaviral GP, NP, Z and L in the second arenavirus particle are each expressed as one ORF.

In certain embodiments, the arenavirus particle comprising the nucleotide sequence described herein is infectious and replication competent. In certain embodiments, the arenavirus particle comprising the nucleotide sequence described herein is attenuated as compared to its parental virus. In certain embodiments, the arenavirus particle comprising the nucleotide sequence described herein is infectious but unable to produce further infectious progeny in non-complementing cells.

### 5.5.3 Compositions and Methods Related to the Nucleotide Sequence Provided Herein

In certain embodiments, provided herein is a translation product of the mRNA transcripts of the genome of the arenavirus particle provided herein.

In certain embodiments, provided herein is a cDNA of the mRNA transcript of the genome of the arenavirus particle provided herein, wherein the cDNA can be transcribed into an arenavirus genomic or antigenomic segment.

In certain embodiments, provided herein is a method of producing an arenavirus genomic or antigenomic segment, wherein the method comprises transcribing the cDNA provided herein.

In certain embodiments, provided herein is a method of generating an arenavirus particle, wherein the method comprises:
a) transfecting into a host cell one or more cDNA of the mRNA transcript of the genome of the arenavirus particle provided herein or one or more RNA sequences each transcribed in vitro from the cDNA of the mRNA transcript of the genome of the arenavirus particle provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more cDNA sequences are transcribed using a bidirectional promoter. In certain embodiments, the one or more cDNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising the DNA sequence encoding the mRNA transcript of the genome of the arenavirus particle provided herein.

In certain embodiments, provided herein is a method of rescuing an arenavirus particle using the mRNA transcript of the genome of the arenavirus particle provided herein or the cDNA sequence thereof.

In certain embodiments, provided herein is a host cell comprising the arenavirus particle provided herein, the translation product provided herein, the cDNA provided herein, or the DNA expression vector provided herein.

In certain embodiments, provided herein is a vaccine comprising the arenavirus particle provided herein, the translation product provided herein, the cDNA provided herein, or the DNA expression vector provided herein, and a pharmaceutically acceptable carrier.

In certain embodiments, provided herein is a pharmaceutical composition comprising the arenavirus particle provided herein, the translation product provided herein, the cDNA provided herein, or the DNA expression vector provided herein, and a pharmaceutically acceptable carrier.

### 5.6 Arenavirus Genomic or Antigenomic Segments Provided Herein

In another aspect, provided herein are arenavirus genomic or antigenomic segments. In certain embodiments, provided herein is an arenavirus genomic or antigenomic segment engineered such that the viral transcription thereof results in a first mRNA transcript and a second mRNA transcript, wherein the first mRNA transcript comprises a nucleotide sequence encoding a functional fragment of a first polypeptide, and the first mRNA transcript does not encode the full-length first polypeptide;

wherein the second mRNA transcript comprises a nucleotide sequence encoding:
a) a second polypeptide; or
b) a functional fragment of the first polypeptide, and the second mRNA transcript does not encode the full-length first polypeptide; or
c) a functional fragment of a second polypeptide, and the second mRNA transcript does not encode the full-length second polypeptide; or
d) a heterologous non-arenaviral polypeptide; and
   wherein the first and second polypeptides are different from each other and selected from the group consisting of arenavirus GP, NP, Z and L.

In certain embodiments, the first mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the second mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, provided herein is an arenavirus genomic or antigenomic segment engineered such that the viral transcription thereof results in an mRNA transcript encoding:
a) a functional fragment of a first polypeptide, and
b) a heterologous non-arenaviral polypeptide or a second polypeptide;
   wherein the mRNA transcript does not encode the full-length first polypeptide; and wherein the first and second polypeptides are different from each other and selected from the group consisting of arenavirus GP, NP, Z and L.

In certain embodiments, the mRNA transcript is a first mRNA transcript and the viral transcription of the arenavirus genomic or antigenomic segment further results in a second mRNA transcript. In certain embodiments, the second mRNA transcript comprises a nucleotide sequence encoding a third polypeptide; a functional fragment of a third polypeptide; a functional fragment of the first polypeptide; or a second heterologous non-arenaviral polypeptide; wherein the third polypeptide is different from the first and the second polypeptide and selected from the group consisting of arenavirus GP, NP, Z and L, wherein the second mRNA transcript does not encode the full-length first polypeptide; wherein the second mRNA transcript does not encode the full-length third polypeptide; and wherein the heterologous non-arenaviral polypeptide and the second heterologous non-arenaviral polypeptide are the same or different from each other.

In certain embodiments, the second mRNA transcript comprises a nucleotide sequence encoding a functional fragment of a first polypeptide, and the functional fragment encoded by the first mRNA transcript is different from the functional fragment encoded by the second mRNA transcript.

In certain embodiments, the viral transcription of the arenavirus genomic or antigenomic segment does not further result in a second mRNA transcript.

In certain embodiments, the mRNA transcript comprises an internal ribosome entry site (IRES).

In certain embodiments, the arenavirus genomic or antigenomic segment is an S segment. In certain embodiments, the arenavirus genomic or antigenomic segment is an L segment.

In certain embodiments, the functional fragment is under control of an arenavirus 3′ UTR. In certain embodiments, the functional fragment is under control of an arenavirus 5′ UTR.

In certain embodiments, the first, second and third polypeptides each comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, or SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

In certain embodiments, the first polypeptide comprises an amino acid sequence identical to SEQ ID NO:1, SEQ ID NO:2, SEQ ID NO:3, or SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134.

In certain embodiments, the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137.

In certain embodiments, the functional fragment is an arenavirus GP signal peptide or a functional fragment thereof.

In certain embodiments, the heterologous non-arenaviral polypeptide, the second heterologous non-arenaviral polypeptide, or both heterologous non-arenaviral polypeptides are each an antigen derived from an infectious organism, tumor, or allergen. In certain embodiments, the antigen is selected from the group consisting of

(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

In certain embodiments, the first mRNA transcript comprises a nucleotide sequence encoding arenavirus GP signal peptide, either alone or fused to a heterologous non-arenaviral polypeptide. In certain embodiments, the second mRNA transcript comprises a nucleotide sequence encoding a heterologous non-arenaviral signal peptide and the arenavirus GP1 and GP2. In certain embodiments, the first mRNA transcript is under control of an arenavirus 3′ UTR and the second mRNA transcript is under control of an arenavirus 5′ UTR.

In certain embodiments, the first mRNA transcript comprises a nucleotide sequence encoding arenavirus GP signal peptide, either alone or fused to a heterologous non-arenaviral polypeptide. In certain embodiments, the second mRNA transcript comprises a nucleotide sequence encoding a heterologous non-arenaviral polypeptide; wherein the heterologous non-arenaviral polypeptide encoded by the first mRNA transcript and the heterologous non-arenaviral polypeptide encoded by the second mRNA transcript are the same or different from each other. In certain embodiments, the first mRNA transcript is under control of an arenavirus 3′ UTR and the second mRNA transcript is under control of an arenavirus 5′ UTR.

In certain embodiments, the first mRNA transcript comprises a nucleotide sequence encoding a heterologous non-arenaviral signal peptide and the arenavirus GP1 and GP2. In certain embodiments, the second mRNA transcript comprises a nucleotide sequence encoding NP. In certain embodiments, the first mRNA transcript is under control of an arenavirus 5′ UTR and the second mRNA transcript is under control of an arenavirus 3′ UTR.

In certain embodiments, the heterologous non-arenaviral polypeptide is expressed under control of an arenavirus 3′ UTR or the second heterologous non-arenaviral polypeptide is expressed under control of an arenavirus 3′ UTR; and the expression level of the heterologous non-arenaviral polypeptide or the expression level of the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or higher than the expression level of the same second heterologous non-arenaviral polypeptide expressed under control of the respective arenavirus 5′ UTR.

In certain embodiments, the expression of one heterologous non-arenaviral polypeptide is under control of an arenavirus 3′ UTR and the expression of the other heterologous non-arenaviral polypeptide is under control of an arenavirus 5′ UTR. In certain embodiments, the expression of one heterologous non-arenaviral polypeptide is under control of an arenavirus 3′ UTR in one S segment and the expression of the other heterologous non-arenaviral polypeptide is under control of an arenavirus 5′ UTR in the other S segment. In certain embodiments, cells that are infected with the arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) express (co-express) both heterologous non-arenaviral polypeptides. In certain embodiments, the combined expression level of the two heterologous non-arenaviral polypeptides (e.g., cells expressing both heterologous non-arenaviral polypeptides) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide is under control of an arenavirus 5′ UTR of one S segment and expression of the other heterologous non-arenaviral polypeptide is under control of an arenavirus 5′ UTR of the other S segment). In certain embodiments, the combined expression level of the two heterologous non-arenaviral polypeptides (e.g., cells expressing both heterologous non-arenaviral polypeptides) is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., one heterologous non-arenaviral polypeptide expressed under control of an arenavirus 5′ UTR of one S segment and the other heterologous non-arenaviral polypeptide expressed under control of an arenavirus 5′ UTR of the other S segment). In certain embodiments, the combined expression level of the two heterologous non-arenaviral polypeptides (e.g., cells expressing both heterologous non-arenaviral polypeptides) is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., one heterologous non-arenaviral polypeptide expressed under control of an arenavirus 5′ UTR of one S segment and the other heterologous non-arenaviral polypeptide expressed under control of an arenavirus 5′ UTR of the other S segment).

In certain embodiments, provided herein is an arenavirus genomic or antigenomic segment. In certain embodiments, the arenavirus genomic or antigenomic segment provided herein is the nucleotide sequence provided herein (see Section 5.4). In certain embodiments, the nucleotide sequence provided herein (see Section 5.4) is derived from the arenavirus genomic or antigenomic segment described in this Section. In certain embodiments, the transcription of the first ORF (see Section 5.4) may result in the first mRNA transcript described in this Section, whereas the transcription of the second ORF (see Section 5.4) may result in the second mRNA transcript described in the Section. In certain embodiments, the arenavirus particles provided herein (see Section 5.5) comprise the arenavirus genomic or antigenomic segment described in this section.

In certain embodiments, the arenavirus genomic or antigenomic segment is an S segment. In certain embodiments, the arenavirus genomic or antigenomic segment is an L segment. In certain embodiments, the functional fragment encoded by the arenavirus genomic or antigenomic segment is under control of a 3′ UTR. In other embodiments, the functional fragment encoded by the arenavirus genomic or antigenomic segment is under control of a 5′ UTR.

In certain embodiments, the first mRNA transcript comprises a nucleotide sequence encoding arenavirus GP signal peptide, either alone or fused to a heterologous non-arenaviral polypeptide, and the second mRNA transcript comprises a nucleotide sequence encoding a heterologous non-arenaviral signal peptide, arenavirus GP1 and arenavirus GP2; and the first mRNA transcript is under control of 5′ UTR and the second mRNA transcript is under control of 3′ UTR. In certain embodiments, the arenavirus genomic or antigenomic segment provided herein is one of the S1 segments depicted in FIG. 4A.

In certain embodiments, the first mRNA transcript comprises a nucleotide sequence encoding arenavirus GP signal peptide, either alone or fused to a heterologous non-arenaviral polypeptide, and the second mRNA transcript comprises a nucleotide sequence encoding a heterologous non-arenaviral polypeptide; and the first mRNA transcript is under control of 5′ UTR and the second mRNA transcript is under control of 3′ UTR. In certain embodiments, the first mRNA transcript comprises a nucleotide sequence encoding arenavirus GP signal peptide, either alone or fused to a heterologous non-arenaviral polypeptide, and there is no second mRNA transcript; and the first mRNA transcript is under control of 5′ UTR. In certain embodiments, the arenavirus genomic or antigenomic segment provided herein is one of the S1 segments depicted in FIG. 4B.

In certain embodiments, the first mRNA transcript comprises a nucleotide sequence encoding a heterologous non-arenaviral signal peptide, arenavirus GP1, and arenavirus GP2, and the second mRNA transcript comprises a nucleotide sequence encoding NP; and the first mRNA transcript is under control of 5′ UTR and the second mRNA transcript is under control of 3′ UTR. In certain embodiments, the arenavirus genomic or antigenomic segment provided herein is one of the S2 segments depicted in FIG. 4B or FIG. 4E.

In certain embodiments, the first mRNA transcript comprises a nucleotide sequence encoding arenavirus GP signal peptide, either alone or fused to a heterologous non-arenaviral polypeptide, and the second mRNA transcript comprises a nucleotide sequence encoding a heterologous non-arenaviral signal peptide, arenavirus GP1 and arenavirus GP2; and the first mRNA transcript is under control of 3′ UTR and the second mRNA transcript is under control of 5′ UTR. In certain embodiments, the arenavirus genomic or antigenomic segment provided herein is one of the S1 segments depicted in FIG. 4C.

In certain embodiments, the first mRNA transcript comprises a nucleotide sequence encoding a heterologous non-arenaviral signal peptide, arenavirus GP1, and arenavirus GP2, and the second mRNA transcript comprises a nucleotide sequence encoding a heterologous non-arenaviral polypeptide; and the first mRNA transcript is under control of 5′ UTR and the second mRNA transcript is under control of 3′ UTR. In certain embodiments, the first mRNA transcript comprises a nucleotide sequence encoding a heterologous non-arenaviral signal peptide, arenavirus GP1, and arenavirus GP2, and there is no second mRNA transcript; and the first mRNA transcript is under control of 5′ UTR. In certain embodiments, the arenavirus genomic or antigenomic segment provided herein is one of the S1 segments depicted in FIG. 4D.

In certain embodiments, the first mRNA transcript comprises a nucleotide sequence encoding arenavirus GP signal peptide, either alone or fused to a heterologous non-arenaviral polypeptide, and the second mRNA transcript comprises a nucleotide sequence encoding NP; and the first mRNA transcript is under control of 5′ UTR and the second mRNA transcript is under control of 3′ UTR. In certain embodiments, the arenavirus genomic or antigenomic segment provided herein is one of the S2 segments depicted in FIG. 4D or FIG. 4F.

In certain embodiments, the first mRNA transcript comprises a nucleotide sequence encoding arenavirus GP signal peptide, either alone or fused to a heterologous non-arenaviral polypeptide, and the second mRNA transcript comprises a nucleotide sequence encoding another heterologous non-arenaviral polypeptide; and the two heterologous non-arenaviral polypeptides are the same or different from each other; and the first mRNA transcript is under control of 3′ UTR and the second mRNA transcript is under control of 5′ UTR. In certain embodiments, the first mRNA transcript comprises a nucleotide sequence encoding arenavirus GP signal peptide fused to a heterologous non-arenaviral polypeptide, and there is no second mRNA transcript; and the first mRNA transcript is under control of 3′ UTR. In certain embodiments, the arenavirus genomic or antigenomic segment provided herein is one of the S1 segments depicted in FIG. 4E.

In certain embodiments, the first mRNA transcript comprises a nucleotide sequence encoding a heterologous non-arenaviral signal peptide, arenavirus GP1, and arenavirus GP2, and the second mRNA transcript comprises a nucleotide sequence encoding a heterologous non-arenaviral polypeptide; and the first mRNA transcript is under control of 3′ UTR and the second mRNA transcript is under control of 5′ UTR. In certain embodiments, the first mRNA transcript comprises a nucleotide sequence encoding a heterologous non-arenaviral signal peptide, arenavirus GP, and arenavirus GP2, and there is no second mRNA transcript; and the first mRNA transcript is under control of 3′ UTR. In certain embodiments, the arenavirus genomic or antigenomic segment provided herein is one of the S1 segments depicted in FIG. 4F.

Non-limiting examples of the polypeptides encoded by the first and second mRNA transcripts are illustrated in Table 3.PG-4T.

TABLE 3
Non-limiting examples of the polypeptides encoded by the first and second
mRNA transcripts.

| | 1^{st} mRNA transcript | | |
|---|---|---|---|
| | Functional fragment | | Functional |
| | of a first polypeptide | | fragment of a first |
| | and a heterologous | | polypeptide and a |
| | non-arenaviral | | third polypeptide |
| | polypeptide (fused | Functional | (fused together or |
| | together or separated | fragment of a first | separated by an |
| 2^{nd} mRNA transcript | by an IRES) | polypeptide | IRES) |
| Functional fragment | Exemplary | Exemplary | Exemplary |
| of a first polypeptide | combination | combination | combination |
| and a heterologous | | | |
| non-arenaviral | | | |
| polypeptide | | | |
| (fused together or | | | |
| separated by an | | | |
| IRES) | | | |
| Functional fragment | Exemplary | Exemplary | Exemplary |
| of a first polypeptide | combination | combination | combination |
| Functional fragment | Exemplary | Exemplary | Exemplary |
| of a first polypeptide | combination | combination | combination |
| and a third | | | |
| polypeptide | | | |
| (fused together or | | | |
| separated by an | | | |
| IRES) | | | |
| A heterologous non- | Exemplary | Exemplary | Exemplary |
| arenaviral | combination | combination | combination |
| polypeptide | | | |
| A second | Exemplary | Exemplary | Exemplary |
| polypeptide | combination | combination | combination |
| Functional fragment | Exemplary | Exemplary | Exemplary |
| of a second | combination | combination | combination |
| polypeptide and a | | | |
| heterologous non- | | | |
| arenaviral | | | |
| polypeptide | | | |
| (fused together or | | | |
| separated by an | | | |
| IRES) | | | |
| Functional fragment | Exemplary | Exemplary | Exemplary |
| of a second | combination | combination | combination |
| polypeptide | | | |
| Functional fragment | Exemplary | Exemplary | Exemplary |
| of a second | combination | combination | combination |
| polypeptide and a | | | |
| third polypeptide | | | |
| (fused together or | | | |
| separated by an | | | |
| IRES) | | | |

In certain embodiments, the functional fragment is selected from the group consisting of arenavirus GP signal peptide, arenavirus GP1 and arenavirus GP2. In certain embodiments, non-limiting examples of the polypeptides encoded by the first and second mRNA transcripts related to functional fragments of GP are illustrated in Table 4.

TABLE 4
Non-limiting examples of the polypeptides encoded by the first and second
mRNA transcripts related to functional fragments of GP.

| First mRNA transcript | Second mRNA transcript |
|---|---|
| GP signal peptide and a heterologous non- | A heterologous non-arenaviral |
| arenaviral polypeptide | signal peptide, arenavirus GP1, |
| | and arenavirus GP2 |
| GP signal peptide and a heterologous non- | A heterologous non-arenaviral |
| arenaviral polypeptide | polypeptide |
| GP signal peptide and a heterologous non- | NP |
| arenaviral polypeptide | |
| GP signal peptide and a heterologous non- | Z |
| arenaviral polypeptide | |
| GP signal peptide and a heterologous non- | L |
| arenaviral polypeptide | |
| GP signal peptide and a heterologous non- | No second mRNA transcript |
| arenaviral polypeptide | |
| GP signal peptide | A heterologous non-arenaviral |
| | signal peptide, arenavirus GP1, |
| | and arenavirus GP2 |
| GP signal peptide | A heterologous non-arenaviral |
| | polypeptide |
| GP signal peptide | NP |
| GP signal peptide | Z |
| GP signal peptide | L |
| GP signal peptide | No second mRNA transcript |
| GP signal peptide and NP | A heterologous non-arenaviral |
| | signal peptide, arenavirus GP1, |
| | and arenavirus GP2 |
| GP signal peptide and NP | A heterologous non-arenaviral |
| | polypeptide |
| GP signal peptide and NP | Z |
| GP signal peptide and NP | L |
| GP signal peptide and NP | No second mRNA transcript |
| GP signal peptide and Z | A heterologous non-arenaviral |
| | signal peptide, arenavirus GP1, |
| | and arenavirus GP2 |
| GP signal peptide and Z | A heterologous non-arenaviral |
| | polypeptide |
| GP signal peptide and Z | NP |
| GP signal peptide and Z | L |
| GP signal peptide and Z | No second mRNA transcript |
| GP signal peptide and L | A heterologous non-arenaviral |
| | signal peptide, arenavirus GP1, |
| | and arenavirus GP2 |
| GP signal peptide and L | A heterologous non-arenaviral |
| | polypeptide |
| GP signal peptide and L | NP |
| GP signal peptide and L | Z |
| GP signal peptide and L | No second mRNA transcript |
| GP1, GP2 and a heterologous non- | GP signal peptide and |
| arenaviral signal peptide | a heterologous non- |
| | arenaviral polypeptide |
| GP1, GP2 and a heterologous non- | GP signal peptide |
| arenaviral signal peptide | |
| GP1, GP2 and a heterologous non- | A heterologous non-arenaviral |
| arenaviral signal peptide | polypeptide |
| GP1, GP2 and a heterologous non- | NP |
| arenaviral signal peptide | |
| GP1, GP2 and a heterologous non- | Z |
| arenaviral signal peptide | |
| GP1, GP2 and a heterologous non- | L |
| arenaviral signal peptide | |
| GP1, GP2 and a heterologous non- | No second mRNA transcript |
| arenaviral signal peptide | |

### 5.6.1 Arenavirus Particle Comprising the Arenavirus Genomic or Antigenomic Segment Provided Herein

In certain embodiments, provided herein is an arenavirus particle comprising the arenavirus genomic or antigenomic segment provided herein.

In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and wherein the two heterologous non-arenaviral polypeptides are the same or different from each other.

In certain embodiments, the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Aporé virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

In certain embodiments, the arenavirus particle is infectious and replication competent. In certain embodiments, the arenavirus particle is attenuated as compared to its parental wild-type virus. In certain embodiments, the arenavirus particle is infectious but unable to produce further infectious progeny in non-complementing cells.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide than another arenavirus particle expressing the same heterologous non-arenaviral polypeptide under control of an arenavirus 5′ UTR. In some embodiments, an immune response (e.g., a higher immune response) is obtained after an arenavirus particle of the present disclosure (e.g., arenavirus particle expressing a heterologous non-arenaviral polypeptide under control of an arenavirus 3′ UTR) is administered to a subject. In some embodiments, an immune response is obtained after an arenavirus particle expressing the heterologous non-arenaviral polypeptide under control of an arenavirus 5′ UTR is administered to a subject or to a comparable subject. In some embodiments, an immune response obtained after an arenavirus particle of the present disclosure is administered to a subject is compared to an immune response obtained after another arenavirus particle (e.g., arenavirus particle expressing the heterologous non-arenaviral polypeptide under control of an arenavirus 5′ UTR) is administered to a subject or to a comparable subject. In some embodiments, the subject and/or the comparable subject is a subject in need of treatment. In some embodiments, the subject and/or the comparable subject is a subject with a disease and/or with symptoms of a disease. In some embodiments, the comparable subject is a healthy subject. In some embodiments, the comparable subject is a subject without the disease. In some embodiments, the comparable subject is a subject not in need of treatment.

In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express the two heterologous non-arenaviral polypeptides after an arenavirus particle of the present disclosure (e.g., expressing one heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptides after an arenavirus particle of the present disclosure (e.g., expressing one heterologous non-arenaviral polypeptide under control of an arenavirus 3′ UTR in one S segment) is introduced to (or infects) a population of cells is higher as compared to the proportion of cells that express the heterologous non-arenaviral polypeptides after an arenavirus particle expressing the same heterologous non-arenaviral polypeptides under control of arenavirus 5′ UTR is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptides after an arenavirus particle of the present disclosure (e.g., expressing one heterologous non-arenaviral polypeptide under control of an arenavirus 3′ UTR in one S segment) is introduced to (or infects) a population of cells as compared to the proportion of cells that express the heterologous non-arenaviral polypeptides after an arenavirus particle expressing the same heterologous non-arenaviral polypeptides under control of arenavirus 5′ UTR is introduced to (or infects) a comparable population of cells.

In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) the cells (e.g., to a population of cells). In certain embodiments, the proportion of cells that express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells is higher as compared to the proportion of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells as compared to the proportion of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a comparable population of cells.

### 5.6.2 Compositions and Methods Related to the Nucleotide Sequence Provided Herein

In certain embodiments, provided herein is a translation product of the arenavirus genomic or antigenomic segment provided herein.

In certain embodiments, provided herein is a cDNA of the arenavirus genomic or antigenomic segment provided herein.

In certain embodiments, provided herein is a method of producing an arenavirus genomic or antigenomic segment, wherein the method comprises transcribing the cDNA provided herein.

In certain embodiments, provided herein is a method of generating an arenavirus particle, wherein the method comprises:
a) transfecting into a host cell one or more cDNA sequences of the arenavirus genomic or antigenomic segment provided herein or one or more RNA sequences each transcribed in vitro from the cDNA sequence of the arenavirus genomic or antigenomic segment provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more cDNA sequences are transcribed using a bidirectional promoter. In certain embodiments, the one or more cDNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising a DNA sequence encoding the arenavirus genomic or antigenomic segment provided herein.

In certain embodiments, provided herein is a method of rescuing an arenavirus particle using the arenavirus genomic or antigenomic segment provided herein or a DNA sequence encoding the arenavirus genomic or antigenomic segment.

In certain embodiments, provided herein is a host cell comprising the arenavirus genomic or antigenomic segment provided herein, the translation product provided herein, the arenavirus particle provided herein, or the DNA expression vector provided herein.

In certain embodiments, provided herein is a vaccine comprising the arenavirus genomic or antigenomic segment provided herein, the translation product provided herein, the arenavirus particle provided herein, or the DNA expression vector provided herein, and a pharmaceutically acceptable carrier.

In certain embodiments, provided herein is a pharmaceutical composition comprising the arenavirus genomic or antigenomic segment provided herein, the translation product provided herein, the arenavirus particle provided herein, or the DNA expression vector provided herein, and a pharmaceutically acceptable carrier.

### 5.7 Translation Product Provided Herein

In certain embodiments, provided herein is a translation product of the nucleotide sequence provided herein (see Section 5.4). In certain embodiments, provided herein is a translation product of the mRNA transcript of the genome of the arenavirus particle provided herein (see Section 5.5). In certain embodiments, provided herein is a translation product of the arenavirus genomic or antigenomic segment provided herein (see Section 5.6).

In certain embodiments, the translation product is a chimeric protein. In certain embodiments, the translation product comprises a functional fragment (see Section 5.2) of a polypeptide provided herein (see Section 5.1) fused to a heterologous non-arenaviral polypeptide (see Section 5.3). In certain embodiments, the translation product comprises a functional fragment (see Section 5.2) of a polypeptide provided herein (see Section 5.1) fused to another polypeptide provided herein (see Section 5.1). In certain embodiments, the translation product comprises a functional fragment (see Section 5.2) of a polypeptide provided herein (see Section 5.1) fused to another functional fragment of the same polypeptide or another polypeptide provided herein (see Section 5.1). In certain embodiments, the translation product comprises a functional fragment (see Section 5.2) of a polypeptide provided herein (see Section 5.1). In certain embodiments, the translation product comprises a heterologous non-arenaviral polypeptide (see Section 5.3). In certain embodiments, the translation product comprises a polypeptide provided herein (see Section 5.1).

In certain embodiments, the translation product provided herein is a translation product of any one of the first or the second ORFs listed in Table 1. In certain embodiments, the translation product provided herein is a translation product of any one of the first or the second ORFs listed in Table 2. In certain embodiments, the translation product provided herein is a translation product of any one of the first or the second mRNA transcripts listed in Table 3. In certain embodiments, the translation product provided herein is a translation product of any one of the first or the second mRNA transcripts listed in Table 4.

In certain embodiments, the translation product provided herein is a translation product of any one of the arenavirus genomic or antigenomic segments depicted in any one of FIGS. 4A-4F.

### 5.8 Vector Systems and Cell Lines

In certain embodiments, provided herein are DNAs comprising or consisting of the nucleotide sequence as described in Section 5.4. In certain embodiments, provided herein is an arenavirus genomic or antigenomic segment as described in Section 5.6. In certain embodiments, provided herein is an arenavirus genomic or antigenomic segment in the arenavirus particle as described in Section 5.5. In certain embodiments, provided herein is a DNA sequence encoding the mRNA transcript in the arenavirus particle as described in Section 5.5. In certain embodiments, the DNA is a cDNA.

In one embodiment, provided herein is a DNA expression vector comprising the nucleotide sequence as described in Section 5.4. In one embodiment, provided herein is a DNA expression vector comprising a cDNA encoding an arenavirus genomic or antigenomic segment as described in Section 5.6. In one embodiment, provided herein is a DNA expression vector comprising a cDNA encoding the arenavirus genomic or antigenomic segment in the arenavirus particle as described in Section 5.5. In certain embodiments, provided herein is a DNA expression vector comprising a cDNA of the mRNA transcript in the arenavirus particle as described in Section 5.5.

In one embodiment, provided herein is a DNA expression vector system that encodes the bi-segmented or tri-segmented arenavirus particle as described herein. Specifically, provided herein is a DNA expression vector system wherein one or more vectors encode two or three arenavirus genomic or antigenomic segments, namely, one L segment and one S section of a bi-segmented arenavirus as described herein, or one L segment and two S segments or two L segments and one S segment of a tri-segmented arenavirus particle described herein. Such a vector system can encode one or more separate DNA molecules.

In another embodiment, provided herein is a DNA or cDNA of the arenavirus S segment(s) as described in Section 5.6, and is part of or incorporated into a DNA expression system. In other embodiments, a DNA or cDNA of the arenavirus L segment(s) as described in Section 5.6 is part of or incorporated into a DNA expression system.

In certain embodiments, the DNA provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In specific embodiments, the cDNA is derived from LCMV Clone 13. In other specific embodiments, the cDNA is derived from LCMV MP strain.

In certain embodiments, the DNA or cDNA provided herein can be derived from a particular strain of Lassa virus. In certain embodiments, the DNA or cDNA provided herein can be derived from a particular strain of Pichinde virus. In certain embodiments, the DNA or cDNA provided herein can be derived from a particular strain of Junin virus. In certain embodiments, the DNA or cDNA provided herein can be derived from a particular strain of Oliveros virus. In certain embodiments, the DNA or cDNA provided herein can be derived from a particular strain of Tamiami virus. In certain embodiments, the DNA or cDNA provided herein can be derived from a particular strain of Mobala virus. In certain embodiments, the DNA or cDNA provided herein can be derived from a particular strain of Mopeia virus. In certain embodiments, the DNA or cDNA provided herein can be derived from a particular strain of Ippy virus. In certain embodiments, the DNA or cDNA provided herein can be derived from a particular strain of Amapari virus. In certain embodiments, the DNA or cDNA provided herein can be derived from a particular strain of Flexal virus. In certain embodiments, the DNA or cDNA provided herein can be derived from a particular strain of Guanarito virus. In certain embodiments, the DNA or cDNA provided herein can be derived from a particular strain of Latino virus. In certain embodiments, the cDNA provided herein can be derived from a particular strain of Machupo virus. In certain embodiments, the DNA or cDNA provided herein can be derived from a particular strain of Parana virus. In certain embodiments, the DNA or cDNA provided herein can be derived from a particular strain of Pirital virus. In certain embodiments, the DNA or cDNA provided herein can be derived from a particular strain of Sabia virus. In certain embodiments, the DNA or cDNA provided herein can be derived from a particular strain of Tacaribe virus. In certain embodiments, the DNA or cDNA provided herein can be derived from a particular strain of Bear Canyon virus. In certain embodiments, the DNA or cDNA provided herein can be derived from a particular strain of Whitewater Arroyo virus. In certain embodiments, the DNA or cDNA provided herein can be derived from a particular strain of Allpahuayo virus (ALLV). In certain embodiments, the DNA or cDNA provided herein can be derived from a particular strain of Alxa virus. In certain embodiments, the DNA or cDNA provided herein can be derived from a particular strain of Chapare virus. In certain embodiments, the DNA or cDNA provided herein can be derived from a particular strain of Lijiang virus. In certain embodiments, the DNA or cDNA provided herein can be derived from a particular strain of Cupixi virus. In certain embodiments, the DNA or cDNA provided herein can be derived from a particular strain of Gairo virus. In certain embodiments, the DNA or cDNA provided herein can be derived from a particular strain of Loei River virus. In certain embodiments, the DNA or cDNA provided herein can be derived from a particular strain of Lujo virus. In certain embodiments, the DNA or cDNA provided herein can be derived from a particular strain of Luna virus. In certain embodiments, the DNA or cDNA provided herein can be derived from a particular strain of Luli virus. In certain embodiments, the DNA or cDNA provided herein can be derived from a particular strain of Lunk virus. In certain embodiments, the DNA or cDNA provided herein can be derived from a particular strain of Mariental virus. In certain embodiments, the DNA or cDNA provided herein can be derived from a particular strain of Merino Walk virus. In certain embodiments, the DNA or cDNA provided herein can be derived from a particular strain of Morogoro virus. In certain embodiments, the DNA or cDNA provided herein can be derived from a particular strain of Okahandja virus. In certain embodiments, the DNA or cDNA provided herein can be derived from a particular strain of Aporé virus. In certain embodiments, the DNA or cDNA provided herein can be derived from a particular strain of Ryukyu virus. In certain embodiments, the DNA or cDNA provided herein can be derived from a particular strain of Solwezi virus. In certain embodiments, the DNA or cDNA provided herein can be derived from a particular strain of souris virus. In certain embodiments, the DNA or cDNA provided herein can be derived from a particular strain of Wenzhou virus. In certain embodiments, the DNA or cDNA provided herein can be derived from a particular strain of Big Brushy Tank virus. In certain embodiments, the DNA or cDNA provided herein can be derived from a particular strain of Catarina virus. In certain embodiments, the DNA or cDNA provided herein can be derived from a particular strain of Skinner Tank virus. In certain embodiments, the DNA or cDNA provided herein can be derived from a particular strain of Tonto Creek virus. In certain embodiments, the DNA or cDNA provided herein can be derived from a particular strain of Xapuri virus.

In certain embodiments, the vector generated to encode an arenavirus particle as described herein may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In certain embodiments, an arenavirus particle or a tri-segmented arenavirus particle as described herein may be based on LCMV Clone 13. In other embodiments, the vector generated to encode an arenavirus particle or a tri-segmented arenavirus particle as described herein may be based on LCMV MP strain.

In certain embodiments, the vector generated to encode an arenavirus particle as described herein may be based on a specific strain of Lassa virus. In certain embodiments, the vector generated to encode an arenavirus particle as described herein may be based on a specific strain of Pichinde virus. In certain embodiments, the vector generated to encode an arenavirus particle as described herein may be based on a specific strain of Junin virus. In certain embodiments, the vector generated to encode an arenavirus particle as described herein may be based on a specific strain of Oliveros virus. In certain embodiments, the vector generated to encode an arenavirus particle as described herein may be based on a specific strain of Tamiami virus. In certain embodiments, the vector generated to encode an arenavirus particle as described herein may be based on a specific strain of Mobala virus. In certain embodiments, the vector generated to encode an arenavirus particle as described herein may be based on a specific strain of Mopeia virus. In certain embodiments, the vector generated to encode an arenavirus particle as described herein may be based on a specific strain of Ippy virus. In certain embodiments, the vector generated to encode an arenavirus particle as described herein may be based on a specific strain of Amapari virus. In certain embodiments, the vector generated to encode an arenavirus particle as described herein may be based on a specific strain of Flexal virus. In certain embodiments, the vector generated to encode an arenavirus particle as described herein may be based on a specific strain of Guanarito virus. In certain embodiments, the vector generated to encode an arenavirus particle as described herein may be based on a specific strain of Latino virus. In certain embodiments, the vector generated to encode an arenavirus particle as described herein may be based on a specific strain of Machupo virus. In certain embodiments, the vector generated to encode an arenavirus particle as described herein may be based on a specific strain of Parana virus. In certain embodiments, the vector generated to encode an arenavirus particle as described herein may be based on a specific strain of Pirital virus. In certain embodiments, the vector generated to encode an arenavirus particle as described herein may be based on a specific strain of Sabia virus. In certain embodiments, the vector generated to encode an arenavirus particle as described herein may be based on a specific strain of Tacaribe virus. In certain embodiments, the vector generated to encode an arenavirus particle as described herein may be based on a specific strain of Bear Canyon virus. In certain embodiments, the vector generated to encode an arenavirus particle as described herein may be based on a specific strain of Whitewater Arroyo virus. In certain embodiments, the vector generated to encode an arenavirus particle as described herein may be based on a specific strain of Allpahuayo virus (ALLV). In certain embodiments, the vector generated to encode an arenavirus particle as described herein may be based on a specific strain of Alxa virus. In certain embodiments, the vector generated to encode an arenavirus particle as described herein may be based on a specific strain of Chapare virus. In certain embodiments, the vector generated to encode an arenavirus particle as described herein may be based on a specific strain of Lijiang virus. In certain embodiments, the vector generated to encode an arenavirus particle as described herein may be based on a specific strain of Cupixi virus. In certain embodiments, the vector generated to encode an arenavirus particle as described herein may be based on a specific strain of Gairo virus. In certain embodiments, the vector generated to encode an arenavirus particle as described herein may be based on a specific strain of Loei River virus. In certain embodiments, the vector generated to encode an arenavirus particle as described herein may be based on a specific strain of Lujo virus. In certain embodiments, the vector generated to encode an arenavirus particle as described herein may be based on a specific strain of Luna virus. In certain embodiments, the vector generated to encode an arenavirus particle as described herein may be based on a specific strain of Luli virus. In certain embodiments, the vector generated to encode an arenavirus particle as described herein may be based on a specific strain of Lunk virus. In certain embodiments, the vector generated to encode an arenavirus particle as described herein may be based on a specific strain of Mariental virus. In certain embodiments, the vector generated to encode an arenavirus particle as described herein may be based on a specific strain of Merino Walk virus. In certain embodiments, the vector generated to encode an arenavirus particle as described herein may be based on a specific strain of Morogoro virus. In certain embodiments, the vector generated to encode an arenavirus particle as described herein may be based on a specific strain of Okahandja virus. In certain embodiments, the vector generated to encode an arenavirus particle as described herein may be based on a specific strain of Aporé virus. In certain embodiments, the vector generated to encode an arenavirus particle as described herein may be based on a specific strain of Ryukyu virus. In certain embodiments, the vector generated to encode an arenavirus particle as described herein may be based on a specific strain of Solwezi virus. In certain embodiments, the vector generated to encode an arenavirus particle as described herein may be based on a specific strain of souris virus. In certain embodiments, the vector generated to encode an arenavirus particle as described herein may be based on a specific strain of Wenzhou virus. In certain embodiments, the vector generated to encode an arenavirus particle as described herein may be based on a specific strain of Big Brushy Tank virus. In certain embodiments, the vector generated to encode an arenavirus particle as described herein may be based on a specific strain of Catarina virus. In certain embodiments, the vector generated to encode an arenavirus particle as described herein may be based on a specific strain of Skinner Tank virus. In certain embodiments, the vector generated to encode an arenavirus particle as described herein may be based on a specific strain of Tonto Creek virus. In certain embodiments, the vector generated to encode an arenavirus particle as described herein may be based on a specific strain of Xapuri virus,

In another embodiment, provided herein is a cell, wherein the cell comprises a DNA or a vector system described above in this section. Cell lines derived from such cells, cultures comprising such cells, methods of culturing such cells are also provided herein. In certain embodiments, provided herein is a cell, wherein the cell comprises a cDNA of the tri-segmented arenavirus particle. In some embodiments, the cell comprises the S segment(s) and/or the L segment(s).

### 5.9 Generation of the Arenavirus Particles

Generally, arenavirus particles can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the arenavirus particles provided herein, these techniques can be applied as described below.

In certain embodiments, the genome of the viruses may comprise the nucleotide sequence described in Section 5.4. The genome of the viruses can be modified as described in Section 5.6. The generation of an arenavirus particle as described in Section 5.5 or an arenavirus particle comprising a genomic or antigenomic segment as described in Section 5.6 can be recombinantly produced by any reverse genetic techniques known to one skilled in the art.

In certain embodiments, an arenavirus particle as described in Section 5.5 or an arenavirus particle comprising a genomic or antigenomic segment as described in Section 5.6 can be tri-segmented. A tri-segmented arenavirus particle can be recombinantly produced by reverse genetic techniques known in the art, for example as described in International Publication No.: WO 2016/075250 A1.

### 5.9.1 Infectious and Replication Competent Arenavirus Particle

In certain embodiments, the method of generating a bi-segmented arenavirus particle comprises (i) transfecting into a host cell the cDNA or RNA sequences each transcribed in vitro from DNA sequences of a first and a second arenavirus genomic or antigenomic segments; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the cDNA is comprised in a plasmid.

In certain embodiments, when the arenavirus particle is tri-segmented, the method of generating the arenavirus particle comprises (i) transfecting into a host cell the cDNAs or RNA sequences each transcribed in vitro from DNA sequences of the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle.

Once generated from cDNA, arenavirus particles (i.e., infectious and replication competent) can be propagated. In certain embodiments, the arenavirus particle can be propagated in any host cell that allows the virus to grow to titers that permit the uses of the virus as described herein. In one embodiment, the host cell allows the arenavirus particle to grow to titers comparable to those determined for the corresponding tri-segmented artLCMV particle expressing the same heterologous non-arenaviral polypeptide(s).

In certain embodiments, the arenavirus particle may be propagated in host cells. Specific examples of host cells that can be used include BHK-21, HEK 293, VERO or other. In a specific embodiment, the arenavirus particle may be propagated in a cell line.

In certain embodiments, the host cells are kept in culture and are transfected with one or more plasmid(s). The plasmid(s) express or encode the arenavirus genomic or antigenomic segment(s) to be generated under control of one or more expression cassettes suitable for expression in mammalian cells, e.g., consisting of a polymerase I promoter and terminator.

In specific embodiments, the host cells are kept in culture and are transfected with one or more plasmid(s). The plasmid(s) express or encode the viral gene(s) to be generated under control of one or more expression cassettes suitable for expression in mammalian cells, e.g., consisting of a polymerase I promoter and terminator.

Plasmids that can be used for the generation of the arenavirus particle can include: i) a plasmid encoding the S genomic segment e.g., pol-I S, ii) a plasmid encoding the L genomic segment e.g., pol-I L. In certain embodiments, the plasmid encoding an arenavirus polymerase that directs intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, when the arenaviral particle is tri-segmented, plasmids that can be used for generating the tri-segmented arenavirus comprising one L segment and two S segments can include: i) two plasmids each encoding the S genome segment e.g., pol-I S, ii) a plasmid encoding the L genome segment e.g., pol-I L. Plasmids needed for the tri-segmented arenavirus comprising two L segments and one S segment are: i) two plasmids each encoding the L genome segment e.g., pol-L, ii) a plasmid encoding the S genome segment e.g., pol-I S.

In certain embodiments, when the arenaviral particle is tri-segmented, plasmids encoding an arenavirus polymerase that direct intracellular synthesis of the viral L and S segments can be incorporated into the transfection mixture. For example, a plasmid encoding the L protein and a plasmid encoding NP (pC-L and pC-NP, respectively). The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, the arenavirus genomic or antigenomic segments are under the control of a promoter. Typically, RNA polymerase I-driven expression cassettes, RNA polymerase II-driven cassettes or T7 bacteriophage RNA polymerase driven cassettes can be used. In certain embodiments, the plasmid(s) encoding the arenavirus genomic or antigenomic segments can be the same, i.e., the genome sequence and trans-acting factors can be transcribed by a promoter from one plasmid. Specific examples of promoters include an RNA polymerase I promoter, an RNA polymerase II promoter, an RNA polymerase III promoter, a T7 promoter, an SP6 promoter or a T3 promoter.

In addition, the plasmid(s) can feature a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

To obtain a cell line stably expressing a desired protein, for example an arenaviral structural protein, transfection of a host cell with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

For recovering the arenavirus particle described herein, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (arenavirus vector preparation) is harvested, aliquoted and stored at 4° C., −20° C., or −80° C., depending on how long the arenavirus vector should be stored prior use. The arenavirus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a heterologous non-arenaviral polypeptide, wherein a plasmid encoding the genomic segment is modified to incorporate a nucleotide encoding a heterologous non-arenaviral polypeptide. The nucleotide encoding a heterologous non-arenaviral polypeptide can be incorporated into the plasmid using restriction enzymes and ligases by molecular cloning techniques as known to those skilled in the art.

### 5.9.2 Infectious, Replication-Defective Arenavirus Particle

Infectious, replication-defective arenavirus particles can be rescued as described above. However, once generated from DNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express or encode the viral gene(s) deleted in the arenavirus vector to be generated under control of one or more expression cassettes suitable for expression in mammalian cells, e.g., a mammalian polymerase II promoter such as the EFlalpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids can be of two types: i) two plasmids, referred to as TF-plasmids for expressing intracellularly in C-cells the minimal trans-acting factors of the arenavirus, is derived from e.g., NP and L proteins of LCMV in the present example; and ii) plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and trans-acting factors can be transcribed by T7, polI and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and trans-acting factors can be transcribed by T7, polI and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of an antigen in a cell culture wherein the cell culture is infected with an infectious, replication-deficient arenavirus expressing an antigen. When used for expression of an antigen in cultured cells, the following two procedures can be used:

i) The cell type of interest is infected with the arenavirus vector preparation described herein at a multiplicity of infection (MOI) of one or more, e.g., two, three or four, resulting in production of the antigen in all cells already shortly after infection.

ii) Alternatively, a lower MOI can be used and individual cell clones can be selected for their level of virally driven antigen expression. Subsequently individual clones can be expanded infinitely owing to the non-cytolytic nature of arenavirus vectors. Irrespective of the approach, the antigen can subsequently be collected (and purified) either from the culture supernatant or from the cells themselves, depending on the properties of the antigen produced. However, the invention is not limited to these two strategies, and other ways of driving expression of antigen using infectious, replication-deficient arenaviruses as vectors may be considered.

### 5.10 Methods of Using the Compositions Provided Herein—Generation and Rescue of Arenavirus Particles

In certain embodiments, the nucleotide sequence as provided herein (see Section 5.4) can be used to generate and/or rescue arenavirus particles. In certain embodiments, the arenavirus particles as provided herein (see Section 5.5) can be used to generate and/or rescue arenavirus particles. In certain embodiments, the arenavirus genomic or antigenomic segment as provided herein (see Section 5.6) can be used to generate and/or rescue arenavirus particles.

Generally, arenavirus particles can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein).

In certain embodiments, an arenavirus particle generated or rescued can be bi-segmented. In other embodiments, an arenavirus particle generated or rescued can be tri-segmented. A tri-segmented arenavirus particle can be recombinantly produced by reverse genetic techniques known in the art, for example as described in International Publication No.: WO 2016/075250 A1.

### 5.10.1 Infectious and Replication Competent Arenavirus Particle

In certain embodiments, the method of generating a bi-segmented arenavirus particle comprises (i) transfecting into a host cell one or more DNA sequences as described in Section 5.4, or one or more RNA sequences as described in Section 5.4, or one or more RNA sequences each transcribed in vitro from the DNA sequence as described in Section 5.4; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, when the arenavirus particle is tri-segmented, the method of generating the arenavirus particle comprises (i) transfecting into a host cell one or more DNA sequences as described in Section 5.4, one or more RNA sequences as described in Section 5.4, or one or more RNA sequences each transcribed in vitro from the DNA sequence as described in Section 5.4, wherein the above nucleotide sequences encode the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, the method of generating a bi-segmented arenavirus particle comprises (i) transfecting into a host cell one or more cDNA sequences of the mRNA transcripts of the genome of the arenavirus particle as described in Section 5.5 or one or more RNA sequences each transcribed in vitro from the cDNA of the mRNA transcript of the genome of the arenavirus particle as described in Section 5.5; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, when the arenavirus particle is tri-segmented, the method of generating the arenavirus particle comprises (i) transfecting into a host cell one or more cDNA sequences of the mRNA transcripts of the genome of the arenavirus particle as described in Section 5.5 or one or more RNA sequences each transcribed in vitro from the cDNA of the mRNA transcript of the genome of the arenavirus particle as described in Section 5.5, wherein the above nucleotide sequences encode the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, the method of generating a bi-segmented arenavirus particle comprises (i) transfecting into a host cell the cDNA sequences of the arenavirus genomic or antigenomic segment as described in Section 5.6 or one or more RNA sequences each transcribed in vitro from the cDNA sequence of the arenavirus genomic or antigenomic segment as described in Section 5.6; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, when the arenavirus particle is tri-segmented, the method of generating the arenavirus particle comprises (i) transfecting into a host cell the cDNA sequences of the arenavirus genomic or antigenomic segment as described in Section 5.6 or one or more RNA sequences each transcribed in vitro from the cDNA sequence of the arenavirus genomic or antigenomic segment as described in Section 5.6, wherein the above nucleotide sequences encode the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

More details on generation of infectious and replication competent arenavirus particles are described in Section 5.9.1.

### 5.10.2 Infectious, Replication-Defective Arenavirus Particle

Infectious, replication-defective arenavirus particles can be rescued as described above. More details on generation of infectious, replication-defective arenavirus particles are described in Section 5.9.2.

### 5.11 Methods of Using the Compositions Provided Herein—Vaccine

The present application furthermore relates to vaccines, immunogenic compositions (e.g., vaccine formulations), and pharmaceutical compositions comprising a nucleotide sequence as described in Section 5.4 and related expression products, arenavirus particles and arenavirus genomic or antigenomic segments. The present application also relates to vaccines, immunogenic compositions (e.g., vaccine formulations), and pharmaceutical compositions comprising an arenavirus particle as described in Section 5.5. The present application further relates to vaccines, immunogenic compositions (e.g., vaccine formulations), and pharmaceutical compositions comprising an arenavirus genomic or antigenomic segment as described in Section 5.6 and related arenavirus particles. The present application further relates to vaccines, immunogenic compositions (e.g., vaccine formulations), and pharmaceutical compositions comprising a translation product as described in Section 5.7. The present application further relates to vaccines, immunogenic compositions (e.g., vaccine formulations), and pharmaceutical compositions comprising a cDNA, DNA sequence and/or DNA expression vector related to the nucleotide sequences as described in Section 5.4, the arenavirus particle as described in Section 5.5, and/or the arenavirus genomic or antigenomic segments as described in Section 5.6, for example a DNA sequence encoding the arenavirus genomic or antigenomic segment as described in Section 5.6. Such vaccines, immunogenic compositions and pharmaceutical compositions can be formulated according to standard procedures in the art.

It will be readily apparent to one of ordinary skill in the relevant arts that suitable modifications and adaptations to the methods and applications described herein can be obvious and can be made without departing from the scope or any embodiment thereof.

In certain embodiments, provided herein are compositions comprising the nucleotide sequences as described in Section 5.4. In another embodiment, provided herein are compositions comprising the arenavirus particle as described in Section 5.5. In another embodiment, provided herein are compositions comprising the arenavirus genomic or antigenomic segments as described in Section 5.6. In another embodiment, provided herein are compositions comprising the translation product as described in Section 5.7. In another embodiment, provided herein are compositions comprising a cDNA, DNA sequence and/or DNA expression vectors related to the nucleotide sequences as described in Section 5.4, the arenavirus particle as described in Section 5.5, and/or the arenavirus genomic or antigenomic segments as described in Section 5.6, for example a DNA sequence encoding the arenavirus genomic or antigenomic segment as described in Section 5.6. Such compositions can be used in methods of treatment and prevention of disease. In a specific embodiment, the compositions described herein are used in the treatment of subjects infected with, or susceptible to, an infection. In other embodiments, the compositions described herein are used in the treatment of subjects susceptible to or exhibiting symptoms characteristic of cancer or tumorigenesis or are diagnosed with cancer. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the prevention or treatment of infection or cancer of subjects (e.g., human subjects). In other embodiments, the vaccine, immunogenic composition or pharmaceutical composition are suitable for veterinary and/or human administration.

In certain embodiments, provided herein are immunogenic compositions comprising an arenavirus vector as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an arenavirus particle or tri-segmented arenavirus particle and, most importantly, the gene products it vectorises, but when the compound is administered alone does not generate an immune response to the arenavirus particle or tri-segmented arenavirus particle and the gene products vectorised by the latter. In some embodiments, the adjuvant generates an immune response to the arenavirus particle or tri-segmented arenavirus particle and the gene products vectorised by the latter and does not produce an allergy or other adverse reaction. Adjuvants can enhance an immune response by several mechanisms including, e.g., lymphocyte recruitment, stimulation of B and/or T cells, and stimulation of macrophages or dendritic cells. When a vaccine or immunogenic composition provided herein comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., 1995, in Vaccine Design: The Subunit and Adjuvant Approach (eds. Powell & Newman, Plenum Press, NY); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., 1997, N. Engl. J. Med. 336, 86-91).

The compositions comprise the bi-segmented arenaviruses particle or tri-segmented arenavirus particle described herein alone or together with a pharmaceutically acceptable carrier. Suspensions or dispersions of the arenavirus particle or tri-segmented arenavirus particle, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use, or alternatively may be lyophilized for storage. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the bi-segmented arenavirus particle or tri-segmented arenavirus particle.

In one embodiment, administration of the pharmaceutical composition is parenteral administration. Parenteral administration can be intravenous or subcutaneous administration. Accordingly, unit dose forms for parenteral administration are, for example, ampoules or vials, e.g., vials containing from about 10³ to 10¹⁰ focus forming units or 10⁵ to 10¹⁵ physical particles of the arenavirus particle or tri-segmented arenavirus particle.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, intra-tumoral, and via scarification (scratching through the top layers of skin, e.g., using a bifurcated needle). Specifically, subcutaneous or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and as suitable powder base such as lactose or starch.

The dosage of the active ingredient depends upon the type of vaccination or immunotherapy and upon the subject, and their age, weight, individual condition, the individual pharmacokinetic data, and the mode of administration. In certain embodiments, an in vitro assay is employed to help identify optimal dosage ranges. Effective doses may be extrapolated from dose response curves derived from in vitro or animal model test systems.

In certain embodiments, the vaccine, immunogenic composition, or pharmaceutical composition comprising an arenavirus particle or the tri-segmented arenavirus particle can be used as a live vaccination or immunotherapy. Exemplary doses for a live arenavirus particle may vary from 10-100, or more, PFU of live virus per dose. In some embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and can be administered to a subject once, twice, three or more times with intervals as often as needed. In another embodiment, a live arenavirus is formulated in a dose that is of a volume selected from the group consisting of 0.2, 0.5, 1, 2, 5, 7, 10, 15, 20, 25, 30, 35, 40, 45, and 50 mL. In certain embodiments, the dose contains 10^{5.5}-10^{9.5} infectious focus forming units of live arenavirus particle. In another embodiment, an inactivated vaccine is formulated such that it contains about 15 μg to about 100 μg, about 15 μg to about 75 μg, about 15 μg to about 50 μg, or about 15 μg to about 30 μg of an arenavirus.

In certain embodiments, for administration to children, two doses of a bi-segmented arenavirus particle or a tri-segmented arenavirus particle described herein or a composition thereof, given at least one month apart, are administered to a child. In specific embodiments for administration to adults, a single dose of the arenavirus particle or tri-segmented arenavirus particle described herein or a composition thereof is given. In another embodiment, two doses of a bi-segmented arenavirus particle or a tri-segmented arenavirus particle described herein or a composition thereof, given at least one month apart, are administered to an adult. In another embodiment, a young child (six months to nine years old) may be administered a bi-segmented arenavirus particle or a tri-segmented arenavirus particle described herein or a composition thereof for the first time in two doses given one month apart. In a particular embodiment, a child who received only one dose in their first year of vaccination or immunotherapy should receive two doses in the following year. In some embodiments, two doses administered 4 weeks apart are preferred for children 2-8 years of age who are administered an immunogenic composition described herein, for the first time. In certain embodiments, for children 6-35 months of age, a half dose (0.25 ml) may be preferred, in contrast to 0.5 ml which may be preferred for subjects over three years of age.

In certain embodiments, the compositions can be administered to the patient in a single dosage comprising a therapeutically effective amount of the arenavirus particle or the tri-segmented arenavirus particle. In some embodiments, the bi-segmented arenavirus particle or tri-segmented arenavirus particle can be administered to the patient in a single dose comprising a therapeutically effective amount of a bi-segmented arenavirus particle or tri-segmented arenavirus particle and, one or more pharmaceutical compositions, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

Also provided herein, are processes and to the use the arenavirus particle or the tri-segmented arenavirus particle for the manufacture of vaccines in the form of pharmaceutical preparations, which comprise the arenavirus particle or tri-segmented arenavirus particle as an active ingredient. The pharmaceutical compositions of the present application are prepared in a manner known per se, for example by means of conventional mixing and/or dispersing processes.

### 5.12 Compositions, Administration, and Dosage

The present application furthermore relates to vaccines, immunogenic compositions (e.g., vaccine formulations), and pharmaceutical compositions comprising an arenavirus particle or a tri-segmented arenavirus particle as described herein. Such vaccines, immunogenic compositions and pharmaceutical compositions can be formulated according to standard procedures in the art.

It will be readily apparent to one of ordinary skill in the relevant arts that suitable modifications and adaptations to the methods and applications described herein can be obvious and can be made without departing from the scope of the scope or any embodiment thereof.

In another embodiment, provided herein are compositions comprising an arenavirus particle or a tri-segmented arenavirus particle described herein. Such compositions can be used in methods of treatment and prevention of disease. In a specific embodiment, the compositions described herein are used in the treatment of subjects infected with and infectious agent, or susceptible to an infection. In other embodiments, the compositions described herein are used in the treatment of subjects susceptible to or exhibiting symptoms characteristic of cancer or tumorigenesis or are diagnosed with cancer. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the prevention of infection or cancer of subjects (e.g., human subjects). In other embodiments, the vaccine, immunogenic composition or pharmaceutical composition are suitable for veterinary and/or human administration.

In certain embodiments, provided herein are immunogenic compositions comprising an arenavirus vector as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a arenavirus particle or tri-segmented arenavirus particle and, most importantly, the gene products it vectorises, but when the compound is administered alone does not generate an immune response to the arenavirus particle or tri-segmented arenavirus particle and the gene products vectorised by the latter. In some embodiments, the adjuvant generates an immune response to the arenavirus particle or tri-segmented arenavirus particle and the gene products vectorised by the latter and does not produce an allergy or other adverse reaction. Adjuvants can enhance an immune response by several mechanisms including, e.g., lymphocyte recruitment, stimulation of B and/or T cells, and stimulation of macrophages or dendritic cells. When a vaccine or immunogenic composition provided herein comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., 1995, in Vaccine Design: The Subunit and Adjuvant Approach (eds. Powell & Newman, Plenum Press, NY); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., 1997, N. Engl. J. Med. 336, 86-91).

The compositions comprise the arenaviruses particle or tri-segmented arenavirus particle described herein alone or together with a pharmaceutically acceptable carrier. Suspensions or dispersions of the arenavirus particle or tri-segmented arenavirus particle, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use, or alternatively may be lyophilized for storage. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the arenavirus particle or tri-segmented arenavirus particle.

In one embodiment, administration of the pharmaceutical composition is parenteral administration. Parenteral administration can be intravenous or subcutaneous administration. Accordingly, unit dose forms for parenteral administration are, for example, ampoules or vials, e.g., vials containing from about 10³ to 10¹⁰ focus forming units or 10⁵ to 10¹⁵ physical particles of the arenavirus particle or tri-segmented arenavirus particle.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and as suitable powder base such as lactose or starch.

The dosage of the active ingredient depends upon the type of vaccination or immunotherapy and upon the subject, and their age, weight, individual condition, the individual pharmacokinetic data, and the mode of administration. In certain embodiments, an in vitro assay is employed to help identify optimal dosage ranges. Effective doses may be extrapolated from dose response curves derived from in vitro or animal model test systems.

In certain embodiments, the vaccine, immunogenic composition, or pharmaceutical composition comprising an arenavirus particle or the tri-segmented arenavirus particle can be used as a live vaccination or immunotherapy. Exemplary doses for a live arenavirus particle may vary from 10-100, or more, PFU of live virus per dose. In some embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, ×1×10¹¹, 5×10¹¹ or 10¹² pfu, and can be administered to a subject once, twice, three or more times with intervals as often as needed. In another embodiment, a live arenavirus is formulated such that a 0.2-mL dose contains 10^{6.5}-10^{7.5} fluorescent focal units of live arenavirus particle. In another embodiment, an inactivated vaccine is formulated such that it contains about 15 μg to about 100 μg, about 15 μg to about 75 μg, about 15 μg to about 50 μg, or about 15 μg to about 30 μg of an arenavirus.

In certain embodiments, for administration to children, two doses of an arenavirus particle or a tri-segmented arenavirus particle described herein or a composition thereof, given at least one month apart, are administered to a child. In specific embodiments for administration to adults, a single dose of the arenavirus particle or tri-segmented arenavirus particle described herein or a composition thereof is given. In another embodiment, two doses of an arenavirus particle or a tri-segmented arenavirus particle described herein or a composition thereof, given at least one month apart, are administered to an adult. In another embodiment, a young child (six months to nine years old) may be administered an arenavirus particle or a tri-segmented arenavirus particle described herein or a composition thereof for the first time in two doses given one month apart. In a particular embodiment, a child who received only one dose in their first year of vaccination or immunotherapy should receive two doses in the following year. In some embodiments, two doses administered 4 weeks apart are preferred for children 2-8 years of age who are administered an immunogenic composition described herein, for the first time. In certain embodiments, for children 6-35 months of age, a half dose (0.25 ml) may be preferred, in contrast to 0.5 ml which may be preferred for subjects over three years of age.

In certain embodiments, the compositions can be administered to the patient in a single dosage comprising a therapeutically effective amount of the arenavirus particle or the tri-segmented arenavirus particle. In some embodiments, the arenavirus particle or tri-segmented arenavirus particle can be administered to the patient in a single dose comprising a therapeutically effective amount of an arenavirus particle or tri-segmented arenavirus particle and, one or more pharmaceutical compositions, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

Also provided herein, are processes and to the use the arenavirus particle or the tri-segmented arenavirus particle for the manufacture of vaccines in the form of pharmaceutical preparations, which comprise the arenavirus particle or tri-segmented arenavirus particle as an active ingredient. The pharmaceutical compositions of the present application are prepared in a manner known per se, for example by means of conventional mixing and/or dispersing processes.

### 5.13 Assays

In certain embodiments, an arenavirus particle described herein comprises the nucleotide sequence provided herein (see Section 5.4.2). In certain embodiments, an arenavirus particle described herein is engineered such that an arenaviral ORF is separated over two or more mRNA transcripts (see Section 5.5). In certain embodiments, an arenavirus particle described herein is derived from an arenavirus genomic or antigenomic segment provided herein (see Section 5.6). In certain embodiments, an arenavirus particle described herein comprises an arenavirus genomic or antigenomic segment provided herein (see Section 5.6).

In certain embodiments, the arenavirus particle provided herein is genetically stable, i.e. the inability of the arenavirus particle to revert to wild-type-like (more virulent) replication behavior in the host (genetic stability). In certain embodiments, the genetic stability may be demonstrated using assays described in this Section and Section 6.

In certain embodiments, the arenavirus particle provided herein shows high-level transgene expression to elicit strong immune responses against the desired target antigen(s) (transgene expression levels). In certain embodiments, the high-level transgene expression may be demonstrated using assays described in this Section and Section 6.

In certain embodiments, the arenavirus particle expresses a heterologous non-arenaviral polypeptide. In certain embodiments, the heterologous non-arenaviral polypeptide is under control of an arenavirus 3′ UTR. In certain embodiments, the expression level of the heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, the expression level of the heterologous non-arenaviral polypeptide is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, the expression level of the heterologous non-arenaviral polypeptide is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express the heterologous non-arenaviral polypeptide after an arenavirus of the present disclosure (e.g., expressing a heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptide after an arenavirus of the present disclosure (e.g., expressing a heterologous non-arenaviral polypeptide under control of an arenavirus 3′ UTR) is introduced to (or infects) a population of cells is higher as compared to the proportion of cells that express the heterologous non-arenaviral polypeptide after an arenavirus expressing the same heterologous non-arenaviral polypeptide under control of an arenavirus 5′ UTR is introduced to (or infects) a population of cells. In certain embodiments, at least about, or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptide after an arenavirus of the present disclosure (e.g., expressing a heterologous non-arenaviral polypeptide under control of an arenavirus 3′ UTR) is introduced to (or infects) a population of cells as compared to the proportion (or amount) of cells that express the heterologous non-arenaviral polypeptides after an arenavirus expressing the same heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express a heterologous non-arenaviral polypeptide and/or express a second heterologous non-arenaviral polypeptide after an arenavirus of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptide and/or express the second heterologous non-arenaviral polypeptide after an arenavirus of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells is higher as compared to the proportion of cells that express the heterologous non-arenaviral polypeptide or the second heterologous non-arenaviral polypeptide after an arenavirus expressing the same heterologous non-arenaviral polypeptide under control of an arenavirus 5′ UTR and/or expressing the same second heterologous non-arenaviral polypeptide under control of an arenavirus 5′ UTR is introduced to (or infects) a population of cells. In certain embodiments, at least about, or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptide and/or express the second heterologous non-arenaviral polypeptide after an arenavirus of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells as compared to the proportion (or amount) of cells that express the heterologous non-arenaviral polypeptides and/or express the second heterologous non-arenaviral polypeptides after an arenavirus expressing the same heterologous non-arenaviral polypeptide under control of an arenavirus 5′ UTR and/or expressing the same second heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the arenavirus particle expresses two heterologous non-arenaviral polypeptides. In certain embodiments, expression of one heterologous non-arenaviral polypeptide is under control of an arenavirus 3′ UTR and expression of the other heterologous non-arenaviral polypeptide is under control of an arenavirus 5′ UTR. In certain embodiments, the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment. In certain embodiments, the expression of one heterologous non-arenaviral polypeptide is under control of an arenavirus 3′ UTR in one S segment and the expression of the other heterologous non-arenaviral polypeptide is under control of an arenavirus 5′ UTR in the other S segment. In certain embodiments, the expression of a first of the two heterologous non-arenaviral polypeptides is under control of an arenavirus 5′ UTR in a first S segment and the expression of a second of the two heterologous non-arenaviral polypeptides is under control of an arenavirus 3′ UTR in a second S segment. In certain embodiments, the expression of one heterologous non-arenaviral polypeptide is under control of an arenavirus 5′ UTR in one S segment and the expression of the other heterologous non-arenaviral polypeptide is under control of an arenavirus 3′ UTR in the other S segment. In certain embodiments, cells that are infected with the arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) express (co-express) both heterologous non-arenaviral polypeptides. In certain embodiments, the combined expression level of the two heterologous non-arenaviral polypeptides (e.g., cells expressing both heterologous non-arenaviral polypeptides) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide is under control of an arenavirus 5′ UTR of one S segment and expression of the other heterologous non-arenaviral polypeptide is under control of an arenavirus 5′ UTR of the other S segment). In certain embodiments, the combined expression level of the two heterologous non-arenaviral polypeptides (e.g., cells expressing both heterologous non-arenaviral polypeptides) is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide under control of an arenavirus 5′ UTR of one S segment and expression of the other heterologous non-arenaviral polypeptide under control of an arenavirus 5′ UTR of the other S segment). In certain embodiments, the combined expression level of the two heterologous non-arenaviral polypeptides (e.g., cells expressing both heterologous non-arenaviral polypeptides) is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide under control of an arenavirus 5′ UTR of one S segment and expression of the other heterologous non-arenaviral polypeptide under control of an arenavirus 5′ UTR of the other S segment). In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express (co-express) the two heterologous non-arenaviral polypeptides after an arenavirus of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) the cells (e.g., to a population of cells). In certain embodiments, the proportion (or amount) of cells that express (co-express) the two heterologous non-arenaviral polypeptides after an arenavirus of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells is higher as compared to the proportion (or amount) of cells that express (co-express) the two heterologous non-arenaviral polypeptides after an arenavirus expressing the same two heterologous non-arenaviral polypeptides under control of arenavirus 5′ UTRs (e.g., one heterologous non-arenaviral polypeptide expressed under control of an arenavirus 5′ UTR of one S segment and the other heterologous non-arenaviral polypeptide expressed under control of an arenavirus 5′ UTR of the other S segment) is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express (co-express) the two heterologous non-arenaviral polypeptides after an arenavirus of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells as compared to the proportion (or amount) of cells that express (co-express) the two heterologous non-arenaviral polypeptides after an arenavirus expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the arenavirus particle provided herein may show good growth in cell culture, enabling the arenavirus particle's production to high titers in industrial fermentation processes (production yields). In certain embodiments, the good growth in cell culture may be demonstrated using the assays described in this Section and in Section 6.

In certain embodiments, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle. In certain embodiments, the titer of the arenavirus particle is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle. In certain embodiments, the titer of the arenavirus particle is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle. In certain embodiments, a persistent infection may be an infection that lasts at least 5 days, 10 days, 14 days, 20 days, 25 days, 30 days, 35 days, 40 days, 45 days, 50 days, 53 days, 55 days, 60 days, 65 days, 70 days, 75 days, 78 days, 80 days, 90 days, or 100 days. In certain embodiments, a persistent infection may be an infection that lasts 5 days, 10 days, 14 days, 20 days, 25 days, 30 days, 35 days, 40 days, 45 days, 50 days, 53 days, 55 days, 60 days, 65 days, 70 days, 75 days, 78 days, 80 days, 90 days, or 100 days.

### 5.13.1 Arenavirus Detection Assays

The skilled artesian could detect an arenavirus genomic or antigenomic segment or tri-segmented arenavirus particle, as described herein using techniques known in the art. For example, RT-PCR can be used with primers that are specific to a genetically engineered arenavirus segment to detect and quantify an arenavirus genomic or antigenomic segment as described herein in section 5.6. Western blot, ELISA, radioimmunoassay, immunoprecipitation, or immunocytochemistry in conjunction with flow cytometry can be used to quantify the gene products of the arenavirus genomic or antigenomic segment or tri-segmented arenavirus particle.

### 5.13.2 Assay to Measure Infectivity

Any assay known to the skilled artisan can be used for measuring the infectivity of an arenavirus vector preparation. For example, determination of the virus/vector titer can be done by a “focus forming unit assay” (FFU assay). In brief, complementing cells are plated and inoculated with different dilutions of a virus/vector sample. For determination of replication-deficient arenavirus vector titers, complementing (e.g. glycoprotein-expressing cells in the case of glycoprotein-deficient arenavirus vectors) BHK-21 of HEK293 cells are used. For replication-competent vectors e.g., MC57, 3T3 or VERO cells are common cell substrates. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and by that countable using antibodies against the respective arenavirus' NP or another protein expressed by the arenavirus particle or the tri-segmented arenavirus particle and a HRP-based color reaction. The titer of a virus/vector can be calculated in focus-forming units per milliliter (FFU/mL).

### 5.13.3 Growth of an Arenavirus Particle

Growth of an arenavirus particle described herein can be assessed by any method known in the art or described herein (e.g., cell culture). Viral growth may be performed by inoculating serial dilutions of an arenavirus particle described herein into cell cultures (e.g., Vero cells or BHK-21 cells or complementing versions thereof in case of replication-deficient vectors). After incubation of the virus for a specified time, the supernatant containing virus is collected and virions can be purified using standard methods.

### 5.13.4 Serum ELISA

Determination of the humoral immune response upon vaccination or immunotherapy of animals (e.g., mice, guinea pigs) can be done by antigen-specific serum ELISA's (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

### 5.13.5 Assay to Measure the Neutralizing Activity of Induced Antibodies

Determination of the neutralizing antibodies in sera can be performed by plaque reduction neutralization tests. Depending on the viral target, suitable cells are used, such as ARPE-19 cells from ATCC for testing cytomegalovirus-neutralizing antibodies and a GFP-tagged virus (for example, cytomegalovirus) as test article. In addition, supplemental guinea pig serum as a source of exogenous complement may be used. The assay is started with seeding of 6.5×10³ cells/well (50 μl/well) in a 384 well plate one or two days before using for neutralization. The neutralization is performed by mixing of a defined virus inoculum in 96-well sterile tissue culture plates without cells but with titrated concentrations of test serum for 1 hour at 37° C. After the neutralization incubation step the mixture is added to the cells and incubated for additional 4 days for GFP-detection with a plate reader. Alternatively, instead of GFP or similar reporters, a viral structural protein can be detected as described in section 5.13.2. A positive neutralizing human sera is used as assay positive control on each plate to check the reliability of all results. Titers (EC50) are determined using a 4 parameter logistic curve fitting. As additional testing the wells are checked with a fluorescence microscope.

### 5.13.6 LCMV Plaque Reduction Neutralization Test

In brief, plaque reduction (neutralization) assays for LCMV can be performed by use of a replication-competent or -deficient LCMV that is tagged with green fluorescent protein, 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples.

5.13.7 Determination of LCMV genome copies

qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### 5.13.8 Western Blotting

Infected cells grown in tissue culture flasks or in suspension are lysed at specific time points post infection using RIPA buffer (Thermo Scientific) or used directly without cell-lysis. Samples are heated to 99° C. for 10 minutes with reducing agent and NuPage LDS Sample buffer (Novex, Thermo Fisher Scientific, USA) and chilled to room temperature before loading on 4-12% SDS-gels for electrophoresis. Proteins are blotted onto membranes using Invitrogens iBlot Gel transfer Device and visualized by Ponceau staining. Finally, the preparations are probed with a primary antibody directed against proteins of interest and alkaline phosphatase conjugated secondary antibodies followed by staining with 1-Step NBT/BCIP solution (Invitrogen, USA).

### 5.13.9 MHC-Peptide Multimer Staining Assay for Detection of Antigen-Specific CD8+ T-Cells

Any assay known to the skilled artisan can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order for a T-cell to detect the peptide to which it is specific, it must both recognize the peptide and the tetramer of MHC molecules custom made for a defined antigen specificity and MHC haplotype of T-cells (typically fluorescently labeled). The tetramer is then detected by flow cytometry via the fluorescent label.

### 5.13.10 ELISPOT Assay for Detection of Antigen-Specific T-Cells.

Any assay known to the skilled artisan can be used to test antigen-specific T-cell responses (CD4+ and CD8+). For example, the ELISPOT assay can be used (see, e.g., Czerkinsky C. C. et al., J Immunol Methods. 1983; 65:109-121; and Hutchings P. R. et al., J Immunol Methods. 1989; 120:1-8). Briefly, the assay comprises the following steps: An immunospot plate is coated with an anti-cytokine antibody. Cells are incubated in the immunospot plate in the presence of specific stimulus e.g. peptides against which reactivity is to be tested. Cells secrete cytokines and are then washed off. Plates are then coated with a second biotyinlated-anticytokine antibody and cytokine secretion spots are visualized with an avidin-HRP system.

### 5.13.11 Intracellular Cytokine Assay for Detection of Functionality of CD8+ and CD4+ T-Cell Responses.

Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: activation of cells via specific peptides or protein, an inhibition of protein transport (e.g., brefeldin A) is added to retain the cytokines within the cell. After a defined period of incubation, typically 5 hours, a washing steps follows, and antibodies to other cellular markers can be added to the cells. Cells are then fixed and permeabilized. The flurochrome-conjugated anti-cytokine antibodies are added and the cells can be analyzed by flow cytometry.

### 5.13.12 Assay for Confirming Replication-Deficiency of Viral Vectors

Any assay known to the skilled artisan that determines concentration of infectious and replication-competent virus particles can also be used as a to measure replication-deficient viral particles in a sample. For example, FFU assays with non-complementing cells can be used for this purpose.

Furthermore, plaque-based assays are a standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32: Immunology of Infection. Academic Press. ISBN 0-12-521532-0), and thereby exerts a cytopathic or lytic effect. Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

5.13.13 Assay for Expression of Viral Antigens Including Transgenically Inserted Non-Arenaviral Polypeptides Expressed from Viral Vectors

Any assay known to the skilled artisan can be used for measuring expression of viral antigens and transgenically inserted non-arenaviral polypeptides expressed from viral vectors. For example, FFU assays can be performed. For detection, mono- or polyclonal antibody preparation(s) against the respective viral antigens are used (transgene-specific FFU). Methods to determine the binding of specific antibodies to proteins expressed by the replication-competent or -deficient virus (viral structural proteins, viral non-structural proteins and transgenically inserted non-arenaviral polypeptides) comprise flow cytometry, immunocytochemistry, Immunofluorescence, Western Blot or dot blot. Fluorescent reporter proteins can be visualized by direct fluorescence microscopy or flow cytometry.

### 5.13.14 Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice, which can be used to investigate recombination and infectivity of an arenavirus particle, are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene expression.

### 6. EXAMPLES

6.1 Split-C and Split-E but not Split-A or Split-F Vectors Grow to High Titers Equivalent to artLCMV, Revealing a Key Role of SP Positioning for Vector Growth

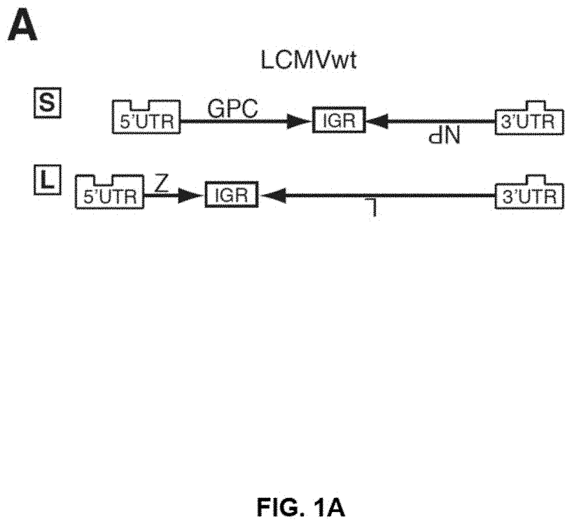

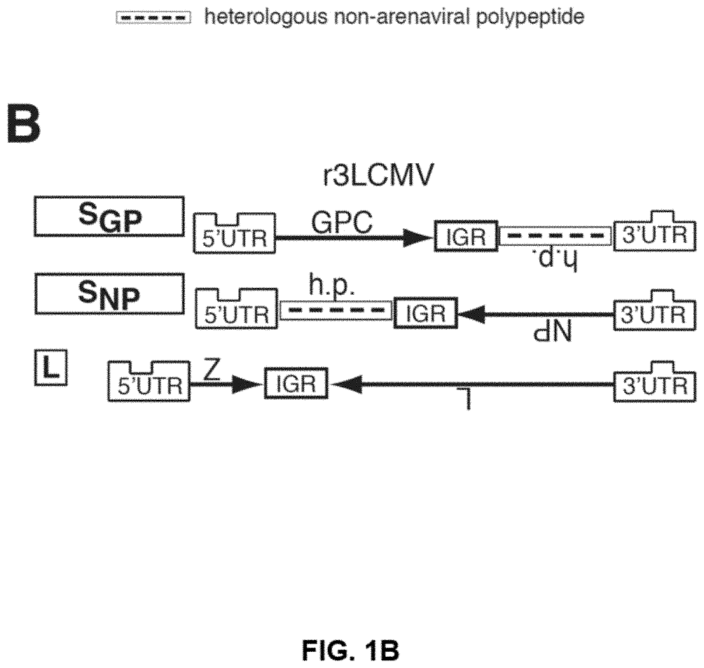

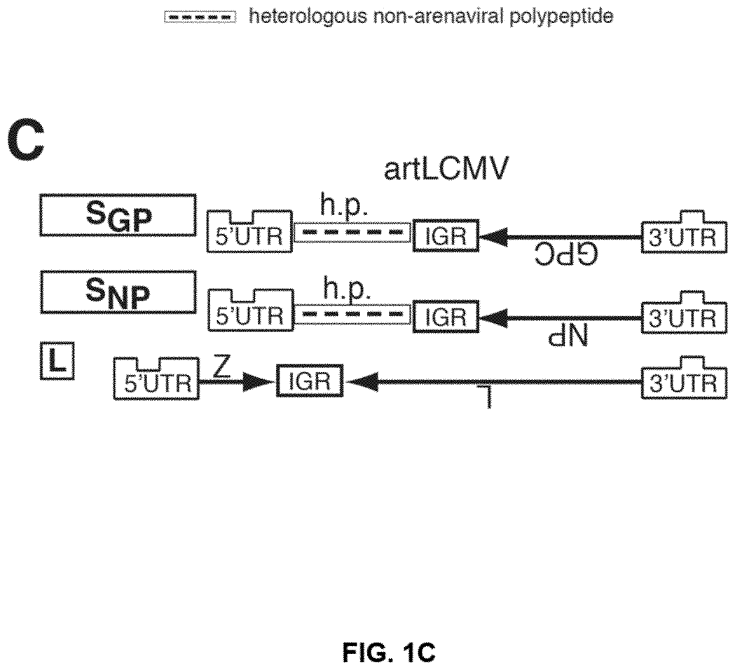

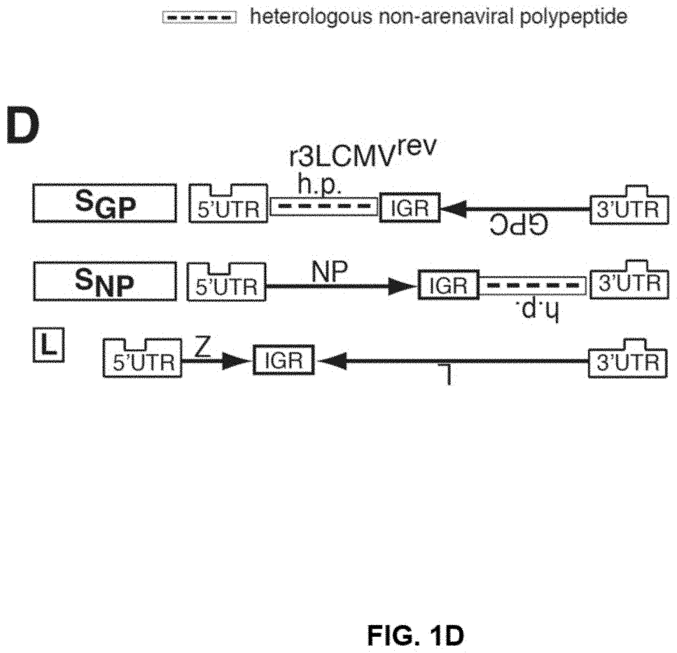

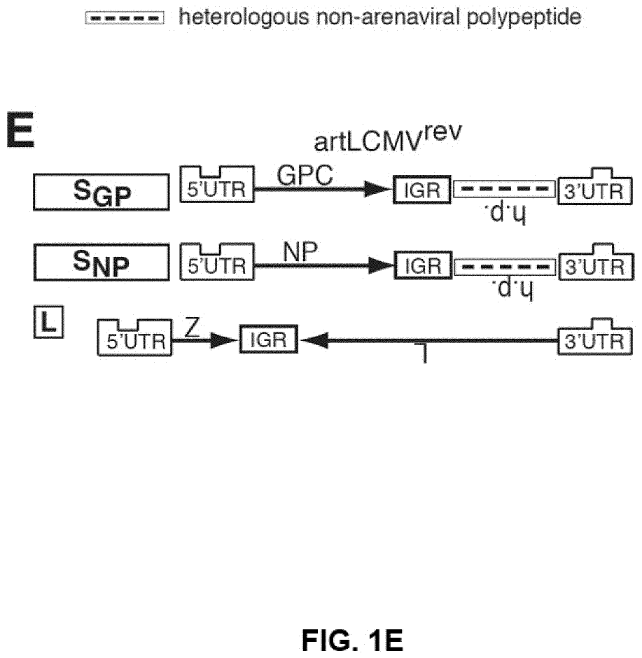

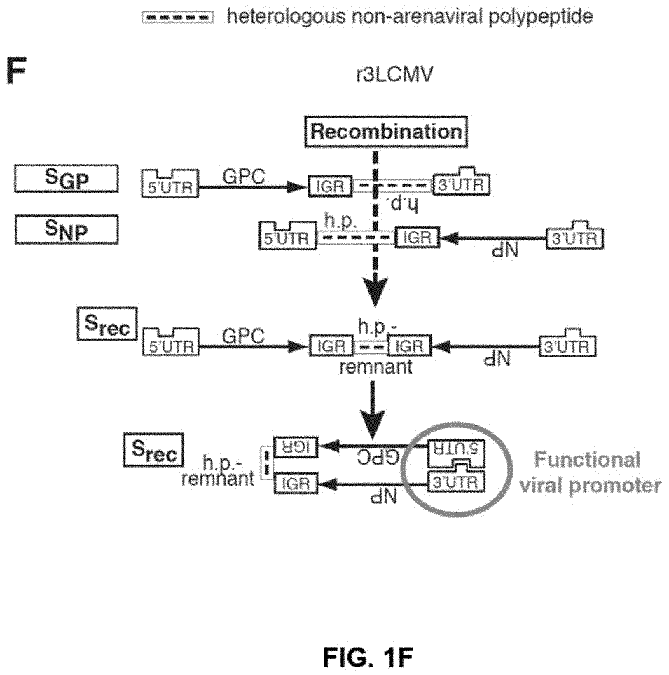

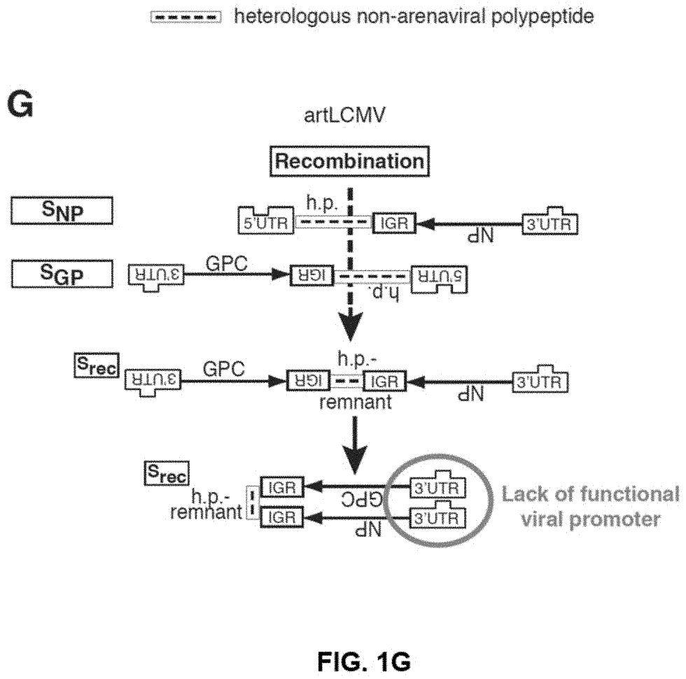

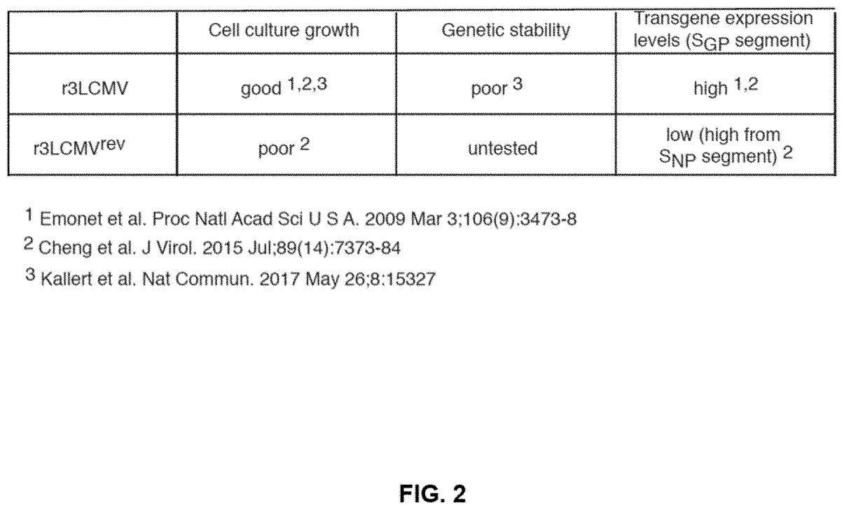

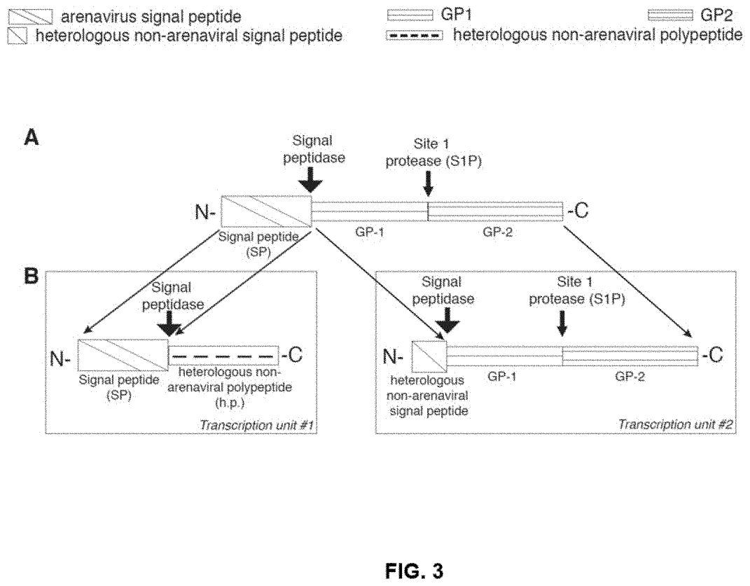

The positioning of GP SP and GP1/GP2, respectively, in the genome arrangements of Split-B and Split-D viruses and their X and Y variants is predicted to enable inter-segmental recombination and reversion to a wild-type-like virus, in a process analogous to the one outlined for r3LCMV in FIG. 1F: One recombination event with a break point at the end of GP SP in one S segment and immediately upstream of GP1 in the other S segment is predicted to yield a bi-segmented wild-type virus. As such, the Split-B and Split-D designs were not considered for experimental testing and validation.

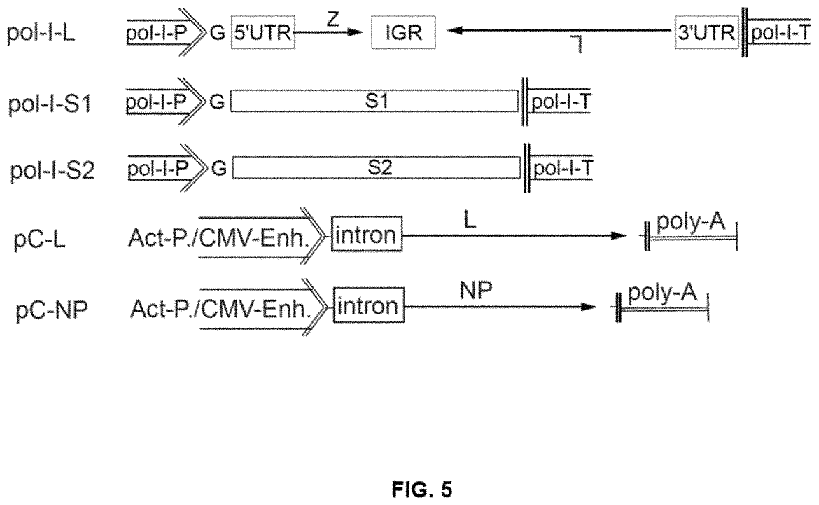

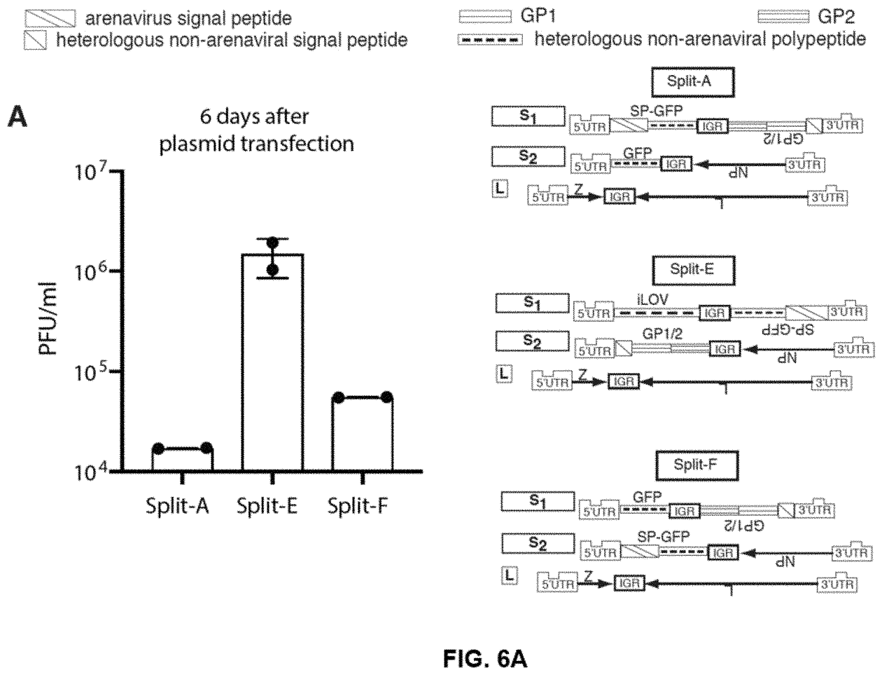

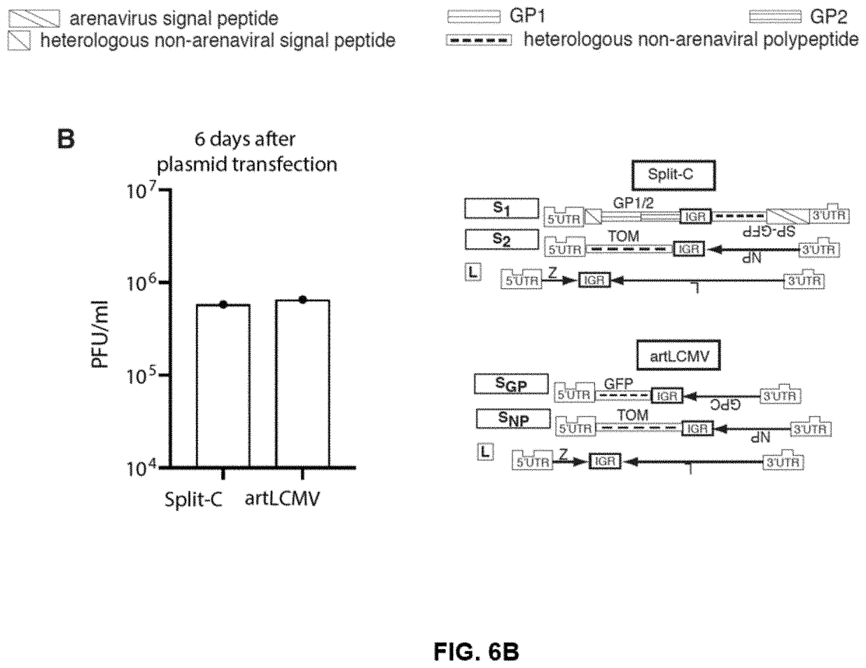

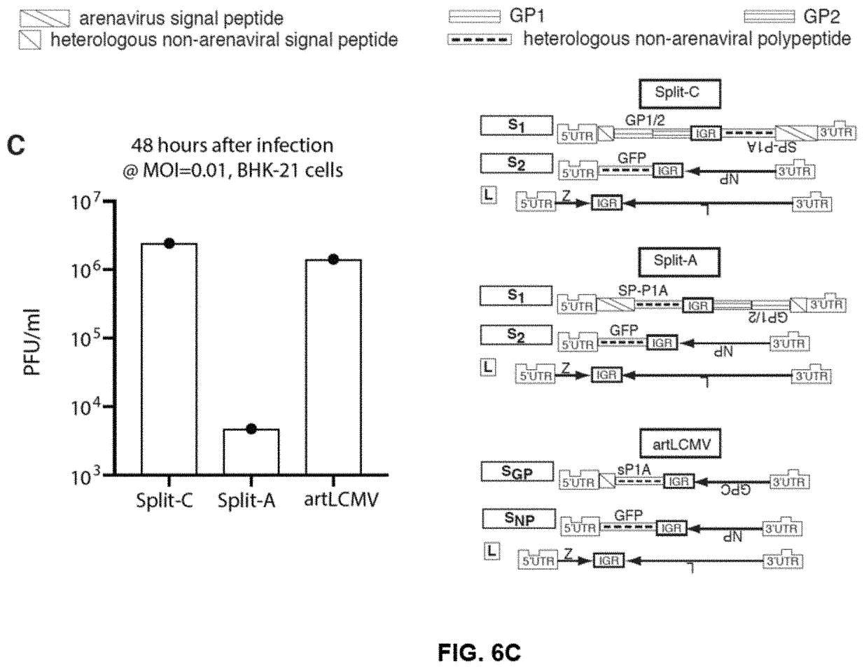

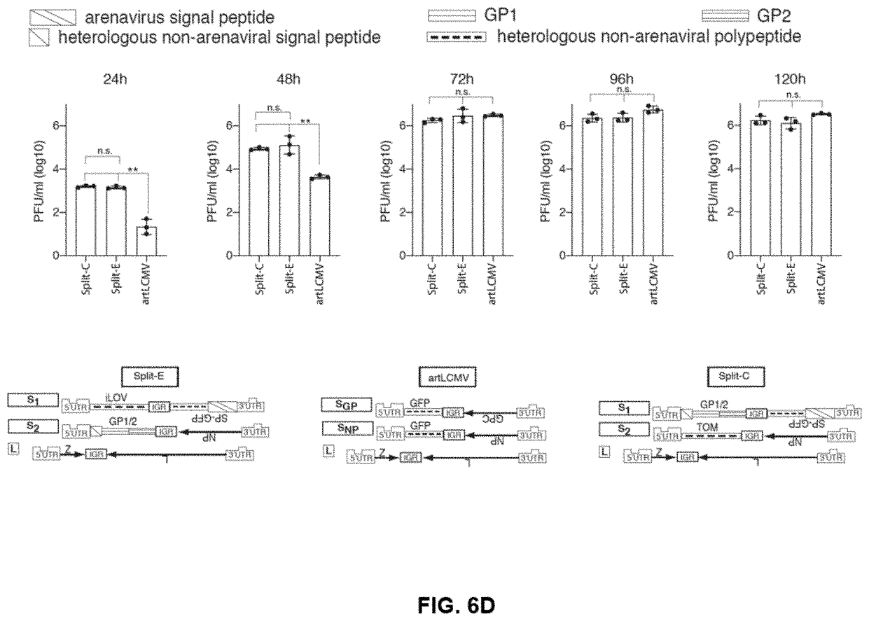

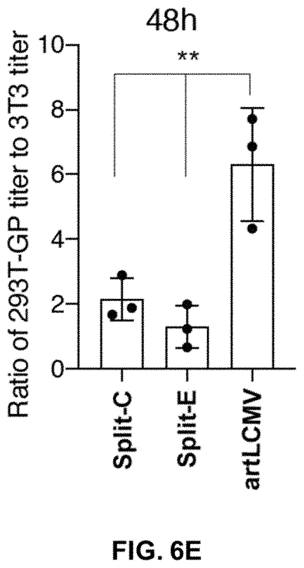

Accordingly, LCMV-based Split-A, Split-C, Split-E and Split-F viruses, each of them carrying two transgenic ORFs, were generated and their cell culture growth properties were analyzed. For this experiment, the murine polymerase-I-/polymerase-II-based arenavirus rescue system as published by Flatz et al. and Kallert et al. (FIG. 5) (Flatz L, et al. Proc Natl Acad Sci USA 2006; 103, 4663-4668; Kallert S M, et al. Nat Commun 2007; 8, 15327) was used. Plasmids for intracellular polymerase-I-driven expression of the newly designed S segments of the respective Split-A, Split-E and Split-F viruses (pol-I-S1, pol-I-S2, see schematic at right of FIG. 6A) were assembled by a commercial vendor based on synthetic DNA, which was ligated into existing expression cassettes (Flatz L, et al. Proc Natl Acad Sci USA 2006; 103, 4663-4668; Kallert S M, et al. Nat Commun 2007; 8, 15327). BHK-21 cells stably expressing the LCMV glycoprotein (Flatz L, et al. Nat Med 2010; 16, 339-345) were transfected with a combination of plasmids consisting of i) polymerase-I-based expression plasmid encoding for a wildtype LCMV L segment (pol-I-L), ii) polymerase-I-based expression plasmids encoding for the respective viruses' S1 and S2 segments (Pol-I-S1, Pol-I-S2) and polymerase-II-driven expression plasmids for the viral trans-acting factors L and NP (pC-L, pC-NP, FIG. 5). Six days after transfection, infectious titers of tri-segmented viruses were determined by immunofocus formation assay (IFF) on non-complementing 3T3 cells (Battegay M, et al. J Virol Methods 1991; 33, 191-198). The Split-E virus reached a titer >10e6 focus-forming units (PFU, FFU) per ml, whereas titers of Split-A and Split-F viruses remained below 10e5 FFU/ml (FIG. 6A). In a separate analogous experiment, side-by-side Split-C and artLCMV viruses were generated to express one GFP and one Tomato (TOM, red fluorescent protein) transgene each (see schematic at the right of FIG. 6B). Both viruses reached comparable titers close to 10e6 FFU/ml on day 6 after plasmid transfection. To determine virus growth upon infection on non-complementing BHK-21 cells (devoid of a GP transgene), Split-C, Split-A and artLCMV viruses were generated to express one GFP ORF and one P1A cancer-testis self-antigen ORF each (see schematic at the right of FIG. 6C). For comparability to the SP-flanked P1A transgenes in the Split-C and Split-A viruses, the P1A ORF in r3LCMV was expressed in-frame with an upstream tissue plasminogen activator signal sequence (“sP1A”). Supernatants were harvested 48 hours after infection of the cells at multiplicity of infection (MOI) of 0.01 and viral titers were determined. The Split-C virus reached titers exceeding 10e6 FFU/ml, which was equivalent or higher than artLCMV, whereas the titer of the Split-A virus remained below 10e4 FFU/ml.

In an additional experiment 293F suspension cells were infected with either a Split-C, a Split-E or an artLCMV vector carrying genomes as schematically outlined in FIG. 6D. Supernatant was harvested to determine viral titers over time (FIG. 6D). At early time points (24 hours, and 48 hours) after infection, the Split-C and Split-E vectors reached significantly higher titers than artLCMV (p<0.01 by 1-way ANOVA with Bonferroni post-test). When titers peaked (72 hours, 96 hours, and 120 hours after infection), the titers of all three viruses were comparable (p>0.05 by 1-way ANOVA with Bonferroni post-test). Cultures infected with artLCMV vectors are known to produce not only tri-segmented particles with a genome as depicted in FIG. 6D but additionally also an excess of bi-segmented particles carrying only the S_{NP} and the L segment but not the S_{GP} segment (Kallert S M, et al. Nat Commun 2017; 8, 15327). The latter bi-segmented particles can be visualized when performing immunofocus formation assays on GP-transgenic trans-complementing 293T cells (293T-GP cells, Flatz L, et al. Nat Med 2010; 16, 339-345). The ratio between infectious titers assessed on GP-pseudotyping 293T-GP cells and the titers detectable on non-pseudotyping 3T3 cells can, therefore, be taken as a measure for the proportion of vector particles carrying all three genome segments of artLCMV or Split vectors. Such ratio was determined for the samples collected at 48h (refer to the experiment displayed in FIG. 6D). It was found that, during the logarithmic growth phase of the vectors, the ratio was significantly higher for artLCMV than for either Split-C or Split-E (FIG. 6E). This finding indicated that compared to artLCMV, Split-C and Split-E vectors were more efficient at packaging all three genomic segments, offering a likely explanation for their more rapid growth and higher output titers in the early phase of cell culture propagation (compare to FIG. 6D).

In summary, viruses with a Split-C and Split-E genome grew to high titers, equivalent to artLCMV, whereas Split-A and Split-F grew to titers >10-fold lower than the aforementioned viruses. The commonality of the Split-C and Split-E vector design, differentiating them from the poorly growing Split-A and Split-F vector design, is their positioning of the arenavirus GP SP under control of the 3′ UTR. Thus, the positioning of the segregated arenavirus GP SP under 3′ UTR control enables growth of Split-C and Split-E vectors to titers equivalent to tri-segmented arenavirus vectors such as artLCMV, rendering the Split-C and Split-E design preferable over Split-A and Split-F. Based on the positioning of GP SP under 5′ UTR control, the Split-B and Split-D vector genomes are not expected to offer favorable growth properties either.

6.2 Several-Fold Higher Levels of Transgene Expression from S1 Segment of LCMV-based Split-C virus than from the S_{GP} segment of artLCMV

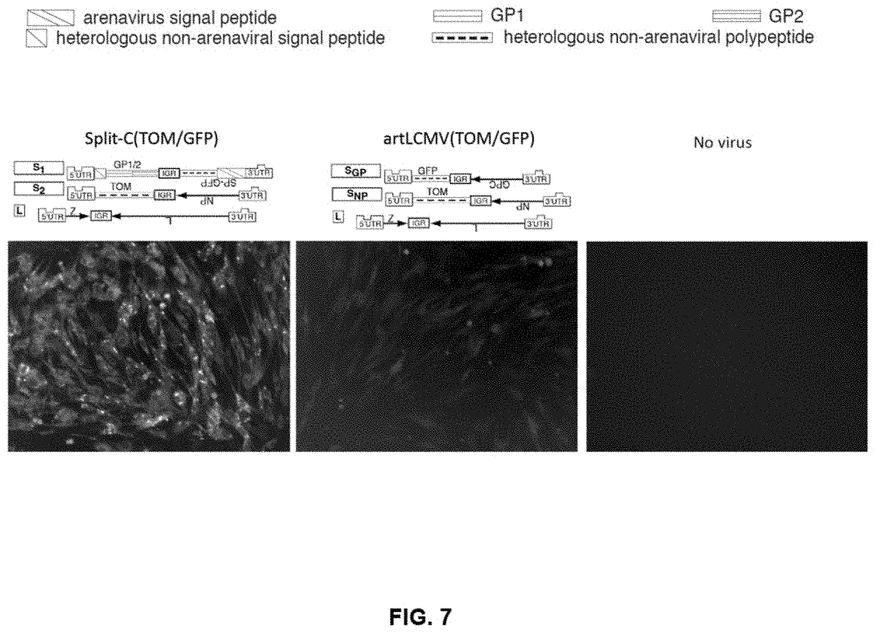

Next, the amount of transgene expressed by the LCMV-based Split-C virus and the artLCMV virus carrying one GFP and one Tomato transgene each (Split-C(TOM/GFP), artLCMV(TOM/GFP); FIG. 7) were compared on a per-cell level. BHK-21 cells were infected at MOI=0.1 and GFP was visualized by fluorescence microscopy 24 hours after infection. It was found that in Split-C(TOM/GFP)-infected cells the GFP levels were visibly higher than in cells infected with artLCMV(TOM/GFP). The GFP levels of artLCMV(TOM/GFP)-infected cells were, however, also clearly above background fluorescence of uninfected control cells (“no virus” in FIG. 7).

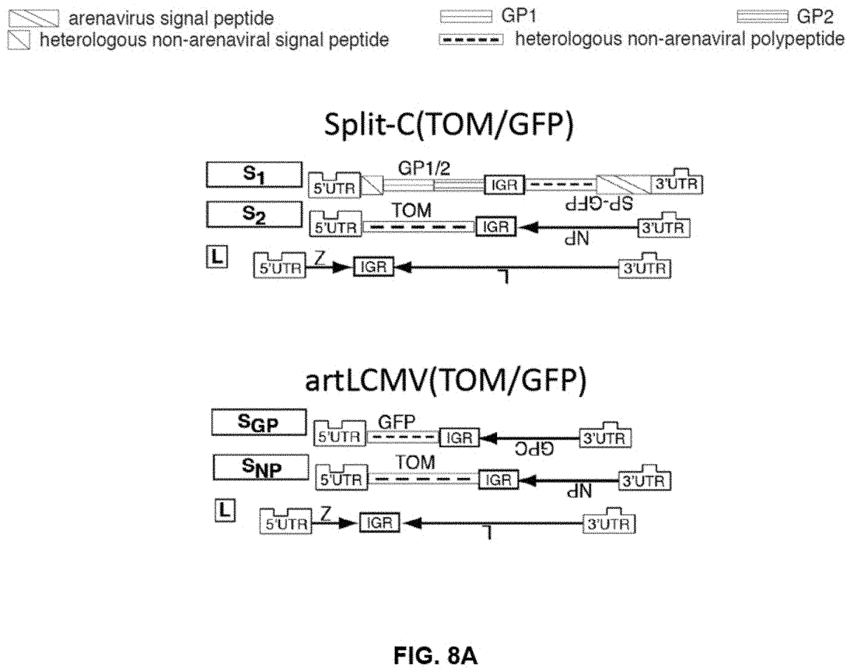

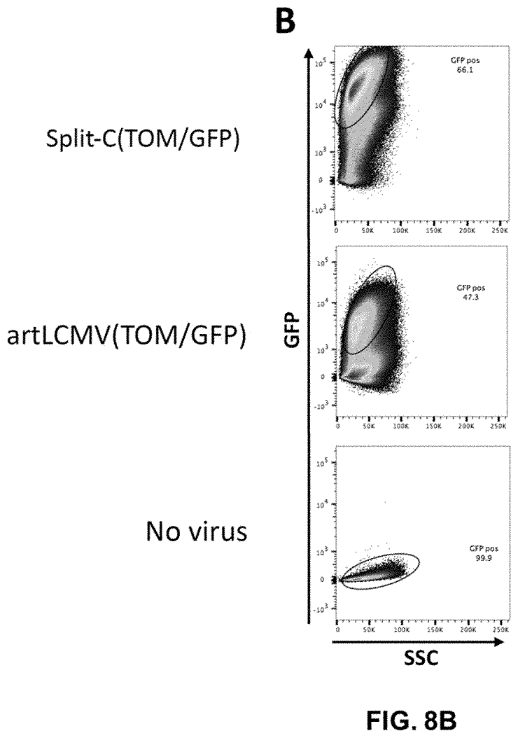

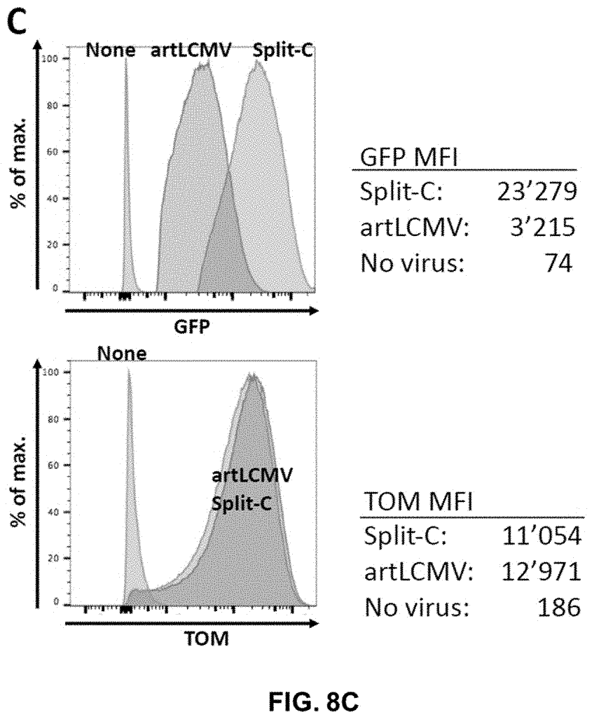

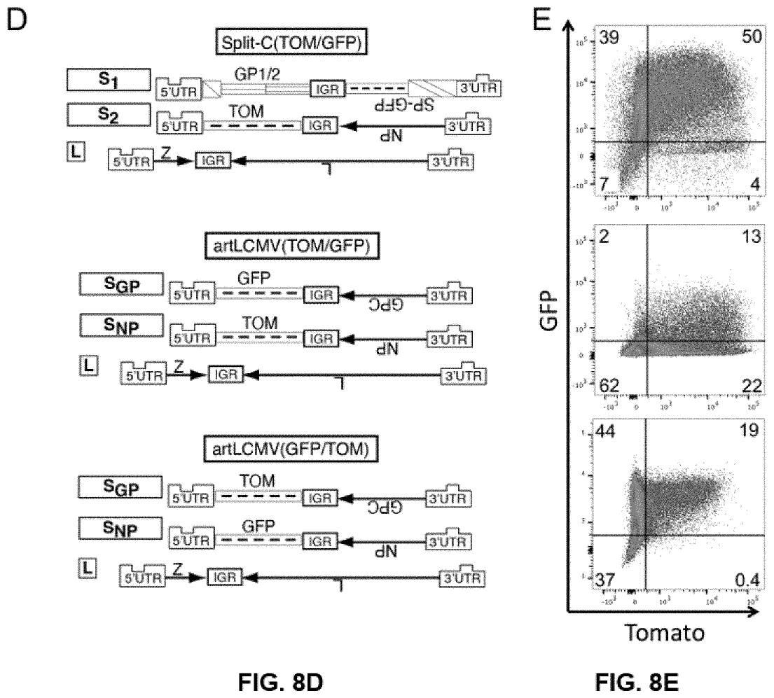

To quantify GFP and TOM expression by the two viruses, flow cytometry from the same cells were performed 8 hours later, thus 32 hours after infection. When plotting GFP against sideward scatter (SSC, FIG. 8B), a clearly separated population of GFP-expressing cells was found, both amongst Split-C(TOM/GFP)- and artLCMV(TOM/GFP)-infected cells. Those cells were gated (see FIG. 8B for gating) for further analysis and compared to the GFP-negative population of uninfected control cells (“no virus”). A histogram analysis of GFP levels in the three gated populations confirmed the visual impression obtained by fluorescence microscopy (FIG. 8C). GFP levels in Split-C(TOM/GFP)-infected cells were approximately 7-fold higher than those in cells infected with artLCMV(TOM/GFP) (FIG. 8C), while also the latter were clearly above uninfected control background. Importantly though, the two viruses expressed virtually identical levels of TOM. This was expected given that the two viruses carry the identical TOM-expressing S2/S_{NP} segments. At the same time, this finding validated the differential levels of GFP expression from the viruses' respective S1 segment by serving as an internal reference standard for the GFP measurements. Thus, it was found that the Split-C design of tri-segmented arenaviruses enabled a several-fold (˜7-fold) higher expression of the transgene encoded in the S1 segment as compared to the S_{GP} segment of artLCMV, while transgene expression levels from the S2 segment were indistinguishable from the artLCMV design's S_{NP} segment.

6.3 Higher Proportion of Cells in Split-C-Infected Cultures Co-Express Both the SNP and SGP-Encoded Transgenes than in artLCMV-Infected Culture

The ability of Split-C and artLCMV-based vectors to co-express their respective two transgenes in cells of infected cultures was analyzed. In brief, BHK-21 cells were infected with either Split-C(TOM/GFP) or artLCMV(TOM/GFP), both expressing GFP from the S1/S_{GP} segment and TOM from the S2/S_{NP} segment. The artLCMV(GFP/TOM) vector expressing GFP from the S2/S_{NP} segment and TOM from the S1/S_{GP} segment (FIG. 8D) was also included for further comparison. An earlier report (Kallert S M, et al. Nat Commun 2017; 8, 15327) has demonstrated that only a minority of cells in artLCMV vector-infected cultures express both the S_{NP}- and S_{GP}-encoded transgenes. In agreement with the earlier report, this experiment showed that 24 hours after infection at MOI=0.1 only 13% and 19% of cells in artLCMV(TOM/GFP)- and artLCMV(GFP/TOM)-infected cultures, respectively, exhibited detectable levels of both vector transgenes (FIG. 8E). Cells expressing only the S_{NP}-encoded TOM (22%) were more abundant than GFP/TOM-coexpressing cells (13%) in artLCMV(TOM/GFP)-infected cultures (FIG. 8E). Analogously, S_{NP}-encoded GFP single-positive cells in artLCMV(GFP/TOM)-infected cultures (44%) were more frequent than GFP/TOM-coexpressing cells (19%) (FIG. 8E). In remarkable contrast, approximately half of the cells in Split-C(TOM/GFP)-infected cultures expressed detectable levels of both, TOM and GFP, thus exceeding the subset of cells expressing only either GFP (39%) or TOM (4%) (FIG. 8E).

6.4 Stable Attenuation of Split-C Virus Even after Prolonged Periods of Replication in Highly Immunodeficient Mice

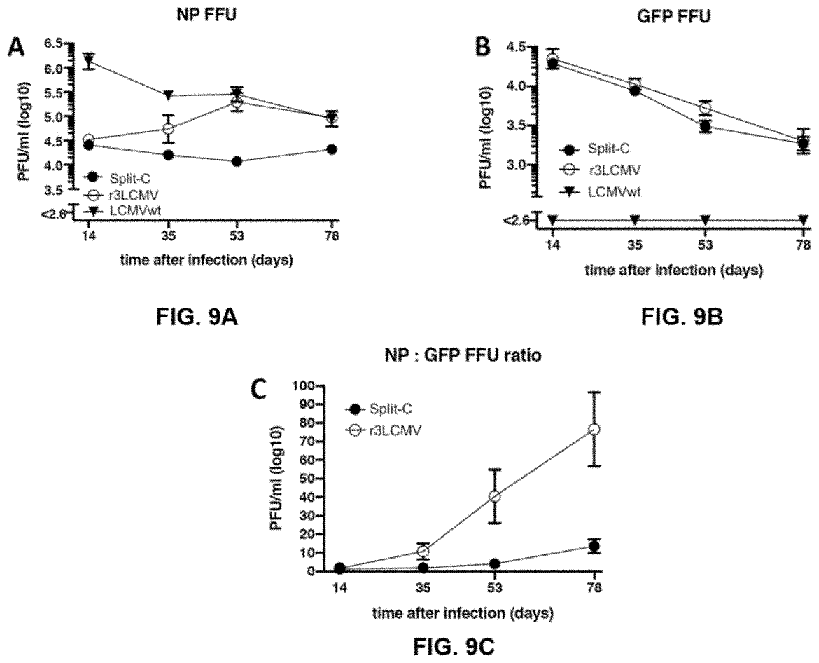

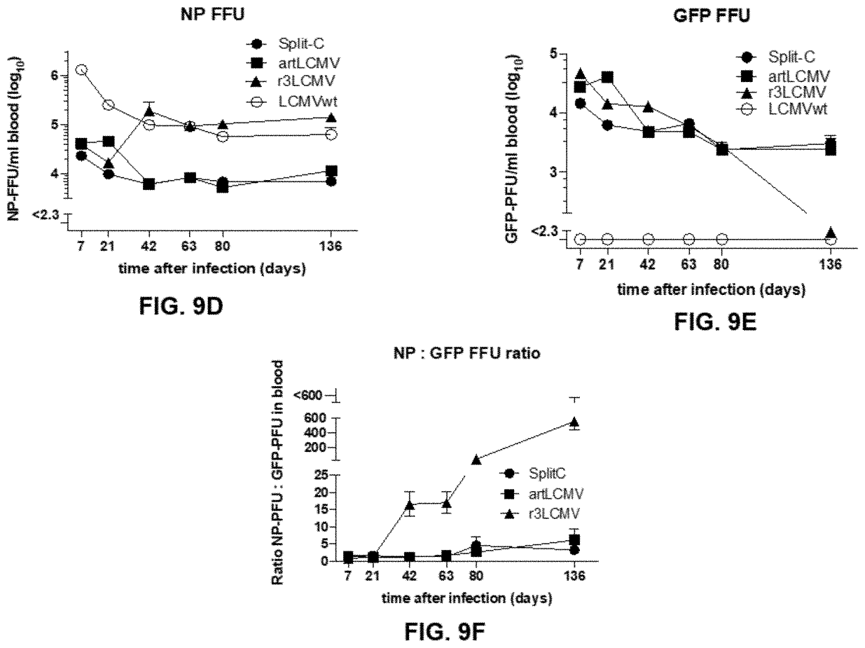

To test whether the Split-C design allowed for stable attenuation, infection experiments were performed in highly immunodeficient AGRAG mice, which lack not only T and B cells owing to RAG1-deficiency but also are homozygous for disrupted type I interferon receptor and the type II interferon receptor genes. A second group of AGRAG mice was infected with a GFP-expressing r3LCMV, which was known to be genetically unstable owing to its ability to undergo inter-segmental recombination (FIG. 1F; Kallert S M, et al. Nat Commun 2017; 8, 15327). A third group of mice was infected with virulent bi-segmented LCMV control virus (LCMVwt). Viremia over time was detected by determining NP-expressing FFU in blood, reflective of the total LCMV infectivity and thus of the replicative capacity and virulence of the three viruses (FIG. 9A). On day 14 after infection, both the Split-C- and r3LCMV-infected mice exhibited substantially lower viral loads than animals infected with virulent bi-segmented LCMVwt. Around day 35 after infection, however, viremia in r3LCMV-infected mice augmented continuously and by day 53 reached levels comparable to LCMVwt-infected animals. This continuous increase in viral loads of r3LCMV-infected mice has previously been shown to result from inter-segmental recombination and reversion to a bi-segmented viral genome (compare FIG. 1F; (Kallert S M, et al. Nat Commun 2017; 8, 15327). In stark contrast, viremia in Split-C-infected mice remained consistently lower than LCMVwt throughout the observation period of 78 days, attesting to the vector's stably attenuated phenotype. GFP-expressing FFU was determined as a measure of transgene-expressing viruses (FIG. 9B). The ratio of total viral infectivity (as determined by NP FFU) and GFP-expressing infectivity (FIG. 9C) was then calculated. For r3LCMV-infected mice this ratio increased dramatically over time, indicating that by day 78 the total number of infectious particles outnumbered transgene-expressing particles by >70-fold. In contrast, this ratio remained substantially lower in Split-C-infected mice.

A second independent infection experiment in AGRAG mice was performed to further test and compare genetic stability. In addition to the three experimental groups included in the experiment to FIGS. 9A-9C, a fourth group of mice was infected with GFP-expressing artLCMV, which was known to be genetically stable (FIG. 1G; Kallert S M, et al. Nat Commun 2017; 8, 15327). Viremia over time was detected by determining NP-expressing FFU in blood, reflective of the total LCMV infectivity and thus of the replicative capacity and virulence of the four viruses (FIG. 9D). On day 7 and 21 after infection, Split-C-, artLCMV- and r3LCMV-infected mice exhibited substantially lower viral loads than animals infected with virulent bi-segmented LCMVwt. Between day 21 and day 42 after infection, however, viremia in r3LCMV-infected mice increased substantially and from day 42 onwards remained at a plateau comparable to LCMVwt-infected animals. This increase in viral loads of r3LCMV-infected mice has previously been shown to result from inter-segmental recombination and reversion to a bi-segmented viral genome (compare FIG. 1F; (Kallert S M, et al. Nat Commun 2017; 8, 15327). In stark contrast, viremia in Split-C- and artLCMV-infected mice remained consistently lower than LCMVwt throughout the observation period of 136 days, attesting to the Split-C vector's stably attenuated phenotype. GFP-expressing FFU was determined as a measure of transgene-expressing viruses (FIG. 9E) and the ratio of total viral infectivity (as determined by NP FFU) and GFP-expressing infectivity was calculated (FIG. 9F). For r3LCMV-infected mice this ratio increased dramatically over time, indicating that by day 136 the total number of infectious particles outnumbered on average the transgene-expressing particles by >600-fold. In contrast, the corresponding average ratios in Split-C- and artLCMV-infected mice remained <10.

These data demonstrated that the segregation of GP SP from GP1/GP2 in the design of tri-segmented arenavirus genomes as exemplified here by the Split-C design, allowed for stable attenuation even after prolonged periods of replication in highly immunodeficient mice.

6.5 Transgene Delivery on the S1 Segment of an LCMV-Based Split-C Vector Results in Higher Immunogenicity than its Expression from the S_{GP} Segment of an artLCMV Vector

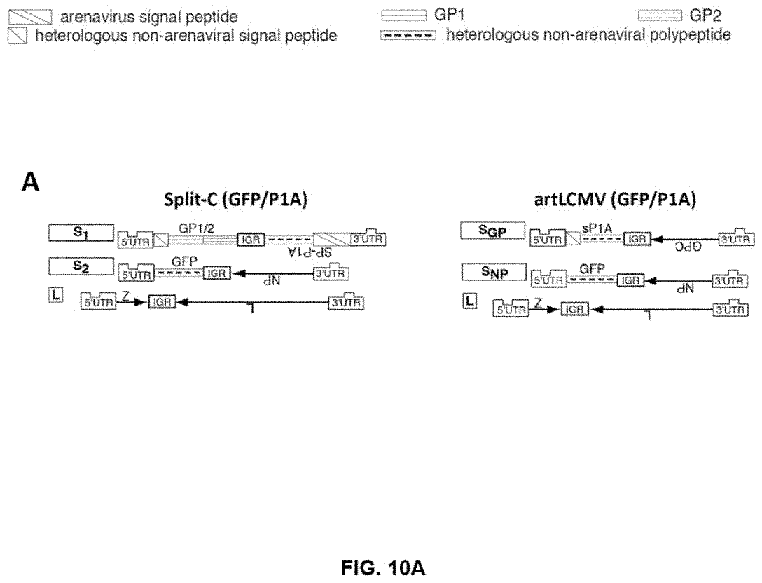

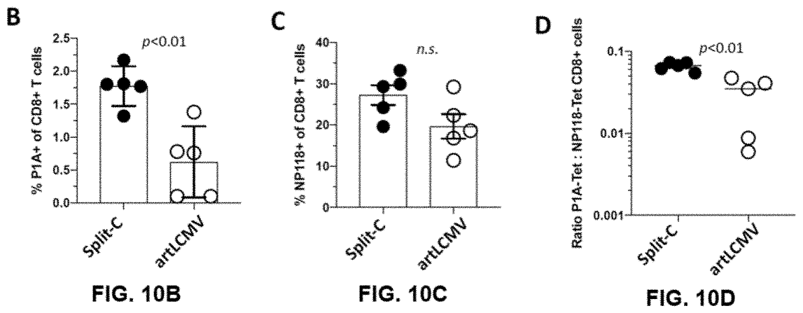

Given that transgene expression levels from the S1 segment of an LCMV-based Split-C virus were approximately 7-fold higher than from the corresponding S_{GP} segment of artLCMV (FIG. 7 and FIGS. 8A-8E), the level of immunogenicity was then evaluated to see if there was a correlation between the transgene expression level and the immunogenicity. First, CD8+ T cell responses to a P1A transgene expressed from the S1 segment of an LCMV-based Split-C virus (Split-C(P1A/GFP)) was assessed and compared to the immunogenicity of an artLCMV vector delivering in its S_{GP} segment the P1A ORF fused to an upstream tissue plasminogen activator signal sequence (“sP1A”, artLCMV(GFP/sP1A)). Thereby, both P1A translation products were ER-inserted, which can augment immunogenicity (Malin A S, et al. Microbes Infect 2000; 2, 1677-1685). The two vectors' genomes both carried a GFP transgene on their identical S2 and S_{NP} segments, respectively (FIG. 10A). On day 9 after immunization, P1A epitope-specific CD8+ T cell frequencies in blood were determined by flow cytometry using MHC class I tetramers. Mean P1A-specific CD8+ T cell frequencies in Split-C(P1A/GFP)-immunized mice were 2.8-fold higher than in controls immunized with artLCMV(GFP/sP1A), and this difference was statistically significant (FIG. 10B, p<0.01 by unpaired two-tailed student's 1-test). In contrast, CD8+ T cell responses to the two vector's common backbone epitope NP118 were not significantly different between the two groups of animals (p>0.05, FIG. 10C). The ratio between P1A epitope-specific and vector backbone-specific CD8+ T cell responses (P1A: NP118 ratio, FIG. 10D) was again significantly different between the two vectors (p<0.01). These results with P1A-expressing vectors demonstrated that the Split-C design of an LCMV vector elicited higher immunogenicity of the transgene encoded in its S1 segment than the responses elicited by a transgene encoded in the S_{GP} segment of artLCMV. Thereby, the Split-C design shifted immunodominance towards transgene-specific responses and away from vector backbone epitopes.

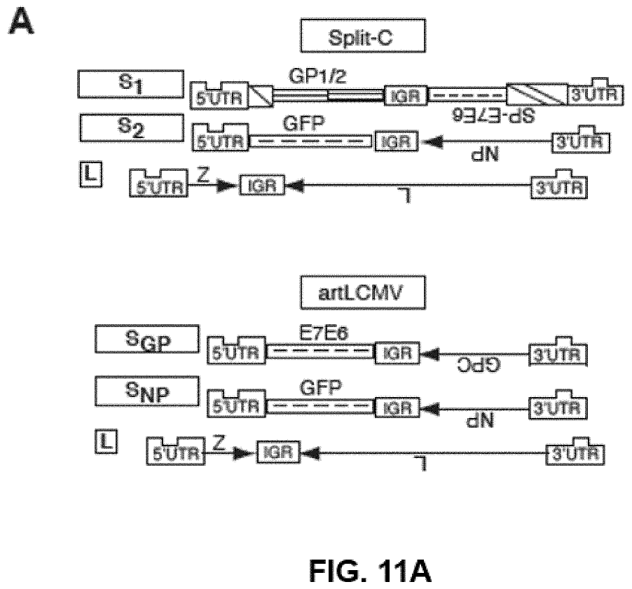

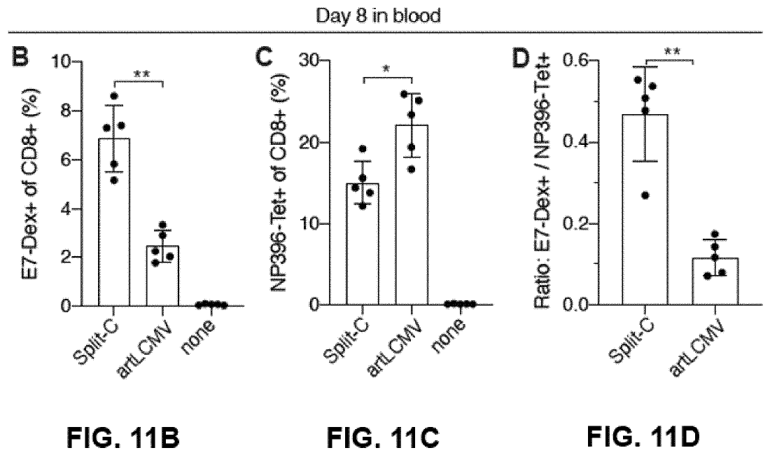

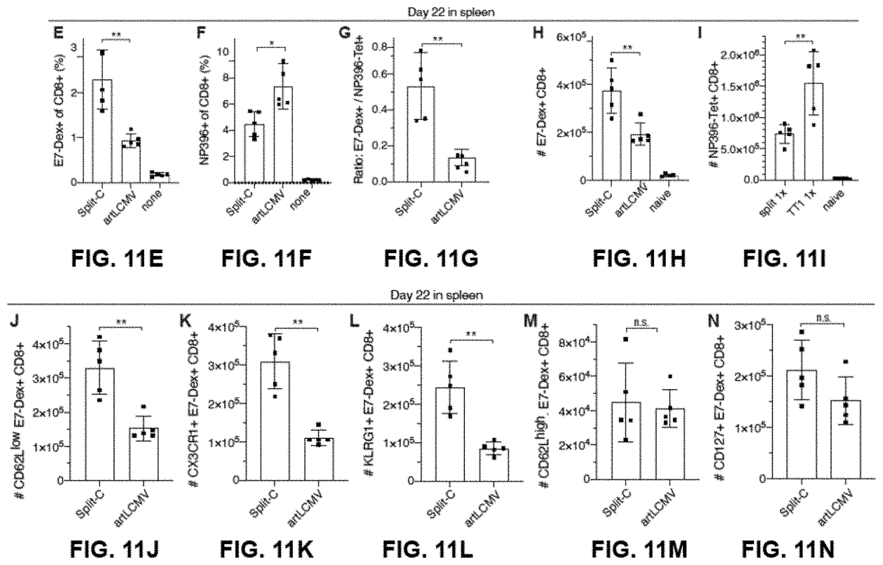

To confirm and extend these observations, CD8+ T cell responses of C57BL6 mice to a non-oncogenic fusion antigen (E7E6) comprising the HPV16 E7 and E6 proteins (Cassetti et al. Vaccine 2004; 3-4, 520-527) were studied. Split-C- and artLCMV-based vectors expressing E7E6 from their respective S1/S_{GP} segments, in conjunction with a GFP transgene on their identical S2/S_{NP} segments were engineered (FIG. 11A). When assessing the acute effector response from blood on day 8 after immunization, the Split-C vector induced significantly higher frequencies of E7-specific CD8+ T cells than the artLCMV vector (FIG. 11B). In contrast, CD8+ T cell responses to the immunodominant LCMV nucleoprotein backbone-derived epitope NP396 were of higher frequency in artLCMV-immunized mice (FIG. 11C). These differences translated into a higher and thus more favorable immunodominance ratio of E7 transgene:NP396 backbone responses in Split-C- as compared to artLCMV-immunized mice (FIG. 11D). CD8+ T cell memory was assessed on day 22 after immunization from the spleen of the same animals. In parallel to the acute effector response, Split-C-immunized mice exhibited higher frequencies of E7-specific CD8+ T cells (FIG. 11E) but lower frequencies of NP396-specific CD8+ T cells (FIG. 11F) than artLCMV-immunized mice. These differences translated again into a higher E7:NP396 epitope dominance ratio in Split-C-vaccinated animals (FIG. 11G). Also in terms of absolute cell counts, E7-specific CD8+ T cells of Split-C-immunized mice exceeded those of artLCMV-immunized animals (FIG. 11H) while NP396-specific CD8+ T cells were higher in the artLCMV-vaccinated group (FIG. 11I). When studying the subset distribution of the vector-induced CD8+ T cell response, it was noted that Split-C-immunized mice harboured significantly higher numbers of E7-specific CD62L^{low} effector memory CD8+ T cells than artLCMV-immunized mice (FIG. 11J). Analogous differences were found for cells expressing the effector markers CX3CR1 (FIG. 11K) or KLRG1 (FIG. 11L). In contrast, E7-specific CD62L^{high} central memory CD8+ T cells (FIG. 11M) and the subset expressing the memory precursor marker CD127+ (FIG. 11N) were similarly abundant in Split-C- and artLCMV-immunized mice. Altogether these findings in C57BL/6 mice confirmed the observations in P1A-immunized BALB/c mice that Split-C vectors induce higher CD8+ T cell responses, both in frequency and absolute number, to S1-/S_{GP}-encoded transgenes than artLCMV vectors, with a concomitant shift in transgene-to-backbone epitope dominance. Furthermore, these latter results indicated that Split-C-induced CD8+ T cell responses excelled primarily in their ability to induce effector/effector-memory CD8+ T cell responses to S_{GP}-encoded transgenes, while central memory responses were comparable in magnitude to artLCMV-induced ones.

### 6.6. Generation and Cell Culture Growth of Junin Candid #1-Based Split-C Vectors

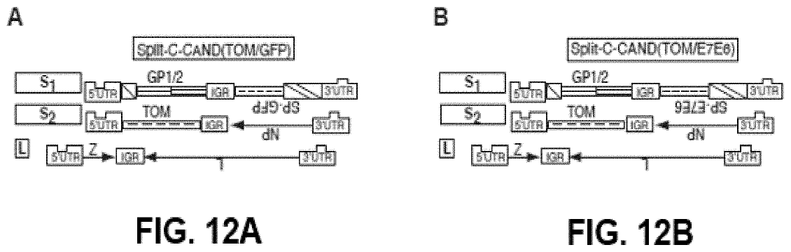

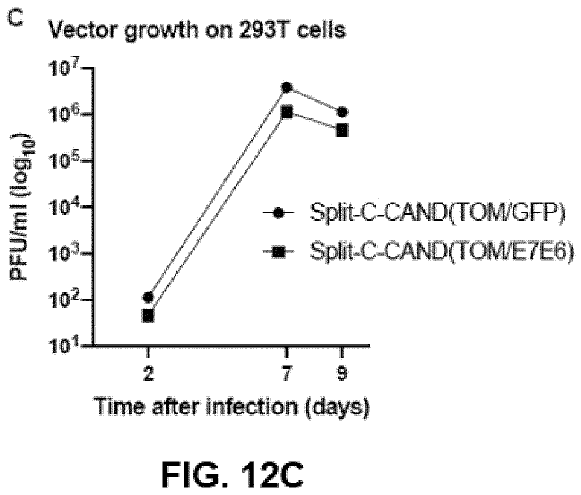

Split-C vectors based on the Junin virus vaccine strain Candid #1 were generated (Split-C-CAND), each of them carrying two transgenic ORFs, and their cell culture growth properties were analyzed. For this experiment, the murine polymerase-I-/polymerase-II-based arenavirus rescue system as published by Flatz et al. and Kallert et al. (FIG. 5) (Flatz L, et al. Proc Natl Acad Sci USA 2006; 103, 4663-4668; Kallert S M, et al. Nat Commun 2007; 8, 15327) was used. Plasmids for intracellular polymerase-I-driven expression of the newly designed S segments of the respective Split-C-CAND (pol-I-S1, pol-I-S2; for intracellular expression of the S1 and S2 segments depicted in FIGS. 12A-12B) were assembled based on existing expression cassettes (Flatz L, et al. Proc Natl Acad Sci USA 2006; 103, 4663-4668; Kallert S M, et al. Nat Commun 2007; 8, 15327). BHK-21 cells stably expressing the LCMV glycoprotein (Flatz L, et al. Nat Med 2010; 16, 339-345) were transfected with a combination of plasmids consisting of i) polymerase-I-based expression plasmid encoding for a wildtype Candid #1 L segment (pol-I-L), ii) polymerase-I-based expression plasmids encoding for the respective viruses' S1 and S2 segments (Pol-I-S1, Pol-I-S2) and polymerase-II-driven expression plasmids for the Candid #1 trans-acting factors L and NP (pC-L, pC-NP, FIG. 5). Five days after transfection, transfection culture supernatants were blindly passaged on 293T cells (devoid of transgenic GP expression). Supernatants from this blind passage were collected 2, 7 and 9 days later and infectious, replication-competent titers of tri-segmented Split-C-CAND(TOM/GFP) and Split-C-CAND(TOM/E7E6) were determined by immunofocus formation assay (IFF) on non-complementing 3T3 cells (Battegay M, et al. J Virol Methods 1991; 33, 191-198) using a Junin virus nucleoprotein-specific antibody for detection. On day 7, both Split-C-CAND(TOM/GFP) and Split-C-CAND(TOM/E7E6) vectors reached peak titers >1′000′000 FFU/ml, documenting that not only Old World arenaviruses such as LCMV but also New World arenaviruses such as Junin Candid #1 can be converted into replication-competent vectors based on the Split-C design.

6.7 Higher Levels of Transgene Expression from S1 Segment of CAND-Based Split-C Virus than from the SGP Segment of artCAND

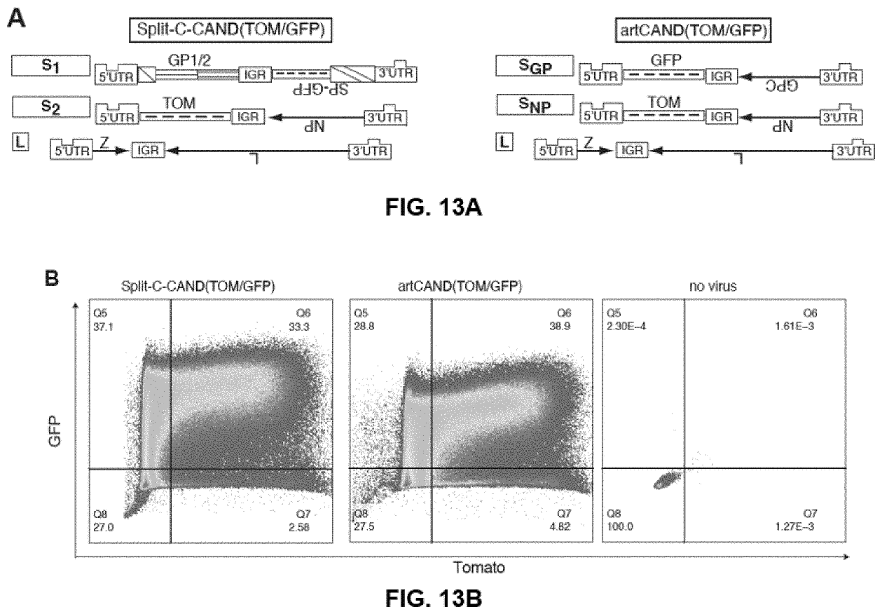

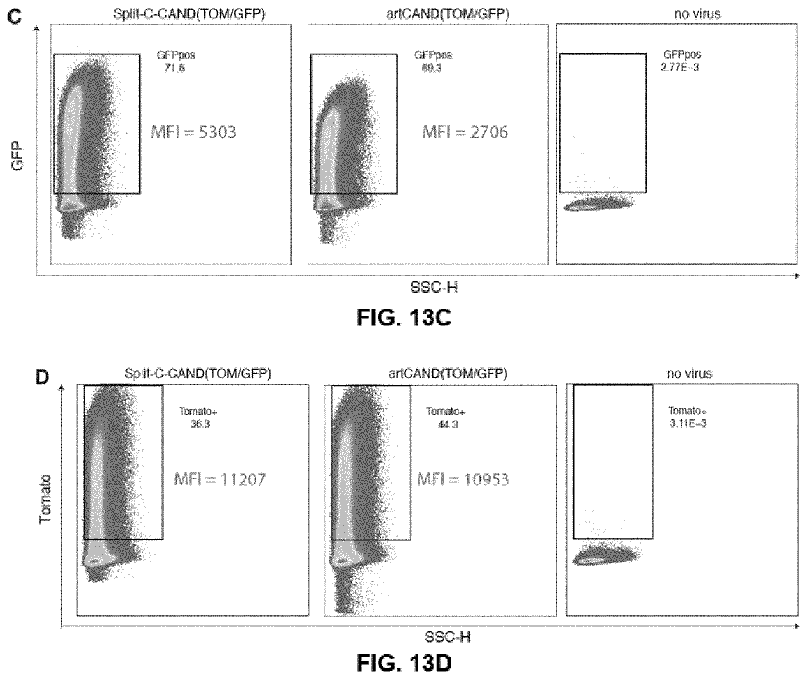

Next, the amount of transgene expressed by the CAND-based Split-C virus and a comparator artCAND virus carrying one GFP and one Tomato transgene each (Split-C-CAND(TOM/GFP), artCAND(TOM/GFP); FIG. 13A) were compared on a per-cell level. 293T cells were infected at MOI=0.1 with either one of the aforementioned viruses or were left uninfected (“no virus”). GFP as well as TOM expression was quantified by flow cytometry 72 hours later (FIGS. 13B-13D). A comparable proportion of GFP/TOM coexpressing population of cells was observed in Split-C-CAND(TOM/GFP)- and artCAND(TOM/GFP-infected cell cultures (FIG. 13B). When plotting GFP against sideward scatter (SSC, FIG. 13C), a clearly separated population of GFP-expressing cells was found, both amongst Split-C-CAND(TOM/GFP)- and amongst artCAND(TOM/GFP)-infected cells, but the GFP mean fluorescence intensity (MFI) of Split-C-CAND(TOM/GFP)-infected cells was approximately 2-fold higher than of artCAND(TOM/GFP)-infected cells. Importantly though, the two viruses expressed virtually identical levels of TOM (FIG. 13D). This was expected given that the two viruses carry the identical TOM-expressing S2/S_{NP} segments. At the same time, this finding validated the differential levels of GFP expression from the viruses' respective S1/S_{GP} segment by serving as an internal reference standard for the GFP measurements. Thus, it was found that the Split-C design of tri-segmented Candid #1 enabled a ˜2-fold higher expression of the transgene encoded in the S1 segment as compared to the S_{GP} segment of artCAND, while transgene expression levels from the S2 segment were indistinguishable from the artCAND design's S_{NP} segment.

6.8 Transgene Delivery on the S1 Segment of a CAND-Based Split-C Vector Results in Higher Immunogenicity than its Expression from the SGP Segment of an artCAND vector

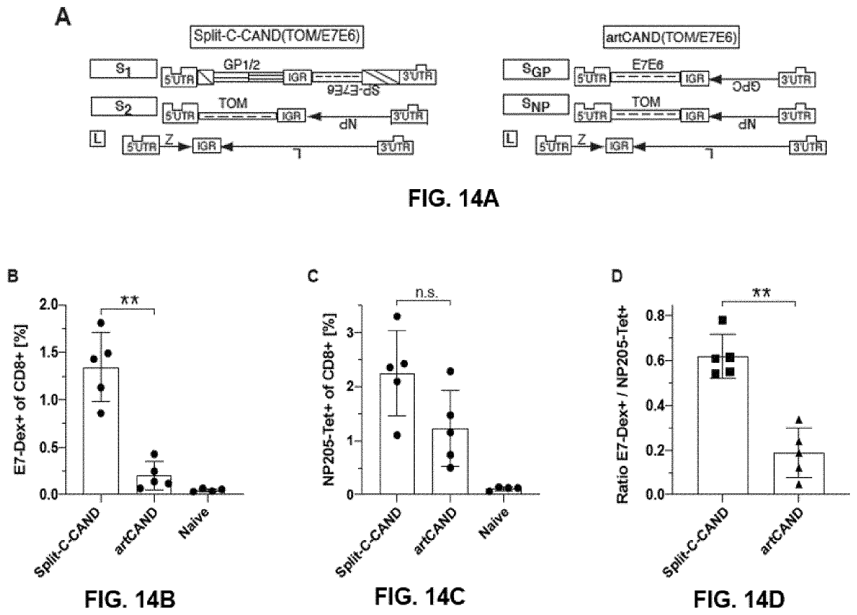

Given that transgene expression levels from the S1 segment of a CAND-based Split-C virus were approximately 2-fold higher than from the corresponding S_{GP} segment of artCAND (FIG. 13C), the level of immunogenicity was then evaluated to see if there was a correlation between the transgene expression level and the immunogenicity. CD8+ T cell responses to an artificial HPV16 E7E6 fusion antigen expressed from the S1 segment of a CAND-based Split-C virus (Split-C-CAND(TOM/E7E6)) was assessed and compared to the immunogenicity of an artCAND(TOM/E7E6) vector delivering in its S_{GP} segment the E7E6 transgene (FIG. 14A). The two vectors' genomes both carried a TOM transgene on their identical S2 and S_{NP} segments, respectively (FIG. 14A). On day 9 after immunization, E7 epitope-specific CD8+ T cell frequencies in blood were determined by flow cytometry using MHC class I tetramers. Mean E7-specific CD8+ T cell frequencies in Split-C-CAND(TOM/E7E6)-immunized mice were ˜7-fold higher than in controls immunized with artCAND(TOM/E7E6), and this difference was statistically significant (FIG. 14B, p<0.01 by unpaired two-tailed student's 1-test). In contrast, CD8+ T cell responses to the two vector's common backbone epitope NP205 (YTVKYPNL; SEQ ID NO: 47) were not significantly different between the two groups of animals (p>0.05, FIG. 14C). The ratio between E7 epitope-specific and NP205 vector backbone-specific CD8+ T cell responses (E7:NP205 ratio, FIG. 14D) was also significantly different between the two vectors (p<0.01). These results with E7E6-expressing vectors demonstrated that the Split-C design of a Candid #1-based vector elicited higher immunogenicity of the transgene encoded in its S1 segment than the responses elicited by a transgene encoded in the S_{GP} segment of artCAND. Thereby, the Split-C design shifted immunodominance towards transgene-specific responses and away from vector backbone epitopes.

### 7. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 8. SEQUENCE LISTING

| SEQ ID | | |
|---|---|---|
| NO. | Description | Sequence |
| 1 | Amino acid sequence of | MGQIVTMFEALPHIIDEVINIVIIVLIIITSIKAVYNFATCGILAL |
| | glycoprotein precursor in | VSFLFLAGRSCGMYGLNGPDIYKGVYQFKSVEFDMSHLNLT |
| | LCMV | MPNACSANNSHHYISMGSSGLELTFTNDSILNHNFCNLTSAF |
| | | NKKTFDHTLMSIVSSLHLSIRGNSNHKAVSCDFNNGITIQYN |
| | | LSFSDPQSAISQCRTFRGRVLDMFRTAFGGKYMRSGWGWA |
| | | GSDGKTTWCSQTSYQYLIIQNRTWENHCRYAGPFGMSRILF |
| | | AQEKTKFLTRRLAGTFTWTLSDSSGVENPGGYCLTKWMIL |
| | | AAELKCFGNTAVAKCNVNHDEEFCDMLRLIDYNKAALSKF |
| | | KQDVESALHVFKTTVNSLISDQLLMRNHLRDLMGVPYCNY |
| | | SKFWYLEHAKTGETSVPKCWLVTNGSYLNETHFSDQIEQEA |
| | | DNMITEMLRKDYIKRQGSTPLALMDLLMFSTSAYLISIFLHL |
| | | VKIPTHRHIKGGSCPKPHRLTNKGICSCGAFKVPGVKTIWKR |
| | | R |
| 2 | Amino acid sequence of | MGQIITFFQEVPHVIEEVMNIVLIALSLLAILKGVYNVATCGII |
| | glycoprotein precursor in | GLVTFLFLCGRSCSLIYKGSYELQTLELNMETLNMTMPLSCT |
| | Lassa virus | KNSSHHYIRVGNETGLELTLTNTSIINHKFCNLSDAHKRNLY |
| | | DHALMSILSTFHLSIPNFNQYEAMSCDFNGGKISVQYNLSHA |
| | | YAVDAAEHCGTVANGVLQTFMRMAWGGSYIALDSGRGN |
| | | WDCIMTSYQYLIIQNTTWEDHCQFSRPSPIGYLGLLSQRTRD |
| | | IYISRRLLGTFTWTLSDSEGNETPGGYCLTRWMLIEAELKCF |
| | | GNTAVAKCNEKHDEEFCDMLRLFDFNKQAIQRLKSEAQMS |
| | | IQLINKAVNALINDQLIMKNHLRDMMGIPYCNYSKYWYLN |
| | | HTSSGRTSLPKCWLVSNGSYLNETHFSDDIEQQADNMITEM |
| | | LQKEYIDRQGKTPLGLVDLFVFSTSFYLISIFLHLIKIPTHRHI |
| | | VGKPCPKPHRLNHMGICSCGLYKQPGVPTRWKR |
| 3 | Amino acid sequence of | MGQVVTLIQSIPEVLQEVFNVALIIVSTLCIIKGFVNLMRCGL |
| | glycoprotein precursor in | FQLITFLILAGRSCDGMMIDRRHNLTHVEFNLTRMFDNLPQS |
| | Pichinde virus | CSKNNTHHYYKGPSNTTWGIELTLTNTSIANETTGNFSNIRS |
| | | LAYGNISNCDKTEEAGHTLKWLLNELHFNVLHVTRHVGAR |
| | | CKTVEGAGVLIQYNLTVGDRGGEVGRHLIASLAQIIGDPKIA |
| | | WVGKCFNNCSGGSCRLTNCEGGTHYNFLIIQNTTWENHCT |
| | | YTPMATIRMALQKTAYSSVSRKLLGFFTWDLSDSTGQHVPG |
| | | GYCLEQWAIVWAGIKCFDNTVMAKCNKDHNEEFCDTMRL |
| | | FDFNQNAIKTLQLNVENSLNLFKKTINGLISDSLVIRNSLKQL |
| | | AKIPYCNYTKFWYINDTITGRHSLPQCWLVHNGSYLNETHF |
| | | KNDWLWESQNLYNEMLIKEYEERQGKTPLALTDICFWSLV |
| | | FYTITVFLHLVGIPTHRHIIGDGCPKPHRITRNSLCSCGYYKIP |
| | | KKPYKWVRLGK |
| 4 | Amino acid sequence of | MGQFISFMQEIPTFLQEALNIALVAVSLIAIIKGIVNLYKSGLF |
| | glycoprotein precursor in | QFFVFLALAGRSCTEEAFKIGLHTEFQTVSFSMVGLFSNNPH |
| | Junin virus | DLPLLCTLNKSHLYIKGGNASFQISFDDIAVLLPQYDVIIQHP |
| | | ADMSWCSKSDDQIWLSQWFMNAVGHDWHLDPPFLCRNRA |
| | | KTEGFIFQVNTSKTGVNGNYAKKFKTGMHHLYREYPDPCL |
| | | NGKLCLMKAQPTSWPLQCPLDHVNTLHFLTRGKNIQLPRRS |
| | | LKAFFSWSLTDSSGKDTPGGYCLEEWMLVAAKMKCFGNT |
| | | AVAKCNLNHDSEFCDMLRLFDYNKNAIKTLNDETKKQVNL |
| | | MGQTINALISDNLLMKNKIRELMSVPYCNYTKFWYVNHTL |
| | | SGQHSLPRCWLIKNNSYLNISDFRNDWILESDFLISEMLSKE |
| | | YSDRQGKTPLTLVDICIWSTVFFTASLFLHLVGIPSHRHIRGE |
| | | ACPLPHRLNSLGGCRCGKYPNLKKPTVWRRGH |
| 5 | Amino acid sequence of | MGQVIGFFQSLPNIINEALNIALICVALIAILKGIVNIWKSGLI |
| | glycoprotein precursor in | QLFIFLILAGRSCSHTFQIGRNHEFQSITLNFTQFLGYAPSSCS |
| | Oliveros virus | VNNTHHYFRGPGNVSWGIELTLTNNSVINASNSLKVFTNIH |
| | | HNITNCVQNIDEQDHLMKWLIETMHLQIMKPGKRLPPILCE |
| | | KDKGLLIEYNLTNIASREEKHSEYWSQLLYGLSKLLGSSKSL |
| | | WFDYCQRADCMMQEHSSHLKCNYSECSGHTTFKYLILQNT |
| | | TWENHCEFNHLNTIHLLMSSTGQSFITRRLQAFLTWTLSDAT |
| | | GNDLPGGYCLEQWAIVWAGIKCFGNTAVAKCNQNHDSEFC |
| | | DMLRLFDYNRNAIKSLNDQSQSRLNLLTNTINSLISDNLLMK |
| | | NKLAEIMNIPYCNYTKFWYINDTRTGRHTLPQCWLISNGSY |
| | | LNETKFRTQWLSESNALYTEMLTEDYDKRQGSTPLSLVDLC |
| | | FWSTLFYVTTLFAHLVGFPTHRHILDGPCPKPHRLTKKGICS |
| | | CGHFGIPGKPVRWVKRSR |
| 6 | Amino acid sequence of | MGQLISFFGEIPTILQEALNIALIAVSIIATIKGVVNVWKSGLI |
| | glycoprotein precursor in | QLLMFVMLAGRSCSVQIGHHLELEHIILNSSSILPFTPTLCKL |
| | Tamiami virus | NKTYFLVRGPFQAHWGVDLAIGSTTVAVENATKTYTLKSK |
| | | NFTGCFEGNPDPDSAALLVTWLFNSLHHDYKNDPSILCERV |
| | | SGENSFRFQINISEPEYCEKILSRMANLFGSFENYCLNNRHIK |
| | | KLIIIRNLTWSQQCHENHMSAMQLITSNIHTQVVRARRILSFF |
| | | TWSLSDAVGNDMPGGYCLEKWMLIASQLKCFGNTAVAKC |
| | | NLNHDSEFCDMLRLFDFNRKAIETLQNKTRSQLNIAINAINS |
| | | LISDNLLMKNRVKELMDIPFCNYTKFWYVNHTKLNHHSLP |
| | | RCWLVKNGSYLNESEFRNDWLLESDHLISEILSREYEERQG |
| | | RTPLSLVDVCFWSTLFYTASIFLHLIRIPTHRHIVGEGCPKPH |
| | | RLRADSTCACGLYKQKRRPLKWVRSN |
| 7 | Amino acid sequence of | MSLSKEVKSFQWTQALRRELQSFTSDVKAAVIKDATNLLN |
| | nucleoprotein in LCMV | GLDFSEVSNVQRIMRKEKRDDKDLQRLRSLNQTVHSLVDL |
| | | KSTSKKNVLKVGRLSAEELMSLAADLEKLKAKIMRSERPQA |
| | | SGVYMGNLTTQQLDQRSQILQIVGMRKPQQGASGVVRVW |
| | | DVKDSSLLNNQFGTMPSLTMACMAKQSQTPLNDVVQALTD |
| | | LGLLYTVKYPNLNDLERLKDKHPVLGVITEQQSSINISGYNF |
| | | SLGAAVKAGAALLDGGNMLESILIKPSNSEDLLKAVLGAKR |
| | | KLNMFVSDQVGDRNPYENILYKVCLSGEGWPYIACRTSIVG |
| | | RAWENTTIDLTSEKPAVNSPRPAPGAAGPPQVGLSYSQTML |
| | | LKDLMGGIDPNAPTWIDIEGRFNDPVEIAIFQPQNGQFIHFYR |
| | | EPVDQKQFKQDSKYSHGMDLADLFNAQPGLTSSVIGALPQ |
| | | GMVLSCQGSDDIRKLLDSQNRKDIKLIDVEMTREASREYED |
| | | KVWDKYGWLCKMHTGIVRDKKKKEITPHCALMDCIIFESA |
| | | SKARLPDLKTVHNILPHDLIFRGPNVVTL |
| 8 | Amino acid sequence of | MSNSKEVKSFLWTQSLRRELSGYCSNIKLQVVKDAQALLH |
| | nucleoprotein in Lassa | GLDFSEVSNVQRLMRKQKRDDGDLKRLRDLNQAVNNLVE |
| | virus | LKSVQQKSILRVGTLTSDDLLTLAADLEKLKSKVTRTERPLS |
| | | SGVYMGNLSSQQLDQRRALLSMIGMSGGNQNSQPKGDGV |
| | | VRVWDVKNADLLNNQFGTMPSLTLACLTKQGQVDLNDAV |
| | | QALTDLGLIYTAKYPNSSDLDRLSQSHPILNMIDTKKSSLNIS |
| | | GYNFSLGAAIKTGACMLDGGNMLETIKVTPQTMDGILKSIL |
| | | KVKRSLGMFISDTPGERNPYENILYKICLSGDGWPYIASRTAI |
| | | IGRAWENTVVDLESDSKPQKIGSNGSNKSLQSAGFAPGLTY |
| | | SQLMTLKDSMLQLDPNAKTWIDIEGRPEDPVEVALFQPISGC |
| | | YIHFFREPTDLKQFKQDAKYSHGIDVTDLFAAQPGLTSAVIE |
| | | ALPRNMVLTCQGSDDIKKLLDSQGRRDIKLIDVSLNKIDSRK |
| | | FENAVWDQYKDLCHMHTGVVVEKKKKGGKEEITPHCALL |
| | | DCIMFDAAVSGGLNATVLRAVLPRDMVFRTSSPKVVL |
| 9 | Amino acid sequence of | MSDNIPSFRWVQSLRRGLSNWTHPVKADVLSDTRALLSAL |
| | nucleoprotein in Pichinde | DFHKVAQVQRMMRKDKRTDSDLTKLRDMNKEVDALMNM |
| | virus | RSIQRDNVLKVGGLAKEELMELASDLDKLRKKVTRTESLSQ |
| | | PGVYGGNLTNTQLEQRAEILRSMGFANARPTGNRDGVVKI |
| | | WDIKDNTLLINQFGSMPALTIACMTEQGGEQLNDVVQALSA |
| | | LGLLYTVKFPNMTDLEKLTQQHSALKIISNEPSAINISGYNLS |
| | | LSAAVKAAACMIDGGNMLETIQVKPSMFSTLIKSLLQIKNRE |
| | | GMFVSTTPGQRNPYENLLYKICLSGDGWPYIGSRSQVQGRA |
| | | WDNTTVDLDSKPSAIQPPVRNGGSPDLKQIPKEKEDTVVSSI |
| | | QMLDSKATTWIDIEGTPNDPVEMAIYQPDTGNYIHCYRFPH |
| | | DEKSFKEQSKYSHGLLLKDLADAQPGLISSIIRHLPQNMVFT |
| | | AQGSDDIISLFEMHGRRDLKVLDVKLSAEQARTFEDEIWER |
| | | YNLLCTKHKGLVIKKKKKGAAQTTANPHCALLDTIMFDAT |
| | | VTGWVRDQKPMRCLPIDTLYRNNTDLINL |
| 10 | Amino acid sequence of | MAHSKEVPSFRWTQSLRRGLSQFTQTVKSDVLKDAKLIADS |
| | nucleoprotein in Junin | IDFNQVAQVQRALRKTKKGEEDLNKLRDLNKEVDRLMSM |
| | virus | RSVQRNTVFKVGDLGRDELMELASDLEKLKNKIRRAETGS |
| | | QGVYMGNLSQSQLAKRSEILRTLGFQQQGTGGNGVVRIWD |
| | | VKDPSKLNNQFGSVPALTIACMTVQGGETMNSVIQALTSLG |
| | | LLYTVKYPNLSDLDRLTQEHDCLQIVTKDESSINISGYNFSLS |
| | | AAVKAGASILDGGNMLETIRVTPENFSSLIKSTIQVKRREGM |
| | | FIDEKPGNRNPYENLLYKLCLSGDGWPYIGSRSQITGRSWD |
| | | NTSIDLTRKPVAGPRQPEKNGQNLRLANLTEIQEAVIREAVG |
| | | KLDPTNTLWLDIEGPATDPVEMALFQPAGKQYIHCFRKPHD |
| | | EKGFKNGSRHSHGILMKDIEDAMPGVLSYVIGLLPPDMVVT |
| | | TQGSDDIRKLFDLHGRRDLKLVDVKLTSEQARQFDQQVWE |
| | | KYGHLCKYHNGVVVNKKKREKDTPFKLASSEPHCALLDCI |
| | | MFQSVLDGKLYEEEPTPLLPPSLLFLPKAAYAL |
| 11 | Amino acid sequence of | MSGASDIPSFRWTQSLRRGLSHYTTQTKGDVLRDAKSLVDG |
| | nucleoprotein in Oliveros | LDFSQVSQVQRVMRKERRSDDDLLKLRDLNKAVDGLMMM |
| | virus | RNKQSNVSLKVGGLSKDELMELATDLEKLKRKINTNERSTP |
| | | GVYQGNLTTAQLDRRSAILRSLGFQSRQGQNNGVVRIWDIK |
| | | DQKQLINQFGSMPALTIACMSVQGAEQMNDVVQGLTTLGL |
| | | LYTVKYPNLDDLEKLSAEHTCLQFITREESANNISGYNLSLA |
| | | AAVKAGACLVDGGNMLETIYVKPDVFADIIKSLLRVKHQER |
| | | MFVSEKPGMRNPYENILYKICLSGEGWPYIGSRSQITGRAW |
| | | DNTTIDFSKDVMYGPPPPVKNGGNIRLNPLTDTQEAVIKEAI |
| | | SKLNPDETIWVDIEGPPTDPVELALYQPTTGYYIHCFRLPHD |
| | | EKGFKNGSKYSHGILLRDIENARSGLLSRILIKLPSKLVLTCQ |
| | | GSDDIKKLMELNGRPDISTIDLSFPTDQARFYESVVWEKFGS |
| | | LCTKHNGVVLSRKKKGGNSGEPHCALLDCLMFQAAFEGNV |
| | | PSIEPKPLLPSALVFKSESVVAM |
| 12 | Amino acid sequence of | MSDQSVPSFRWVQSLRRGLSAWTTPVKADVLNDTRALLSG |
| | nucleoprotein in Tamiami | LDFSKVASVQRMMRREKRDDNDLTNLRDLNKEVDSLMTM |
| | virus | RSSQKNMFLKVGSLSKDELMELSSDLNKLKEKVQRSERISG |
| | | SSGVYQGNLTTTQLTRRSEILQLVGIQRPGLNRRGGVVKIW |
| | | DIKEPEHSLINQFGSTPAVTISCMAEQGGETLNDVVARPNRS |
| | | VGYFTQQSFLIWVTWRALSSKHSCLKVITQEESQINISGFNLS |
| | | LSAAVKAGACLVDGGNMLETIKVEESTFTTIIKTLLEIKSKE |
| | | RMFVDITPGQRNPYENLLYKLCLSGEGWPYIASRSQIKGRA |
| | | WDNTVIEFDVSPRKPPVPIRNGGSPVLTTLKPEVEEQIKRSIE |
| | | SLSVHDTTWIDIEGPPFDPVEMAIYQPDSLKYIHCYRKPNDV |
| | | KSFKDQSKYCHGILLKDIEYCSSLALYQPSLRHLPKSMVFTA |
| | | QGSEDIRRLFDMHGRQDLKIVDVKFTAEQSRVFEELTWKRF |
| | | EHLCDKHKGIVIKSKKKGTTPASTNAHCALMDCIMFSGVLL |
| | | GAIPNDKPRRLLPLDILFREPDTTVVL |
| 13 | Amino acid sequence of Z | MGQGKSREEKGTNSTNRAEILPDTTYLGPLSCKSCWQKFDS |
| | protein in LCMV | LVRCHDHYLCRHCLNLLLSVSDRCPLCKYPLPTRLKISTAPS |
| | | SPPPYEE. |
| 14 | Amino acid sequence of Z | MGNKQAKAPESKDSPRASLIPDATHLGPQFCKSCWFENKGL |
| | protein in Lassa virus | VECNNHYLCLNCLTLLLSVSNRCPICKMPLPTKLRPSAAPTA |
| | | PPTGAADSIRPPPYSP. |
| 15 | Amino acid sequence of Z | MGLRYSKEVRKRHGDEDVVGRVPMTLNLPQGLYGRFNCK |
| | protein in Pichinde virus | SCWFVNKGLIRCKDHYLCLGCLTKMHSRGNLCEICGHSLPT |
| | | KMEFLESPSAPPYEP |
| 16 | Amino acid sequence of Z | MGNCNGASKSNQPDSSRATQPAAEFRRVAHSSLYGRYNCK |
| | protein in Junin virus | CCWFADTNLITCNDHYLCLRCHQGMLRNSDLCNICWKPLP |
| | | TTITVPVEPTAPPP |
| 17 | Amino acid sequence of Z | MGSKSSKSSGFENVPSLGLSHTNQPRVSLIREARPSLYGRYN |
| | protein in Oliveros virus | CKCCWFQNKNLVECSDHYLCLKCISSMLRRGQNCEICGKPI |
| | | PTHIAVTTAPTAPPEP |
| 18 | Amino acid sequence of Z | MGLRYSKEVRDRHGDKDPEGRIPITQTMPQTLYGRYNCKSC |
| | protein in Tamiami virus | WFANKGLIKCSNHYICLRCLTSMLNRTDYCEICGEVLPKRL |
| | | TFETTPTAPPYTP |
| 19 | Amino acid sequence of L | MDEIISELRELCLNYIEQDERLSRQKLNFLGQREPRMVLIEGL |
| | protein in LCMV | KLLSRCIEIDSADKSGCTHNHDDKSVETILVESGIVCPGLPLII |
| | | PDGYKLIDNSLILLECFVRSTPASFEKKFIEDTNKLACIREDL |
| | | AVAGVTLVPIVDGRCDYDNSFMPEWANFKFRDLLFKLLEY |
| | | SNQNEKVFEESEYFRLCESLKTTIDKRSGMDSMKILKDARST |
| | | HNDEIMRMCHEGINPNMSCDDVVFGINSLFSRFRRDLESGK |
| | | LKRNFQKVNPEGLIKEFSELYENLADSDDILTLSREAVESCP |
| | | LMRFITAETHGHERGSETSTEYERLLSMLNKVKSLKLLNTR |
| | | RRQLLNLDVLCLSSLIKQSKFKGLKNDKHWVGCCYSSVND |
| | | RLVSFHSTKEEFIRLLRNRKKSKVFRKVSFEELFRASISEFIAK |
| | | IQKCLLVVGLSFEHYGLSEHLEQECHIPFTEFENFMKIGAHPI |
| | | MYYTKFEDYNFQPSTEQLKNIQSLRRLSSVCLALTNSMKTS |
| | | SVARLRQNQIGSVRYQVVECKEVFCQVIKLDSEEYHLLYQK |
| | | TGESSRCYSIQGPDGHLISFYADPKRFFLPIFSDEVLYNMIDI |
| | | MISWIRSCPDLKDCLTDIEVALRTLLLLMLTNPTKRNQKQV |
| | | QSVRYLVMAIVSDFSSTSLMDKLREDLITPAEKVVYKLLRFL |
| | | IKTIFGTGEKVLLSAKFKFMLNVSYLCHLITKETPDRLTDQIK |
| | | CFEKFFEPKSQFGFFVNPKEAITPEEECVFYEQMKRFTSKEID |
| | | CQHTTPGVNLEAFSLMVSSFNNGTLIFKGEKKLNSLDPMTN |
| | | SGCATALDLASNKSVVVNKHLNGERLLEYDFNKLLVSAVS |
| | | QITESFVRKQKYKLSHSDYEYKVSKLVSRLVIGSKGEETGRS |
| | | EDNLAEICFDGEEETSFFKSLEEKVNTTIARYRRGRRANDKG |
| | | DGEKLTNTKGLHHLQLILTGKMAHLRKVILSEISFHLVEDFD |
| | | PSCLTNDDMKFICEAVEGSTELSPLYFTSVIKDQCGLDEMAK |
| | | NLCRKFFSENDWFSCMKMILLQMNANAYSGKYRHMQRQG |
| | | LNFKFDWDKLEEDVRISERESNSESLSKALSLTQCMSAALK |
| | | NLCFYSEESPTSYTSVGPDSGRLKFALSYKEQVGGNRELYIG |
| | | DLRTKMFTRLIEDYFESFSSFFSGSCLNNDKEFENAILSMTIN |
| | | VREGFLNYSMDHSKWGPMMCPFLFLMFLQNLKLGDDQYV |
| | | RSGKDHVSTLLTWHMHKLVEVPFPVVNAMMKSYVKSKLK |
| | | LLRGSETTVTERIFRQYFEMGIVPSHISSLIDMGQGILHNASD |
| | | FYGLLSERFINYCIGVIFGERPEAYTSSDDQITLFDRRLSDLV |
| | | VSDPEEVLVLLEFQSHLSGLLNKFISPKSVAGRFAAEFKSRF |
| | | YVWGEEVPLLTKFVSAALHNVKCKEPHQLCETIDTIADQAI |
| | | ANGVPVSLVNSIQRRTLDLLKYANFPLDPFLLNTNTDVKDW |
| | | LDGSRGYRIQRLIEELCPNETKVVRKLVRKLHHKLKNGEFN |
| | | EEFFLDLFNRDKKEAILQLGDLLGLEEDLNQLADVNWLNLN |
| | | EMFPLRMVLRQKVVYPSVMTFQEERIPSLIKTLQNKLCSKFT |
| | | RGAQKLLSEAINKSAFQSCISSGFIGLCKTLGSRCVRNKNRE |
| | | NLYIKKLLEDLTTDDHVTRVCNRDGITLYICDKQSHPEAHR |
| | | DHICLLRPLLWDYICISLSNSFELGVWVLAEPTKGKNNSENL |
| | | TLKHLNPCDYVARKPESSRLLEDKVNLNQVIQSVRRLYPKIF |
| | | EDQLLPFMSDMSSKNMRWSPRIKFLDLCVLIDINSESLSLISH |
| | | VVKWKRDEHYTVLFSDLANSHQRSDSSLVDEFVVSTRDVC |
| | | KNFLKQVYFESFVREFVATTRTLGNFSWFPHKEMMPSEDG |
| | | AEALGPFQSFVSKVVNKNVERPMFRNDLQFGFGWFSYRMG |
| | | DVVCNAAMLIRQGLTNPKAFKSLKDLWDYMLNYTKGVLE |
| | | FSISVDFTHNQNNTDCLRKFSLIFLVRCQLQNPGVAELLSCS |
| | | HLFKGEIDRRMLDECLHLLRTDSVFKVNDGVFDIRSEEFED |
| | | YMEDPLILGDSLELELLGSKRILDGIRSIDFERVGPEWEPVPL |
| | | TVKMGALFEGRNLVQNIIVKLETKDMKVFLAGLEGYEKISD |
| | | VLGNLFLHRFRTGEHLLGSEISVILQELCIDRSILLIPLSLLPD |
| | | WFAFKDCRLCFSKSRSTLMYETVGGRFRLKGRSCDDWLGG |
| | | SVAEDID |
| 20 | Amino acid sequence of L | MEEDIACVKDLVSKYLVDNERLSRQKLAFLVQTEPRMLLM |
| | protein in Lassa virus | EGLKLLSLCIEVDSCNANGCEHNSEDKSVERILHDHGILTPS |
| | | LCFVVPDGYKLTGNVLILLECFVRSSPANFEQKYIEDFKKLE |
| | | QLKEDLKSVDINLIPLIDGRTSFYNEQIPDWVNDKLRDTLFS |
| | | LLKYAQESNSLFEESEYSRLCESLSMTSGRLSGVESLNVLMD |
| | | NRSNHYEEVIASCHQGINNKLTAHEVKLQIEEEYQVFRNRL |
| | | RKGEIEGQFLKVDKSQLLNELNNLYADKVVAEDNIEHLIYQ |
| | | FKRASPILRFLYANVDEGNEKRENQTIGECQVQCWRSFLNK |
| | | VKSLRILNTRRKLLLIFDALILLASKHDLMKQKCLKGWLGS |
| | | CFLSVNDRLVSLESTKRDLEKWVERRQQAERSRITQSSQCLS |
| | | KNQILNSIFQKTILKATTALKDVGISVDHYKIDMEVICLNSY |
| | | DLIMDFDVSGVVPTISYQRTEEETFPYVMGDVELLGTTDLE |
| | | RLSSLSLALVNSMKTSSTVKLRQNEFGPARYQVVRCKEAYC |
| | | QEFSLGNTELQLIYQKTGECSKCYAINDNKVGEVCSFYADP |
| | | KRYFPAIFSAEVLQTTISTMISWIEDCNELEGQLNNIRSLTKM |
| | | ILVLILAHPSKRSQKLLQNLRYFVMAYVSDYHHKDLIDKIRE |
| | | ELITDVEFLLYRLIRTLMNLVLSEDVKSMMTNRFKFILNVSY |
| | | MCHFITKETPDRLTDQIKCFEKFLEPKVRFGHVSTNPADTAT |
| | | EEELDDMVYNAKKFLSKDGCTSIEGPDYKRPGVSKKYLSLL |
| | | TSSFNNGSLFKEREVKREIKDPLITSGCATALDLASNKSVVV |
| | | NKYTDGSRILNYDFNKLTALAVSQLTEVFSRKGKYLLNKQD |
| | | YEYKVQQAMSNLVLGSGQLKSDADGADLDEILLDGGASDY |
| | | FDQLKETVEKIVDQYREPVKLGSGPNGDGQPSINDLDEIVSN |
| | | KFYIRLIKGELSNHMVEDFDHDILPGKFYEEFCNAVYENSRL |
| | | KQKYFYCGHMSQCPIGELTKAVSTRTYFNHEYFQCFKSILLI |
| | | MNANTLMGRYTHYKSRNLNFKFDMGKLSDDVRISERESNS |
| | | EALSKALSLTNCTTAMLKNLCFYSQESPQSYDSVGPDTGRL |
| | | KFSLSYKEQVGGNRELYIGDLRTKMFTRLIEDYFEAISLQLS |
| | | GSCLNNEKEFENAILSMKLNVSLAHVSYSMDHSKWGPMMC |
| | | PFLFLTVLQNLIFLSKDLQADIKGRDYLSTLLMWHMHKMVE |
| | | IPFNVVTAMMKSFIKAQLGLRKKTKQSITEDFFYSNFQVGV |
| | | VPSHISSILDMGQGILHNTSDFYALISERFINYAISCICGGTID |
| | | AYTSSDDQISLFDQSLTELLQRDPEEFRTLIEFHYYMSDQLN |
| | | KFVSPKSVIGRFVAEFKSRFFVWGDEVPLLTKFVAAALHNIK |
| | | CKEPHQLAETIDTIIDQSVANGVPVHLCNLIQKRTLNLLQYA |
| | | RYPIDPFLLNCETDVRDWVDGNRSYRIMRQIEGLIPNACSKI |
| | | RSMLRKLYNRLKTGQLHEEFTTNYLSSEHLSSLRNLCELLD |
| | | VEPPSESDLEYSWLNLAAHHPLRMVLRQKIIYSGAVNLDDE |
| | | KIPTIVKTIQNKLSSTFTRGAQKLLSEAINKSAFQSSIASGFVG |
| | | LCRTLGSKCVRGPNKENLYIKSIQSLISDVKGIKLLTNSNGIQ |
| | | YWQVPLELRNGSGSESVVSYFRPLLWDYMCISLSTAIELGA |
| | | WVLGEPKTVKVLDFFKHNPCDYFPLKPTASKLLEDRVGLN |
| | | HIIHSLRRLYPSVFEKHILPFMSDLASTKMKWSPRIKFLDLCV |
| | | ALDVNCEALSLVSHIVKWKREEHYIVLSSELRLSHSRTHEP |
| | | MVEERVVSTSDAVDNFMRQIYFESYVRPFVATTRTLGSFTW |
| | | FPHKTSVPEGEGLHRMGPFSSFVEKVIHKGVERPMFKHDLM |
| | | MGYAWIDFDIEPARFNQNQLIASGLVDPKFDSLEDFFDAVA |
| | | SLPPGSAKLSQTVRFRVKSQDASFKESFAIHLEYTGSMNQQ |
| | | AKYLVHDVTVMYSGAVSPCVLSDCWRLVLSGPTFKGKSA |
| | | WYVDTEIINEFLIDTNQLGHVTPVEIVVDMERLQFTEYDFVL |
| | | VGPCTEPTPLVVHRGGLWECGKKLASFTPVIQDQDLEIFVRE |
| | | VGDTSSDLLIGALSDMMIDRLGLRMQWSGVDIVSTLRAAAP |
| | | SCEGILSAVLEAVDNWVEFKGYALCYSKSRGKVMVQSSGG |
| | | KLRLKGRTCEELTRKDECIEDIE. |
| 21 | Amino acid sequence of L | MEEYVFELKDIVRKWVPEWEELSEQKNNVLAQVKDRAITIE |
| | protein in Pichinde virus | GLKLLSMLVEVDSCKKHSCKHNTKMTVNAILRELRVTCPTL |
| | | PDVTPDGYCMVGDVLILLEVFVRTSQEAFEKKYNQDFLKLL |
| | | QLSSDLKRQNITLVPVIDGRSSYYVEFVPDWVVERLRWLLL |
| | | KLMDGLRTSGEEVEELEYERLISSLSSLENQSLGLESLLAVK |
| | | ERGLPYKVRLEKALMSGINNKLTTDQCRTKIMEIFQQFKML |
| | | QLAGQLDRKLQATDREDMISRLQNHEFIQCSVKDVPKSEIR |
| | | LCEFCSVHILGIIGQLRQSEVKHSSTESREYFRVLSICNKIKSQ |
| | | KVFNTRRNTMLVLDLIMYNILCDLDKSSPGAVFREVLLMQG |
| | | LPSVNDRLINVDFLMEQITKKFIKNPNWLEKAKKRLSSVCG |
| | | ELPLDDILPLLREPDVEYYFNLKTSVLDEWGAKPCLQYKTK |
| | | SQCMCGGRPGRGQPDYTIMGESEFEELLKTLSSLSLSLINSM |
| | | KTAAVPKMKVNNADEFYGKVYCDEVFFQRFGEGGSLTLLY |
| | | QKTGERSRCYAVAYRSKSGGLYETKASFYCDPKRFFLPIFSA |
| | | DVIQRTCVEMLSWLDFMSQPLLDSVSDLLRRLILCILCTPSK |
| | | RIQVYLQGFRYYIMAFVNEVHFKELFEKLKVVMLTPSEWQT |
| | | AMLIDDLILLVLSNSREEDMAKIFKFVLNVSYLCHFITKETP |
| | | DRLTDQIKCFEKFLEPKLKFDSVLVNPSNSMELPTEEEEKMV |
| | | HDIERLLGKKLESKCEGRPGLNKDVLSVCLSLFNSSSLEVKP |
| | | LLPCDPMTPSFTSTALDMSSNKSVVVPKLNEVGEVITEYDYS |
| | | SIVSAVVVEMIEHFKTKGKYKLDPKEVNFKILKRLSSLIQIKK |
| | | ESIEPDGVEELLSEDQGDCLKEIETRVAKVLSKVDTNVKTNL |
| | | KTSCPLERLWPKSTMVVIKRETSLHDVKDFDYSLFSAEVYE |
| | | DLVNLIYEDVTARSVYFADRLMNPCPLEFLIKNLTLKAYKE |
| | | ADYFECFKYILIASDYDNRVGRYDHKSRSRLGFTDAALQIRE |
| | | TSRISSRESNSESIAKRLDQSFFTNSSLRNLCFYSDESPTERSG |
| | | VSTNVGRLKFGLSYKEQVGGNRELYVGDLNTKLTTRLIEDY |
| | | SESLMQNMRYTCLNNEKEFERALLDMKSVVRQSGLAVSM |
| | | DHSKWGPHMSPVIFAALLKGLEFKLKDGSEVPNAAVINILL |
| | | WHIHKMVEVPFNVVEAYMKGFLKRGLGMMDKGGCTIAEE |
| | | FMFGYFEKGKVPSHISSVLDMGQGILHNTSDLYGLITEQFIN |
| | | YALELCYGARFISYTSSDDEIMLSLNEGFKFKDRDELNVELV |
| | | LDCMEFHYFLSDKLNKFVSPKTVVGTFASEFKSRFFIWSQEV |
| | | PLLTKFVAAALHNIKAKAPNQQADTIDTILDQCVANGVSIEV |
| | | VGAIAKRTNSMIIYSGFPNDPFLCLEEMDVLDWVNGSRGYR |
| | | LQRSIETLFPDDLLLSIIRKACRKIFYKIQSGALEESYIVTTLQ |
| | | QSPDDCLKQLLETCDVETEAIEDALNIRWLNLRVHGDLRLV |
| | | LRTKLMSTTRTVQREEIPSLVKSVQSKLSKNYVRGAKKILA |
| | | DAINKSAFQSSIASGFIGVCKSMGSKCVRDGKGGFKYIRDIT |
| | | SKIILHRDCHFCNQRKGVYCKAALGEVSEYSRPLIWDYFAL |
| | | VLTNACELGNWVFQKAEVPKIVTHLNNPNHFWPIKPSTHSE |
| | | LEDKVGINHILYSIRRNFPTLFDEHISPFLSDLNMLRLSWVQR |
| | | IKFLDLCVAIDITSECLGIVSHIIKHRREELYIVKQNELAMSHS |
| | | RESHPLERGFNLEPEEVCTNFLIQILFESMLVPVIMSTSQFKK |
| | | YFWFGELELLPNNAQHDLKQLTQFICDCKKNNTSRTMNLD |
| | | DLDVGFVSSKLILSCVNLNISVFINELDWVNRDNYENIEQLIL |
| | | ASPSEVIPIELNLTFSHKRVSHKFRYERSTNYILKLRFLIERES |
| | | LLDSLDSDGYLLLNPHSVEYYVSQSSGNHISLDGVSLLVLNP |
| | | LINGKDVLDFNDLLEGQDIHFKSRSTVFQKVRIDLKNRFKDL |
| | | KNKFSYKLIGPDVGMQPLILEGGLIKEGNRVVSRLEVNLDS |
| | | KVVIIALEALEPEKRPRFIANLFQYLSSAQSHNKGISMNEQD |
| | | LRLMIENFPEVFEHMLHDAKDWLNCGHFSIIRSKTLGSVMIA |
| | | DETGPFKIKGIRCRKLFEDNESVEIE |
| 22 | Amino acid sequence of L | MEESVNEIKSLIRKHFPERQELAYQRDIFLSQHHPSSLLLEGF |
| | protein in Junin virus | KLLSSLVELESCEAHACQINSDQKFVDVILSDYGILCPTLPKV |
| | | IPDGFKLTGKTLILLETFVRVNPDEFERKWKSDMSKLLNLKS |
| | | DLLRAGITLVPVVDGRSSYSNRFLADWVVERVRWLLIDILK |
| | | KSKFMQEINIEEQEYQRLIHSLSNTKNQSLGLENIECLKKNSL |
| | | GYDERLNESLFVGVRGDIRESVIREELIKLRFWFKKEIFDKQ |
| | | LGKFKFSQKSKLINDLILLGSHKDSDVPSCPFCANKLMDVV |
| | | YSIALHPIDEVNMESQSDENSISIDAVERCYLQALSVCNKVK |
| | | GLKVFNTRRNTLLFLDLVLLNLLCDLFKTHDDAIVRLRNAG |
| | | IVVGQMLMLVNDRLLDILEAIKLIRKKLMTSPKWVQACSRT |
| | | LKNSHQDLWSQLEKLIKHPDMDSLMILAQALVSDRPVMRY |
| | | TINKEFEKICRHQPFSSLVEGEQKKLFRILSSISLALVNSMKTS |
| | | FSSRLLINEREYSRYFGNVRLRECYVQRFHLTKNTFGLLFYQ |
| | | KTGEKSRCYSIYLSINGVLEEQGSFYCDPKRFFLPIFSEDVLIE |
| | | MCEEMTSWLDFSHELMTMTRPILRLLVLAVLCSPSKRNQTF |
| | | LQGLRYFLMAYANQIHHVDLMSKLRVDCMSGSEVLIQRMA |
| | | VELFQTILGEGEDADLYFARRFKYLLNVSYLCHLVTKETPD |
| | | RLTDQIKCFEKFIEPKVKFDCVVVNPPLSGSLTLEQEDTMIR |
| | | GLDRFFSKEAKTSSDTQIPGVCKEILSFCISLFNRGRLKVTGE |
| | | LKSNPYRPNITSTALDLSSNKSVVIPKLDELGNILSVYDKEKL |
| | | VSTCVSTMAERFKTKGRYNLDPDSMDYLILKNLTGLVSTGS |
| | | RTRTNQEELSVMYESLTEDQVRAFEGIRNDVQMTLAKMAN |
| | | SEGTKVEITKLKSKNPSVDERESLESLWAPFGVLREIKAEVS |
| | | MHEVKDFDPDVLRSDVYKELCDAVYLSPFKLTYFLEAPQDI |
| | | CPLGLLLKNLTTIAYQEDEFFECFKYLLIQGHYDQKLGSYEH |
| | | RSRSRLGFSSEVLRLKDEVRLSTRESNSEAIADKLDKSYFTN |
| | | AALRNLCFYSDDSPTEFTSISSNTGNLKFGLSYKEQVGSNRE |
| | | LYVGDLNTKLMTRLVEDFSEAVGSSMKYTCLNSEKEFERAI |
| | | CDMKMAVNNGDLSCSYDHSKWGPTMSPALF |
| | | LSFLYTLELKNPRDRTKVNLEPVMNILKWHLHKVVEVPINV |
| | | AEAYCVGKLKRSLGLMGCDCTSVGEEFFHQYLQSRDQVPS |
| | | HtMSVLDMGQGILHNTSDLYGLITEQFLCYALDLLYDVIPVT |
| | | YTSSDDQVSLIKIPCLSDEKFQDRTELLEMVCFHEFLSSKLN |
| | | KFISPKSVIGTFVAEFKSRFFVMGEETPLLTKFVSAALHNVK |
| | | CKTPTQLSETIDTICDQCIANGVSTHIVSKISIRVNQLIRYSGY |
| | | RETPFGAIEEQDVKDWVDGSRGYRLQRKIEAIFSDDKETMFI |
| | | RNCARKVFNDIKRGKIFEENLINLISRGGDEALSGFLQYAGC |
| | | SEDEIRQTLDYRWVNLASFGDLRLVLRTKLMTSRRVLEKEE |
| | | MPTLIRTIQSRLSRNFTKGVKKILAESINKSAFQSSVASGFIGF |
| | | CKSMGSKCVRDGKGGFLYIKDIFTRIMPCLCGICERKPKVIY |
| | | CQKSLKEVNQFSKPILWDYFSLVLTNACELGEWVFSAIKSP |
| | | QAPLVLCNKNFFWAVKPKAVRQIEDQLGMNHVLHSIRRNY |
| | | PKLFEEHLAPFMNDLQVNRSLDSGRLKFLDVCVALDMMNE |
| | | NLGIISHLLKVRDNNVYIVKQSDCASAHVRQSEYTNWEVGI |
| | | SPQQVCRNFMVQVVLSSMINPLVMSTSCLKSFFWFNEVLDL |
| | | EDDSQVDLAELTDFTLSVRNNKVSRAMFVEDIAMGYVVSS |
| | | FDNIKVFLEGVSVDNISLLPQEDMMDLHTVLRNVACQEAV |
| | | KLKLIIQVEHTRVSTKFKLRRKMVYSYTIVSSLGVDDVGTPE |
| | | LELNVDAMSQCVSGSEGSHSLLDGALVIASLPLFTGHESFDL |
| | | AGLFIDAGYAATNDDNILSHVKFNFGDFYSELSNKYAYDLI |
| | | GPDNPGEPLVLKEGIFYRGNERLSTYRVELSGDVIVKAIGAL |
| | | EDIDSVETLLSQLWPYLKTTSQTILFQQEDFVLVYDLHKEQL |
| | | IRSLDKFGDWLEFSNFKVAFSRSLNDLLISDPQGQFRLKGVT |
| | | CRPLKHKVEIKDID. |
| 23 | Amino acid sequence of L | MDESVSSLFDLLRKHFPAKEEISRQITVVTSQTEMRMILTEG |
| | protein in Oliveros virus | FKLLSLLIELDSCEVNNCSHNKEDLTVEAILSKDNILTIALPRI |
| | | VPDGYSLYGNVLILLETFVRVNPSSFEQKYNQDMNKLLSLK |
| | | NDLQLCGITLVPLVDGRTNYYNKFVDDWVIERFRWLLTQI |
| | | MKVAKESGESIEELEYQRLVTSLSKLENQSLGFENIIKMPQT |
| | | GIDYRDKLKARMFANLSNKMKESEINQSLLSLKLAFDEAYN |
| | | DESHLKKFQKTNKEDLIFKLGQQINLSDEKLSCMSCSSKLFSI |
| | | VSSITQNRDKLDSHVMSVSNAKLWHHESGIANVNEYLRILS |
| | | VCNKIKSAKILNTRRNTLLFLDMIVLNFIDDCWKNDPTILFQ |
| | | FKKSGLLVGQLAYFVNDRFFDLLLLKELLSKKLKSSPDWIH |
| | | RCLCNIRKQEFFDISGVEFWIRQPDYESVEELCCALEPVKPK |
| | | LQYCRDEDNHENHKLDLADKDNYFTCLSVLSSVCLGLVNS |
| | | MKTSFTSKMVINEKSPNNFYGEVELKECYCQRFYVSDEITG |
| | | LLFYQKTGEKSRCYSIGVTMHGSYKYIGSFYCDPKRFFLPIFS |
| | | QVVLFQMTEEMMSWLPEEPSYKEPVVANLRKLILMLLCNP |
| | | SKRNQNFLQGMRYFIMAYVNQFHSVELMSKLEVPCKSVSE |
| | | ECVQKLTYNLLVDVLTKGDVNEHMTRKFKFCLNVSYLCHL |
| | | ITKETPDRLTDQIKCFEKFLEPKLKFKSVIINPNLTGDLTEEQE |
| | | EQLLNSIEKLLGKGLQDINDSSEPGISRELLSMCISAFNRDLL |
| | | RVNGKLKNDPYKPNFTSTALDLSSNKSVVIPKLDELGNPISK |
| | | YDYELLVSSCIASMAESFKTKGKYKLDPTSQEFLILKNLYSL |
| | | MSKSKRDDHMKDSEDSKQNLSSDLENLSEEQVLILEQVKRD |
| | | VNLALSKMRETKLKEKTEARQSSSGSSLKNQQKRQAELQE |
| | | RLSELWSEFMCMKIITVEVSLHEIKDFDPDLIDHTTLKSMLD |
| | | KLYNSDLASEFFLEEILNPCPLEFLVKNMTTSSYLEGDLFECF |
| | | KYTLISAGFDQKLGTYEHKNKTRLGFKYEALKVREEGRMS |
| | | LRESNSEAIARRLDRSVFSNSALRNLCFYSDESPISYSHVSSD |
| | | TGKLKFGLSYKEQVGSNRELYVGDLNTKLMTRLIEDFSESV |
| | | VSNMNYSCLNSEKEFERSVMEMKMSVNLGEMNFSLDHSK |
| | | WGPYMSPVIFAAFLQGLKLEQGSMCTPVSVEPIITLLSWHIH |
| | | KVVEVPYNVIHAYMTGMIKRQLGLMSPGESSKTEAFIHRLL |
| | | VDEREPLSHVMSVLDMGQGILHNTSDLYGLVTEQFINYAM |
| | | RILYDVSMTSFTSSDDQITMVKLNEDLKDMDNPEVISNWER |
| | | MINFHTFISSKFNKFVSPKTVIGTFAAEFKSRFFVWGEEVPLL |
| | | TKFVSAALHNIKCKTPIQLSETIDTISDQCVANGVSVEIVSCIS |
| | | NRTNKLVRYSGFPDNPFLSVENMDVKDWVDGNRGYRLQR |
| | | NIESHLEVDGCTRFVRQAARKVFRNIKSGKIMEQTLVNLVQ |
| | | EDGDKAFQGFMKSVDVSDDDIKLLQNFRWINLSTHGDMRL |
| | | VLRTKLMSSRRIIEQEEIPGLIKSIQSKLSKNFVRGAKRILADS |
| | | INKSAFQSSIASGFIGFCKSMGSKCVRLGGGGFGYIKDIKNK |
| | | VKHDCLCDICFRWRGCVYCPSSCADVFEFSRPLMWDYFTL |
| | | VLTNACELGEWVFEDVEIPKDLYFLRNPNLFWLVKPRVTCQ |
| | | LEERLGLSHILQSIRKNYPTLFETHLSPFMSDFMVGKTLGSLT |
| | | VKFLDLCVALDLANENLGITKHFLKERRHEIYVVKQDESSQ |
| | | SHIRNVKGIESSVELNSMQVCNNFLTQLLMSSFIQPLVLTSSV |
| | | FKKFNWFAEVLCLKTEEEVCLKQLTDFVLQVKKFNVDRAM |
| | | HIEDLSAGYISSTINVTSFSLSVPTFLECVDSDFINKEGNEPGD |
| | | FKDLLSSEFTKDTLTLDFCIQVSHIKRSVKFNVKRTLVYTLA |
| | | VRTQIEKKIILEAIGTDDQISLIVSELDLFCSGHTGNHFVLDA |
| | | APLIYSEPLIAGSLKFDLLSMLRDQELSLTSSEKMPTFNFDFS |
| | | SQKHHIVNKFAYKLVGPSVYDEPLVLNKGIVYSGERKLTSL |
| | | GVDVSGERIVQAMGELDSISEQELFLTNLWGYSKETDIKVRI |
| | | IQDNLKILTDNYFVQLKNSLKTFAEWLNLGNYMLCYSKTLD |
| | | TIMISDVSGRIKLKGVICRKLIEDEVMEVE |
| 24 | Amino acid sequence of L | MSECLTYFNELKDLVRKWVPDEDTYVEQKTVLLSQVNFSSI |
| | protein in Tamiami virus | VTEGLKLLSTLIEVDSCVKHGCIHNRSKTVNQILYDHRIVGP |
| | | TLPDVVPDGYRVSGSTLILLETFVRVNQESFECKYRHDFEKL |
| | | MQLSKDLAKCGLTLVPVIDGRSSYYIERLPDWVIERMRWLL |
| | | LRIMSNLRDSGEKIEEMEYERLVHSLSNMENQNLGLESLAS |
| | | LREEGLDYKTRLTKTLKEGIYSNMTTSECRVGIAKLYDHFC |
| | | LLRDSGQYEDVYTTTSRSEMITWLKTHELVQMSSSERETLIE |
| | | AETCKFCQIHMYAVLKDLVLLRKGWKGSRCRDANEILAHK |
| | | SLLSDCNKIKGLKVLNTRRNTLLCLDIIVLNSLINLIKLQYTD |
| | | LQYLINNHFKSVNDRLVSVDLIINKLDKRLTSDPNWLCKLR |
| | | TKIGHKLKIYDLDHVISWLRPIEVSHWYEFKLERDNSGECV |
| | | KPTIKYKKSGVGDCQGEDCNKDVITDDSTFSDYLDALSTLS |
| | | MGLMNSMKTSSATKLVVNDERNYFGTVQCDECYFQDLDIN |
| | | YGTTLIYQKTGEGTRCYGKVSKEGGGPDVYKVGKSFYADP |
| | | KRYFLPIMSSEVILKMCREMLSWLDWLSEKEMMDVRTKLY |
| | | TLVISILTVPSKRVQIYLQGFRYFIMAYVNEFHVKELVCKLK |
| | | VKPLTRAELSVFTQMDDLVALLLTGTSEEHMTKSFKFILNLS |
| | | YLCHLITKETPDRLTDQIKCFEKFLEPKLTFNSVILNLDSSPQ |
| | | LTEGTEEKIIGDLKKLFSKDLGVLDLKEPGVSKEVLSLCSSCF |
| | | NNGMLSLPKVLSRDPQSPSFTSTALDISSNKSVVVPKLNEVG |
| | | ETITQYDYQSLLSSVVVEMAQSFKDKLRFKLDRRSLQYAIY |
| | | KRLTNMVSKNEFRSKDDPNDSGILEDIEDLVDEGTHKLINEI |
| | | EANVSDCLSKMSSGCNKSNQSSKGLKKFEKVDLLQKLWSR |
| | | EYMSLILSETSFHEVKDFDPSLLPSESYQEMCDAVYDSVYR |
| | | NEFFTEKFLKLCPLELLIKNLATKHYEEGDYFECFKYLLIGA |
| | | GCDNRVGRFDHRSRARLGFKDTATLVKEESRISSRESNSEAI |
| | | SKRLDKSFFTNSSLRNLCFYSEESPTYRSSVSSSVGKLKFGLS |
| | | YKEQVGSNRELYIGDLNTKLTSRLIEDYFESLTSECRFSCLN |
| | | NDSEFERALLDMKSVVRLSGLAVSLDHSKWGPYMSPAIFN |
| | | ALFSNLDLQLKDGGLIDKSPIENLLNWHLHKIVEVPYNVVE |
| | | AYLKGYTKRSLGLMDRSSSSMTEDYFFRQFAKGVVPSHITS |
| | | VLDMGQGILHNASDYYGLLTEQFITLCLELCFDVKMTAYTS |
| | | SDDEIMLSNSYSLKRESDDDLLDMEKCKEILEFHYYLSSKLN |
| | | KFISPKTVAGSFASEFKSRFFIWSQEVPLLTKFVAAALHNVK |
| | | AKSPHQLAETIDTILDQCAANGVSIEIINELSKRTNRLISYSGH |
| | | PVDPFLCVFTTDLKDWVDGSRGYRLQRSIESIINSEEILSTIRD |
| | | SCRQLFYMIRSGRIQEEYLISALQSSPDDCLRQMLKITGTNDS |
| | | LIEEALTTRWLNLRAFGDLRLVLRTKIMTGTRILDKEEVPSLI |
| | | KSVQSKLSKNFVRGAKKIITDAINKSAFQSSICSGFIGLCKSM |
| | | GSKCVRDGSGGFIYIKDLLKKIDRHTNCEVCCPLLSVFCEHS |
| | | LRQVAPYSRPLLWDYFSLTFSNACELGNWVFSKVELPRPPL |
| | | GSMNPNFFWPVKPGSHSELEDKVNMNHVLYSIKRNFPDLFD |
| | | EHIAPFLSDLNSLKVSWVQRIKFLDLCVAMDMSSECLGIISHI |
| | | MRKRREELYIVKQEELSVCHIRESCSLEKGLQLNSVEICQNF |
| | | LTQLLFESMLNPVLLSTSQFKKYFWYGEVEFLPNDADHDLG |
| | | QLTQFIMDCKLLNISRCMCLDDLDVGYVHSKIELSQVFINLS |
| | | TFINLVDWENRESYQSFDEVLIHSNADHIPLEIGIILSHTRKSF |
| | | KFRYERKTNYYVKCGITIQKSEISSFSTTLSDGFELHVEEIDC |
| | | YVSGSEGDHISLDGVGLVPLHPLFSGKEALDLNKLLSDQDIE |
| | | FKQISLVFSKVKLDFKDHVKDLKNKFSYKLQGPEQGLEPLH |
| | | LDKGQIMERNTVVSRLEVPVTSRSLFLALEALDPGNRPRFLS |
| | | SLHEYMRKRPGKKDPCFVRMTQQDLCLLVELYEAAFMQVL |
| | | SAVSDWIEFGCFALCFSKTLNCIMIADDGGDYRLKGRPCKT |
| | | LSAQKTLTDIE. |
| 25 | Amino acid sequence of | MGQIVTMFEALPHIIDEVINIVIIVLIIITSIKAVYNFATCGILAL |
| | GP signal peptide in | VSFLFLAGRSCG |
| | LCMV | |
| 26 | Amino acid sequence of | MGQIITFFQEVPHVIEEVMNIVLIALSLLAILKGVYNVATCGII |
| | GP signal peptide in Lassa | GLVTFLFLCGRSCS |
| | virus | |
| 27 | Amino acid sequence of | MGQVVTLIQSIPEVLQEVFNVALIIVSTLCIIKGFVNLMRCGL |
| | GP signal peptide Pichinde | FQLITFLILAGRSCD |
| | virus | |
| 28 | Amino acid sequence of | MGQFISFMQEIPTFLQEALNIALVAVSLIAIIKGIVNLYKSGLF |
| | GP signal peptide in Junin | QFFVFLALAGRSCT |
| | virus | |
| 29 | Amino acid sequence of | MGQVIGFFQSLPNIINEALNIALICVALIAILKGIVNIWKSGLI |
| | GP signal peptide in | QLFIFLILAGRSCS |
| | Oliveros virus | |
| 30 | Amino acid sequence of | MGQLISFFGEIPTILQEALNIALIAVSIIATIKGVVNVWKSGLI |
| | GP signal peptide in | QLLMFVMLAGRSCS |
| | Tamiami virus | |
| 31 | Amino acid sequence of | MYGLNGPDIYKGVYQFKSVEFDMSHLNLTMPNACSANNSH |
| | GP1 in LCMV | HYISMGSSGLELTFTNDSILNHNFCNLTSAFNKKTFDHTLMS |
| | | IVSSLHLSIRGNSNHKAVSCDFNNGITIQYNLSFSDPQSAISQ |
| | | CRTFRGRVLDMFRTAFGGKYMRSGWGWAGSDGKTTWCS |
| | | QTSYQYLIIQNRTWENHCRYAGPFGMSRILFAQEKTKFLTR |
| | | RLA |
| 32 | Amino acid sequence of | LIYKGSYELQTLELNMETLNMTMPLSCTKNSSHHYIRVGNE |
| | GP1 in Lassa virus | TGLELTLTNTSIINHKFCNLSDAHKRNLYDHALMSILSTFHL |
| | | SIPNFNQYEAMCDFNGGKISVQYNLSHAYAVDAAEHCGTV |
| | | ANGVLQTFMRMAWGGSYIALDSGRGNWDCIMTSYQYLIIQ |
| | | NTTWEDHCQFSRPSPIGYLGLLSQRTRDIYISRRLL |
| 33 | Amino acid sequence of | GMMIDRRHNLTHVEFNLTRMFDNLPQSCSKNNTHHYYKGP |
| | GP1 Pichinde virus | SNTTWGIELTLTNTSIANETTGNFSNIRSLAYGNISNCDKTEE |
| | | AGHTLKWLLNELHFNVLHVTRHVGARCKTVEGAGVLIQYN |
| | | LTVGDRGGEVGRHLIASLAQIIGDPKIAWVGKCFNNCSGGS |
| | | CRLTNCEGGTHYNFLIIQNTTWENHCTYTPMATIRMALQKT |
| | | AYSSVSRKLL |
| 34 | Amino acid sequence of | EEAFKIGLHTEFQTVSFSMVGLFSNNPHDLPLLCTLNKSHLY |
| | GP1 in Junin virus | IKGGNASFQISFDDIAVLLPQYDVIIQHPADMSWCSKSDDQI |
| | | WLSQWFMNAVGHDWHLDPPFLCRNRAKTEGFIFQVNTSKT |
| | | GVNGNYAKKFKTGMHHLYREYPDPCLNGKLCLMKAQPTS |
| | | WPLQCPLDHVNTLHFLTRGKNIQLPRRSLK |
| 35 | Amino acid sequence of | HTFQIGRNHEFQSITLNFTQFLGYAPSSCSVNNTHHYFRGPG |
| | GP1 in Oliveros virus | NVSWGIELTLTNNSVINASNSLKVFTNIHHNITNCVQNIDEQ |
| | | DHLMKWLIETMHLQIMKPGKRLPPILCEKDKGLLIEYNLTNI |
| | | ASREEKHSEYWSQLLYGLSKLLGSSKSLWFDYCQRADCMM |
| | | QEHSSHLKCNYSECSGHTTFKYLILQNTTWENHCEFNHLNTI |
| | | HLLMSSTGQSFITRRLQ |
| 36 | Amino acid sequence of | VQIGHHLELEHIILNSSSILPFTPTLCKLNKTYFLVRGPFQAH |
| | GP1 in Tamiami virus | WGVDLAIGSTTVAVENATKTYTLKSKNFTGCFEGNPDPDSA |
| | | ALLVTWLFNSLHHDYKNDPSILCERVSGENSFRFQINISEPEY |
| | | CEKILSRMANLFGSFENYCLNNRHIKKLIIIRNLTWSQQCHE |
| | | NHMSAMQLITSNIHTQVVRARRIL |
| 37 | Amino acid sequence of | GTFTWTLSDSSGVENPGGYCLTKWMILAAELKCFGNTAVA |
| | GP2 in LCMV | KCNVNHDEEFCDMLRLIDYNKAALSKFKQDVESALHVFKT |
| | | TVNSLISDQLLMRNHLRDLMGVPYCNYSKFWYLEHAKTGE |
| | | TSVPKCWLVTNGSYLNETHFSDQIEQEADNMITEMLRKDYI |
| | | KRQGSTPLALMDLLMFSTSAYLISIFLHLVKIPTHRHIKGGSC |
| | | PKPHRLTNKGICSCGAFKVPGVKTIWKRR |
| 38 | Amino acid sequence of | GTFTWTLSDSEGNETPGGYCLTRWMLIEAELKCFGNTAVA |
| | GP2 in Lassa virus | KCNEKHDEEFCDMLRLFDFNKQAIQRLKSEAQMSIQLINKA |
| | | VNALINDQLIMKNHLRDMMGIPYCNYSKYWYLNHTS SGRT |
| | | SLPKCWLVSNGSYLNETHFSDDIEQQADNMITEMLQKEYID |
| | | RQGKTPLGLVDLFVFSTSFYLISIFLHLIKIPTHRHIVGKPCPK |
| | | PHRLNHMGICSCGLYKQPGVPTRWKR |
| 39 | Amino acid sequence of | GFFTWDLSDSTGQHVPGGYCLEQWAIVWAGIKCFDNTVMA |
| | GP2 Pichinde virus | KCNKDHNEEFCDTMRLFDFNQNAIKTLQLNVENSLNLFKKT |
| | | INGLISDSLVIRNSLKQLAKIPYCNYTKFWYINDTITGRHSLP |
| | | QCWLVHNGSYLNETHFKNDWLWESQNLYNEMLIKEYEER |
| | | QGKTPLALTDICFWSLVFYTITVFLHLVGIPTHRHIIGDGCPK |
| | | PHRITRNSLCSCGYYKIPKKPYKWVRLGK |
| 40 | Amino acid sequence of | AFFSWSLTDSSGKDTPGGYCLEEWMLVAAKMKCFGNTAV |
| | GP2 in Junin virus | AKCNLNHDSEFCDMLRLFDYNKNAIKTLNDETKKQVNLMG |
| | | QTINALISDNLLMKNKIRELMSVPYCNYTKFWYVNHTLSGQ |
| | | HSLPRCWLIKNNSYLNISDFRNDWILESDFLISEMLSKEYSD |
| | | RQGKTPLTLVDICIWSTVFFTASLFLHLVGIPSHRHIRGEACP |
| | | LPHRLNSLGGCRCGKYPNLKKPTVWRRGH |
| 41 | Amino acid sequence of | AFLTWTLSDATGNDLPGGYCLEQWAIVWAGIKCFGNTAVA |
| | GP2 in Oliveros virus | KCNQNHDSEFCDMLRLFDYNRNAIKSLNDQSQSRLNLLTNT |
| | | INSLISDNLLMKNKLAEIMNIPYCNYTKFWYINDTRTGRHTL |
| | | PQCWLISNGSYLNETKFRTQWLSESNALYTEMLTEDYDKR |
| | | QGSTPLSLVDLCFWSTLFYVTTLFAHLVGFPTHRHILDGPCP |
| | | KPHRLTKKGICSCGHFGIPGKPVRWVKRSR |
| 42 | Amino acid sequence of | SFFTWSLSDAVGNDMPGGYCLEKWMLIASQLKCFGNTAVA |
| | GP2 in Tamiami virus | KCNLNHDSEFCDMLRLFDFNRKAIETLQNKTRSQLNIAINAI |
| | | NSLISDNLLMKNRVKELMDIPFCNYTKFWYVNHTKLNHHS |
| | | LPRCWLVKNGSYLNESEFRNDWLLESDHLISEILSREYEERQ |
| | | GRTPLSLVDVCFWSTLFYTASIFLHLIRIPTHRHIVGEGCPKP |
| | | HRLRADSTCACGLYKQKRRPLKWVRSN |
| 43 | Peptide epitope derived | LPYLGWLVF |
| | from P1A | |
| 44 | Immunodominant NP- | RPQASGVYM |
| | derived backbone epitope | |
| 45 | Peptide epitope derived | RAHYNIVTF |
| | from HPV16 E7 protein | |
| 46 | Immunodominant NP- | FQPQNGQFI |
| | derived backbone epitope | |
| 47 | NP-derived backbone | YTVKYPNL |
| | epitope | |
| 48 | Amino acid sequence of | MSSENVPSFRWTQSLRRGLSNWTHAVKGDVLADARAIVSA |
| | glycoprotein precursor | LDFHQVAQVQRMMRKDKRSEADLTRLRDMNKEVDALMM |
| | (GPC) protein in | MRSAQKDNILKVGGLSKDELMELASDLDKLRKKVQRTEGG |
| | Allpahuayo virus (ALLV) | GQPGVYAGNLTSSQLNQRSEILKMMGMGTGPRGPVGGVV |
| | | KVWDIKDSSLLVNQFGSMPALTIACMTQQGGEQMNDVVQ |
| | | ALTSLGLVYTVKYPNLSDLEKLTEKHPCLKLITQEPAQINISG |
| | | YNLSLSAAVKADACMIDGGNMLETLQVKPSMFSTLIKTILE |
| | | VKNREGMFVSPSPGQRNPYENILYKVCLSGDGWPYIGSRSQ |
| | | IKGRAWENTTVDLEGKPSVNHPPVRNGGSPDLKQIPKTKED |
| | | EVIRAIEQLDPRGTTWVDIEGPPGDPVELALFQPETGNYLHC |
| | | YRRPHNENAFKDQSKFSHGLLLKDLADTQPGLISCIIRHLPN |
| | | NMVLTAQGNDDIIKLLEMHGRRDIKVLDVKLSSDQARLME |
| | | DVVWERYNMLCVKHTGLVIKKKKKGAAPGSANPHCALLD |
| | | CIMFDATVTGYLRDQKPKRLLPLDTLYRDNANLINL |
| 49 | Amino acid sequence of | MSSENVPSFRWTQSLRRGLSNWTHAVKGDVLADARAIVSA |
| | NP protein in ALLV | LDFHQVAQVQRMMRKDKRSEADLTRLRDMNKEVDALMM |
| | | MRSAQKDNILKVGGLSKDELMELASDLDKLRKKVQRTEGG |
| | | GQPGVYAGNLTSSQLNQRSEILKMMGMGTGPRGPVGGVV |
| | | KVWDIKDSSLLVNQFGSMPALTIACMTQQGGEQMNDVVQ |
| | | ALTSLGLVYTVKYPNLSDLEKLTEKHPCLKLITQEPAQINISG |
| | | YNLSLSAAVKADACMIDGGNMLETLQVKPSMFSTLIKTILE |
| | | VKNREGMFVSPSPGQRNPYENILYKVCLSGDGWPYIGSRSQ |
| | | IKGRAWENTTVDLEGKPSVNHPPVRNGGSPDLKQIPKTKED |
| | | EVIRAIEQLDPRGTTWVDIEGPPGDPVELALFQPETGNYLHC |
| | | YRRPHNENAFKDQSKFSHGLLLKDLADTQPGLISCIIRHLPN |
| | | NMVLTAQGNDDIIKLLEMHGRRDIKVLDVKLSSDQARLME |
| | | DVVWERYNMLCVKHTGLVIKKKKKGAAPGSANPHCALLD |
| | | CIMFDATVTGYLRDQKPKRLLPLDTLYRDNANLINL |
| 50 | Amino acid sequence of Z | MGLRYSKEVRDRHGDKDIEGRVPMTLNLPQGLYGRFNCKS |
| | protein in ALLV | CWFVNKGLIACGDHYLCLGCLTRMLSRTDFCEICSKPLPKKI |
| | | IFEDSPSAPPYEP |
| 51 | Amino acid sequence of L | MDVHLIELRDLVRKWVPDDLELSEQKNIMLAQTQIRATVVE |
| | protein in ALLV | SLKLLSTIVEVDSCKKHSCVHNTSKTVNAILREHKIIGPTLPD |
| | | VTPDGYCVIGDVLILLEVFVRTNQESFEKKFNQDFEKLMQM |
| | | SADLKKCGVTLVPTIDGRSTYYVDFIPDWVVERLRWLISRL |
| | | MSSLREDGQEIEELEYERLISSLSSLENQSLGLESLLAMREKG |
| | | LSYKETLDKLFLEGMENKLTVDESRTRIMKMFQIFRTLLESG |
| | | YLERKYQTTDREDMLKRLRDHEFIVCSKSVEYTFDCPNCSV |
| | | HLYKVLNLLLNQGSRGAPHQCLGEYMKTLSICNKIKSMKIL |
| | | NTRRNTLLILDTIMLNKFLDLEKVFGHVVVERVMIMQSLMT |
| | | VNDRLLSIDVLMEMLEKKMTRNPLWFLKVNEKLRKLCPPE |
| | | VYQSIEEYVHEVDRDHWFELKLTLHQTWPAKPLIDYKGKM |
| | | RCTCVEKDSNNKNQLSDLTEEKFQLLLKKLSSFCLGITNSLK |
| | | TSAVAKLRVNQPDDYYGKVTCSEVFFQSLDKEHSAVLLYQ |
| | | KTGEKSRAYGLAFNNVVTGQYTTEASFYCDPKRFFLPIMSD |
| | | VVLFRMCNEMLSWLDYLSDDVMLEVRTCLYRLVLSILCTPS |
| | | KRVQVYIQGLRYFIMAFVNEFHCTGLLDKLKVTALTESERY |
| | | CMKLCDDLVVKVLNSVEDENMAKAFKFVLNTSYLCHLITK |
| | | ETPDRLTDQIKCFEKFLEPKLDFGSVIVNPDSSCELTAGQEEQ |
| | | FYQGLEKLFTDKKLESSYANKPGVCKEVLNVCMSLFNSGA |
| | | LEVKPLLNHDPITPSFTSTALDLSSNKSVVVPKLDELGEVLT |
| | | EYDYSKLVSSVVVDLVEHFKTKGKYVVSPRSLQYKIYKRLS |
| | | NLVQQRAGKGNKESELTEEEFLEQVTAEQLEVINKVETKVS |
| | | RTLSGIKLSSDTENAKHDDDYHLKKLWSKDIMVRIKAETSL |
| | | HEVKDFNVDTLPFDLYRELVDAIYNDPAANSHYFSERIFNPC |
| | | PLELLIKNLTLKAYKEEDFFECFKYILISSNFDNKVGKYDHK |
| | | NRSRLGLSSAALLVKDEARISMRESNSESIAKRLDKSFFTNSS |
| | | LRNLCFYSDESPTERTSVSSNVGKLKFGLSYKEQVGGNREL |
| | | YVGDLNTKLTTRLVEDYAESLTSDMKYTCLNNENEFERAL |
| | | LDMKSVVRQSGLAVSMDHSKWGPHMSPALFSLMLRGLDF |
| | | RLKDGTLIDKEAVVNILSWHIHKMVEVPFNVVEAYLKGFIK |
| | | RGLGLMDRGGATRVEEFMFGYFDQGIVPSHISSVIDMGQGI |
| | | LHNLSDLYGLITEQFIVYALDLCYSSSFMAYTSSDDEILLSIS |
| | | NSFKRNDGSMDMDLAIEALEFHYFLSDRLNKFVSPKTVAGT |
| | | FASEFKSRFFIWSQEVPLLTKFVAASLHNVKAKAPSQLAETI |
| | | DTILDQSVANGVSIEIIGAIAPRTNALITYSGHPFNLFLCLEET |
| | | DVRDWVDGSRGYRLQRSIENAFPDDVLPEIIRSACRKIFYRI |
| | | QSGTLEEDYIVTTLQQSPDDCLKQMLTSCDVEKEAIDDICNY |
| | | RWLNLRAHGDLRLVLRTKIMTSTRTLQKEEVPSLIKSVQSK |
| | | LSKNFVRGAKKILADAINKSAFQSCISSGFVGVCKSMGSKC |
| | | VRDGKGGFKYIKDILKEIKHHEKPDCHFCKELKGIYCSELLE |
| | | NISEFSRPLFWDYFSLVLSNACELGNWVFCKIEIPKSVYHLN |
| | | NPNHFWPIKPSSHAELEEKVGMNHVLYSIRRNFPVLFDEHIS |
| | | PYLSDLNMLKLNWVQKIRFLDICVAIDMTSECLGIISHIIRRK |
| | | REELYIVKQSELSMSHTRVSLPLERGFNIEPDEVCHNFLLQIL |
| | | FESMIHPVLLTTSQFKRYFWYSEVELLPKEALHDLGQFTQFII |
| | | DCKVLNSSRAMCLDDLDVGYVSSKVKRTDTYLNLSTFMTN |
| | | LDWENRHEYSSFEDLILSSPSEVFLFEITFTFSHIRRSHKFRYD |
| | | RSTNYILKTKLVIEKSELVNGEDGVYCVTPHSIEYYVSQSSG |
| | | NHISLDGVSLLVLDPLISGRELVNMDELLQNQDVTFSAPSQI |
| | | LSKIKLDFKPFTKEIKNKFSYKLIGPDVDMSPLHLDKGAIKE |
| | | GDRIVSQIEIQVSFKSVITAIELLDEDQRKIFVGNLFVYLTSLK |
| | | SVNRALSMSESDLRLLVENYPSVIEYMLSGCDGWLNCGSFS |
| | | LIKSKTLQCIMLADERGPYRIKGQNCRRLFPTEEAIEIE |
| 52 | Amino acid sequence of | MGQIVTFFQEVPHIIEEVMNIVLITLSLLAILKGIYNVMTCGLI |
| | glycoprotein precursor | GLLTFLFLCGKSCSTIYKDNYRLMQLNLDMSGLNATMPLSC |
| | (GPC) protein in Mobala | SKNNSHHYIQVFNTTGLELTLTNDSLIGHKWCNLSDAHKKD |
| | virus (MOBV) | TYDHTLMSIISTFHLSIPNFNHYEAMACDFNGGKISIQYNLSH |
| | | SSETDAMNHCGTVANGVLEVFRRMTWCTHCDTPLGASIAG |
| | | FNCVRTSYKYLIIQNTTWEDHCTMSRPSPMGYLSLLSQRAR |
| | | EIYISRRLMGTFTWTLSDSEGNDLPGGYCLQRWMLIEAEMK |
| | | CFGNTAVAKCNQQHDEEFCDMLRLFDFNKEAIHRLRVEAE |
| | | KSISLINKAVNSLINDQLIMRNHLRDIMGIPYCNYSRFWYLN |
| | | DTRSGRTSLPKCWMVSNGSYLNETHFSSDIEQEANNMITEM |
| | | LRKEYERRQGTTPLGLVDLFVFSTSFYLISVFLHLIKIPTHRH |
| | | LVGKPCPKPHRLNHMGVCSCGLYKQPGLPTKWKR |
| 53 | Amino acid sequence of | MGQIVTFFQEVPHIIEEVMNIVLITLSLLAILKGIYNVMTCGLI |
| | GP signal peptide in | GLLTFLFLCGKSCS |
| | MOBV | |
| 54 | Amino acid sequence of | TIYKDNYRLMQLNLDMSGLNATMPLSCSKNNSHHYIQVFN |
| | GP1 protein in MOBV | TTGLELTLTNDSLIGHKWCNLSDAHKKDTYDHTLMSIISTFH |
| | | LSIPNFNHYEAMACDFNGGKISIQYNLSHSSETDAMNHCGT |
| | | VANGVLEVFRRMTWCTHCDTPLGASIAGFNCVRTSYKYLII |
| | | QNTTWEDHCTMSRPSPMGYLSLLSQRAREIYISRRLM |
| 55 | Amino acid sequence of | GTFTWTLSDSEGNDLPGGYCLQRWMLIEAEMKCFGNTAVA |
| | GP2 protein in MOBV | KCNQQHDEEFCDMLRLFDFNKEAIHRLRVEAEKSISLINKA |
| | | VNSLINDQLIMRNHLRDIMGIPYCNYSRFWYLNDTRSGRTS |
| | | LPKCWMVSNGSYLNETHFSSDIEQEANNMITEMLRKEYERR |
| | | QGTTPLGLVDLFVFSTSFYLISVFLHLIKIPTHRHLVGKPCPK |
| | | PHRLNHMGVCSCGLYKQPGLPTKWKR |
| 56 | Amino acid sequence of | MSNSKEIKSFLWTQSLRRELSGFCTNTRVQVIKDAQSLLHGL |
| | NP protein in MOBV | DFSEVSNIQRLMRKEKRDDSDLKRLRDLNQTVNNLVELKSS |
| | | QQKNTLRVGALTSDDLLVLAADLDRLKAKVNRSERPLTGG |
| | | VYMGNLTQQQLDQRKILLQLVGMGGSRVPPRGGDGIVRV |
| | | WDVRNPDLLNNQFGTMPSLTLACLCKQGQEDLSDVVKALT |
| | | DLGLVYTAKYPNLSDLDKLTHTHPVLGLIDGNKSAINISGY |
| | | NFSLNAAVKAGASLLDGGNMLETIKVTPKNIDTILKCVLKV |
| | | KRSVGMFVSDTPGERNPYENILYKICLSGDGWPYIACRTSIS |
| | | GRAWDNTEVDLGTNKDPINKGPPTSNKTAGAAGFNAGLTY |
| | | SQMMELKDSMLQIDPTAKTWVDIEGRADDPVEIAIYQPSNG |
| | | HYIHFYREPTDIKQFRQDAKYSHGIDVQDLFTTQPGLTSAVL |
| | | ENLPKNMVLTCQGVEDIRKLLDSQGRKDIKLIDVSMQKAD |
| | | ARKFEHQIWDEYKHLCSMHTGIVVEKKKRGGKEEITPHCAL |
| | | LDCLMFEATTRNSLDIVIPRPVLSKDLVFRSATPKVIL |
| 57 | Amino acid sequence of Z | MGQKPSKPKAPPTTYESPRSSLTPDATGFGPEFCKSCWFERK |
| | protein in MOBV | GLIKCQNHYLCMTCLTLLLTVSNRCPVCKYPLPTKLRLEKSP |
| | | TAPPPEATNPPPYSP |
| 58 | Amino acid sequence of L | MEEQISEVKDIISKYLSNDDRLAKQKLAFLVQSEPKLLLIEGL |
| | protein in MOBV | KLLSLCIEIDSCEANGCDHNTKELSVENFLSENRVLCPGLPM |
| | | VVPDGFKLNGNVLMILECFVRSSPANFEQKYREDLVKLNSL |
| | | KEDLMTVGITMLPLIDGRTNFQTDRLPEWANERFRTLLFSLL |
| | | AFSQESSRMFEEAEYSRLCESLNVSGGKRSGIENINILSDHRS |
| | | EHFDELLKLCHVGINNHMSSLDVKREIIQEFQAFRNKLQNG |
| | | VIERQFLRVNREELIKAFNEMYTLRVGDKPELLDSLLNDYY |
| | | HSCPLITMLYCELPNGKSCQSDISHVRGWRSLLNKVKSLRLI |
| | | NTRRKLMLIFDSILLLAHMKDLSVNGHLVESEWMGSSFLSV |
| | | NDRLVSLPATQKDLKTWLQRRTNRLSHSHQSQSAYEVFST |
| | | MVNRVLNKAKEVLLLVNLTFKDYNVDEDILSESSFTEMMS |
| | | LEVNGVEPTINYEKNPIDRFSYNIQAMDPDNQSDLKRLSSISL |
| | | ALVNSMKTSSTVKLRQNEHGKLRYKCVRCKEAYYQDFLIE |
| | | GHRLMLIYQKTGECSKCYSVNDAVVGELCSFYADPKRYFP |
| | | AIFSDSVLQEMIDTMISWLTECSELKEFIKEIKSLLKMVVMV |
| | | VLTNPTKRIQKFLQNLRYFTMAYVSEYHHKDLLEKLREDLI |
| | | TNCEFLLYRITRSILNIVFNVNVTTMITNRFKFILNLSYLCHLI |
| | | TKETPDRLTDQIKCFEKFIEPKMKFDSVNVNPLEPADQEELR |
| | | SLLMSADKFLSKPDCFGDEGILFKTPGVSRKIFSMMVSSFNN |
| | | GSLFKQAELKNGVKDPLVVSGCATALDLASNKSVVVNKYT |
| | | DGDRIIEYDYDKLVATAVCQLSEVFSRKGKYVLSKEDYDFK |
| | | IQQIMSDLVIGRSKLHGSEIGLNSCEEVDEVLIEGGAADYFDS |
| | | IKQSVDTVMSKFSWSGSESSATLKSECSIDDLSLALQDKAQL |
| | | RLIRNELSCHMVEDFDVMTLPYDTYEEICKSVYSDPSLRSKY |
| | | FYLESLESCPLTKMAQAVCTRTFHDEEYFQCFKSLLLQMNA |
| | | NKLSGKFNHYKSKCLNFKLDRDRLFNETRISERESNSEALSK |
| | | ALSLTNCTTAALKNLCFYSQESPQSYNSQGPDTGRLKFSLSY |
| | | KEQVGGNRELYIGDLRTKMFTRLIEDYFEALTSQFKGSCLN |
| | | DEHEFENAVFSMKFNVSLGLLSYSLDHSKWGPMMTPFLFL |
| | | ATLQNINWPSLDTLSDAKSRDYVSSMLSWHIHKLVEVPFNV |
| | | VTAMMKSFIKSKLGLKKNLSETMTERFFFEHFRLGKVPSHIS |
| | | SILDKGQGILQNTSDFYGLISERFINYCISCLYEGNVDAFTSS |
| | | DDQISLFDKSLSDLLEKDPDEFEYILEFHNYLSDQLNKFISPK |
| | | SVKGNFAAEFKSRFFVWGDEVPLLTKFVAASLHNIKCKEPH |
| | | QLAETIDTIIDQAVANGVPVKLCNIVQERTLNLLRYAQYPID |
| | | PFLMFCSSDVKDWVDGNRGYRIMRNIEMLEPNGTRKVRSF |
| | | LRRLYNNLKTGLLHEEFTAAYLSGDPYQSLAKLSKIFDTEIL |
| | | NDEELGLSWLNLSAYYPLRMVLRQKVIYTGAVNVEEEKLP |
| | | TIVKTLQNKLSSNFTRGAQKLLSEAINRSAFQSCIASGFVGLC |
| | | RTLGSKCVRGPERENFYIKSIMNQSMMMEGVSRELVMGVD |
| | | VWRVRNPLDNSRAQQKWGNYFRPILWDYLCIALSTALEIGS |
| | | WVLGEPKLKSPLPQMKFRPCDYFPMKPSVTRLLEDKVGFN |
| | | HIIHSFRRLYPDIFEKHLLPFMSDLASTKMKWSPRVKFLDLC |
| | | VMLDVNCEAMSLVSHIVKWKREEHYVILSDELSISHDRSHE |
| | | SLADERVVSTEDVSENFLRQIYFESFARPFVATSRTLGSFTW |
| | | FPHKTSLPESEGLASLGPFGTFIEKVIFKGIERPMYRHDLFSG |
| | | YAWLDFDFGEFYINSSKLIQYGLTEMRYFEDLSEFMSMLSSL |
| | | KPGSIEISLTVNFQVKSQGESLREKFFIHCKFYGSFDVDGKFE |
| | | FNNIGVQYSGAINRSAVLDCWRLILTNSHFLGDKVIWHLNT |
| | | ANIKDYLKDGSMVGEVVPIEVIINRDALRLDTLDFERVGPD |
| | | VNVVPLVVKDGYIFEGDKKLVPFNPSIHDQDFEILVKELCID |
| | | DKELLKDMIQKMITVRGSQGLQWHSLDIVAVLTKNMPTNY |
| | | KDFITESLSVLDSWTGFKGYSLCFSKTKNTLMIHTSEGNLRL |
| | | KGKLCRKLFDDPVHVEDIE |
| 59 | Amino acid sequence of | MGQIVTFFQEVPHILEEVMNIVLMTLSILAILKGIYNVMTCGI |
| | glycoprotein precursor | IGLITFLFLCGRSCSSIYKDNYEFFSFDLDMSSLNATMPLSCS |
| | (GPC) protein in Mopeia | KNNSHHYIQVGNETGLELTLTNTSIIDHKFCNLSDAHRRNLY |
| | virus (MOPV) | DKALMSILTTFHLSIPDFNQHEAMSCDFNGGKISVQYNLSHS |
| | | NYVDAGNHCGTIANGIMDVFRRMYWSTSLSVASDISGTQCI |
| | | QTDYKYLIIQNTSWEDHCMFSRPSPMGFLSLLSQRTRNFYIS |
| | | RRLLGLFTWTLSDSEGNDMPGGYCLTRSMLIGLDLKCFGNT |
| | | AIAKCNQAHDEEFCDMLRLFDFNKQAISKLRSEVQQSINLIN |
| | | KAVNALINDQLVMRNHLRDLMGIPYCNYSKFWYLNDTRTG |
| | | RTSLPKCWLVTNGSYLNETQFSTEIEQEANNMFTDMLRKEY |
| | | EKRQSTTPLGLVDLFVFSTSFYLISVFLHLIKIPTHRHIKGKPC |
| | | PKPHRLNHMAICSCGFYKQPGLPTQWKR |
| 60 | Amino acid sequence of | MGQIVTFFQEVPHILEEVMNIVLMTLSILAILKGIYNVMTCGI |
| | GP signal peptide in | IGLITFLFLCGRSCS |
| | MOPV | |
| 61 | Amino acid sequence of | SIYKDNYEFFSFDLDMSSLNATMPLSCSKNNSHHYIQVGNE |
| | GP1 protein in MOPV | TGLELTLTNTSIIDHKFCNLSDAHRRNLYDKALMSILTTFHL |
| | | SIPDFNQHEAMSCDFNGGKISVQYNLSHSNYVDAGNHCGTI |
| | | ANGIMDVFRRMYWSTSLSVASDISGTQCIQTDYKYLIIQNTS |
| | | WEDHCMFSRPSPMGFLSLLSQRTRNFYISRRLL |
| 62 | Amino acid sequence of | GLFTWTLSDSEGNDMPGGYCLTRSMLIGLDLKCFGNTAIAK |
| | GP2 protein in MOPV | CNQAHDEEFCDMLRLFDFNKQAISKLRSEVQQSINLINKAV |
| | | NALINDQLVMRNHLRDLMGIPYCNYSKFWYLNDTRTGRTS |
| | | LPKCWLVTNGSYLNETQFSTEIEQEANNMFTDMLRKEYEK |
| | | RQSTTPLGLVDLFVFSTSFYLISVFLHLIKIPTHRHIKGKPCPK |
| | | PHRLNHMAICSCGFYKQPGLPTQWKR |
| 63 | Amino acid sequence of | MSNSKEVKSFLWTQSLRRELSGYCSNIKIQVIKDAQALLHG |
| | NP protein in MOPV | LDFSEVANVQRLMRKEKRDDSDLKRLRDLNQAVNNLVELK |
| | | SVQQKNVLRVGTLTSDDLLVLAADLDRLKAKVIRGERPLA |
| | | AGVYMGNLTAQQLEQRRVLLQMVGMGGGFRAGNTLGDGI |
| | | VRVWDVRNPELLNNQFGTMPSLTIACMCKQGQADLNDVIQ |
| | | SLSDLGLVYTAKYPNMSDLDKLSQTHPILGIIEPKKSAINISG |
| | | YNFSLSAAVKAGACLIDGGNMLETIKVTKSNLEGILKAALK |
| | | VKRSLGMFVSDTPGERNPYENLLYKLCLSGEGWPYIASRTSI |
| | | VGRAWDNTTVDLSGDVQQNAKPDKGNSNRLAQAQGMPA |
| | | GLTYSQTMELKDSMLQLDPNAKTWIDIEGRPEDPVEIAIYQP |
| | | NNGQYIHFYREPTDIKQFKQDSKHSHGIDIQDLFSVQPGLTS |
| | | AVIESLPKNMVLSCQGADDIRKLLDSQNRRDIKLIDVSMQK |
| | | DDARKFEDKIWDEYKHLCRMHTGIVTQKKKRGGKEEVTPH |
| | | CALLDCLMFEAAVIGSPQIPTPRPVLSRDLVFRTGPPRVVL |
| 64 | Amino acid sequence of Z | MGKTQSKGQPSTNLLKETEPRHPVIPDARGTGPEFCKSCWF |
| | protein in MOPV | ERRGLVRCNDHYLCLNCLTLLHTVSDRCPICKHKLPFRLEL |
| | | QTQPTAPPEIPPSQNPPPYSP |
| 65 | Amino acid sequence of L | MEELLSESKDLVSRYLLEDERLSKQKLAFLVQTEPRMLLIEG |
| | protein in MOPV | LKLLSLCIEIDSCKANGCEHNSEDLSVEILLQRQGVLCPGLPF |
| | | VVPDGFKFSGNTLILLECFVRTSPINFEQKYKEDTIKLESLKP |
| | | DLSSVDIILLPLIDGRTNFYTDLFPEWANERFRHILFSLLEFSQ |
| | | QSSKMFEESEYSRLCESLTKAGVRTSGIESLNVLTDSRSDHY |
| | | ERVLELCHRGINNKMSILDVKKEIVSEFHAFRNKLKEGEIER |
| | | QFVRTDRRQLLRDFNNLYIDREGDTPSEIDPLKERFVKSSPM |
| | | VTALYGDYDRYRQEGVDRDSCLQNHFQSSVPGWKSLLNKI |
| | | KSLKLLNTRRKLMLTFDAIILLAHLKDLKCHGELLGSEWLG |
| | | SSFLSVNDRLVSLQETQKDLKKWIERRMVSAMKKKGGVGT |
| | | LCQRSELIFFDIINKLLTKAKEALSSASLCFRDYVKEEDILEE |
| | | DSYERLMLMEKRGIQPTMSYEKEEGNQFPYPLIELEADSIED |
| | | LRRLSSISLALVNSMKTSSVAKVRQNEYGAARYKRVRCKE |
| | | AFNQSFIMGSGNFNLIYQKTGECSKCYAINNPEKGEICSFYA |
| | | DPKRFFPAIFSHCVIYETINTMMSWLSECIELRDQQKTLKLLL |
| | | KITMILILVNPSKRAQKFLQGLRYFIMAFVSDFHHKQLMEKL |
| | | REDLITEPEHLLYSVVRSILNIILGEGVSTMLTNRFKFVLNLS |
| | | YMCHFITKETPDRLTDQIKCFEKYLEPKLEFDSININPSEEGD |
| | | EDERMLLLESANKFLSKETSMSNNRISYKVPGVSRKFFSMM |
| | | TSSFNNGSLFKKGDDLSGFKDPLVTAGCATALDLASNKSVV |
| | | VNKYTDGERILYYDHDKLVAASVCQLSEVFQRKTKYLLSK |
| | | EDYDYKVQKAISDLVVGKKSGSSNPNSQGAPDELDELFLDS |
| | | CALDCLEDVKKSVDVVLEKYRYDRKFPVGNGSEEKSLTDL |
| | | RKVLGTEDVGCVYYRLIQAEIAHHMVEDFDESLLPGDAYE |
| | | MICKGFFKDLELRSKYFYLDSLDSCPITCITQAVSTRTFNDQ |
| | | QFFQCFKSLLLQMNAGKLAGKYSHYKNKCLNFKIDRERLM |
| | | NDVRISERESNSEALGKALSLTNCTTAVLKNLCFYSQESPQS |
| | | YTSLGPDTGRLKFSLSYKEQVGGNRELYIGDLRTKMFTRLIE |
| | | DYFEALTKQYRGSCLNNEKEFHNAILAMKLNVSLGQVSYSL |
| | | DHSKWGPMMSPFLFLVFLQNLRWETRDDIEDIKSKDYVSTL |
| | | LSWHIHKLIEVPFNVVNAMMRSYLKSRLGLKKSLHQTSTEA |
| | | FFFEYFKQNRIPSHLSSIIDMGQGILHNASDFYGLVSERFINY |
| | | CIKCLFEDEVDSYTSSDDQISLFGKDLSDLLSNEPEEFQAILE |
| | | FHYFLSDQLNKFISPKSVIGSFVAEFKSRFYVWGDEVPLLTK |
| | | FVAAALHNVKCKEPHQLAETIDTIIDQSVANGVPVTLCNAIQ |
| | | ERTLNLLRYAQYPIDPFLLFLDSDVKDWVDGNRGYRIMRNI |
| | | EAILPESTQKVRKVLRTVFNKLKLGELHEEFTAIYLSGDPAD |
| | | SFKKLTSLVGDDTLSEEDLSVCWLNLTTHHPLKMVMRQKV |
| | | IYTGAVELGEEKLPTLVKTLQSKLSSNFTRGAQKLLCEAVN |
| | | KSAFQSGIASGFIGLCKTLGSKCVRFSDRSTAYIKSLVSRLSA |
| | | LDSVSSLKVKGVDLWILGKEHTKAAEEALGFLRPVLWDYF |
| | | CIALSTSLELGSWVLGEPKVKEKTSSIPFKPCDYFPMKPTTT |
| | | KLLEDKVGFNHIIHSFRRLYPSLFERHLLPFMSDLASTKMRW |
| | | TPRIKFLDLCVVLDVNCEAMSLISHVVKWKREEHYVVLSSD |
| | | LAIAHERSHLPITDERVVTTYDVVQNFLRQIYFESFIRPFVAT |
| | | SRTLGSFTWFPHRSSIPESEGLDNLGPFSSFIEKVIYKGVERP |
| | | MYRHDLYSGYAWLDFECAPAILNLGQLIASGLTEQHVFESV |
| | | SELLEAFADLSVGSVQISVTVNFQVRSQGESLKEKFSLHLLF |
| | | KGVVLEGGLFKPHSLDVTYSGSVQRSAIKDCWRVAQTSTW |
| | | FKRETTSIWLLSTENICDYLRDSSPIPDVIPLSVLLNEEILDLE |
| | | EHDFTHIGPEHVEIPLVVDSGYLIEGTRKLLPFNPNIHDQDLN |
| | | VFIGELMEDHSEILERSLSKMLRSRMDQGLHWLQLDIIGVV |
| | | GRCMPEGYENFLTRVFSGIDFWADFKGYSLCYSRSQASLMI |
| | | QSSEGKFRLRGRLCRPLFEEVGPPLDIE |
| 66 | Amino acid sequence of | MGQIITFFQEVPHIIEEVMNIVLITLSLLAILKGVYNVMTCGLI |
| | glycoprotein precursor | GLISFLLLCGKSCSLIYKDTYNFSSIELDLSHLNMTLPMSCSR |
| | (GPC) protein of Ippy | NNSHHYVFFNGSGLEMTFTNDSLLNHKFCNLSDAHKKNLY |
| | virus (IPPV) | DHALMGIVTTFHLSIPNFNQYEAMACDFNGGNISIQYNLSH |
| | | NDRTDAMNHCGTVANGVLDAFYRFHWGRNITYIAQLPNG |
| | | DGTGRWTFCYATSYKYLVIQNISWADHCQMSRPTPIGFASIL |
| | | SQRIRSIYISRRLMSTFTWSLSDSSGTENPGGYCLTRWMLFA |
| | | ADLKCFGNTAIAKCNLNHDEEFCDMLRLIDFNKQALKTFKS |
| | | EVNHGLQLITKAINALINDQLIMKNHLRDLMGIPYCNYSKF |
| | | WYLNDTRTGRVSLPKCWMISNGTYLNETHFSDEIEQEADN |
| | | MITEMLRKEYQERQGKTPLGLVDLFIFSTSFYSITVFLHLIKIP |
| | | THRHIVGQGCPKPHRLNSRAICSCGAYKQPGLPTKWKR |
| 67 | Amino acid sequence of | MGQIITFFQEVPHIIEEVMNIVLITLSLLAILKGVYNVMTCGLI |
| | the GP signal peptide of | GLISFLLLCGKSCS |
| | IPPV | |
| 68 | Amino acid sequence of | LIYKDTYNFSSIELDLSHLNMTLPMSCSRNNSHHYVFFNGSG |
| | GP1 of IPPV | LEMTFTNDSLLNHKFCNLSDAHKKNLYDHALMGIVTTFHLS |
| | | IPNFNQYEAMACDFNGGNISIQYNLSHNDRTDAMNHCGTV |
| | | ANGVLDAFYRFHWGRNITYIAQLPNGDGTGRWTFCYATSY |
| | | KYLVIQNISWADHCQMSRPTPIGFASILSQRIRSIYISRRLM |
| 69 | Amino acid sequence of | STFTWSLSDSSGTENPGGYCLTRWMLFAADLKCFGNTAIAK |
| | GP2 of IPPV | CNLNHDEEFCDMLRLIDFNKQALKTFKSEVNHGLQLITKAI |
| | | NALINDQLIMKNHLRDLMGIPYCNYSKFWYLNDTRTGRVS |
| | | LPKCWMISNGTYLNETHFSDEIEQEADNMITEMLRKEYQER |
| | | QGKTPLGLVDLFIFSTSFYSITVFLHLIKIPTHRHIVGQGCPKP |
| | | HRLNSRAICSCGAYKQPGLPTKWKR |
| 70 | Amino acid sequence of | MANSKEVKSFLWTQALRRELGQYCSTVKSSIIKDAQSLLHS |
| | nucleoprotein of IPPV | LDFSEVSNIQRLMRKDKRNDSDLKRLRDLNQAVFNLVELKS |
| | | TQQKNVLRVGKLTSDDLLVLAADLDRLKNKVMRTERPQTL |
| | | GVYMGNLTNQQLDQRKRLLDMIGISNARNAPRPGADGVVR |
| | | VWDVKDSSLLNNQFGTMPSLTLACMSKQGQYELNDVVQS |
| | | LTDLGLVYAAKYPNAMDLEKLTQAHPVLSIIDVSKSSINVSG |
| | | YNFSLSAAVKAGACMLDGGNMLETLKVTPQNLEDILASML |
| | | KVKRAHSMFVSDTPGDRNPYENLLYKVCLSGNGWPYIACR |
| | | TSLTGRAWDNTVVDLGPPIDLSQNKQMSPAKPKGAGHGMP |
| | | SGLTMSQILALKDLMAAVDPNAKTWIDIEGRAEDPVEIAFY |
| | | QPQTGAYIHFYREPTDAKQFKQDSKYSHGIDIGDLFNVQPG |
| | | LTSAVLELLPPNMVLTCQGSEDIRRLLDSQGRKDIKLIDVLM |
| | | SKSEARKFEDEVWDKFGFLCKIHTGHVVEKKKRGNKEEITP |
| | | HCALLDCLMYEAASTGRFSPGSIRAVLPRDMVFRAVTEKVA |
| | | L |
| 71 | Amino acid sequence of Z | QCQAVHAKVVIVTSNKTPTLKPAALAKLWPHACCIWNDVC |
| | protein of IPPV | SVPCVFGLILDGLLFVSALVLSHAGLGDGLAIWRDCWRVLV |
| | | GVVVAGLV |
| 72 | Amino acid sequence of L | MEESLRETKLLISRYLRQDERIARQKLAFLGQSEPRSLLIEGL |
| | protein of IPPV | KLLSLCIEIDSCDTNCCTHNTEGQSVENFLFQNHILCPSLPLV |
| | | VPDGMKLNGNILIILECFVRSNPTNFQQKYQEDSVKLDSLKG |
| | | DLERAGISLIPIIDGRTSYYNSLMEEWVCDQFRHNLFKLLEFE |
| | | QENNALFEESEYLRLCESLNVSGGRASGAQGLHSLLDCRGE |
| | | HYNEILKACHIGIDPSIGGVELKGQIENLYQVFRQKLKKGVI |
| | | KHQFRKVDQKSLLKEYCEMYKGIGICGVEETTVDALAAELP |
| | | NISPILRYIHLRIDSESNAEVNEISNLPTGLRSAFNKVKSLKVL |
| | | NTRRKLLLLIDTIILMSHCYVRELFPTLCERDWLGSSFFSVGD |
| | | RLVSVGAIQHDLSKWLKRRLKANGGVGQKSTELHKMINTM |
| | | IQKSSKALGDVGLSFESYGVSFDFLNKVGLEEIMRFKIVGVT |
| | | PTISYIKTNQQPPIPLREFSAEDDSDLKMLSSLSLSLVNSMKT |
| | | SSTVKTRQNAMGRERYRVVQCKECYYQELGNEYRDLVLL |
| | | YQKTGEGSKCYSVNSKRVGEICSFYADPKRYFCPIFSENVIT |
| | | KVIDTMMTWLMGIVELEDSLRDIKKLTKMILLVILCQPSKRS |
| | | QKLLQNLRYFIMAFVSDYHHVELFDKLREELITDAEFFLFKL |
| | | LGKILTILLNDEVSTMLNNRFKFILNISYFCHFITKETPDRLTD |
| | | QIKCFEKYLEPKIQFGSLTVNPKETPTDEEKDDILHGVNMFL |
| | | SKKTCDEVDDPPSKKPGVSKKVFSLMLSAFNSGLLFKESEL |
| | | KKGMKDPLEDSGSATALDLASNKSVVINKYTKDGRVLDYN |
| | | YDKLVSVAVCQLSEIFSRKGKYLLNKEDYDYKIQEVLSSLVI |
| | | GSSKSEQPEEILDVDSDYMDQLKASVERVLDQYKPNRGVRS |
| | | QNNDKSVNDLKIIVEDELSRRLILGELSYHLVEDFDKGLLSE |
| | | NFYKEVCEKAFNNKDFRTKYFYDSEAGLCPIEKMTQALATR |
| | | TYMSGEYFHCFKSLLLQMDANKLSGKYSHYKSQNLNFRFD |
| | | HGRLMDDSRISERESNSEALSKALSLVNCLTSALKNLCFYSQ |
| | | ESPSSYTETGPDTGRMKFSLSYKEQVGGNRELYIGDLRTKM |
| | | FTRFVEDYFESYTKQLEGSCLNNEKEFEKAILGMKLGVSLA |
| | | HASYSLDHSKWGPMMCPFLFLMLYRNLSPKLKGTEVELKG |
| | | CDNISTILSWHIHKLVEVPFNVVTAMMRSYIKRKLGIMKDTS |
| | | QTITESLFFSEFERGVIPSHFSSVLDMGQGILHNTSDFYGLISE |
| | | RFINYALRLVSGNPIEAYTSSDDQISLFSHKFTELMDTDPEEF |
| | | LIYLEFHNYLSSLLNKFISPKSVVGRFVAEFKSRFYVWGDEV |
| | | PLLSKFVAASLHNIKCKEPHQLAETVDTIIDQAVANGVPVSV |
| | | CNEVQKRTLRLLEFSKYPIDPFLLHSDSDVKDWVDGNRGYR |
| | | IMRVIEQTLPEGTASVRSLLRILYNKLKSNELHEEFASAYLSQ |
| | | NRSETLVGLAELMGVKPPSTEDLMICWLNLTACHPLRMVL |
| | | RQKVIYPSALNLEEEKVPTLIRTLQNKLSSGFTRGAQKLLSE |
| | | AVNKSAFQSSIASGFVGLCKTLGSKCVRDPERESHYIKSIIQY |
| | | LQTHCNVKPLNKGHLNLWVYESKTDDTQSASVKPWQIELL |
| | | RPLLWDYLCIALSTSLEIGPWVLGEPVFKVKSDFWKPRPCD |
| | | YFPLRPAHNRILEDRIGMNHIIHAVRRLYPEMFEKHLLPYMS |
| | | DLAAMKLKWSPRIKFLDLCVTLDVNCEALSLISHVVKWKR |
| | | EEHYIVLSDDLLVSHDRKHTTLMDETVVSTSDVADNFLKQI |
| | | YFESFVKPFVATSRTLGSFSWFPHRSSLPQGEGIERLGPFSTFI |
| | | EKVVFKGIERPMYRYDLFMGYSWLDYEIELAHLNQSQLIAS |
| | | GLTEESCFEDVDQFWHYLSTLKVGSVKLSKTVRLTQKTQG |
| | | KLQGQKFSVHLNFTGFITNSCTFVPKQLEVLYSGPVDEHFVI |
| | | DCWSLLKSDREFKAGASEWFVHSDVVDAYISTASPSSEAYP |
| | | LDVWLEPDLLELSVSDISKVGPEVNIVPLVVEDGHLLELKEK |
| | | VAIINPVILDQDIEVFINELKEDHWDLLVCKFADILKHRQCC |
| | | NLYLINVDILTIALRILNDKAEEFISKSMQEIDQWFDFKGYSL |
| | | CFSKSRRQVMRHSSTGTMRLKGRLCQPAFYVEVVEEID |
| 73 | Amino acid sequence of | MGQLVSFFQDIQLFFQEALNVALAVVTVLAIIKGLVNLWKS |
| | glycoprotein precursor | GLFQFLFFLILAGRSCSFRIGHHTTFESVTMSVGGVFHELPAL |
| | (GPC) protein in Amapari | CRINNSHSLIQLSHNSSLALSVEYVDLCVVLESDQYLVAGD |
| | virus (AMAV) | YSNCTGEATGYNWVIDWTLKGLGHGLEGDPKLHCQPKRST |
| | | NAEFTLQLNISRRHTNDHYRERIETGIRHMFGPFKILTKEGK |
| | | DCVILRNTTWKEQCVKSHYNTLAFLLKNTANSLPKRRPLAF |
| | | FSWSLSDSSGNDMPGGYCLEEWMLIAAKLKCFGNTALAKC |
| | | NLNHDSEFCDMLKLYEFNKNAISKLNNQTREAVNALTHSIN |
| | | SLISDDLLMKNKLREFLKVPYCNYTKFWYVNHTKSGEHSLP |
| | | KCWLVNNGSFLNESEFRNEWILESDHLIAEILSKEYQDRQG |
| | | KTPITLVDMCFWSAIFFDNKSLLHLVGFPTHRHIVGEACPLP |
| | | HKINRHGACACGLYQKLKKKTAWRKRHQ |
| 74 | Amino acid sequence of | MGQLVSFFQDIQLFFQEALNVALAVVTVLAIIKGLVNLWKS |
| | GP signal peptide in | GLFQFLFFLILAGRSCS |
| | AMAV | |
| 75 | Amino acid sequence of | FRIGHHTTFESVTMSVGGVFHELPALCRINNSHSLIQLSHNSS |
| | GP1 protein in AMAV | LALSVEYVDLCVVLESDQYLVAGDYSNCTGEATGYNWVID |
| | | WTLKGLGHGLEGDPKLHCQPKRSTNAEFTLQLNISRRHTND |
| | | HYRERIETGIRHMFGPFKILTKEGKDCVILRNTTWKEQCVKS |
| | | HYNTLAFLLKNTANSLPKRRPL |
| 76 | Amino acid sequence of | AFFSWSLSDSSGNDMPGGYCLEEWMLIAAKLKCFGNTALA |
| | GP2 protein in AMAV | KCNLNHDSEFCDMLKLYEFNKNAISKLNNQTREAVNALTH |
| | | SINSLISDDLLMKNKLREFLKVPYCNYTKFWYVNHTKSGEH |
| | | SLPKCWLVNNGSFLNESEFRNEWILESDHLIAEILSKEYQDR |
| | | QGKTPITLVDMCFWSAIFFDNKSLLHLVGFPTHRHIVGEACP |
| | | LPHKINRHGACACGLYQKLKKKTAWRKRHQ |
| 77 | Amino acid sequence of | MANSKEIPSFRWTQALRRELGSFTEPTKGSVLKDAKLIADSL |
| | NP protein in AMAV | DFTQVSQVQRLLRKTKRTDADLDKLRDLNKEVDKLMSMK |
| | | SAQRNTVLKVGDLGKDELMDLASDLEKLKRKIGERPNGGP |
| | | RLYMGNLSQSQLDKRSDILRSLGFQQQRGPNQGIVRLWDVS |
| | | DPSKLNNQFGSMPALTIACMTVQGGETMNNVVQALTSLGL |
| | | LYTVKYPNLDDLEKLTVEHDCLQIITRDESALNISGYNFSLS |
| | | AATKAGASLIDGGNMLETIKVTPDNFSSIIMATLKVKKREG |
| | | MFVDEKPGNRNPYENLLYKLCLSGEGWPYIGSRSQIVGRSW |
| | | DNTSVDLNTKPTVGPRGPERNGQNMRLSNLSELQEAIVREA |
| | | MQKLDPSNTIWMDIEGPPTDPVELAVLQPSTGYYIHCYRKP |
| | | HDEKGFKNGSRHSHGILLKDLEDAQPGLLSYIIGLLPQNTVI |
| | | TVQGADDIKRLFDIHGRKDLKLVDVRLTGEQSRIFEQEVWE |
| | | RYGELCKAHNGVIVPKKKHKDNGPQKEPHCALLDCIMFQS |
| | | VLDGHLPELSLKPLLPGSLVYQARNAFVM |
| 78 | Amino acid sequence of Z | MGNCNVKQETQPQSTRPKTTSTETELLRTPPVSLHGRYNCK |
| | protein in AMAV | CCWFADKNLVVCSDHYLCLRCLNLMLRTSDLCNICWKPLP |
| | | TRIAIPVEPSAPPE |
| 79 | Amino acid sequence of L | MDEKINSLKDFVRKQVPEIPELSYQRELLLSQVEMGMILME |
| | protein in AMAV | GFKLLSCLVEIESCKKNSCEHNSSQKFVDVILSDNGVVTPTL |
| | | PKVIPDGFRFFNKTLILLETFVRVNPEEFEKKWKRDMSKLLT |
| | | LKEDVQKAGITLVPIVDGRCNYNTNLMPDWATERFRWLLI |
| | | DLLRESRGDSKIDIEEQEYQRLVHSLSKTGNQSLGFENIECL |
| | | KRHCLNYESRLDESLLTGFNNDLRESKIREGLIKMKNWYRK |
| | | EVFVKGMGNFVKTDKAKLLQSLESLGLHANSGTSECPFCCC |
| | | KILDICYKLMQKLKHGHSLSEGIPDEFIAKSQIEKEYLLVLSV |
| | | CNKIKGKKVFNTRRNTLLFLDLIMLNFVIDVFENNPTELCFL |
| | | KESGLIIGQMLLFSNDRVLDILSARKLLKKKLEISAHWVKKC |
| | | NStLKRAEPDLWDYVSKYITEPKFDSLTSLAEELCTERPVMR |
| | | YKVQIHSGDGCSHKEFETLSEQQQICLFKCLSHVSLSLTNSM |
| | | KTSFSSRLLVNEVDHKKYFGTVRLKECYVQKFFLTNELYGL |
| | | LFYQKTGERSRCYSLYLSDKGQLKEVGSFYCDPKRFFLPIFS |
| | | DTVLLLMCAEMVSWLDFCEELAREVDPLLRLLVLSILCSPS |
| | | KRNQTFLQGLRYFIMAFVNQAHHVQLMSKLVVECKSASDV |
| | | LIQRLSVRMFRMVLDFGSDPDAFMSRKFKFLLNVSYLCHLV |
| | | TKETPDRLTDQIKCFEKFLEPKLEFGFFVVNPSLNGTLTKEQ |
| | | EDAMVGGVSKFFSKDIFNIEDMKQPGVSRDLLSYCLTLFNK |
| | | GKLKVNGSLKVNPFRPSFTSTALDLSSNKSVVIPKLDELGNI |
| | | VSVYDKQKLVSTCVASLVERFKTKGKFNLDPNEIEFLIMDN |
| | | LTNLLAIKGSAVKEREELSMLYEQLSDDAIKAFEELRQDVEF |
| | | TLGRMRTPEKKKKTTNFYGNCTLESLWAPFNVMKAIRTETS |
| | | IHEIRDFDPNILPPEIYEELCTSVFESSLKESFFLNEVLDICPLE |
| | | LLLKNLTTRCFEEQEFFDCFKYLLIHAGFDQRLGTYEHKSKS |
| | | RLGLSEDVFKLRDDVRMSQRESNSEAIARRLDKSFFTSAAL |
| | | RNLCFYSEESPTEYTCISPNTGNLKFGLSYKEQVGSNRELYV |
| | | GDLNTKMMTRLVEDFAEAVAKSMNYTCLNSEKEFEKAICD |
| | | MKMAVNAGDICCSLDHSKWGPFMSPALFLSFINELKLKDHE |
| | | TSALIDCRPVLSVLRWHLHKAVEVPFNVAEAYCTGMLKRR |
| | | LGLMSLQSQSVSEEFFHQQLLMGKEVPSHIMSVLDMGQGIL |
| | | HNLSDLYGLITEQFLNYCLDLLFDVVPISYTSSDDQITMIKFP |
| | | TTNAPEGGESQSDWLEVLCFHDFLSSKLNKFVSPKSVCGSF |
| | | AAEFKSRFFVMGEETPLLTKFVSAALHNVKCKTPTQLAETI |
| | | DTICDQCVANGVGVTIVGEISKRVNRLIKYSGYPQTPFLAVE |
| | | VQDVKDWVDGSRGYRLQRNVENCLSDHPQLELIRKSAKKV |
| | | LTKIKRGLIFEEHLVQLIGKGGDNAMEAFLSYIDCSEIEKREA |
| | | LRYRWLNLSSNGDLRLVLRTKLMSARRVLEKEQIPTLIKTL |
| | | QSKLSKNFSRGAKKILAESINKSAFQSSVASGFIGFCKSMGS |
| | | KCVRDGSGGFLYIKDVISKVKICSCDICSLAPGIVFCDEALEN |
| | | VSMFSRPILWDYFSLVLTNACELGEWVFSCVQIPPKPTLLSN |
| | | PNLFWAVKPRGVRLVEDQLGLNHVLQSVKRNYPKIFEEHL |
| | | VPFMSDLQVSRTTDFTKLKYLDVCIALDMMNENLGIISHLL |
| | | KGRDNSLYIVKQNECASAHVRQVEYVDYDVGLSPQQICSNF |
| | | KIQLTFSSFINPLVMTTSTLRSFFWFGEVLRLEDENQIDVGEL |
| | | TDFVLLIKKYNVDRAMMLDDLTMGFVVSEIGEPKFRLLDFE |
| | | WSGVEEQTSFPLSENSNNNEIKFTLNLQLEHKRLSTKYRLTR |
| | | LVVYSYTMVCVMLVNDSRGVLKVENLTLRASGDVKEHRF |
| | | LDGVTLVSHHPTLCGKRGINILDLFRDAELPIPETRMFPEEVF |
| | | LDLSDYQSEVEDKYAYEIVGPEFSDVPLVMSGGCLVVGDK |
| | | KLSHLLTELTGNVILKALGALETDGEIGSFLMGLWPYIKATK |
| | | QRVKISQEEFLFIYETHRRSLLMSFEAYNDWLEFLDFSVCFS |
| | | KTLGDLVVSDSSGNMRLRGVVCRPLRQVGTVMEIE |
| 80 | Amino acid sequence of | MGQVVTFFQSIPEIIQEAINIALIAVSVICIIKGCVNLWKCGLI |
| | glycoprotein precursor | QLMVFLLLAGKRCDALNIDRRHVLSSVELNLTRMFDNFPQS |
| | (GPC) protein in Flexal | CSKNNTHHYYKGPEGTNWGIELTLTNTSVANYTSMNRIRSL |
| | virus (FELV) | AFGNITNCDKTGEAGHTLKWLLNELHFNVLHVTRHVGARC |
| | | MTTEGAGLLIQYNLTIGDHGGEVGRHLIASLAQIIGDNKAA |
| | | WVGKCDSRCSNDGKCNYTNCEGYTHYNYLIIQNTTWAQH |
| | | CTYSPLPSIRMALNKVAYSSVSRSLLGFFTWDISDSSGNHVP |
| | | GGYCLEQWAVVWAGIKCFDNAVMAKCNKEHDVEFCDTM |
| | | RLFDYNQNAIKTLQLNTENAVNLLKRSINGLISDSLVIRNSL |
| | | KQLARIPYCNYTKFWYINDTVSGKHSLPQCWKVHNGSYLN |
| | | ESQFKNEWLLESDHLYSEMLLKEYEERQGRTPLALTDICFW |
| | | SLVFFTSTVFLHLVGIPSHRHIVGDACPKPHRITKNALCSCGY |
| | | YNLPGKQVRWVRKGK |
| 81 | Amino acid sequence of | MSDSNIPAFRWTQALRRGLSNWTQTVKADVIKDAKAIMSA |
| | NP protein in FLEV | LDFNQVAQVQRMMRKDKRSEADLTRLRDMNKEVDALMT |
| | | MRSVQRDVVLKIGGLSKDELLELSSDLEKLRKKVMRAEGSS |
| | | TPGVYQGNLTTSQLQQRADLLKLVGMKPLQQSRGGVVKV |
| | | WDVKDSSLMINQFGSMPALTIACMTEQGGEQMNDVVQGL |
| | | TALGLVYTVKFPNLDDLEKLTEQHPCLKLITQEQSQINISGY |
| | | NLSLSAAVKAGACMIDGGNMLETIKVSPSMFSTLIKTVLQV |
| | | KNREGMFIGNAGPQRNPYENLLYKICLSGEGWPYIGSRSQV |
| | | SGRAWDNTSIDIEGKPSPNHPPVRNGGTPQINPLSRDQEDQV |
| | | KGAVRLLDPKVTTWVDIEGPPGDLVEFAIFQPNSGKYLHCY |
| | | RRPHNEQSFKDQSKFSHGLLLKDLESAQPGLVSAIIRALPEG |
| | | MVLTAQGSDDIEKLFLMHGRRDLKVVDVALTSEQARVYED |
| | | TVWERFNPLCKKHKGLVIKKKKKGAAPTSTNAHCALLDCI |
| | | MFDATITGYIADAKPQQLLPIDLLYRTENLIHL |
| 82 | Amino acid sequence of Z | MGLRYSKAVRDRYGERETVGRVPMTLNLPQGLYGRFNCKS |
| | protein in FLEV | CWFANKGLIACSDHYLCLNCLTRMLSRSEFCEICNRPLPTKII |
| | | FEESPSAPPYEP |
| 83 | Amino acid sequence of L | MDETLNEMRDLIRKWVPDEPEFIEQKASSLSQVHLRAVVIE |
| | protein in FLEV | GLKLLSLLVEVDSCKKNRCIHNRSKTVNAILREYKMVAPTL |
| | | PDLVPDGYLVTGDIVVLLEAFVRVNQSSFEVKYNHDFGKL |
| | | MMLSQDLSKVGITLVPVVDGRSNYYVDYIPDWVIERLRWLI |
| | | ITIMKQLKDDGEDIEEVEYERLVCSLSTMENQGLGLESLTQ |
| | | MRECGASYKQRLESVLSLGVNGQLSVSDCKTAILKLFTEYQ |
| | | TLKEQGYLAESFKSTDRLELQEALRSHSLIEHGNLGASPHCE |
| | | LCQNHMIEVLTKLKGSKSSSNTKLVSQEVSEYFSLLSVCNKI |
| | | KGQKILNTRRLTFLSLDVIMFNKFLQIIKKDCTQDVHFLVGG |
| | | CLRSVNDRLVCPDLIVKAYERKMISSPKWLTKVDQKLVHG |
| | | LPEPLRQNYELQVQPLLTELDLETWAEYYDSYRENWGNKP |
| | | SINYNAIATPCNCEENDRLDYVNLSDESFLQYLEALSTLSLG |
| | | LVNSMKTASTTKLTVNQPDNYYGFVHCNECYYQEFQEAYR |
| | | SVLLYQKTGEKNRCYSIFKTVGGGDSEEHLASFYCDPKRFF |
| | | LPIMSSDVIHSMACEMLSWLDFMSDSDKRKVSCNLRKLLLC |
| | | VMCTPTKRLQTYLQGMRYFIMAYVNELHHVQLLNKLKIVA |
| | | KSKAEFHTMCLTDDLILSVLTSSDEVNMTKAFKFILNVSYLC |
| | | HLITKETPDRLTDQIKCFEKFMEPKLNFDSVLINPSMSLHLEP |
| | | KQEDKFLNDMTRLLSKDVKGAQGSDPGTDPLLMSICSSLFN |
| | | HGELGLPNKLSRDPQSPSFTSTALDLSSNKSVVVPKLNELGE |
| | | PITTYDYQTLVSSVVVELSESFKNKLQYKLDRKSLVYKIYDR |
| | | LMGLVSNKVKHPIDEPFDSEDDILDSVSDEVKDVILKIESDV |
| | | TSCLSRMESSKCNHGCEKKVGTGKADHNPLDSLWSEEVRM |
| | | RVNIETSHHEVKDFDFNTFPPETYEELVQIVFESRFKELYFTE |
| | | RVFSPCPLEMLLRNLTRKYYEEQDFFECFKYILVSTGYDNR |
| | | VGRYDHKKINRLGFKEPALNISEVVRISTRESNSESILKRLDK |
| | | SFFTNSSLRNLCFYSDESATERSCVGTNIGRLKFGLSYKEQV |
| | | GGNRELYVGDLNTKLTTRLIEDYFESIVGDMRYSCLNNEKE |
| | | FEKALLDMKAIIRQSGFVVSMDHSKWGPHMSPVIFSQFLRL |
| | | LKLSLLDGSVIDNRPILELLNWHIHKMVEVPFNVVRAYMKG |
| | | YIKRCTGVMEKNSMTMVEDFMHKQFETGVVPSHISSVIDM |
| | | GQGILHNVSDFYGLVTEQFINYCIKLCYDTPCLSYTSSDDEIL |
| | | MSSSFILKKNDGELDVELAKDILDFHDFLSRGLNKFVSPKTV |
| | | AGTFACEFKSRFFIWSQEVPLLTKFVAAALHNVKAKAPNQL |
| | | AETIDTILDQCVANGVSIEVVGRIAKRTNALLRYSGHPYNLF |
| | | LCLEETDVKDWVDGSRGYRLQRSVENVFPDDEVPGIVRAA |
| | | ARKVFHLIRSGTIEEEYLVSTIQTDPDDCLRRILEIADVTNEQI |
| | | ERILDFRWLNLRAHGDYRMVLRTKLMNSARIIEREEIPSLIK |
| | | SVQSKLSKNFVRGAKKIITDAVNKSAFQSCIASGFVGVCKS |
| | | MGSKCVRDGKGSFLYIKDVLKDIIKHVNCHSCRNYCNIYCR |
| | | EALKEVSEYSRPLFWDYFALVLTNACELGNWVFSKAVLPK |
| | | SVYKLDNPSQFWLCKPSSHTELEDKVNLNHVLFSIKRNFPSL |
| | | FEEHVAPYLSDLNTLKISWVQRIKFLDICVAVDMTSESLGIIS |
| | | HMIKRKREELYVVKQNEQSMSHLREASTFEEGLQLNSYEIC |
| | | YNFLLQILFESMLTPVLLTTSQLKKYFWYGEVELLPNTEPHE |
| | | LQQLTQFVMDCKMLNISKAMTIDDLDLGFVQSTMKMTDV |
| | | NLNLSTFLTKVDWANRYLYETFESMFIESPDSEFSMELVLVF |
| | | SHIRKSYKHKYEHTTTYTVKASFVLETSLFSDQDDQDVLVIP |
| | | VKDVECFVSNSPGNHLQLDGAGLIPLVPVVSGKEVLNFDLL |
| | | LKDQDVSFSGTSPHLSKVRLDFSAHIKELKNKFSYKIIGPEM |
| | | GFTPLHLDKGIIKEGDRIVSKLNVNVTSKSLFMALGLLSNDK |
| | | VSEFLESLFYYLKSSGKTGALLSMTTSDLQNLVDNYNEDFK |
| | | QILKRESDWVSFGAFKLAYSNSLSAIMIQDERGPYRLKGLSC |
| | | EKLLKSHDERVEID |
| 84 | Amino acid sequence of | MGQLISFFQDIPIFFEEALNVALAVVTLLAIIKGIVNVWKSGI |
| | glycoprotein precursor | LQLFVFLVLAGRSCSFKVGHHTNFESFTVKLGGVFHELPSLC |
| | (GPC) protein in Guanarito | RVNNSYSLIRLSHNSNQALSVEYVDVHPVLCSSSPTILDNYT |
| | virus (GTOV) | QCIKGSPEFDWILGWTIKGLGHDFLRDPRICCEPKKTTNAEF |
| | | TFQLNLTDSPETHHYRSKIEVGIRHLFGNYITNDSYSKMSVV |
| | | MRNTTWEGQCSNSHVNTLRFLVKNAGYLVGRKPLAFFSWS |
| | | LSDPKGNDMPGGYCLERWMLVAGDLKCFGNTAVAKCNLN |
| | | HDSEFCDMLRLFDFNKNAIEKLNNQTKTAVNMLTHSINSLIS |
| | | DNLLMRNKLKEILKVPYCNYTRFWYINHTKSGEHSLPRCW |
| | | LVSNGSYLNESDFRNEWILESDHLIAEMLSKEYQDRQGKTP |
| | | LTLVDLCFWSAIFFTTSLFLHLVGFPTHRHIQGDPCPLPHRLD |
| | | RNGACRCGRFQKLGKQVTWKRKH |
| 85 | Amino acid sequence of | MGQLISFFQDIPIFFEEALNVALAVVTLLAIIKGIVNVWKSGI |
| | GP signal peptide in | LQLFVFLVLAGRSCS |
| | GTOV | |
| 86 | Amino acid sequence of | FKVGHHTNFESFTVKLGGVFHELPSLCRVNNSYSLIRLSHNS |
| | GP1 protein in GTOV | NQALSVEYVDVHPVLCSSSPTILDNYTQCIKGSPEFDWILGW |
| | | TIKGLGHDFLRDPRICCEPKKTTNAEFTFQLNLTDSPETHHY |
| | | RSKIEVGIRHLFGNYITNDSYSKMSVVMRNTTWEGQCSNSH |
| | | VNTLRFLVKNAGYLVGRKPL |
| 87 | Amino acid sequence of | AFFSWSLSDPKGNDMPGGYCLERWMLVAGDLKCFGNTAV |
| | GP2 protein in GTOV | AKCNLNHDSEFCDMLRLFDFNKNAIEKLNNQTKTAVNMLT |
| | | HSINSLISDNLLMRNKLKEILKVPYCNYTRFWYINHTKSGEH |
| | | SLPRCWLVSNGSYLNESDFRNEWILESDHLIAEMLSKEYQD |
| | | RQGKTPLTLVDLCFWSAIFFTTSLFLHLVGFPTHRHIQGDPC |
| | | PLPHRLDRNGACRCGRFQKLGKQVTWKRKH |
| 88 | Amino acid sequence of | MAHSKEIPSFRWTQSLRRELGMFTEPTKSSVLNDAKLIADSL |
| | NP protein in GTOV | DFTQVSQVQRLLRKSKRGDTDLDKLRDLNKEVDRLMSMKS |
| | | VQNNTVLKVGDLGKDELMDLASDLEKLKKKIGDRESNSPR |
| | | MYMGNLTQSQLEKRAGILRTLGFQQQRGAAGGVVRLWDV |
| | | SDPSKLNNQFGSMPALTIACMTVQGGETMNNVVQALTSLG |
| | | LLYTVKYPNLDDLEKLTLEHDCLQIITKDESALNISGYNFSLS |
| | | AAVKAGASLIDGGNMLETIKVTPNNFSSIVKAALNVKRREG |
| | | MFIDERPGNRNPYENLLYKLCLSGEGWPYIGSRSQILGRSW |
| | | DNTSVDLNARPVTGPRAPEKNGQNIRLSNLSEMQEAIVKEA |
| | | MRKLDSSDTIWMDIEGPPTDPVELAVFQPSSGNYVHCFRKP |
| | | HDEKGFKNGSRHSHGILLKDLEDAQPGLLSYVIGLLPQGSVI |
| | | TVQGADDIKKLFDIHGRKDLKLVDVRLTGEQSRIFEQEVWE |
| | | KFGHLCRAHNGVIVPKKKNKEANSTKEPHCALLDCIMFQSV |
| | | LDGHLPDTIPIQLLPNTLVFQAKSAFVM |
| 89 | Amino acid sequence of Z | MGNSKSKSNPSSSSESQKGAPTVTEFRRTAIHSLYGRYNCKC |
| | protein in GTOV | CWFADKNLIKCSDHYLCLRCLNVMLKNSDLCNICWEQLPT |
| | | CITVPEEPSAPPE |
| 90 | Amino acid sequence of L | MDEKVFVLKDFIRRQVPDIPELSYQKEALLSQVEVPMVLTE |
| | protein in GTOV | GFKLLSCLVEIESCRKNSCECNFEQKFVDTILSENGVVAPTLP |
| | | KVIPDGYRFFNKTLILLETFVRVNPEEFEKKWKTDMAKLLS |
| | | LKEDIHRTGITLVPVVDGRGNYNTDLLPDWATERFRWLLID |
| | | LLRESRGAPTMEIEDQEYHRLIHSLSKTSNQSLGFENIECLKR |
| | | VHLNYEERLNEQLLKDIVGEVRESKIREELIKLKTWYREEIY |
| | | RKGLGNFVQTDRKSLLQTLVLSSAHSDSLAPECPMCCSKIL |
| | | DLCYQLSMRIANQTSLENNFDEPPLPTTQIEKVYLSLLSACN |
| | | KIKGKKVFNTRRNTLLFLDLIILNFVAHVYKTQPSEMETLKK |
| | | AGLIIGEMLLLPNDRVLDILVARRLLLKKVESCCNWLDRCR |
| | | HLLRKEEPVLWDCVSEFTNVPDFELLLSLAEELCSEKPVMH |
| | | YKPPSSLIGDCAHKDLMSMSDGEFESLFKCLSHISLSLVNSM |
| | | KTSFSSRLLVNEKDYKRYYGTVRLKECYVQRFFLRVGLYGL |
| | | LFYQKTGEKSRCYSLYLSDKGNLVELGSFYSDPKRFFLPIFS |
| | | EFVLLATCAEMLSWLDFDEKLVDAVTPLLKILVLSILSSPTK |
| | | RSQTFLQGLRYFIMAYVNQAHHIQLMSKLAVECKSASDVLI |
| | | QRLSVKIVDMVLSDGSDPDMHMTRKFKFVLNVSYLCHLIT |
| | | KETPDRLTDQIKCFEKFMEPKLEFGSLIVNPSLNGFLSKEQE |
| | | DVMIEGVEKFFSKELLTVEDLKRPGVSRELLSYCVSLFNKG |
| | | RLRVNGTLGTDPYRPSFTSTALDLSSNKSVVIPKLNEVGEIV |
| | | SEYDKQKLVSTCITSMAERFKTKGRYNLDPDTIDFLIMRNLT |
| | | NLLSARKLDSSKKEELSLLYEHLSEDVMKAFEEIKYEVEITL |
| | | SKMRLSRELECGHKKPCTLEGVWAPFNVLKVIRSETSVHEI |
| | | RDFDPDLLGEDVYEKLCVAVYDSPLRPTFFLEKPLDICPLEL |
| | | LLKNLTTKSYEDDEFFDCFKYILIQAGFDQRLGAYEHKNRS |
| | | RLGLSEEAFRLKEDVRVSNRQSNSEAIADRLDKSFFTSAALR |
| | | NLCFYSEESPTEYTCISPNVGNLKFGLSYKEQVGSNRELYVG |
| | | DLNTKMMTRLVEDFTEAVANSMNYTCLNSEKEFERAICDM |
| | | KMAVNNGDLCCSLDHSKWGPFMSPALFHAFFGALKFKISK |
| | | TGEQVDLGPVLNVLKWHLHKAVEVPISVAEAYCTGMLKRR |
| | | LGLMSLSCQSVCEEFFHQKLLLEEGVPSHIMSVLDMGQGIL |
| | | HNSSDLYGLITEQFINYCLDFLFDVIPVSYTSSDDQITTFKLPT |
| | | MSSSEDGLDGFDWLELLCFHDFLSSKFNKFVSPKSVSGTFV |
| | | AEFKSRFFVMGEETPLLTKFVSAALHNVKCKTPTQLAETIDT |
| | | ICDQCVANGVGIEIVTKISERVNRLIRYSGYPQTPFLAVEKQ |
| | | DVKDWTDGSRGYRLQRNIEHYLQGSEQLEFVRKCAKKVLL |
| | | KIKKGQVFEEYLVQLIGKDGDDALKGFLSYAGCESDEIKDV |
| | | LKYRWLNLSANGDLRLVLRTKLMSTRRVLEREQIPTLIKTL |
| | | QSKLSKNFTKGVKKILAESINKSAFQSSVASGFIGFCKSMGS |
| | | KCVRDGSGGFMYIREVLNKQRVCPCEICAQNPGIIFCSDALT |
| | | LIPEFSRSILWDYFSLVLTNACELGEWVFSSVQPPKVPILLNN |
| | | PNLFWAVKPRGTRLIEDQLGLGHVLQSVRRSYPKVFEEHLV |
| | | PFMNDLQVSRTTDFTRLRYLDVCVALDMMNENLGIVSHLL |
| | | KAKDNSIYIVKQSECAVAHIRQVEYVNQELGLSPQQICSNFK |
| | | IQLVFSSMINPLVITTSVLKSFFWFNEVLNLEDESQIDVGELT |
| | | DFTILIKKYNLNRAMMLDDLTMGYVVSTISEPTIHLVSLKRN |
| | | SNSIVGEQNSEMLHGEQVEDMYSIVLHIQLEHKRHSTKYHL |
| | | SRTVVYSYTVECETNITDIEKEPSLATVKNVVLRASGSIEGH |
| | | QFLDGVNLVASQPIFTGKKVINLSELLADSEnETYKEGDAV |
| | | GSILLNFGTFYEHIDDRYAYEIVGPECSDSPLVLDGGSILADG |
| | | KKLSSIKVELTGDVILKALGALESEKEVQSLLTGLWPFIRINN |
| | | LKVKMAQEDFLLMYEMHRESLLKSLEVFSEWCEFVDFSVC |
| | | YSKSLRDLVISDSSGSLRLKGITCKPINLSNSVTEIE |
| 91 | Amino acid sequence of | MGQVIGFFQSLPEIINEALNIALICVALLATIKGMVNIWKSGL |
| | glycoprotein precursor | IQLLFFLTLAGRSCSHSFTIGRFHEFQSVTVNFTQFMSYAPSS |
| | (GPC) protein in Latino | CSVNNTHHYFKGPQNTTWGLELTLTNESMINITNSMRVFTN |
| | virus (LATV) | IHHNVTNCVQNISEHEGVLKWLLETMHLSISKPGKHIAPVM |
| | | CERQKGLLIEYNLTMTKDHHPNYWNQVLYGLAKLLGSSKR |
| | | LWFGACNKADCQMQSDHQHIKCNYSNCKGYTSFKYLIIQN |
| | | TTWENHCEYNHLNTIHLLMSSIGQSFITRRLQAFLTWTLSDA |
| | | LGNDLPGGYCLEQWAVVWFGIKCFDNTAMAKCNQNHDSE |
| | | FCDMLRLFDYNRNAIQSLNDQSQARLNLLTNTINSLVSDNL |
| | | LMKNKLRELMNVPYCNYTRFWFINDTKNGRHTLPQCWLVS |
| | | DGSYLNETRFRTQWLSESNSLYTEMLTEEYEKRQGRTPLSL |
| | | VDLCFWSTLFYISTLFAHLVGFPTHRHLIGEGCPKPHRLTGS |
| | | GICSCGHYGIPGKPVRWTKMSR |
| 92 | Amino acid sequence of | MGQVIGFFQSLPEIINEALNIALICVALLATIKGMVNIWKSGL |
| | GP signal peptide in | IQLLFFLTLAGRSCS |
| | LATV | |
| 93 | Amino acid sequence of | HSFTIGRFHEFQSVTVNFTQFMSYAPSSCSVNNTHHYFKGPQ |
| | GP1 protein in LATV | NTTWGLELTLTNESMINITNSMRVFTNIHHNVTNCVQNISEH |
| | | EGVLKWLLETMHLSISKPGKHIAPVMCERQKGLLIEYNLTM |
| | | TKDHHPNYWNQVLYGLAKLLGSSKRLWFGACNKADCQM |
| | | QSDHQHIKCNYSNCKGYTSFKYLIIQNTTWENHCEYNHLNT |
| | | IHLLMSSIGQSFITRRLQ |
| 94 | Amino acid sequence of | AFLTWTLSDALGNDLPGGYCLEQWAVVWFGIKCFDNTAM |
| | GP2 protein in LATV | AKCNQNHDSEFCDMLRLFDYNRNAIQSLNDQSQARLNLLT |
| | | NTINSLVSDNLLMKNKLRELMNVPYCNYTRFWFINDTKNG |
| | | RHTLPQCWLVSDGSYLNETRFRTQWLSESNSLYTEMLTEEY |
| | | EKRQGRTPLSLVDLCFWSTLFYISTLFAHLVGFPTHRHLIGE |
| | | GCPKPHRLTGSGICSCGHYGIPGKPVRWTKMSR |
| 95 | Amino acid sequence of | MSGASEVPSFRWTQSLRRGLSHFTTSAKGDVLRDAKSLVD |
| | NP protein in LATV | GLDFNQVSQVQRVMRKDKRSDDDLSKLRDLNRSVDSLMV |
| | | MKNKQNNVSLKIGSLSKDELMDLATDLEKLKRKINLGDRQ |
| | | GPGVYQGNLTSAQLEKRSEILKSLGFQPRANQNGVVKVWDI |
| | | KNPKLLINQFGSIPALTIACMSVQGAEQMNDVVQGLTSLGL |
| | | LYTVKYPNLDDLNKLSKDHPCLEFITKEESANNISGYNLSLS |
| | | AAVKAGACLVDGGNMLETILVKPDNFQDIVKSLLVIKRQEK |
| | | MFVNEKPGLRNPYENILYKLCLSGEGWPYIGSRSQIVGRAW |
| | | ENTTVDLSKEVVYGPSAPVKNGGNMRLSPLSDTQEAVIKEA |
| | | IGKLDMDETIWIDIEGPPNDPVELAIYQPSTGNYIHCFRVPHD |
| | | EKGFKNGSKYSHGILLRDIENARSGLLSRILMRLPQKVVFTC |
| | | QGSDDIQKLLQMNGRPDIATIDMSFSSEQARFFEGVVWEKF |
| | | GHLCTRHNGVVLSRKKKGGNSGEPHCALLDCIIFQAAFEGQ |
| | | VTGQIPKPLLPNSLIFKDEPRVAM |
| 96 | Amino acid sequence of Z | MGSKQSAPPKPLQLPQPRVSLLREAKPSLYGRYNCKCCWFQ |
| | protein in LATV | DKNLVECSDHYLCLKCISSMLKRGKNCEICGKAIPTYIEVGI |
| | | TPTAPQLN |
| 97 | Amino acid sequence of L | MDDIVNQLFDLLRKHFPARPKVTEQITLVTCQNDAKMILTE |
| | protein in LATV | GFKLLSLLVELDSAEANNCTHNSDSLTIEGILRKEGIMSIALP |
| | | RIVPDGFSLYGNVLILLETFVRVNPVSFEQKYNQDMSKLLSL |
| | | KDDLSLCGITLVPLVDGRTNYYNRFVDDWVIERFRWLLLQL |
| | | IKFVRESGEEIEELEYQRLITSLSKLENQSLGFENIEKLPQTGL |
| | | KYRDELKKHMFGNLSSKMKESEIQENLINVLKEFFIKEYKN |
| | | NKSLHKFVFTNRDGLLAKLDQITHHSEHPVDCMSCSSKLYSI |
| | | IDKLGTLKRQPLHSDYHPIYAKMWHSDSLSQAEQIYLKLLS |
| | | QCNKIKSAKLLNTRRNTLLFLDLIMVNFIVHSWKQNPEVLT |
| | | EYRRCGLMAGQLALFSNDRYFDLNELRNKLINKLKNCENW |
| | | IAKCVHQLKKQEFVALDDVLVWATVPDFESLELITTSLELK |
| | | RFKLQYGKDKVDHNEHPIGPLSEETFFRNLNVLSSVCLALV |
| | | NSMKTSFTSKTVINERRASNHFGEVDLIECYCQRFFLSKDLV |
| | | GILSYQKTGEKSRCYSISLISNGELEYIGSFYCDPKRFFLPIFS |
| | | QIVLLNMSREMMLWLADLNLNDSLVGDKLRKLILLIVTNPS |
| | | KRNQTFLQGLRYFIMAYVNQFHHVELMDRLIVPVKSYCESC |
| | | LQRISFDIFRLILEGDYDNEHMTRKFKFLLNVSYLCHLITKET |
| | | PDRLTDQIKCFEKFMEPKLKFESVIVNPSLTENMTEDEEAQV |
| | | LKGVDKLLGKSLSCSTDLTSPGVSKTLLSMCVSSFNRGLLN |
| | | VNGHLRQDPYRPNFTSTALDLSSNKSVVVPKLDELGNPISR |
| | | YDYELLVSSCVTNLAEGFKTKGKFKLDINCQEYTIMRNLTN |
| | | LVLKNEDKSDAKIKGEKPCSFELSQWMETLSEEQLEVLEKL |
| | | KGDVNIALGKLKEKGRSKSNSTLKEGVKRLDSGNTLAGCA |
| | | DPQQVLVNLWSEFGVMKQILVEVSLHEIKDFDPDIIPPQMIQ |
| | | KLVFKVNNSNYKSLFFLDSVINPCPLELLIKNMTTATFDDGE |
| | | LFECFKYLLITAGFDQKLGTYEHKNRSRFGFKFEALKVREE |
| | | GRMSSRESNSEAIARRLDKSVFTNSALRNLCFYSDESPISYSH |
| | | VSPDVGKLKFGLSYKEQVGSNRELYVGDLNTKLMTRLIEDF |
| | | SESVVSNMSYSCLNNEAEFEKAITDMKMCVNLGDMSLSLD |
| | | HSKWGPHMSPVIFAAFLQGLDLKYGPSLCKLNTDPIITLLSW |
| | | HIHKVVEVPYNVIHAYVTGMIKRQLGLMNMSGSTITESFVH |
| | | RLLKEKREPLSHVMSVIDMGQGILHNMSDLYGLVTEQFINY |
| | | AIHFLFDMNTTSYTSSDDQISMIKIGSGMCNFESLKVIEEWET |
| | | ILNFHAFISTKFNKFVSPKTVAGTFAAEFKSRFFVWGEEVPL |
| | | LTKFVSAALHNVKCKTPVQLSETVDTICDQCVANGVSVEIV |
| | | SYICNRTNRLIRYSGFGEHPFLNVENLDVKDWVDGSRGYRL |
| | | QRNIELHLESDGCTSFIRQAARKVFSNIKSGKIVEQALVDLV |
| | | QEDGDKAITGFLRSVGVSEEDIALLCRIRWINLCAHGDLRLV |
| | | LRTKLMSSRRIIETEEIPSLIKSIQSKLSKNFVKGAKKILAESIN |
| | | KSAFQSSIASGFIGFCQSVGSKCVRTGEGGFYYIKELKSKVD |
| | | LYCPCEVCARWKGVTYCSSSCLKIESFTRPLMWDYFSLVLS |
| | | NACELGEWVFEDVEYPKDINFLRNPNLFWLVKPRVSCQIEE |
| | | KLGLTHILQSIRRNYPQLFETHLSPFMSDFQAGRTLGTMTVK |
| | | FLDVCVALDLANENLGIVKHFLKNRRHDIYIVKQDESSQSHI |
| | | RCQKSICVDVELTSTQVCQNFMTQLIMSSLVQPLVLTSSELK |
| | | KFNWFQQVLTLETDEDVDLGLLTDFALQVKKFNVDRAMHS |
| | | EDLSAGYISSTVSVTTFSLSKPIFLQQIDSDFIGGTEDRKDFIQ |
| | | MIKSEFTKNSIDLQFVIQISHVKRALRFNLKRTTVYTLIVRTSI |
| | | LKEVILNSLGQEDQSVELVVDDLELFCSGHDGNHFTLDAAP |
| | | LIVQEPLINGNLKFDLVSRLEEEDLTFSYSESLPSFHFNFEKY |
| | | KHELCNKFSYHLSGPVIVDEPLVLDRGVILHGGRKLTTLQFD |
| | | FSADRIMQALSELESLSSRDLFLFNLWVYSDQTKSKLYIHQD |
| | | KLLLLVESYLSELNSSLARYDSWLNLGNYMICYSKSFKCLM |
| | | ISDTNGRNRLKGILCRRLIEEEVQDIE |
| 98 | Amino acid sequence of | MGQLISFFQEIPVFLQEALNIALVAVSLIAVIKGIINLYKSGLF |
| | glycoprotein precursor | QFIFFLLLAGRSCSDGTFKIGLHTEFQSVTLTMQRLLANHSN |
| | (GPC) protein in Machupo | ELPSLCMLNNSFYYMRGGVNTFLIRVSDISVLMKEYDVSIYE |
| | virus (MACV) | PEDLGNCLNKSDSSWAIHWFSNALGHDWLMDPPMLCRNK |
| | | TKKEGSNIQFNISKADDARVYGKKIRNGMRHLFRGFHDPCE |
| | | EGKVCYLTINQCGDPSSFDYCGVNHLSKCQFDHVNTLHFLV |
| | | RSKTHLNFERSLKAFFSWSLTDSSGKDMPGGYCLEEWMLIA |
| | | AKMKCFGNTAVAKCNQNHDSEFCDMLRLFDYNKNAIKTL |
| | | NDESKKEINLLSQTVNALISDNLLMKNKIKELMSIPYCNYTK |
| | | FWYVNHTLTGQHTLPRCWLIRNGSYLNTSEFRNDWILESDH |
| | | LISEMLSKEYAERQGKTPITLVDICFWSTIFFTASLFLHLVGIP |
| | | THRHLKGEACPLPHKLDSFGGCRCGKYPRLKKPTIWHKRH |
| 99 | Amino acid sequence of | MGQLISFFQEIPVFLQEALNIALVAVSLIAVIKGIINLYKSGLF |
| | GP signal peptide in | QFIFFLLLAGRSCS |
| | MACV | |
| 100 | Amino acid sequence of | DGTFKIGLHTEFQSVTLTMQRLLANHSNELPSLCMLNNSFY |
| | GP1 protein in MACV | YMRGGVNTFLIRVSDISVLMKEYDVSIYEPEDLGNCLNKSD |
| | | SSWAIHWFSNALGHDWLMDPPMLCRNKTKKEGSNIQFNIS |
| | | KADDARVYGKKIRNGMRHLFRGFHDPCEEGKVCYLTINQC |
| | | GDPSSFDYCGVNHLSKCQFDHVNTLHFLVRSKTHLNFERSL |
| | | K |
| 101 | Amino acid sequence of | AFFSWSLTDSSGKDMPGGYCLEEWMLIAAKMKCFGNTAV |
| | GP2 protein in MACV | AKCNQNHDSEFCDMLRLFDYNKNAIKTLNDESKKEINLLSQ |
| | | TVNALISDNLLMKNKIKELMSIPYCNYTKFWYVNHTLTGQH |
| | | TLPRCWLIRNGSYLNTSEFRNDWILESDHLISEMLSKEYAER |
| | | QGKTPITLVDICFWSTIFFTASLFLHLVGIPTHRHLKGEACPL |
| | | PHKLDSFGGCRCGKYPRLKKPTIWHKRH |
| 102 | Amino acid sequence of | MAHSKEIPSFRWTQSLRRGLSQFTHTVKTDVLKDAKLIADSI |
| | NP protein in MACV | DFNQVSQVQRALRKNKRGEEDLNKLRDLNKEVDRLMSMK |
| | | SIQKNTIFKIGDLGRDELMELASDLEKLKNKIKRTESGPQGL |
| | | YMGNLSQLQLTKRSEILKTLGFQQQRGAGNGVVRIWDVSD |
| | | PSKLNNQFGSMPALTIACMTVQGGETMNSVVQALTSLGLL |
| | | YTVKYPNLNDLDKLTLEHECLQIVTKDESSINISGYNFSLSA |
| | | AVKAGASILDGGNMLETIRVTPDNFSSLIKSTLQVKRKEGM |
| | | FIDEKPGNRNPYENLLYKLCLSGDGWPYIGSRSQILGRSWD |
| | | NTSVDLTKKPQVGPRQPEKNGQNLRLANLTEMQEAVIKEA |
| | | VKKLDPTNTLWLDIEGPPTDPVELALYQPANKHYIHCFRKP |
| | | HDEKGFKNGSRHSHGILMQDIEDAMPGVLSYVIGLLPQDM |
| | | VITTQGSDDIRKLLDIHGRKDLKLVDVKLTSDQARLYDQQI |
| | | WEKFGHLCKHHNGVVVNKKKREKDSPFKLSSGEPHCALLD |
| | | CIMYQSVMDGKMVDEEPVALLPLSLLFLPKAAFAL |
| 103 | Amino acid sequence of Z | MGNCNKPPKRPPNTQTSSNQPSAEFRRTAPPSLYGRYNCKC |
| | protein in MACV | CWFADTNLITCNDHYLCLRCHQTMLRNSELCHICWKPLPTS |
| | | ITVPVEPSAPPP |
| 104 | Amino acid sequence of L | MDEYVQELKGLIRKHIPERCEFGHQKVTFLSQVHPSPLLTEG |
| | protein in MACV | FKLLSSLVELESCEAHACQANTDQRFVDVILSDNGILCPTLP |
| | | KVIPDGFKLTGKTLILLETFVRVNPDEFEKKWKADMSKLLN |
| | | LKHDLQKSGVTLVPIVDGRSNYNNRFVADWVIERIRWLLIGI |
| | | LKASKSMLEIDIEDQEYQRLIHSLSNVKNQSLGLENLEHLKR |
| | | NSLDYDERLNESLFIGLKGDIRESTVREELIKLKLWFKDEVF |
| | | SKGLGKFKLTDRRELLESLSSLGAHLDSDVSSCPFCNNKLM |
| | | EIVYNVTFSCVERTDGVATVDQQFSTTHSNIEKHYLSVLSLC |
| | | NKIKGLKVFNTRRNTLLFLDLIMVNLMVDISDSCQDAIESLR |
| | | KSGLIVGQMVMLVNDRVLDILEAVKLIRKKIGTNPNWVKN |
| | | CSKILERSHPEIWHHLSTLIKQPDFNSLISIAQHLVSDRPIMRY |
| | | SVERGSDKICRHKLFQEMSSFEQMRLFKTLSSISLSLINSMKT |
| | | SFSSRLLVNEREFSKYFGNVRLRECYAQRFYLAESLVGFLFY |
| | | QKTGERSRCYSVYLSDNGVMSEQGSFYCDPKRFFLPVFSDE |
| | | VLAGMCEEMTSWLDFDTGLMNDTGPILRLLVLAILCSPSKR |
| | | NQTFLQGLRYFLMAFANQIHHIDLTSKLVVECKSSSEVVVQ |
| | | RLAVGLFIRLLSGESDASLFFSRRFKYLLNVSYLCHLITKETP |
| | | DRLTDQMKCFEKFIEPKVKFGCAVVNPSLNGKLTVDQEDIM |
| | | INGLKKFFSKSLRDTEDVQTPGVCKELLNYCVSLFNRGKLK |
| | | VSGELKNNPFRPNITSTALDLSSNKSVVIPKLDELGNILSTYD |
| | | KEKLVSACVSSMAERFKTKGRYNLDPDSTDYLILKNLTGLV |
| | | SAGPKAKSTQEELSLMYEALTEEQVESFNEIKHDVQVALAK |
| | | MADNSVNTRTKNLGRADNSVKNGNNPLDNLWSPFGVMKE |
| | | IRAEVSLHEVKDFDPDVLPPEVYKELCDAVYKSSEKCNFFLE |
| | | GVLDVCPLGLLLKNLTTSSYVDEEYFMCFKYLLIQGHFDQK |
| | | LGSYEHKSRSRLGFTDETLRLKDEVRLSIRESNSEAIADKLD |
| | | KSYFTNAALRNLCFYSEDSPTEFTSISSNSGNLKFGLSYKEQ |
| | | VGSNRELYVGDLNTKLMTRLVEDFSEAVGNSMKYTCLNSE |
| | | KEFERAICDMKMAVNNGDLSCSYDHSKWGPTMSPALFLAL |
| | | LQMLELRTPVDRSKIDLDSVKSILKWHLHKVVEVPINVAEA |
| | | YCIGKLKRSLGLMGCGSTSLSEEFFHQTMQLNGQIPSHIMSV |
| | | LDMGQGILHNTSDLYGLITEQFLCYALDLLYDVIPVSYTSSD |
| | | DQITLIKTPSLDIEGGSDAAEWLEMICFHEFLSSKLNKFVSPK |
| | | SVIGTFVAEFKSRFFVMGEETPLLTKFVAAALHNVKCKTPT |
| | | QLSETIDTICDQCIANGVSTKIVTRISKRVNQLIRYSGYGETPF |
| | | GAIEDQDVKDWVDGSRGYRLQRKIEAIFHDDKETSFIRNCA |
| | | RKVFNDIKRGRIFEENLINLIGRGGDEALTGFLQYAGCSEQE |
| | | VNRVLNYRWVNLSSFGDLRLVLRTKLMTSRRVLEREEVPT |
| | | LIKTLQSKLSRNFTKGVKKILAESINKSAFQSSVASGFIGFCK |
| | | SMGSKCVRDGKGGFLYIKEVYSGVSACTCEICALKPKIIYCN |
| | | NSLNKVSQFSKPILWDYFSLVLTNACELGEWVFSTVKEPQK |
| | | PLVLNNQNFFWAVKPKVVRQIEDQLGMNHVLQSIRRNYPV |
| | | LFDEHLTPFMNDLQVSRTMDSGRLKFLDVCIALDMMNENL |
| | | GIISHLLKTRDNSVYIVKQSDCALAHIRQSSYTDWELGLSPQ |
| | | QICTNFKTQLVLSSMVNPLVLSTSCLKSFFWFNEVLELEDDS |
| | | QIELAELTDFALMVKNQNVSRAMFVEDIAMGYVVSNFEGV |
| | | RISLSNVMVDGVQLPPQEKAPDIGELFGLKAENVIVGLVVQI |
| | | DHVRMSTKFKLKRKMVYSFSLECIMDVGEIQNKEVILKVVA |
| | | VDQSVSGSGGNHMLLDGVSVVASLPLFTGQASFDLAAMLIE |
| | | SNLAGSNDNFLMRNVTLDLGGFSPELSDKYSYRLSGPENQE |
| | | DPLVLKDGAFYVGGERLSTYKVEFTGDLVVKALGALEDDE |
| | | SVVSMLHQLWPYLKATSQVILFQQEDFTIVHDLYKKQLTKS |
| | | IESFGEWIEFTNFKVAYSKSLKELVISDTQGSFRLKGVMCRP |
| | | LASTPQVEDIE |
| 105 | Amino acid sequence of | MGQLVTLFQSIPEIIEEAVNIALIAVAIMCIVKGTVNLWKCGI |
| | glycoprotein precursor | VQLCIFLLLAGKRCDGFQIDRRHKLESVEFNLTRMFNNLPM |
| | (GPC) protein in Parana | SCSKNNTHHYYKGPEGTNWGIELTLTNESVANYSNMSAIRS |
| | virus (PARV) | LAYGNITNCDKTNEAGHTLKWLLNELHFNVLHVTRHIGAR |
| | | CLTTDSAGILIQYNLTVGDYGGEVGRHLIASLAQIIGDDKAA |
| | | WVGKCFNNCSANGTCRLTNCEGYTHYNYLIIQNTTWENHC |
| | | SYSPMSTIRMALNKVAYSSVSRKLLGFFTWDISDSSGRHVP |
| | | GGYCLEQWALVWAGIKCFDNSVMAKCNKDHNEEFCDTMR |
| | | LFDFNQNAIKTLQLNTENSINLLKRSINGLISDSLVIRNSLKQ |
| | | LARIPYCNYTKFWYVNDTITKRHSLPQCWLTYNGSYLNETH |
| | | FRNDWLLESQQLYNDMLVKEYEERQGKTPIALTDICFWSLV |
| | | YFTVSVFLQLVGIPSHRHIVGQGCPKPHRISRNGLCSCGYYN |
| | | IPMKPVRWVRKGK |
| 106 | Amino acid sequence of | MSEKQVPSFRWTQALRRGLSNWTEPVKVDVIKDARAIISAL |
| | nucleoprotein (NP) protein | DFNQVAQVQRIMRKEKRTDSDLTRLRDMNKEVDALMSMR |
| | in PARV | STQHNVVLRAGGLSKDELLELSADLEKLRKKVIRAEGGNPG |
| | | VYQGNLTATQLNQRAELMKLVGMGPGLRSGNGVVRVWD |
| | | VKDSSLMINQFGSMPALTIACMTEQGGETMNDVVQGLSAL |
| | | GLVYTVKFPNLDDLEKLSEQHPCLKSITQEQSQINISGYNLSL |
| | | SAAVKAGACMIDGGNMLETIKMSPPMFSSIIKAVLQVKNRE |
| | | QMFVGSVGVQRNPYENLLYKLCLSGEGWPYIGSRSQIVGRA |
| | | WDNTLIDLEGKPAVSPPPVKNGGPINLSPLSKGQEDLINQAV |
| | | QKLSPKETTWIDIEGPAGDPVELAIYQPESGNYLHCYRAPHN |
| | | ESAFKDQSRYSHGLLLKDLKAARPGLISAIIKALPKGMVLTA |
| | | QGSDDIEQLILMHGRRDIKVVDVKLTSEHARVFEDPVWDRF |
| | | NPLCEKHTGLVIKKKKKGAPPSSTNPHCALMDCIMFDATVT |
| | | GYIRDVKPRQLIPIDLLFKDDLNLINL |
| 107 | Amino acid sequence of Z | MGLRYSKAVKDKYGDREIEGRATMTLNLPQGLYGRFNCKR |
| | protein in PARV | CWFATKGLIACSDHYLCLNCLTIMLSDGNFCEVCGKTLPKK |
| | | IVFEESPSAPPYDG |
| 108 | Amino acid sequence of L | MTEIISELKDLIRKWVPDEEHYIEQKSLTLSQVHTRAVVIEGL |
| | protein in PARV | KLLSLIVEVDSCLKHKCIPNRNKTVNMILKDHKLVGPTLPEC |
| | | TPDGYYLCGDVLILLEVFVRSNQSAFEKKYAADFEKLMSLA |
| | | KDLTSYGITLVPVIDGRSNYYVEAIPDWVIERLRWLLLKVM |
| | | DSLKDEGEDIEETEYSRLIHSLSTMENQNLGLESISQLKQTGL |
| | | TYKKKLQALFTKNIRPNMSLGECRLKLIEIFNEFRLRLESGT |
| | | VERAYTQTNQDFLLNKLKEHSLLKVSRVSKFDSKGDCHLCS |
| | | NHLIKVLGLLKRQTTDEKEAPRIGMIRREYGLILSTCNKIKG |
| | | QKILNTRRNTLLSLDVVMFNAFLSLLRTYGEIVFDLMVGGC |
| | | LQSVNDRLVSPSLIIDLYNKKCTRNPQWLSKVMLKLNLLPG |
| | | YIQEDFKVFVRPHLVELDLDLWSNYLELYSTTFERRLEIQYT |
| | | VSEEEVHEKTDPEVIEWLEMKESTFKQYLDSLSTLSLGLVNS |
| | | MKTSGTSRFHINQPNDYYGTVKCEECFFQSLHHAYGVSLLY |
| | | QKTGERNRCYSLCSNSGKVGHIVSFYCDPKRFFLPIMSQQVL |
| | | LSMTQEMLSWLDFIKGDDLNLISSMLRRLILSVLACPSKRVQ |
| | | LYLQGLRYFLMAYVNEIHHVQLLAKLEVEVKSRSEWATMT |
| | | LADDLVVALLNMSNQPNMSKTFKFLLNVSYLCHLITKETPD |
| | | RLTDQIKCFEKFMEPKIDFGSVIVNSSLNGNMTEEEESKILLD |
| | | IDRLLSKQLNTSDQISKPGVNPTVMSLCCSLFNLGELDVNGK |
| | | LKRDPQSPSFTSTALDMSSNKSVVVPKLDELGNPLSTYDYA |
| | | AVVSSVIVDLSEGFKNKLRYKLDPHTLKYKIYKRFLSLVSD |
| | | KTPKENKLEMSQDEFLDEITDEQLEMIEKIEMEVNECLSKAA |
| | | NVEPRIGTGPDDKNPLKSLWAKEILCVIESETSKHEVKDFDY |
| | | TLFHHETYKELVELVFNSNSRNQYFTDRILNPCPLEYLMKN |
| | | LCRKYYEEEDYFECFKYILVSTGFDNKVGRYDHKNMCRLG |
| | | FKQSATRIREDARISCRESNSESILKRLDKSFFTNSSLRNLCFY |
| | | SEESPTAFDSVSSDLGRLKFGLSYKEQVGGNRELYVGDLNT |
| | | KLMTRLIEDYFECLMEKMKYTCLNNEAEFERALLDMKSVV |
| | | RRSGFTVSMDHSKWGPHMSPVIFSQLFKALQFSLPDGSQID |
| | | KEPILNLLSWHIHKVVEVPFNVVHAYTKGYLKRLCGLMDN |
| | | RTSKTEDFMDKFFADEVIPSHISSVLDMGQGILHNVSDFYGL |
| | | ITEQFISYALQTCVGVLSMAFTSSDDEILLGVTNDLKNQDES |
| | | LDIDKSLEFLEFHNYLSATLNKFISPKTVAGTFACEFKSRFYI |
| | | WSQEVPLLTKFTAAALHNVKAKAPNQLCETIDTIQDQCVAN |
| | | GVSVEVVGAISKRTNNIIKYSGHPSNPFLCLNDMDVKDWVD |
| | | GSRGYRLQRSVENVYNDDEIPEYVRDMAARLFYLIRNGQV |
| | | QEEYLISSMQSDPDECFTLLAGILGVPEQNISRLLDIRWLNLR |
| | | AHGDLRMVLRTKLMGSNRVIQREEVPSLIKSVQSKLSKNFV |
| | | RGARKIITDAVNKSAFQSCIAAGFVGICKSMGSKCVRDGKG |
| | | GFMYIKDILSKIQYHKNCDVCKVSSGIYCKESLKAVSDFSRP |
| | | LFWDYFALVLTNACELGNWVFSKPKIPDVVYKLDNPNFFW |
| | | PVKPASHTELEDKIGMNHVLYSIKRNYPDIFEEHLAPYLTDL |
| | | NTLKLSWVQKVKFLDICVAIDMVSESLGIISHMIKRKREELY |
| | | IVKQNEQSMSHTRESQELAGGFRVTNEQICHNFLLQILFDSM |
| | | ITPVLLTSSQFKKYFWYGEVELLPNDCEHPLGQLTQFIMDCK |
| | | KLNLSRAMNLDDLDVGFVHSTIKLSDVFLNFSTFLTKVDWE |
| | | NRKDYNNLEELLKSTLESQLVLSIGLTFTHLRRSLKYKYERS |
| | | TVYTIIAKVILDIEQLTLNEDDQICLIVQEVECYVSQSGGDHI |
| | | SLDGAALIPLTPIISGTETLCLDEVAIRQDDMLKGVSKHLGN |
| | | VKLDFSSHIKELKNKFSYKIQGPQVGMNPLHIDKGIIMEGDK |
| | | VVSRLDVNVTAKSLFMALELLTDDELIRKFLRSLFYYLKSV |
| | | KKGTALISLLSSDLKDIAEVYFNHFKDILKEEADWVSFGSFQ |
| | | LVYSKSLDTIMIGDEKGEFRLKGVNCKRLIPVIPAVQEIE |
| 109 | Amino acid sequence of | MGQFITLMQSIPEALNMAFNVALVIVSLLCVTKGLINLWKC |
| | glycoprotein precursor | GIIQLLMFLALAGRSCDGEYKIDRRHVLSHVEFNLTRMFDN |
| | (GPC) protein of Pirital | LPQSCSINNTHHYYKGPENTTWGVELTLTNTSVMNRSDENV |
| | virus (PIRV) | TSIRSLGFGNITNCDKTGEAGHTLKWLLNELHFTVLHVTRHI |
| | | GALCRTTAGAGLLIQYNLTTSDKGGEVGRHLIASLAQIIGDN |
| | | KAAWVGKCYNNCTSSGKCSLTNCEGGTHYKFLVIQNTTWP |
| | | NHCSYSPMSTVRMIIQKTAYSSVSRKLLGFFTWDISDSSGQH |
| | | VPGGYCLEQWAIVWAGIKCFDNSVMAKCNKDHNEEFCDT |
| | | MRLFDFNQNAIKTLQLNVENSLNLMKKSINGLISDSLVIRNS |
| | | LKQLAKIPYCNYTKFWYVNDTITGKHSLPQCWLVSNGSYL |
| | | NETHFKNEWLWESQKLYNDMLLKEYEERQGNTPLALADL |
| | | CFWSLVFFTTTVFFQLIGIPTHRHLIGEGCPKPHRLTSNSLCS |
| | | CGFYKIPKKPFRWVRKGK |
| 110 | Amino acid sequence of | MASDNVASFRWTQALRRGLSNWTNPVKSDVITDTRALLAA |
| | nucleoprotein of PIRV | LDFDRVAQVQRLMRKDKRTDTDLTKLRDLNKEVDALMNM |
| | | RTTQKDNVLRVGGLSKDELMELASDLQKLKKKVLRVEGSG |
| | | QPGVYAGNLTTTQLEQRSKILRDMGFAQLRGNPSGVVKVW |
| | | DIKDSSLLINQFGSMPAVTMACMTEQGGESLNDVVQGLSAL |
| | | GLLYTVKYPNMSDLEKLADQYPCLGYITQEQSQINVSGYNL |
| | | SLSAAVKAGACMLDGGNMLETIQVKPTMFSSMIKAVLEVK |
| | | SKERMFVSEAPGQRNPYENLLYKLCLSGDGWPYIGSRSQVK |
| | | GRAWDNTTVDLTDTGSPNHPPVRNGGSPRLSQLSHAKEEQI |
| | | LEGLKRLDSKATTWIDIEGTPNDPVELAIFQPESGNYIHCYR |
| | | EPHDVKSFKDQSKYSHGMLLKDLTNTQPGLISFIIKNLPAGI |
| | | VLTAQGSDDIEKLLEMHARRDISIIDVRLTSEQARQFEDKVW |
| | | DKFGILCNKHKGIVLARKKKGSPPGSKNPHCALLDCIMFCST |
| | | IGGFVDDKKPTRLLPLDLLYREQASLIEL |
| 111 | Amino acid sequence of Z | MGLRYSKEVRERHGDKDLEGRVPMTLNLPQGLYGRFNCKS |
| | protein of PIRV | CWFANRGLIACSDHYLCLNCLTRLRSQSQFCGICGKPLPTKI |
| | | RFEESPSAPPYEP |
| 112 | Amino acid sequence of L | MEEHVNELHDLVRKWVSDDENFAEQKAIFLSQTKLRAITIE |
| | protein of PIRV | GLKLLSTIVEVDSCQKNSCIHNREKTLNSILRDNKIVCPTLPE |
| | | IVPDGYRLIGDVLILLEVFVRSNQESFEKKYEQDYTKLMQV |
| | | KKDLQSHGITLVPMIDGRSSYYVEFMPDWVVEKIRWHLIKL |
| | | MDLLKEDGESVEELEYERLVSSLSALENQSLGLESLLSIKER |
| | | GIEYIDRLTKIMYGNLNNNMSVDECKGEILRIYQNFRQLFDQ |
| | | GQFKPKYRKTDREFILKTLREHGLIKCAIMSEEDSCKNCMIH |
| | | MFKVLTIIKQSFVSNKNIESSFILKEYNQLLSVCNKVKSLKVL |
| | | NTRRGTLMVLDLIMLNKLLSLIKIYGIKAALTILRMQCIPAV |
| | | NDRLLSIDFLISIYERKMIKSPKWLEKVHGKLKRVVQDCMF |
| | | KALEDYLVEIDFDTWFSIKDELLMTQQFKPSICYRSSKGCVC |
| | | NAETLKNLSMMTEEDFLSYLKILSSLSLSLVNSMKTSSAPKS |
| | | KINQANDFYGIVHCEEVYFQGFGDNNACTLLYQKTGEKSRC |
| | | YSVAFSDNEQQIDYGSKISFYADPKRFFLPIMSQDVLNRMCN |
| | | EMLSWLDFLSDDNIKVVADLLRKLILCVLCTPSKRVQVYLQ |
| | | GFRYLIMAYVNEIHCNDLFAKLEVDALTASERQVMIWMDD |
| | | LTRIVLEMSKEADMAKSFKFILNLSYLCHLITKETPDRLTDQI |
| | | KCFEKFLEPKLKFGSLMVNPDSTPELTSEQEDQVCEGLHRLL |
| | | NKKIFSKCENIPGVSKELVSLCSSLFNSSNLEVKPLLNHDPLT |
| | | PSFTSTALDLSSNKSVVVPKLNEIGETLTEYDFGKIVSSVVV |
| | | DLTEHFKTKGKYKLDPRDLRFRIFKKLSSLVEVNPTKKSNR |
| | | KSESGEVVAPDESFMDELTEEQQLMLSEIEVKVSKTFEGMS |
| | | KDELNRKQSKEKGAEAHLKRLWSKEVRDKISSETSLHEVK |
| | | DFDVQLFPFDTYEELVTIVFNDKSAHDFYFLEKYLNPCPLD |
| | | MLMKNLTLKAFNEGDFFECFKYILIASEFDNKIGRYDHKIRT |
| | | RLGLKDPALKIREEARISTRESNSESIAKRLDKSFFTNSSLRN |
| | | LCFYSDESPTTRTGVATDVGKLKFGLSYKEQVGGNRELYV |
| | | GDLNTKLITRLVEDYAESICSNMKYTCLNSESEFERALLDM |
| | | KSVVRQGGFAVSMDHSKWGPHMSPAIFAQLLRCLKFRLKD |
| | | GSEIDKKAVLNILYWHLHKIVEVPFNVVQAFMSGFVKRGLG |
| | | LMDRGGATLSEEFMFGFFEKGVVPSHLSSVVDMGQGILHN |
| | | MSDLYGLITEQFINYVLDFCYNVSMTSYTSSDDEIMLSTSSA |
| | | LNHEDGSLNVDVALEILEFHNFLSDKLNKFVSPKTVAGTFA |
| | | SEFKSRFFIWSQEVPLLTKFVAAALHNIKAKAPNQLAETVDT |
| | | ILDQCVANGVSIEIVGAISKRTNSLVCYSGHPLNPFLCLEESD |
| | | VRDWVDGSRGYRLQRSIENIFPDDLCPNLIRDACRKVFHRIQ |
| | | SGKIEEEFLVASIQGSPDECLNSMLTIADVDEDIKKDLAGYR |
| | | WLNLRAYGDLRLVLRTKLMSSTRTLQREEIPSLVRSVQSKL |
| | | SKNFVRGAKRILTDAINKSAFQSCISSGFIGVCKSMGSKCVR |
| | | DNTGGFVYIKEITKHVMPHTTSYCPYCKPSKNIYCEDALRSV |
| | | SEYSRPIFWDYFSLVLSNACELGNWVFGAPILPKTVFHLDNP |
| | | NHFWPIKPSSQTELEDKVGMNHVLYSIRRNYPSIFDEHISPY |
| | | MSDLNMLRLSWVQKIKFLDLCVALDMSSECLGIISHIMRRK |
| | | REELYIVKQQELSMSHTRESTNLESGLSLEPQEVCKNFLLQV |
| | | LFDSMVNPVLLTTSQFRKYFWYGEVLQLPNDASHHLAQFT |
| | | QFILDCKQLNSSRAMTLDDLDVGYVTSRVKRTTTFVALSTFI |
| | | TSLDWENRHEYKSFQELILSSPCDVFKFEFSMTFSHIRSSHKF |
| | | RYERCTSYILKVHVVFDKRVLNSNMLEDQSLLITPHSVEYF |
| | | VSQSGGNHISLDGVGLLPLDPLISGKEVLNIDDVLRHEDVNF |
| | | SAESPLFSKMRFDFKPFLKELKNKFSYKLIGPDIIMEPLVLDK |
| | | GQIKEGSRIVSQLKLRLDFKAVFVALGCLEEESRSTFISNLFM |
| | | YIGSLRGEEHRISMTESNLVQLIDNYPQVFDSMLDATNDWL |
| | | NCGSFSLCKSKSLGCVMIADERGPFKLKGVNCRRLLPDSQA |
| | | VEID |
| 113 | Amino acid sequence of | MGQLFSFFEEVPNIIHEAINIALIAVSLIAALKGMINLWKSGL |
| | glycoprotein precursor | FQLIFFLTLAGRSCSFRIGRSTELQNITFDMLKVFEDHPTSCM |
| | (GPC) protein of Sabia | VNHSTYYVHENKNATWCLEVSVTDVTLLMAEHDRQVLNN |
| | virus (SABV) | LSNCVHPAVEHRSRMVGLLEWIFRALKYDFNHDPTPLCQK |
| | | QTSTVNETRVQINITEGFGSHGFEDTILQRLGVLFGSRIAFSNI |
| | | QDLGKKRFLLIRNSTWKNQCEMNHVNSMHLMLANAGRSS |
| | | GSRRPLGIFSWTITDAVGNDMPGGYCLERWMLVTSDLKCF |
| | | GNTALAKCNLDHDSEFCDMLKLFEFNKKAIETLNDNTKNK |
| | | VNLLTHSINALISDNLLMKNRLKELLNTPYCNYTKFWYVNH |
| | | TASGEHSLPRCWLVRNNSYLNESEFRNDWIIESDHLLSEML |
| | | NKEYIDRQGKTPLTLVDICFWSTLFFTTTLFLHLVGFPTHRHI |
| | | RGEPCPLPHRLNSRGGCRCGKYPELKKPITWHKNH |
| 114 | Amino acid sequence of | MGQLFSFFEEVPNIIHEAINIALIAVSLIAALKGMINLWKSGL |
| | GP signal peptide of | FQLIFFLTLAGRSCS |
| | SABV | |
| 115 | Amino acid sequence of | FRIGRSTELQNITFDMLKVFEDHPTSCMVNHSTYYVHENKN |
| | GP1 of SABV | ATWCLEVSVTDVTLLMAEHDRQVLNNLSNCVHPAVEHRSR |
| | | MVGLLEWIFRALKYDFNHDPTPLCQKQTSTVNETRVQINIT |
| | | EGFGSHGFEDTILQRLGVLFGSRIAFSNIQDLGKKRFLLIRNS |
| | | TWKNQCEMNHVNSMHLMLANAGRSSGSRRPL |
| 116 | Amino acid sequence of | GIFSWTITDAVGNDMPGGYCLERWMLVTSDLKCFGNTALA |
| | GP2 of SABV | KCNLDHDSEFCDMLKLFEFNKKAIETLNDNTKNKVNLLTHS |
| | | INALISDNLLMKNRLKELLNTPYCNYTKFWYVNHTASGEHS |
| | | LPRCWLVRNNSYLNESEFRNDWIIESDHLLSEMLNKEYIDR |
| | | QGKTPLTLVDICFWSTLFFTTTLFLHLVGFPTHRHIRGEPCPL |
| | | PHRLNSRGGCRCGKYPELKKPITWHKNH |
| 117 | Amino acid sequence of | MSNSKEIPSFRWTQSLRRGLSEFTTPVKTDVLRDAKMILDGL |
| | nucleoprotein of SABV | DFNQVSLVQRILRKSKRNDGDLDKLRDLNKEVDNLMSMKS |
| | | SQRDTILKLGDLNKSELMDLASDLEKLKRKVGQTERSASGG |
| | | VYLGNLSQSQLTKRSDLLRKLGFQQQQVRSPGVVRIWDVA |
| | | DPNRLNNQFGSVPALTIACMTKQSDNTMGDVVQALTSLGL |
| | | LYTVKFPNLIDLEKLTAEHDCLQIVTKDESGLNISGYNYSLS |
| | | AAVKAGATLLDGGNMLETIRITPDNFSQIIKTTLSIKKKEGM |
| | | FVDEKPGNRNPYENLLYKICLSGEGWPYIGSRSQIKGRSWE |
| | | NTTVDLSTKPQQGPRTPEKAGQNIRLSHLTELQESVVREAM |
| | | GKIDPTLTTWIDIEGTSNDPVELALYQPDTGNYILCYRKPHD |
| | | EKGFKNGSRHSHGMLLKDLESAQPGLLSYVIGLLPQNMVLT |
| | | TQGSDDIRRLVDTHGRKDLKIVDIKLASEQARKFEEPIWSDF |
| | | GHLCKKHNGVIVPKKKKDKDIPQSSEPHCALLDCLMFQSAI |
| | | AGQPPQTKLEGLLPDALLFTLEAAFTI |
| 118 | Amino acid sequence of Z | MGNSKSKSKLSANQYEQQTVNSTKQVAILKRQAEPSLYGR |
| | protein of SABV | HNCRCCWFANTNLIKCSDHYICLKCLNIMLGKSSFCDICGEE |
| | | LPTSIVVPIEPSAPPPED |
| 119 | Amino acid sequence of L | MQDPLLGTLSELKDLVRKTIPDVIELAYQKDALLSQVHPRS |
| | protein of SABV | VLIEGFKLLSLLVELESCKVNACHHNYEQKFIDVILSDGGILC |
| | | PTLPKVVPDGYNLMGKTLILLETFVRVNPDDFEKKWKADM |
| | | SKLISLKTDLGKIGVTLVPVVDGRSNYNTSFVSDWTTERLR |
| | | WLLIEVLKGMKTTSELEIEEQEYHRLIHSLAKTNNQSLGFEN |
| | | LECLKRNMLSYDQLLDSSLLVGVKNDVKESKVMEELIRLKI |
| | | WYKSEVYEKGLGKFVKTDKKVLLSQLITLGSHEENDSLDC |
| | | AFCSSRILELCFKLSVKMHEDVLTRGLNLDGTKTLHSSVQS |
| | | YLNVLSMCNKIKGSKIFNTRRNTLLFLDLIMLNFVVDEMVK |
| | | DSTVIRNLKNAGLIVGQMILLVNDRVLDILTANKLIRQKLTT |
| | | NEKWLSICSSVLKRYDLELWEKLCYLIRVPDFNELFQLAKE |
| | | LVSDRPMMRYSVHKAEERQCCHKAMENFTDDDFKIMLKA |
| | | LSHLSLGLINSMKTSFSSRLLINERDYSRYFGNVRLRECYIQR |
| | | FPITNNIIGLLFYQKTGERSRCYSLYIAENGELTEIGSFYCDPK |
| | | RYFVPIFSEAVITSMCEEMINWLNFDSELVRIVSTQLKTLML |
| | | LLLCSPSKRNQTFLQGLRYFIMAYVNQAHHIDLMSKLAVEC |
| | | KSSSEIQLQRLCVRLFVSILSGDNEIEYGFTRRFKFLLNISYLC |
| | | HFITKETPDRLTDQIKCFEKFLEPKLKFNSVIVNPSLNGTLTE |
| | | SQEHQMISSIDRFFSKELLDQSDVKEPGVSRELLGYCVSLFN |
| | | RGKLRVSGDLKVDPFRPTFTSTALDISSNKSVVVPKLDELGN |
| | | IVDKYNKQLMVSSCVTSLVEMFKTKGRYNLDPDSIDFLVLK |
| | | NLTNLVSANVPQEKSQEELSTLYEALTEDQISAFEQVRDEV |
| | | QLALHKMKSSDAREERLQDPKRNEKNASKGKILESLWSPH |
| | | QVNRAIKNETSIHEIKDFDPDILDSHLVEKLCHEVYNSSQKS |
| | | LFFLDEPLKSVPLEMLLINLTTIAYEEEEFFECFKYLLIQGDF |
| | | DQKLGTYEHKSRSRLGLSSEALKVQENARVSTRESNAEAIA |
| | | KKLDRTFFTSAALRNLCFYSEDSPTEFTSVSTNTGNLKFGLS |
| | | YKEQVGSNRELYVGDLNTKLMTRLVEDFSEVVTGSMRFSC |
| | | LNSEKEFERAICDMKMAVNNGDFSLSMDHSKWGPHMSPAL |
| | | FFTFLANLNLTEPKSRTRLNLDPLLNILKWHLHKTVEVPFNV |
| | | AQAYCIGKLKRSLGLMECQCSSLTEEFYHSYLQIQDEIPSHI |
| | | MSVLDMGQGILHNLSDLYALITEQFLNYVIHKLFDIDVTSYT |
| | | SSDDQISIMKLPLSTKENDEDFDWLEIICFHEYLSSKLNKFVS |
| | | PKSVVGNFVAEFKSRFFVMGEETPLLTKFVAAALHNVKCKT |
| | | PTQLAETIDTICDQCVANGVGVDIVSRISERVNRLISYSGYKE |
| | | TPFLTIVNQDVKDWTDGSRGYRLQRNIENSFGNQELLRLIRR |
| | | GARKVFLEIKKGHVFEENLIGLIGRGGDEALRGFLLYAGFAE |
| | | NDIVEALRHKWLNPSTFGDLRLVLRTKIMSSKRILERESVPS |
| | | LIKTLQSRMSKNFIKGAKKILAESINKSAFQSSVASGFIGFCK |
| | | SMGSKCVRDGKGGFMYLKELYNNVNKCGCCICLEWPGVV |
| | | YCQDSLAKISQFARSILWDYFTLVLTNACEIGEWVFSDVKSP |
| | | SAPPILSNPNLFWAVKPKIQKHIEDRLSLNHILHSIKRNYPYL |
| | | FEEHLAPFMSDLQFNQMMNPSHVKFLDVCIALDMMNENLG |
| | | IIGHLLRGRNHFIYIVKQSECASAHIRQSDYVDHELGLSPQQ |
| | | VCYNFKVQFLFSSMIDPLIVSTSTLKTFFWFNEVLSIEEEDQI |
| | | DLGELTDFTLFIKTGHLNRAMTADDITMGYVCSNLAEEIITL |
| | | NSYGSFQEFRSNHPSKNDLSDILKTLTSESIKLTLDIQIVHMR |
| | | NSTKYNISRKIVYTLKALCALPLEDCFTKDPVALVESLELFA |
| | | SGVNGGHLQLDGVTMVSVLPLLRGKKAVNLAQILMDNDL |
| | | AATNDHNVMESVTLDFTKFHDELGDKFCYSLVGPEDQGNPI |
| | | VLHNGMFMIDNQKLSYLKVEIFGDTIIKALGALDSPREIGSL |
| | | LHGLWPYLKATKQIINFDQTDFEMIYDLHRVVLLESIAQFGD |
| | | WVEFASFKVAFSKHYKDIVVADNLGNLRLKGVTCRLFRQQ |
| | | QSVEDIE |
| 120 | Amino acid sequence of | MGQFISFMQEIPIFLQEALNIALVAVSLICIVKGLVNLYRCGL |
| | glycoprotein precursor | FQLMVFLVLAGRSCSEETFKIGMHTKFQEVSLSLSALLTNQS |
| | (GPC) protein of Tacaribe | HELPMLCLANKTHLYLKSGRSSFKINIDSVTVLTRSEVNLTSI |
| | virus (TCRV) | NLTRSIDVFVHSPKLGSCFESDEEWVVAWWIEAIGHRWDQ |
| | | DPGLLCRNKTKTEGKLIQINISRADGNVHYGWRLKNGLDHI |
| | | YRGREEPCFEGEQCLIKIQPEDWPTDCKADHTNTFRFLSRSQ |
| | | KSIAVGRTLKAFFSWSLTDPLGNEAPGGYCLEKWMLVASEL |
| | | KCFGNTAIAKCNQNHDSEFCDMLRLFDYNKNAIKTLNEETK |
| | | TRVNVLSHTINALISDNLLMKNKIRELMSVPYCNYTRFWYV |
| | | NHTLSGQHSLPRCWMIRNNSYLNSSEFRNEWILESDFLISEM |
| | | LGKEYSERQGRTPITLVDICFWSTVFFTSTLFLHLIGFPTHEHI |
| | | RGEGCPLPHRLNSMGGCRCGKYLPLKKPTIWHRRH |
| 121 | Amino acid sequence of | MGQFISFMQEIPIFLQEALNIALVAVSLICIVKGLVNLYRCGL |
| | GP signal peptide of | FQLMVFLVLAGRSCS |
| | TCRV | |
| 122 | Amino acid sequence of | EETFKIGMHTKFQEVSLSLSALLTNQSHELPMLCLANKTHL |
| | GP1 of TCRV | YLKSGRSSFKINIDSVTVLTRSEVNLTSINLTRSIDVFVHSPKL |
| | | GSCFESDEEWVVAWWIEAIGHRWDQDPGLLCRNKTKTEGK |
| | | LIQINISRADGNVHYGWRLKNGLDHIYRGREEPCFEGEQCLI |
| | | KIQPEDWPTDCKADHTNTFRFLSRSQKSIAVGRTLK |
| 123 | Amino acid sequence of | AFFSWSLTDPLGNEAPGGYCLEKWMLVASELKCFGNTAIA |
| | GP2 of TCRV | KCNQNHDSEFCDMLRLFDYNKNAIKTLNEETKTRVNVLSH |
| | | TINALISDNLLMKNKIRELMSVPYCNYTRFWYVNHTLSGQH |
| | | SLPRCWMIRNNSYLNSSEFRNEWILESDFLISEMLGKEYSER |
| | | QGRTPITLVDICFWSTVFFTSTLFLHLIGFPTHEHIRGEGCPLP |
| | | HRLNSMGGCRCGKYLPLKKPTIWHRRH |
| 124 | Amino acid sequence of | MAQSKEVPSFRWTQSLRKGLSQFTQTVKSDILKDAKLIADSI |
| | nucleoprotein of TCRV | DFNQVAQVQRVLRKTKRTDDDLNKLRDLNIEVDRLMSMKS |
| | | VQKNTIFKVGDLARDELMELASDLEKLKDKIKRTESNGTNA |
| | | YMGNLPQSQLNRRSEILRTLGFAQQGGRPNGIVRVWDVKD |
| | | SSKLNNQFGSMPALTIACMTVQGGETMNNVVQALTSLGLL |
| | | YTVKYPNLSDLDKLIPNHECLQIITKEESSINISGYNLSLLAA |
| | | VKAGASILDGGNMLETIRVSPDNFSSLIKNTLQVKRREGMFI |
| | | DDRPGSRNPYENLLYKLCLSGDGWPYIGSRSQIMGRSWDNT |
| | | SVDLTKKPDAVPEPGAAPRPAERKGQNLRLASLTEGQELIV |
| | | RAAISELDPSNTIWLDIEDLQLDPVELALYQPAKKQYIHCFR |
| | | KPHDEKGFKNGSRHSHGILMKDIEDAVPGVLSYVIGLLPPN |
| | | MVITTQGSDDIRKLLDIHGRKDLKLIDVKFTSDQARLFEHQV |
| | | WDKFGHLCKQHNGVIISKKNKSKDSPPSPSPDEPHCALLDCI |
| | | MFHSAVSGELPKEEPIPLLPKEFLFFPKTAFAL |
| 125 | Amino acid sequence of Z | MGNCNRTQKPSSSSNNLEKPPQAAEFRRTAEPSLYGRYNCK |
| | protein of TCRV | CCWFADKNLITCSDHYLCLRCHQIMLRNSELCNICWKPLPT |
| | | SIRVPLEASAPDL |
| 126 | Amino acid sequence of L | MDETVSELKDLVRKHIPNRHEFAHQKDAFLSHCHSGSLLQE |
| | protein of TCRV | GFKLLSNLVELESCESHACHLNTCQKYVDVILSDHGIPCPTL |
| | | PKVIPDGFKLTGKTLILLETFVRVNPEEFERKWKSDMTKLLN |
| | | LKQDLLRSGITLVPVVDGRTNYSNRFTPEWVVERIRWLLIEI |
| | | LRKSRSSAEIDIEDQEYQRLIHSLSNVRNQSLGFENIECLKRN |
| | | LLEYDDRLAKSLFVGVKGDVRESVIREELMKLRLWYKKEV |
| | | FDKNLGKFRITNRSELLNNLIRLGKHEDNTTSDCPFCVNKFM |
| | | DIIYSLTFTALKRQDREKSNSELDQYVVCPHEKAYLGVLSIC |
| | | NKIKGLKVFNTRRNTLLFLDLIMVNFLDDLFTAKPEALDSLR |
| | | RSGLILGQMVTLVNDRALDFLEAVKLIKKKIETNVKWVENC |
| | | SKILRRSQQDIWSQISVWARYPDLSKLISIAQTISSDRPIMRYS |
| | | AGGNFNTECKHKTFHMMSDAEQVEAFKILSSVSLSLINSMK |
| | | TSFSSRLLINEKEYSRYFGNVRLRECYQQRFFLTDGLIVILFY |
| | | QKTGERSGCYSIYTCEDGVLVEKGSFYCDPKRFFLPIFSQEV |
| | | LVEMCDEMTTWLDFNSDLMVISKEKLRLLLLSILCAPSKRN |
| | | QVFLQGLRYFLMAYSNQFHHVDLLSKLKVECMSGSEVIVQ |
| | | RLAVDLFQCLLGEGVDSDPYFARRFKYLLNVSYLCHLITKE |
| | | TPDRLTDQIKCFEKFIEPKIDFNCVIVNPSLNGQLTEAQEGM |
| | | MLDGLDKFYSKTLKDCSDTKLPGVSNELLSYCISLFNKGKL |
| | | KVTGELKNDPFKPNITSTALDLSSNKSVVVPKLDELGNVLS |
| | | VYDREKMISSCVSSMAERFKTKGRYNIDPSTLDYLILKNLTG |
| | | LVSIGSKTQRDCEELSMMFEGLTEEQAEAFNDIKNSVQLAM |
| | | VKMKDSKSGDVNLSPNQKEGRVKSSTGTLEELWGPFGIMR |
| | | EIRTEVSLHEVKDFDPDVLASDLYKELCDVVYYSSSKPEYFL |
| | | ERPLEVCPLGLLLKNLTTSAYFDEEYFECFKYLLIQGHYDQK |
| | | LGSYEHRSRSRLGFTNEALRVKDEVRLSMRESNSEAIADKL |
| | | DRSYFTNAALRNLCFYSDDSPTEFTSISSNNGNLKFGLSYKE |
| | | QVGSNRELYVGDLNTKLITRLVEDFAEAVGSSMRYTCLSSE |
| | | KEFDRAICDMKLAVNNGDLSCSLDHSKWGPTMSPALFLTFL |
| | | QFLELRTPKERNIINLEPVLNVLRWHLHKVIEVPVNVAEAYC |
| | | TGNLKRSLGLMGCGSSSVGEEFFHQFMPVQGEIPSHIMSVL |
| | | DMGQGILHNMSDLYGLITEQFLNYVLDLLYDVIPTSYTSSD |
| | | DQVTLIKLPCASDDNQVNDEWLEMLCFHEYLSSKLNKFVSP |
| | | KSVAGTFVAEFKSRFFVMGEETPLLTQFVAAALHNVKCKTP |
| | | TQLSETIDTICDQCVANGVSVQIVSKISQRVNQLIKYSGFKET |
| | | PFGAVEKQDVKDWVDGTRGYRLQRKIESIFSDDEMTGFIRS |
| | | CAKRVFNDIKRGKVFEENLISLIGRDGDDALVGFLRYSSCSE |
| | | QDIMRALGFRWVNLSSFGDLRLVLRTKLMTSRRVLEREEVP |
| | | TLIKTLQSRLSRNFTKGVKKILAESINKSAFQSSVASGFIGFC |
| | | KSIGSKCVRDGEGGFLYIKDIYTKVKPCLCEVCNMKRGVIY |
| | | CRPSLEKIEKFSKPILWDYFSLVLTNACEIGEWVFSSVKEPQI |
| | | PVVLSNRNLFWAVKPRIVRQLEDQLGMNHVLYSIRKNYPK |
| | | LFDEHLSPFMSDLQVNRTLDGRKLKFLDVCIALDLMNENLG |
| | | IVSHLLKARDNSVYIVKQSDCAMAHVRQSDYVDKEVGLSP |
| | | QQVCYNFMVQIILSSMVNPLVMSTSCLKSFFWFNEVLELED |
| | | DGQIELGELTDFTFLVRDQKISRAMFIEDIAMGYVISNLEDV |
| | | RLYIDKITIGEQPLAPGRHINDLLDLLGNFDDHEDCDLRFLIQ |
| | | VEHSRTSTKYRFKRKMTYSFSVTCVSKVIDLKEASVELQVV |
| | | DVTQSVSGSGGSHLLLDGVSMIAGLPIFTGQGTFNMASLMM |
| | | DADLVETNDNLILTDVRFSFGGFLSELSDKYAYTLNGPVDQ |
| | | GEPLVLRDGHFFMGTEKVSTYRVELTGDIIVKAIGALDDPED |
| | | VNALLNQLWPYLKSTAQVMLFQQEDFVLVYDLHRSGLIRS |
| | | LELIGDWVEFVNFKVAYSKSLKDLVVSDNQGSLRLRGIMCR |
| | | PLARRNTVEDIE |
| 127 | Amino acid sequence of | MGQLVSFIGEIPAIVHEALNVALIAVSIIAIMKGLINIWKSGLF |
| | glycoprotein precursor | QLIMFLILAGRSCSISIGHHLELQHFIINSTSLLPSMPTLCRINA |
| | (GPC) protein in Bear | TNSLIRGPFSAQWGLDIFIGDLTILVNPEPGSKTKRMTATNIT |
| | Canyon virus (BCNV) | GCFPNNEDPDSVAQVLSWFFRGVHHDFHLDPTILCDESVTV |
| | | FRIQMNLTERMYCDRIVSKLARLFGSFGDYCSKVGKKLVIIR |
| | | NVTWSNQCHEDHVGSMQLILQNAHNQVMRFRKLQNFFSW |
| | | SLVDSAGNSMPGGYCLEKWMLVASELKCFGNTAVAKCNIN |
| | | HDSEFCDMLRLFDYNKKAIVNLQDKTKAQLDSLIDAVNSLI |
| | | SDNLITKNKIRELMNIPYCNYTKFWYVNHTGLNVHSLPKCW |
| | | HVRNGSYLNESDFRNEWIIESDHLVSEILAKEYEERQKRTPL |
| | | SLVDLCFWSTLFYTASIFLHLLHIPTHRHIIGEGCPKPHRLTS |
| | | DSLCACGFFQLKGRPTRWARIP |
| 128 | Amino acid sequence of | MGQLVSFIGEIPAIVHEALNVALIAVSIIAIMKGLINIWKSGLF |
| | GP signal peptide in | QLIMFLILAGRSCS |
| | BCNV | |
| 129 | Amino acid sequence of | ISIGHHLELQHFIINSTSLLPSMPTLCRINATNSLIRGPFSAQW |
| | GP1 protein in BCNV | GLDIFIGDLTILVNPEPGSKTKRMTATNITGCFPNNEDPDSVA |
| | | QVLSWFFRGVHHDFHLDPTILCDESVTVFRIQMNLTERMYC |
| | | DRIVSKLARLFGSFGDYCSKVGKKLVIIRNVTWSNQCHEDH |
| | | VGSMQLILQNAHNQVMRFRKLQ |
| 130 | Amino acid sequence of | NFFSWSLVDSAGNSMPGGYCLEKWMLVASELKCFGNTAV |
| | GP2 protein in BCNV | AKCNINHDSEFCDMLRLFDYNKKAIVNLQDKTKAQLDSLID |
| | | AVNSLISDNLITKNKIRELMNIPYCNYTKFWYVNHTGLNVH |
| | | SLPKCWHVRNGSYLNESDFRNEWIIESDHLVSEILAKEYEER |
| | | QKRTPLSLVDLCFWSTLFYTASIFLHLLHIPTHRHIIGEGCPK |
| | | PHRLTSDSLCACGFFQLKGRPTRWARIP |
| 131 | Amino acid sequence of | MSDQVVHSFRWTQSLRRGLSNWTCPVKADVLNDTRALLSG |
| | NP protein in BCNV | LDFAKVASVQRMMRRDKRDESDLTSLRDLNKEVDSLMTM |
| | | KSTQKNMFLKVGSLSKGELMELSGDLNKLKDKVQRTERPP |
| | | GSGGQYQGNLTTTQLTRRGELLQFIGIQKAGRVGMNGVVK |
| | | VWDVKDSSLMINQFGSMPALTISCMAEQGGETLNDVVQGL |
| | | TDLGLLYTAKYPNLNDLEALSEKHPCLKVITQEESQINISGY |
| | | NLSLSAAVKAGACLIDGGNMLETIKIDTSTFTTVIKTLLEVK |
| | | ARERMFVSSVPGQRNPYENILYKLCLSGEGWPYIASRSQIKG |
| | | RAWDNTVVEFDSAPPRAPVPVRNGGAPLLGPLRPELEDQVR |
| | | KGVEGLSPNLTTWIDIEGPPNDPVELAIYQPETQKYLHCYRR |
| | | PNDIKSFKDQSKYCHGILLKDVENARPGLISTIIRYLPKSMVF |
| | | TAQGEDDIKRLFDMHGRQDLKIVDVKLSAEQSRVFEELVW |
| | | KKFEHLCDRHKGIVIKSKKKGSKPASTNAHCALMDCIMFNA |
| | | VLVGFVADEKPKRLLPIDILFREPDTTVVL |
| 132 | Amino acid sequence of Z | MGLRYSREVKQRYGEKELEGRIPITLDMPQTLYGRYNCKSC |
| | protein in BCNV | WFANKGLIKCSNHYLCLKCLTAMLSRSDYCGICGGILPKKL |
| | | VFETTPSAPPYTP |
| 133 | Amino acid sequence of L | MSEYLDELKELIRKWIPDEEMYIEQKTSFLSQVNLRSVVIEG |
| | protein in BCNV | LKLLSIIIEIDSCKKHGCVHNKNKTVNQILRDHRIVGPTLPDV |
| | | VPDGYRVIGSTIILLEAFVRVSHESFEIKYKSDFEKLMQLSKD |
| | | LSRCGLTLIPVVDGRSNYYTEHFPDWTIERMRWLILKITNFL |
| | | RDNGEEIEELEYSRLVYSLSNMENKNLGLESLKILKEEGLDY |
| | | KAKLMSVMRDGVNSNMSASECRVEMAKIYDQFSFLRKNG |
| | | LYKDVYCKTSRTEIINWLKDHKLILLSGETRTAMLDERQCG |
| | | YCRNHMFRILASLIKNKRHYQSLTNPKKCGSIQSHKKLLSDC |
| | | NKIKGLKVLNTRRFTLLCLDVIILNSLLELIDAGEIDNEFLVN |
| | | NHFKSVNDRLVSIDLIIDRLNKKLMSKPNWIGSVKYKMKRT |
| | | LEIHGLYYVSKWLKQVDIDSWYEFKMMREHSDKCVKPTLK |
| | | YKKDAARKCGQPEFGSSTILDDEVFLEYLEALSTLSLGLVNS |
| | | MKTSSAAKFLINDKSNYFGTVQCNECYFQDLDKSYNSLLIY |
| | | QKTGERSRCYGLMFKSEQFENVYEVGESFYADPKRFFLPIM |
| | | SSEVILKMCVEMLSWLDWLSEQELKAFKSKLYTLIINILTVP |
| | | SKRVQVYLQGFRYLIMAFVNELHFKELQNKLKVQPLTISEC |
| | | YVFTLMDDLVHLLLTEAQEENMSKVFRFVLNLSYLCHLVT |
| | | KETPDRLTDQIKCFEKFLEPKVDFNSVFVNLDSSPHLSGEVE |
| | | EKFIKDLNRLFSKDLGVEDLKDPGISKELISLCASCFNCGLLP |
| | | MSKVLKHDPQSPSFTSTALDISSNKSVVVPKLDEVGETVTQ |
| | | YDYQSLLSSTVVDMAQSFKDKLKYKLDRKSIQFAIFKRLTN |
| | | MVLKRKTDHDVKDDLDDELSEIVDDDTLRVINDVEANVSE |
| | | CLSKMGKISRAATVGGQNNLGRFEKIDTLKRLWDRESMNFI |
| | | LMETSLHEVKDFDPSIFPIEKYKSMCELVYDSKMKSEFFTDE |
| | | VLKFCPLDLLVKNLATKCYLEEDFFECFKYILISAGFDNRVG |
| | | RYDHRSRSRLGFKDEAILIKENSRISSRESNSEAISRRLDKSFF |
| | | TNSSLRNLCFYSEESPTYRSTVSSSVGKLKFGLSYKEQVGSN |
| | | RELYVGDLNTKLTSRLIEDYFESLTSECKFSCLNNDAEFERA |
| | | LLDMKCVVRLSGLAVSMDHSKWGPYMSPAIFNILFSNLNLE |
| | | LNDGVFIDKAPIENLLNWHLHKIVEVPYNVIDAYLKGYTKR |
| | | RLGLMDRSSTSITEDFIFNWFAKGVVPSHISSVLDMGQGILH |
| | | NTSDYYGLLTEQFILQCLDFIFDIKSTAYTSSDDEILLSNSPSL |
| | | KKVDEDSLDINKCQEVLEFHNYLSSKFNKFVSPKTVAGSFA |
| | | SEFKSRFFIWSQEVPLLTKFVAAALHNVKAKSPHQLAETVD |
| | | TILDQCIANGVSIEVVKAISRRTNKLITYSGHPKNPFLCVENT |
| | | DLKDWVDGSRGYRLQRSVESLFNDDDLPLTIRNSCRSLFHRI |
| | | RSGDIQEEFLINALQTSPDECLAKMLRLSDVDESTIDKVLEF |
| | | RWLNLRAHGDLRLVLRTKVMSGTRILDREEVPSLVKSVQS |
| | | KLSKNFVRGAKKIITDAINKSAFQSSICSGFIGFCKSMGSKCV |
| | | RDGNGSFQYIKHFLKSIILHSHCEVCKPEMSVFCRAALEELK |
| | | PFSRPIFWDYFSLTFSNACELGNWVFSNVTIPKRTPTTVNPNF |
| | | FWPVKPGSHTELEDKVNMNHVLYSIKRNFPDLFDEHIAPFLS |
| | | DLNSLKISWIQRIKFLDLCVAMDMSSECLGIISHIMRRKREEL |
| | | YIVKQNELSVAHMRDSSPMEAGYQLNSSEICHNFLCQLVFE |
| | | SMLHPVLLTTGQFKKYFWFGEVELLPNEADHDLGQLTQFV |
| | | MDCKTLNISRCMSLDDLDVGYVHSSILMGDIYVNFSSFLHL |
| | | LDWENRRNYKTFDEIILCSREDTIPMEIDFTISHSRKSFKFKY |
| | | ERKTNYHIKSKVLVQKVDIEEAQNQGFDILELEVHEIECFVS |
| | | GSQGNHISLDGVGLIPLHPLFSGKEFLDVNKLLIKQDENFEST |
| | | HSVFSKVKLNFSNHTKDLKNKYSYKLQGPEYNMNPLHLYR |
| | | GQIMENNFVISRLDVQITSRSVFLALEALESEDRIPFLISLHIY |
| | | TRSNNKKENSCFIRMTQSDLCLLIDSYEKEFTEVLKSLSDW |
| | | MDFGDFALCFSNNLNCIMIADPDGQFKLKGRQCRKVSSASA |
| | | PLEID |
| 134 | Amino acid sequence of | MGQLISFFGEIPSIIHEALNIALIAVSIISILKGVINIWGSGLLQF |
| | the glycoprotein precursor | IVFLLLAGRSCSYKIGHHVELQHIILNASYITPYVPMPCMIND |
| | (GPC) protein of | THFLLRGPFEASWAIKLEITDVTTLVVDTDNVANPTNISKCF |
| | Whitewater Arroyo virus | ANNQDERLLGFTMEWFLSGLEHDHHFTPQIICGNVSKGEVN |
| | (WWAV) | AQVNITMEDHCSQVFLKMRRIFGVFKNPCTSHGKQNVLISV |
| | | SNWTNQCSGNHLSSMHLIVQNAYKQMIKSRTLKSFFAWSLS |
| | | DATGTDMPGGYCLEKWMLISSELKCFGNTAIAKCNLDHSSE |
| | | FCDMLKLFEFNRNAIKTLQNDSKHQLDMIITAVNSLISDNTL |
| | | MKNRLKELINIPYCNYTKFWYVNHTGFNVHSLPRCWLTKN |
| | | GSYLNVSDFRNQWLLESDHLISEILSREYEARQGKTPLGLVD |
| | | VCFWSTLFYVSSIFLHLLRIPTHRHIIGEGCPKPHRLSSNSVC |
| | | ACGLFKQKGRPLRWAGKV |
| 135 | Amino acid sequence of | MGQLISFFGEIPSIIHEALNIALIAVSIISILKGVINIWGSGLLQF |
| | the GP signal peptide of | IVFLLLAGRSCS |
| | WWAV | |
| 136 | Amino acid sequence of | YKIGHHVELQHIILNASYITPYVPMPCMINDTHFLLRGPFEAS |
| | the GP1 of WWAV | WAIKLEITDVTTLVVDTDNVANPTNISKCFANNQDERLLGF |
| | | TMEWFLSGLEHDHHFTPQIICGNVSKGEVNAQVNITMEDHC |
| | | SQVFLKMRRIFGVFKNPCTSHGKQNVLISVSNWTNQCSGNH |
| | | LSSMHLIVQNAYKQMIKSRTLK |
| 137 | Amino acid sequence of | SFFAWSLSDATGTDMPGGYCLEKWMLISSELKCFGNTAIAK |
| | GP2 of WWAV | CNLDHSSEFCDMLKLFEFNRNAIKTLQNDSKHQLDMIITAV |
| | | NSLISDNTLMKNRLKELINIPYCNYTKFWYVNHTGFNVHSL |
| | | PRCWLTKNGSYLNVSDFRNQWLLESDHLISEILSREYEARQ |
| | | GKTPLGLVDVCFWSTLFYVSSIFLHLLRIPTHRHIIGEGCPKP |
| | | HRLSSNSVCACGLFKQKGRPLRWAGKV |
| 138 | Amino acid sequence of | MSDQSVPSFRWTQSLRRGLSAWTTSVKADVLNDTRALLSG |
| | nucleoprotein of WWAV | LDFAKVASVQRMMRRVKRDDSDLVGLRDLNKEVDSLMIM |
| | | KSNQKNMFLKVGSLSKDELMELSSDLEKLKQKVQRTERVG |
| | | NGTGQYQGNLSNTQLTRRSEILQLVGIQRAGLAPTGGVVKI |
| | | WDIKDPSLLVNQFGSVPAVTISCMTEQGGESLNDVVQGLTD |
| | | LGLLYTAKYPNLNDLKALTTKHPSLNIITQEESQINISGYNLS |
| | | LSAAVKAGACLIDGGNMLETIKIEESTFTTVIKTLLEVKNKE |
| | | KMFVSPTPGQRNPYENVLYKLCLSGDGWPYIASRSQIKGRA |
| | | WDNTVVEFDTATVKEPIPIRNGGAPLLTTLKPEIENQVKRSV |
| | | ESLLINDTTWIDIEGPPNDPVEFAIYQPESQRYIHCYRRPNDI |
| | | KSFKDQSKYCHGILLKDVENARPGLISSIIRSLPKSMVFTAQG |
| | | ADDIRKLFDMHGRQDLKIVDVKLSAEESRIFEDLVWKRFEH |
| | | LCDKHKGIVIKSKKKGSTPATTNAHCALLDGVMFSAVISGS |
| | | VSNEKPKRMLPIDLLFREPETTVVL |
| 139 | Amino acid sequence of | MGLRYSKEVRDRYGDKEPEGRIPITLNMPQTLYGRYNCKSC |
| | the Z protein of WWAV | WFANKGLLKCSNHYLCLKCLTLMLGRSDYCGICGEVLPKK |
| | | LVFENSPSAPPYEA |
| 140 | Amino acid sequence of | MIDCSDRINELKDLVRRWVPDEEAYTEQKTIVLSQVNPSSVI |
| | the L protein of WWAV | TEGLKLLSMLIEIDSCLKHGCVFNKNKTVNQILKDHKIVGPT |
| | | LPDVVPDGFKVSGSTIILLETFVRVNQESFEQKYKYDFEKLM |
| | | QLSKDLNKCGLILVPVIDGRSNYYVDRFPDWVIERIRWLLL |
| | | KLMDSVKDSGESIEELEYNRLISSLSNMENQNLGLESLKALR |
| | | EEGLDYKAKLMGVIKDGTMSKMTASECRIGIAKIYDQFCLL |
| | | RDSGQYQDVYCETSRADMIKWLKTHKLISPISNGEGGPLNS |
| | | ERCGFCQNHMLRVIAELVQSKRASCTQLPAESKETLRHKKL |
| | | LSDCNKVKGLKVLNTRRHAILCLDVIVLNSLIEVIKSGVDSS |
| | | HFLINNHYKSVNDRLLSVDLIINKLERKLLKQPDWLKIVGRK |
| | | LNKSVKEQSLDYVTVWLKELDYEFWYEFKFEREHSGKCEK |
| | | PTLRYKKQNQDTCYQVKFGTDKVLNEGMFVDYLDALSSLS |
| | | LSMMNSMKTSSAPKLIINDEKNFYGTIQCEECYYQDLDNLY |
| | | NSILLYQKTGEKSRCYGLMLKDEEMTNAYKTGPSFYADPK |
| | | RFFLPIMSSTVILKTCLEMLSWLDWLSTAEINDVKTKLFTLV |
| | | VNILITPSKRVQIYLQGFRYFIMAFVNEFHFKKLDQKLSVQA |
| | | LTSAEQHVFVLMDELVVYLLEEALEENMAKIFKFVLNLSYL |
| | | CHFITKETPDRLTDQIKCFEKFLEPKIQFGSSFVNLDSSPNLC |
| | | KEDEEKFIMNLNKLFSKDLGVDDMENPGICKTILSLCVSCFN |
| | | CGILPINKVLDRDPQSPSFSSTALDISSNKSVVVPKLDEAGEV |
| | | ITHYDYQSLLSSVVVEMAQSFKDKLRFKLDRKSIQFAIYKRL |
| | | TNMVLKRRSHPKDHDDECAEEFEELLDEGTYKLINDIESNV |
| | | LECLNGMVTSPMKKDVKEQGAVRRYEGSDLLSTLWPREL |
| | | MGPILAETSLHEVKDFDPSIFSDGTYQDLCHSVFNSKFKKHF |
| | | FLDDVLRFCPLESLVKNLATKNYIEKDYFECFKYILISAGFD |
| | | NRVGRYDHRSRSRLGFKDAAYHVKEASRISLRESNSEAISK |
| | | RLDKSFFTNSSLRNLCFYSEESPTFQSTVSSSTGKLKFGLSYK |
| | | EQVGSNRELYVGDLNTKLTSRLIEDYFESITSESKFSCLNNEL |
| | | EFEKAILDMKSVVRLSGLAVSMDHSKWGPYMSPAIFNALFS |
| | | NLDLQLKDGTSIDKGPIENLLNWHLHKLVEVPYNVIEAYLK |
| | | GYTKRGLGLMDRMSNTICENFIFNWFARGVVPSHISSVLDM |
| | | GQGILHNTSDYYGLVTEQFIMLCLEQCFDVKMSAYTSSDDE |
| | | IILSNSFSLRNSDNESLNNLKCKELLEFHYYLSSKLNKFVSPK |
| | | TVAGSFASEFKSRFFIWSQEVPLLTKFVAAALHNVKAKSPH |
| | | QLAETIDTILDQCVANGVSIEIVKEISKRTNRLIKYSGHPIDPF |
| | | LCVVDTDLKDWVDGSRGYRLQRSIESAIGDDTQLSIIRNSCK |
| | | KLFFKIRSGNIQEEYLISALQSSPDECLRQMLSITEVNEQEIED |
| | | LVETRWLNLRTFGDLRLVLRTKIMSGTRILDREEIPSLIKSVQ |
| | | SKLSKNFVRGAKKIVTDAINKSAFQSSICSGFIGLCKSMGSK |
| | | CVRDGTGGFVYIKMLLREIKDHQACGICKPKLSVFCKSALD |
| | | RLPKYSRSLLWDYYSLVFTNACELGNWVFSEAMLPKRVPT |
| | | MVNPNFFWCVKPGSHTELEDKVNMNHVLYSIKRNFPDLFD |
| | | EHIAPFLSDLASLKISWVQRIKFLDLCVAMDMSSECLGVISHI |
| | | MRRKREESYIVKQNELSHAHVRDSNPLEGGFQLNSLEICRN |
| | | FLYQIIFESMLHPVLLTTSQFKKYFWYGEVELLPNDADHDL |
| | | GQLTQFIMDCKTLNISRCMSLDDLDVGYVHSKITLSDIFINLS |
| | | SFMHLLDWGSLNDYESFDDIILKTGQKQTPIEIGIVLSHIRRS |
| | | FKFKYDRKTNYHIECRLIIDKDEMMMNRWDEDNILEIEVSE |
| | | VQCFVSGSEGHHISLDGVGLIPLHPLFSGKELIDFNKLLVDQS |
| | | VEFRQTSTVFQRVRLDFRQHTKDLRNKFSYKFQGPEQGLSP |
| | | LHLYKGQIMERNTIVSRLDVPISSKSVFLALEALDPAEHTTFL |
| | | TSLHTYMKTRMSKSNPCFIRMTQEDLCLLIESYEAAFIGVLK |
| | | LESDWIEFGDFALCFSNSLNCIMIADDGGQFKLKGRKCRSAS |
| | | TCPRPLEIE |

TABLE 5
Assession number for specific arenaviruses

| | | GenBank | |
|---|---|---|---|
| | | Accession | |
| Virus name | Isolate | number | Ref. Seq. number |
| Allpahuayo virus | CHLP-2472 | L: AY216502; | L: NC_010249; |
| | | S: AY012687 | S: NC_010253 |
| Alxa virus | RtDs-AreV-IM2014 | L: KY432892; | |
| | | S: KY432893 | |
| Junín virus | XJ13 | L: AY358022; | L: NC_005080; |
| | | S: AY358023 | S: NC_005081 |
| Bear Canyon virus | AV A0070039 | L: AY924390; | L: NC_010255; |
| | | S: AY924391 | S: NC_010256 |
| Sabiá virus | SPH114202 | L: AY358026; | L: NC_006313; |
| | | S: U41071 | S: NC_006317 |
| Pichindé virus | AN3739 | L: AF427517; | L: NC_006439; |
| | | S: K02734 | S: NC_006447 |
| Chapare virus | 810419 | L: EU260464; | L: NC_010563; |
| | | S: EU260463 | S: NC_010562 |
| Lìjiāng virus | KS4 | L: MF414201; | |
| | | S: MF414202 | |
| Cupixi virus | BeAn 119303 | L: AY216519; | L: NC_010252; |
| | | S: AF512832 | S: NC_010254 |
| Flexal virus | BeAn 293022 | L: EU627611; | L: NC_010759; |
| | | S: AF512831 | S: NC_010757 |
| Gairo virus | TZ-27421 | L: KJ855307; | L: NC_026247; |
| | | S: KJ855308 | S: NC_026246 |
| Guanarito virus | INH-95551 | L: AY358024; | L: NC_005082; |
| | | S: AY129247 | S: NC_005077 |
| Ippy virus | Dak An B 188d | L: DQ328878; | L: NC_007906; |
| | | S: DQ328877 | S: NC_007905 |
| Lassa virus | Josiah | L: U73034; | L: NC_004297; |
| | | S: J04324 | S: NC_004296 |
| Latino virus | MARU 10924 | L: EU627612; | L: NC_010760; |
| | | S: AF512830 | S: NC_010758 |
| Loei River virus | R5074 | L: KC669693; | L: NC_038365; |
| | | S: KC669698 | S: NC_038364 |
| Lujo virus | R4356 | L: FJ952385; | L: NC_012777; |
| | | S: FJ952384 | S: NC_012776 |
| Luna virus | LSK-1 | L: AB586645; | L: NC_016153; |
| | | S: AB586644 | S: NC_016152 |
| Luli virus | SLW-1 | L: AB972431; | |
| | | S: AB972430 | |
| Lunk virus | NKS-1 | L: AB693151; | L: NC_018711; |
| | | S: AB693150 | S: NC_018710 |
| lymphocytic | Armstrong 53b | L: AY847351; | L: NC_004291; |
| choriomeningitis | | S: AY847350 | S: NC_004294 |
| virus | | | |
| Machupo virus | Carvallo | L: AY358021; | L: NC_005079; |
| | | S: AY129248 | S: NC_005078 |
| Mariental virus | N27 MrMi.n9 | L: KP867641; | L: NC_027136; |
| | | S: KM272987 | S: NC_027134 |
| Merino Walk virus | Merino Walk | L: GU078661; | L: NC_023763; |
| | | S: GU078660 | S: NC_023764 |
| mobala virus | Acar 3080 | L: DQ328876; | L: NC_007904; |
| | | S: AY342390 | S: NC_007903 |
| Mopeia virus | AN20410 | L: AY772169; | L: NC_006574; |
| | | S: AY772170 | S: NC_006575 |
| Morogoro virus | 3017/2004 | L: EU914104; | |
| | | S: EU914103 | |
| Okahandja virus | N73 OkhMi.n4 | L: KP867642; | L: NC_027137; |
| | | S: KM272988 | S: NC_027135 |
| Oliveros virus | 3229 | L: AY216514; | L: NC_010250; |
| | | S: U34248 | S: NC_010248 |
| Paraná virus | 12056 | L: EU627613; | L: NC_010761; |
| | | S: AF485261 | S: NC_010756 |
| Pirital virus | VAV-488 | L: AY494081; | L: NC_005897; |
| | | S: AF485262 | S: NC_005894 |
| Aporé virus | LBCE 12071 | L: MF317491; | L: NC_040763; |
| | | S: MF317490 | S: NC_040762 |
| Ryukyu virus | YN2013 | L: KM020190; | L: NC_039010; |
| | | S: KM020191 | S: NC_039009 |
| Amaparí virus | BeAn 70563 | L: AY216517; | L: NC_010251; |
| | | S: AF485256 | S: NC_010247 |
| Solwezi virus | 13ZR68 | L: AB972429; | L: NC_038366; |
| | | S: AB972428 | S: NC_038367 |
| souris virus | PREDICT-05775- | L: KP050226; | L: NC_039011; |
| | 5302-5304 | S: KP050227 | S: NC_039012 |
| Tacaribe virus | T.RVL.II 573 | L: J04340; | L: NC_004292; |
| | | S: M20304 | S: NC_004293 |
| Tamiami virus | W10777 | L: AY924393; | L: NC_010702; |
| | | S: AF485263 | S: NC_010701 |
| Wēnzhōu virus | Rn-242 | L: KJ909795; | L: NC_026019; |
| | | S: KJ909794 | S: NC_026018 |
| Whitewater Arroyo | AV 9310135 | L: AY924395; | L: NC_010703; |
| virus | | S: AF228063 | S: NC_010700 |
| Big Brushy Tank | AV D0390174 | L: EU938665; | |
| virus | | S: EF619035 | |
| Catarina virus | AV N0010001 | S: JX237768 | |
| Skinner Tank virus | AV D1000090 | L: EU938659; | |
| | | S: EU123328 | |
| Tonto Creek virus | AV D0390060 | L: EU938663; | |
| | | S: EF619034 | |
| Xapuri virus | LBCE 19881 | S: MG976578; | |
| | | L: MG976577 | |

S indicates the GenBank accession number or the sequence number of the S segment; L indicates GenBank accession number or the sequence number of the L segment

What is claimed:

1. A nucleotide sequence comprising a first open reading frame (ORF) and a second ORF, wherein one of the two ORFs is in sense orientation and the other ORF is in antisense orientation;
wherein the first ORF comprises a nucleotide sequence encoding a functional fragment of a first polypeptide, and the first ORF does not encode the full-length first polypeptide;
wherein the second ORF comprises a nucleotide sequence encoding:
a) a second polypeptide; or
b) a functional fragment of the first polypeptide, and the second ORF does not encode the full-length first polypeptide; or
c) a functional fragment of a second polypeptide, and the second ORF does not encode the full-length second polypeptide; or
d) a heterologous non-arenaviral polypeptide; and
wherein the first and second polypeptides are different from each other and selected from the group consisting of arenavirus GP, NP, Z and L.

2. The nucleotide sequence of claim 1, wherein the first ORF and the second ORF are separated by an arenavirus intergenic region (IGR) and each ORF is under control of an arenavirus 3′ untranslated region (UTR) or an arenavirus 5′ UTR.

3. The nucleotide sequence of any one of claims 1-2, wherein the first ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

4. The nucleotide sequence of any one of claims 1-2, wherein the second ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

5. The nucleotide sequence of any one of the claims above, wherein the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

6. A nucleotide sequence comprising an open reading frame (ORF), wherein the ORF comprises a nucleotide sequence encoding
a) a functional fragment of a first polypeptide, and
b) a heterologous non-arenaviral polypeptide or a second polypeptide;
wherein the ORF does not encode the full-length first polypeptide; and wherein the first and second polypeptides are different from each other and selected from the group consisting of arenavirus GP, NP, Z and L that are not from Lassa virus.

7. The nucleotide sequence of claim 6, wherein the first and second polypeptides are selected from the group consisting of arenavirus GP, NP, Z and L of LCMV, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

8. The nucleotide sequence of any one of claims 6-7, wherein the ORF is a first ORF and the nucleotide sequence further comprises a second ORF comprising a nucleotide sequence encoding a third polypeptide, a functional fragment of the first polypeptide, a functional fragment of a third polypeptide, or a second heterologous non-arenaviral polypeptide; wherein the third polypeptide is different from the first polypeptide and the second polypeptide and selected from the group consisting of arenavirus GP, NP, Z and L; wherein one of the two ORFs is in sense orientation and the other ORF is in antisense orientation; wherein the second ORF does not encode the full-length first polypeptide; wherein the second ORF does not encode the full-length third polypeptide; and wherein the heterologous non-arenaviral polypeptide and the second heterologous non-arenaviral polypeptide are the same or different from each other.

9. The nucleotide sequence of any one of claims 1-5 and 8, wherein the second ORF comprises a nucleotide sequence encoding a functional fragment of the first polypeptide, and wherein the functional fragment encoded by the first ORF is different from the functional fragment encoded by the second ORF.

10. The nucleotide sequence of claim 6, wherein the nucleotide sequence does not further comprise a second ORF.

11. The nucleotide sequence of any one of the preceding claims, wherein the nucleotide sequence is an arenavirus genomic or antigenomic S segment.

12. The nucleotide sequence of any one of the preceding claims, wherein the nucleotide sequence is an arenavirus genomic or antigenomic L segment.

13. The nucleotide sequence of any one of claims 1-5, 8-9, and 11-12, wherein the first ORF is under control of an arenavirus 3′ UTR, and the second ORF is under control of an arenavirus 5′ UTR.

14. The nucleotide sequence of any one of claims 1-5, 8-9, and 11-12, wherein the first ORF is under control of an arenavirus 5′ UTR, and the second ORF is under control of an arenavirus 3′ UTR.

15. The nucleotide sequence of any one of claims 1-5, 8-9, and 11-12, wherein the first ORF comprises a nucleotide sequence encoding arenavirus GP signal peptide, either alone or fused to a heterologous non-arenaviral polypeptide.

16. The nucleotide sequence of claim 15, wherein the second ORF comprises a nucleotide sequence encoding a heterologous non-arenaviral signal peptide and the arenavirus GP1 and GP2.

17. The nucleotide sequence of claim 15, wherein the second ORF comprises a nucleotide sequence encoding a heterologous non-arenaviral polypeptide; wherein the heterologous non-arenaviral polypeptide encoded by the first ORF and the heterologous non-arenaviral polypeptide encoded by the second ORF are the same or different from each other.

18. The nucleotide sequence of any one of claims 16 and 17, wherein the first ORF is under control of an arenavirus 3′ UTR and the second ORF is under control of an arenavirus 5′ UTR.

19. The nucleotide sequence of any one of claims 1-5, 8-9, and 11-12, wherein the first ORF comprises a nucleotide sequence encoding a heterologous non-arenaviral signal peptide and the arenavirus GP1 and GP2.

20. The nucleotide sequence of claim 19, wherein the second ORF comprises a nucleotide sequence encoding NP.

21. The nucleotide sequence of claim 20, wherein the first ORF is under control of an arenavirus 5′ UTR and the second ORF is under control of an arenavirus 3′ UTR.

22. The nucleotide sequence of any one of the claims above, wherein the first polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140; wherein the second polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140; and wherein the third polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

23. The nucleotide sequence of any one of the claims above, wherein the first polypeptide comprises an amino acid sequence identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134.

24. The nucleotide sequence of any one of the claims above, wherein the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, or SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137.

25. The nucleotide sequence of any one of the claims above, wherein the functional fragment encoded by the first ORF or the second ORF is an arenavirus GP signal peptide or a functional fragment thereof.

26. The nucleotide sequence of any one of the claims above, wherein the heterologous non-arenaviral polypeptide, the second heterologous non-arenaviral polypeptide, or both heterologous non-arenaviral polypeptides are each an antigen derived from an infectious organism, tumor, or allergen.

27. The nucleotide sequence of claim 26, wherein the antigen is selected from the group consisting of
(a) viral antigens, wherein the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, wherein the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and wherein the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FN1, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-DI, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

28. The nucleotide sequence of any one of claims above, wherein the expression of the heterologous non-arenaviral polypeptide or the expression of the second heterologous non-arenaviral polypeptide is under control of an arenavirus 3′ UTR; and wherein the expression level of the heterologous non-arenaviral polypeptide or the expression level of the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide or the expression level of the same second heterologous non-arenaviral polypeptide expressed under control of the respective arenavirus 5′ UTR.

29. A translation product of the nucleotide sequence of any one of claims 1-28.

30. An arenavirus particle containing a genome comprising the nucleotide sequence of any one of claims 1-28.

31. The arenavirus particle of claim 30, wherein the genome of the arenavirus particle consists of an S segment and an L segment.

32. The arenavirus particle of claim 30, wherein the arenavirus particle is tri-segmented.

33. The arenavirus particle of claim 32, wherein the tri-segmented arenavirus particle comprises two S segments and an L segment.

34. The arenavirus particle of claim 33, wherein the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
wherein the two heterologous non-arenaviral polypeptides are the same or different from each other.

35. The arenavirus particle of claim 33, wherein the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
wherein the two heterologous non-arenaviral polypeptides are the same or different from each other.

36. The arenavirus particle of claim 32, wherein the tri-segmented arenavirus particle comprises an S segment and two L segments.

37. The arenavirus particle of any one of claims 30-36, wherein the arenavirus particle is derived from a Lassa virus.

38. The arenavirus particle of any one of claims 30-36, wherein the arenavirus particle is derived from a Lymphocytic choriomeningitis virus (LCMV).

39. The arenavirus particle of claim 38, wherein the LCMV is a MP strain, a WE strain, an Armstrong strain, or an Armstrong Clone 13 strain.

40. The arenavirus particle of any one of claims 30-36, wherein the arenavirus particle is derived from a Pichinde virus (PICV).

41. The arenavirus particle of any one of claims 30-36, wherein the arenavirus particle is derived from a Junin virus vaccine Candid #1, or a Junin virus vaccine XJ Clone 3 strain.

42. The arenavirus particle of any one of claims 30-36, wherein the arenavirus particle is derived from an Oliveros virus.

43. The arenavirus particle of any one of claims 30-36, wherein the arenavirus particle is derived from a Tamiami virus.

44. The arenavirus particle of any one of claims 30-43, wherein the genome of the arenavirus particle encodes the heterologous non-arenaviral polypeptide and/or the second heterologous non-arenaviral polypeptide; wherein the growth or infectivity of the arenavirus particle is not inferior to a second arenavirus particle; wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

45. The arenavirus particle of any one of claims 30-44, wherein the arenavirus particle is infectious and replication competent.

46. The arenavirus particle of any one of claims 30-45, wherein the arenavirus particle is attenuated as compared to its parental virus.

47. The arenavirus particle of any one of claims 30-44 wherein the arenavirus particle is infectious but unable to produce further infectious progeny in non-complementing cells.

48. The arenavirus particle of any one of claims 30-46, wherein the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

49. The arenavirus particle of any one of claims 30-48, wherein the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to the subject as compared to after another arenavirus particle expressing the same heterologous non-arenaviral polypeptide under control of an arenavirus 5′ UTR is administered to the subject or to a comparable subject.

50. The nucleotide sequence of any one of claims 1-10 and 13-28, wherein the nucleotide sequence is a DNA sequence; and wherein the DNA sequence can be transcribed into an arenavirus genomic or antigenomic segment.

51. A method of producing an arenavirus genomic or antigenomic RNA segment, wherein the method comprises transcribing the DNA sequence of claim 50.

52. A method of generating an arenavirus particle, wherein the method comprises:
a) transfecting into a host cell one or more DNA sequences of claim 50 or one or more RNA sequences each transcribed in vitro from the DNA sequence of claim 50;
b) transfecting into the host cell nucleotide sequences encoding arenavirus trans-acting factors;
c) maintaining the host cell under conditions suitable for virus formation; and
d) harvesting the arenavirus particle.

53. The method of claim 52, wherein the one or more DNA sequences are transcribed using a bidirectional promoter.

54. The method of any one of claims 52-53, wherein the one or more DNA sequences are transcribed under the control of a promoter selected from the group consisting of:
a) a RNA polymerase I promoter;
b) a RNA polymerase II promoter; and
c) a T7 promoter.

55. A DNA expression vector comprising the nucleotide sequence of claim 50.

56. A method of rescuing an arenavirus particle using the nucleotide sequence of any one of the claims 1-28 and 50.

57. A host cell comprising the nucleotide sequence of any one of the claims 1-28 and 50, the translation product of claim 29, the arenavirus particle of claims 30-49, or the DNA expression vector of claim 55.

58. A vaccine comprising the nucleotide sequence of any one of the claims 1-28 and 50, the translation product of claim 29, the arenavirus particle of claims 30-49, or the DNA expression vector of claim 55, and a pharmaceutically acceptable carrier.

59. A pharmaceutical composition comprising the nucleotide sequence of any one of the claims 1-28 and 50, the translation product of claim 29, the arenavirus particle of claims 30-49, or the DNA expression vector of claim 55, and a pharmaceutically acceptable carrier.

60. An arenavirus particle engineered such that an arenaviral ORF is separated over two or more mRNA transcripts.

61. The arenavirus particle of claim 60, wherein at least one of the mRNA transcripts comprises an internal ribosome entry site (IRES).

62. The arenavirus particle of any one of claims 60-61, wherein the mRNA transcripts can be transcribed from the arenavirus genomic or antigenomic segment.

63. The arenavirus particle of claim 62, wherein the arenavirus genomic or antigenomic segment is an S segment.

64. The arenavirus particle of claim 62, wherein the arenavirus genomic or antigenomic segment is an L segment.

65. The arenavirus particle of any one of claims 60-64, wherein the two or more mRNA transcripts are under control of an arenavirus 3′ UTR or an arenavirus 5′ UTR.

66. The arenavirus particle of any one of claims 60-65, wherein the arenaviral ORF encodes a polypeptide comprising an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

67. The arenavirus particle of any one of claims 60-66, wherein the arenaviral ORF encodes a polypeptide comprising an amino acid sequence that is identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

68. The arenavirus particle of any one of claims 60-66, wherein the arenaviral ORF encodes arenavirus GP signal peptide, arenavirus GP1 and GP2 and wherein the arenavirus GP signal peptide or a functional fragment thereof is expressed from a first mRNA transcript and arenavirus GP1 and GP2 are expressed from a second mRNA transcript.

69. The arenavirus particle of claim 68, wherein the first mRNA transcript is under control of an arenavirus 3′ UTR.

70. The arenavirus particle of any one of claims 68-69, wherein the second mRNA transcript further encodes a heterologous non-arenaviral signal peptide.

71. The arenavirus particle of claim 70, wherein the heterologous non-arenaviral signal peptide is the signal peptide of the vesicular stomatitis virus serotype Indiana glycoprotein.

72. The arenavirus particle of any one of claims 68-71, wherein the first mRNA transcript further comprises a nucleotide sequence encoding a heterologous non-arenaviral polypeptide or arenavirus GP, NP, Z and L.

73. The arenavirus particle of claim 72, wherein the heterologous non-arenaviral polypeptide is an antigen derived from an infectious organism, tumor, or allergen.

74. The arenavirus particle of claim 73, wherein the antigen is selected from the group consisting of
(a) viral antigens, wherein the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, wherein the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and wherein the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-DI, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and RORL.

75. The arenavirus particle of any one of claims 72-74, wherein the expression level of the heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR.

76. The arenavirus particle of any one of claims 72-74, wherein the genome of the arenavirus particle encodes the heterologous non-arenaviral polypeptide; wherein the growth or infectivity of the arenavirus particle is not inferior to a second arenavirus particle; wherein the genome of the second arenavirus particle encodes the same heterologous non-arenaviral polypeptide; and wherein all arenaviral GP, NP, Z and L in the second arenavirus particle are each expressed as one ORF.

77. The arenavirus particle of any one of claims 60-76, wherein the genome of the arenavirus particle consists of an S segment and an L segment.

78. The arenavirus particle of any one of claims 60-76, wherein the arenavirus particle is tri-segmented.

79. The arenavirus particle of claim 78, wherein the tri-segmented arenavirus particle comprises two S segments and an L segment.

80. The arenavirus particle of claim 79, wherein the arenavirus particle comprises a genome organization as outlined in FIG. 4C.

81. The arenavirus particle of claim 79, wherein the arenavirus particle comprises a genome organization as outlined in FIG. 4E.

82. The arenavirus particle of claim 79, wherein the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
wherein the two heterologous non-arenaviral polypeptides are the same or different from each other.

83. The arenavirus particle of claim 79, wherein the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
wherein the two heterologous non-arenaviral polypeptides are the same or different from each other.

84. The arenavirus particle of claim 78, wherein the tri-segmented arenavirus particle comprises an S segment and two L segments.

85. The arenavirus particle of any one of claims 60-84, wherein the arenavirus particle is derived from a Lassa virus.

86. The arenavirus particle of any one of claims 60-84, wherein the arenavirus particle is derived from a Lymphocytic choriomeningitis virus (LCMV).

87. The arenavirus particle of claim 86, wherein the LCMV is a MP strain, a WE strain, an Armstrong strain, or an Armstrong Clone 13 strain.

88. The arenavirus particle of any one of claims 60-84, wherein the arenavirus particle is derived from a Pichinde virus (PICV).

89. The arenavirus particle of any one of claims 60-84, wherein the arenavirus particle is derived from a Junin virus vaccine Candid #1, or a Junin virus vaccine XJ Clone 3 strain.

90. The arenavirus particle of any one of claims 60-84, wherein the arenavirus particle is derived from an Oliveros virus.

91. The arenavirus particle of any one of claims 60-84, wherein the arenavirus particle is derived from a Tamiami virus.

92. The arenavirus particle of any one of claims 60-91, wherein the arenavirus particle is infectious and replication competent.

93. The arenavirus particle of any one of claims 60-92, wherein the arenavirus particle is attenuated as compared to its parental wild-type virus.

94. The arenavirus particle of any one of claims 60-91, wherein the arenavirus particle is infectious but unable to produce further infectious progeny in non-complementing cells.

95. The arenavirus particle of any one of claims 60-93, wherein the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

96. The arenavirus particle of any one of claims 60-95, wherein the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after administration of the arenavirus particle to the subject as compared to after another arenavirus particle expressing the same heterologous non-arenaviral polypeptide under control of an arenavirus 5′ UTR is administered to the subject or to a comparable subject.

97. A translation product of any one of the mRNA transcripts of the genome of the arenavirus particle of any one of claims 60-96.

98. A cDNA of the mRNA transcript of the genome of the arenavirus particle of any one of claims 60-96, wherein the cDNA can be transcribed into an arenavirus genomic or antigenomic segment.

99. A method of producing an arenavirus genomic or antigenomic segment, wherein the method comprises transcribing the cDNA of claim 98.

100. A method of generating an arenavirus particle, wherein the method comprises:
a) transfecting into a host cell one or more cDNA sequences of claim 98 or one or more RNA sequences each transcribed in vitro from the cDNA sequence of claim 98;
b) transfecting into the host cell nucleotide sequences encoding arenavirus trans-acting factors;
c) maintaining the host cell under conditions suitable for virus formation; and
d) harvesting the arenavirus particle.

101. The method of claim 100, wherein the one or more cDNA sequences are transcribed using a bidirectional promoter.

102. The method of any one of claims 100-101, wherein the one or more cDNA sequences are transcribed under the control of a promoter selected from the group consisting of:
a) a RNA polymerase I promoter;
b) a RNA polymerase II promoter; and
c) a T7 promoter.

103. A DNA expression vector comprising the DNA sequence encoding the mRNA transcript of the genome of the arenavirus particle of any one of claims 60-96.

104. A method of rescuing an arenavirus particle using the mRNA transcript of the genome of the arenavirus particle of any one of the claims 60-96 or the cDNA sequence thereof.

105. A host cell comprising the arenavirus particle of any one of claims 60-96, the translation product of claim 97, the cDNA of claim 98, or the DNA expression vector of claim 103.

106. A vaccine comprising the arenavirus particle of any one of claims 60-96, the translation product of claim 97, the cDNA of claim 98, or the DNA expression vector of claim 103, and a pharmaceutically acceptable carrier.

107. A pharmaceutical composition comprising the arenavirus particle of any one of claims 60-96, the translation product of claim 97, the cDNA of claim 98, or the DNA expression vector of claim 103, and a pharmaceutically acceptable carrier.

108. An arenavirus genomic or antigenomic segment engineered such that the viral transcription thereof results in a first mRNA transcript and a second mRNA transcript, wherein the first mRNA transcript comprises a nucleotide sequence encoding a functional fragment of a first polypeptide, and the first mRNA transcript does not encode the full-length first polypeptide;
wherein the second mRNA transcript comprises a nucleotide sequence encoding:
a) a second polypeptide; or
b) a functional fragment of the first polypeptide, and the second mRNA transcript does not encode the full-length first polypeptide; or
c) a functional fragment of a second polypeptide, and the second mRNA transcript does not encode the full-length second polypeptide; or
d) a heterologous non-arenaviral polypeptide; and
wherein the first and second polypeptides are different from each other and selected from the group consisting of arenavirus GP, NP, Z and L.

109. The arenavirus genomic or antigenomic segment of claim 108, wherein the first mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

110. The arenavirus genomic or antigenomic segment of claim 108, wherein the second mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

111. The arenavirus genomic or antigenomic segment of any one of claims 108-110, wherein the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Aporé virus, Ryukyu virus, Solwezi virus, souris virus, Wenzhou virus, Big Brushy Tank virus, Catarina virus, Skinner Tank virus, Tonto Creek virus, Xapuri virus, or Junin virus.

112. An arenavirus genomic or antigenomic segment engineered such that the viral transcription thereof results in an mRNA transcript encoding:
a) a functional fragment of a first polypeptide, and
b) a heterologous non-arenaviral polypeptide or a second polypeptide;
wherein the mRNA transcript does not encode the full-length first polypeptide; and wherein the first and second polypeptides are different from each other and selected from the group consisting of arenavirus GP, NP, Z and L.

113. The arenavirus genomic or antigenomic segment of claim 112, wherein the mRNA transcript is a first mRNA transcript; wherein the viral transcription of the arenavirus genomic or antigenomic segment further results in a second mRNA transcript; wherein the second mRNA transcript comprises a nucleotide sequence encoding a third polypeptide; a functional fragment of the first polypeptide; a functional fragment of a third polypeptide; or a second heterologous non-arenaviral polypeptide; wherein the third polypeptide is different from the first and the second polypeptide and selected from the group consisting of arenavirus GP, NP, Z and L, wherein the second mRNA transcript does not encode the full-length first polypeptide, wherein the second mRNA transcript does not encode the full-length third polypeptide and wherein the heterologous non-arenaviral polypeptide and the second heterologous non-arenaviral polypeptide are the same or different from each other.

114. The arenavirus genomic or antigenomic segment of any one of claims 108-111 and 113, wherein the second mRNA transcript comprises a nucleotide sequence encoding a functional fragment of a first polypeptide, and wherein the functional fragment encoded by the first mRNA transcript is different from the functional fragment encoded by the second mRNA transcript.

115. The arenavirus genomic or antigenomic segment of claim 112, wherein the viral transcription thereof does not further result in a second mRNA transcript.

116. The arenavirus genomic or antigenomic segment of any one of claims 108-115, wherein the mRNA transcript comprises an internal ribosome entry site (IRES).

117. The arenavirus genomic or antigenomic segment of any one of claims 108-116, wherein the arenavirus genomic or antigenomic segment is an S segment.

118. The arenavirus genomic or antigenomic segment of any one of claims 108-116, wherein the arenavirus genomic or antigenomic segment is an L segment.

119. The arenavirus genomic or antigenomic segment of any one of claims 108-118, wherein the functional fragment is under control of an arenavirus 3′ UTR.

120. The arenavirus genomic or antigenomic segment of any one of claims 108-118, wherein the functional fragment is under control of an arenavirus 5′ UTR.

121. The arenavirus genomic or antigenomic segment of any one of claims 108-120, wherein the first, second and third polypeptide each comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

122. The arenavirus genomic or antigenomic segment of any one of the claims 108-121, wherein the first polypeptide comprises an amino acid sequence identical to SEQ ID NO:1, SEQ ID NO:2, SEQ ID NO:3, or SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

123. The arenavirus genomic or antigenomic segment of any one of the claims 108-122, wherein the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, or SEQ ID NO:137.

124. The arenavirus genomic or antigenomic segment of any one of the claims 108-123, wherein the functional fragment is an arenavirus GP signal peptide or a functional fragment thereof.

125. The arenavirus genomic or antigenomic segment of any one of claims 108-124, wherein the heterologous non-arenaviral polypeptide, the second heterologous non-arenaviral polypeptide, or both heterologous non-arenaviral polypeptides are each an antigen derived from an infectious organism, tumor, or allergen.

126. The arenavirus genomic or antigenomic segment of claim 125, wherein the antigen is selected from the group consisting of
(a) viral antigens, wherein the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, wherein the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and wherein the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSX1 or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29\BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68\KP1, CO-029, HTgp-175, M344, MG7-Ag, MOV18, NB\70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avβ3 (CD61), galactin, or Ral-B, CD123, CLL-1, CD38, CS-1, CD138, and ROR1.

127. The arenavirus genomic or antigenomic segment of any one of claims 108-111 and 113-126, wherein the first mRNA transcript comprises a nucleotide sequence encoding arenavirus GP signal peptide, either alone or fused to a heterologous non-arenaviral polypeptide.

128. The arenavirus genomic or antigenomic segment of claim 127, wherein the second mRNA transcript comprises a nucleotide sequence encoding a heterologous non-arenaviral signal peptide and the arenavirus GP1 and GP2.

129. The arenavirus genomic or antigenomic segment of any one of claims 127-128, wherein the first mRNA transcript is under control of an arenavirus 3′ UTR and the second mRNA transcript is under control of an arenavirus 5′ UTR.

130. The arenavirus genomic or antigenomic segment of claim 127, wherein the second mRNA transcript comprises a nucleotide sequence encoding a heterologous non-arenaviral polypeptide; wherein the heterologous non-arenaviral polypeptide encoded by the first mRNA transcript and the heterologous non-arenaviral polypeptide encoded by the second mRNA transcript are the same or different from each other.

131. The arenavirus genomic or antigenomic segment of any one of claims 127 and 130, wherein the first mRNA transcript is under control of an arenavirus 3′ UTR and the second mRNA transcript is under control of an arenavirus 5′ UTR.

132. The arenavirus genomic or antigenomic segment of any one of claims 108-111 and 113-126, wherein the first mRNA transcript comprises a nucleotide sequence encoding a heterologous non-arenaviral signal peptide and the arenavirus GP1 and GP2.

133. The arenavirus genomic or antigenomic segment of claim 132, wherein the second mRNA transcript comprises a nucleotide sequence encoding NP.

134. The arenavirus genomic or antigenomic segment of any one of claims 132-133, wherein the first mRNA transcript is under control of an arenavirus 5′ UTR and the second mRNA transcript is under control of an arenavirus 3′ UTR.

135. The arenavirus genomic or antigenomic segment of any one of claims 108-134, wherein the expression level of the heterologous non-arenaviral polypeptide expressed under control of an arenavirus 3′ UTR or the expression level of the second heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or higher than the expression level of the same second heterologous non-arenaviral polypeptide expressed under control of the respective arenavirus 5′ UTR.

136. A translation product of the arenavirus genomic or antigenomic segment of any one of claims 108-135.

137. An arenavirus particle comprising the arenavirus genomic or antigenomic segment of any one of claims 108-135.

138. The arenavirus particle of claim 137, wherein the genome of the arenavirus particle consists of an S segment and an L segment.

139. The arenavirus particle of claim 137, wherein the arenavirus particle is tri-segmented.

140. The arenavirus particle of claim 139, wherein the tri-segmented arenavirus particle comprises two S segments and an L segment.

141. The arenavirus particle of claim 140, wherein the genome of the arenavirus particle consists of
a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
wherein the two heterologous non-arenaviral polypeptides are the same or different from each other.

142. The arenavirus particle of claim 140, wherein the genome of the arenavirus particle consists of
a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
wherein the two heterologous non-arenaviral polypeptides are the same or different from each other.

143. The arenavirus particle of claim 139, wherein the tri-segmented arenavirus particle comprises an S segment and two L segments.

144. The arenavirus particle of any one of claims 137-143, wherein the arenavirus particle is derived from a Lassa virus.

145. The arenavirus particle of any one of claims 137-143, wherein the arenavirus particle is derived from a Lymphocytic choriomeningitis virus (LCMV).

146. The arenavirus particle of claim 145, wherein the LCMV is a MP strain, a WE strain, an Armstrong strain, or an Armstrong Clone 13 strain.

147. The arenavirus particle of any one of claims 137-143, wherein the arenavirus particle is derived from a Pichinde virus (PICV).

148. The arenavirus particle of any one of claims 137-143, wherein the arenavirus particle is derived from a Junin virus vaccine Candid #1, or a Junin virus vaccine XJ Clone 3 strain.

149. The arenavirus particle of any one of claims 137-143, wherein the arenavirus particle is derived from an Oliveros virus.

150. The arenavirus particle of any one of claims 137-143, wherein the arenavirus particle is derived from a Tamiami virus.

151. The arenavirus particle of any one of claims 137-150, wherein the genome of the arenavirus particle encodes the heterologous non-arenaviral polypeptide and/or the second heterologous non-arenaviral polypeptide; wherein the growth or infectivity of the arenavirus particle is not inferior to a second arenavirus particle; wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF.

152. The arenavirus particle of any one of claims 137-151, wherein the arenavirus particle is infectious and replication competent.

153. The arenavirus particle of any one of claims 137-152, wherein the arenavirus particle is attenuated as compared to its parental wild-type virus.

154. The arenavirus particle of any one of claims 137-151, wherein the arenavirus particle is infectious but unable to produce further infectious progeny in non-complementing cells.

155. The arenavirus particle of any one of claims 137-153, wherein the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

156. The arenavirus particle of any one of claims 137-155, wherein the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to a subject than after another arenavirus particle expressing the same heterologous non-arenaviral polypeptide under control of an arenavirus 5′ UTR is administered to a subject or to a comparable subject.

157. A cDNA of the arenavirus genomic or antigenomic segment of any one of claims 108-135.

158. A method of producing an arenavirus genomic or antigenomic segment, wherein the method comprises transcribing the cDNA of claim 157.

159. A method of generating an arenavirus particle, wherein the method comprises:
a) transfecting into a host cell one or more cDNA sequences of claim 157 or one or more RNA sequences each transcribed in vitro from the cDNA sequence of claim 157;
b) transfecting into the host cell nucleotide sequences encoding arenavirus trans-acting factors;
c) maintaining the host cell under conditions suitable for virus formation; and
d) harvesting the arenavirus particle.

160. The method of claim 159, wherein the one or more cDNA sequences are transcribed using a bidirectional promoter.

161. The method of any one of claims 159-160, wherein the one or more cDNA sequences are transcribed under the control of a promoter selected from the group consisting of:
a) a RNA polymerase I promoter;
b) a RNA polymerase II promoter; and
c) a T7 promoter.

162. A DNA expression vector comprising a DNA sequence encoding the arenavirus genomic or antigenomic segment of any one of claims 108-135.

163. A method of rescuing an arenavirus particle using the arenavirus genomic or antigenomic segment of any one of claims 108-135 or a DNA sequence encoding the arenavirus genomic or antigenomic segment.

164. A host cell comprising the arenavirus genomic or antigenomic segment of any one of claims 108-135, the translation product of claim 136, the arenavirus particle of any one of claims 137-156, or the DNA expression vector of claim 162.

165. A vaccine comprising the arenavirus genomic or antigenomic segment of any one of claims 108-135, the translation product of claim 136, the arenavirus particle of any one of claims 137-156, or the DNA expression vector of claim 162, and a pharmaceutically acceptable carrier.

166. A pharmaceutical composition comprising the arenavirus genomic or antigenomic segment of any one of claims 108-135, the translation product of claim 136, the arenavirus particle of any one of claims 137-156, or the DNA expression vector of claim 162, and a pharmaceutically acceptable carrier.

167. The nucleotide sequence of any one of claims 1-27, wherein the expression of the heterologous non-arenaviral polypeptide is under control of an arenavirus 3′ UTR and the expression of the second heterologous non-arenaviral polypeptide is under control of an arenavirus 5′ UTR, wherein the proportion of cells expressing both the heterologous non-arenaviral polypeptide and the second heterologous non-arenaviral polypeptide, after an arenavirus particle containing a genome comprising the nucleotide sequence infects a population of cells, is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells expressing both the same heterologous non-arenaviral polypeptide and the same second heterologous non-arenaviral polypeptide expressed under control of arenavirus 5′ UTRs, after an arenavirus particle containing a genome comprising a nucleotide sequence encoding the same heterologous non-arenaviral polypeptide and the same second heterologous non-arenaviral polypeptide expressed under control of arenavirus 5′ UTRs infects a comparable population of cells.

168. The arenavirus particle of any one of claims 60-74, wherein the arenavirus particle expresses two heterologous non-arenaviral polypeptides, wherein the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment, wherein the proportion of cells expressing both the two heterologous non-arenaviral polypeptides after the arenavirus particle infects a population of cells is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells expressing both the same two heterologous non-arenaviral polypeptides after an arenavirus particle expressing the same two heterologous non-arenaviral polypeptides under control of arenavirus 5′ UTRs infects a comparable population of cells, and wherein the expression of a first of the same two heterologous non-arenaviral polypeptides is under control of an arenavirus 5′ UTR in a first S segment and the expression of a second of the same two heterologous non-arenaviral polypeptides is under control of an arenavirus 5′ UTR in a second S segment.

169. The arenavirus particle of any one of claims 30-36, 60-84, 137-143, or 168, wherein the arenavirus particle is derived from a Mobala virus.

170. The arenavirus particle of any one of claims 30-36, 60-84, 137-143, or 168, wherein the arenavirus particle is derived from a Mopeia virus.

171. The arenavirus particle of any one of claims 30-36, 60-84, 137-143, or 168, wherein the arenavirus particle is derived from a Ippy virus.

172. The arenavirus particle of any one of claims 30-36, 60-84, 137-143, or 168, wherein the arenavirus particle is derived from a Amapari virus.

173. The arenavirus particle of any one of claims 30-36, 60-84, 137-143, or 168, wherein the arenavirus particle is derived from a Flexal virus.

174. The arenavirus particle of any one of claims 30-36, 60-84, 137-143, or 168, wherein the arenavirus particle is derived from a Guanarito virus.

175. The arenavirus particle of any one of claims 30-36, 60-84, 137-143, or 168, wherein the arenavirus particle is derived from a Latino virus.

176. The arenavirus particle of any one of claims 30-36, 60-84, 137-143, or 168, wherein the arenavirus particle is derived from a Machupo virus.

177. The arenavirus particle of any one of claims 30-36, 60-84, 137-143, or 168, wherein the arenavirus particle is derived from a Parana virus.

178. The arenavirus particle of any one of claims 30-36, 60-84, 137-143, or 168, wherein the arenavirus particle is derived from a Pirital virus.

179. The arenavirus particle of any one of claims 30-36, 60-84, 137-143, or 168, wherein the arenavirus particle is derived from a Sabia virus.

180. The arenavirus particle of any one of claims 30-36, 60-84, 137-143, or 168, wherein the arenavirus particle is derived from a Tacaribe virus.

181. The arenavirus particle of any one of claims 30-36, 60-84, 137-143, or 168, wherein the arenavirus particle is derived from a Bear Canyon virus.

182. The arenavirus particle of any one of claims 30-36, 60-84, 137-143, or 168, wherein the arenavirus particle is derived from a Whitewater Arroyo virus.

183. The arenavirus particle of any one of claims 30-36, 60-84, 137-143, or 168, wherein the arenavirus particle is derived from an Allpahuayo virus.

184. The arenavirus particle of any one of claims 30-36, 60-84, 137-143, or 168, wherein the arenavirus particle is derived from an Alxa virus.

185. The arenavirus particle of any one of claims 30-36, 60-84, 137-143, or 168, wherein the arenavirus particle is derived from a Chapare virus.

186. The arenavirus particle of any one of claims 30-36, 60-84, 137-143, or 168, wherein the arenavirus particle is derived from a Lijiang virus.

187. The arenavirus particle of any one of claims 30-36, 60-84, 137-143, or 168, wherein the arenavirus particle is derived from a Cupixi virus.

188. The arenavirus particle of any one of claims 30-36, 60-84, 137-143, or 168, wherein the arenavirus particle is derived from a Gairo virus.

189. The arenavirus particle of any one of claims 30-36, 60-84, 137-143, or 168, wherein the arenavirus particle is derived from a Loei River virus.

190. The arenavirus particle of any one of claims 30-36, 60-84, 137-143, or 168, wherein the arenavirus particle is derived from a Lujo virus.

191. The arenavirus particle of any one of claims 30-36, 60-84, 137-143, or 168, wherein the arenavirus particle is derived from a Luna virus.

192. The arenavirus particle of any one of claims 30-36, 60-84, 137-143, or 168, wherein the arenavirus particle is derived from a Luli virus.

193. The arenavirus particle of any one of claims 30-36, 60-84, 137-143, or 168, wherein the arenavirus particle is derived from a Lunk virus.

194. The arenavirus particle of any one of claims 30-36, 60-84, 137-143, or 168, wherein the arenavirus particle is derived from a Mariental virus.

195. The arenavirus particle of any one of claims 30-36, 60-84, 137-143, or 168, wherein the arenavirus particle is derived from a Merino Walk virus.

196. The arenavirus particle of any one of claims 30-36, 60-84, 137-143, or 168, wherein the arenavirus particle is derived from a Morogoro virus.

197. The arenavirus particle of any one of claims 30-36, 60-84, 137-143, or 168, wherein the arenavirus particle is derived from a Okahandja virus.

198. The arenavirus particle of any one of claims 30-36, 60-84, 137-143, or 168, wherein the arenavirus particle is derived from an Aporé virus.

199. The arenavirus particle of any one of claims 30-36, 60-84, 137-143, or 168, wherein the arenavirus particle is derived from a Ryukyu virus.

200. The arenavirus particle of any one of claims 30-36, 60-84, 137-143, or 168, wherein the arenavirus particle is derived from a Solwezi virus.

201. The arenavirus particle of any one of claims 30-36, 60-84, 137-143, or 168, wherein the arenavirus particle is derived from a souris virus.

202. The arenavirus particle of any one of claims 30-36, 60-84, 137-143, or 168, wherein the arenavirus particle is derived from a Wenzhou virus.

203. The arenavirus particle of any one of claims 30-36, 60-84, 137-143, or 168, wherein the arenavirus particle is derived from a Big Brushy Tank virus.

204. The arenavirus particle of any one of claims 30-36, 60-84, 137-143, or 168, wherein the arenavirus particle is derived from a Catarina virus.

205. The arenavirus particle of any one of claims 30-36, 60-84, 137-143, or 168, wherein the arenavirus particle is derived from a Skinner Tank virus.

206. The arenavirus particle of any one of claims 30-36, 60-84, 137-143, or 168, wherein the arenavirus particle is derived from a Tonto Creek virus.

207. The arenavirus particle of any one of claims 30-36, 60-84, 137-143, or 168, wherein the arenavirus particle is derived from a Xapuri virus.
